(19) United States
(12) Patent Application Publication
(10) Pub. No.: US 2010/0143915 A1
(43) Pub. Date: Jun. 10, 2010

(54) Cells Modified or Altered for a Rice-diverged Glycosyltransferase

(75) Inventors: Pamela C. Ronald, Davis, CA (US); Peijian Cao, Davis, CA (US); Laura E. Bartley, Davis, CA (US); Ki-Hong Jung, Yongin (KR)

Correspondence Address: LAWRENCE BERKELEY NATIONAL LABORATORY, Technology Transfer & Intellectual Propery Managem, One Cyolotron Road MS 56A-120, BERKELEY, CA, 94720, US

(73) Assignee: THE REGENTS OF THE UNIVERSITY OF CALIFORNIA, Oakland, CA (US)

(21) Appl. No.: 12/552,155

(22) Filed: Sep. 1, 2009

Related U.S. Application Data
(60) Provisional application No. 61/095,591, filed on Sep. 9, 2008.

(51) Int. Cl. C12Q 1/68; C12N 5/10

(52) U.S. Cl. 435/6; 435/419

(57) ABSTRACT

The present invention provides for a cell comprising a modified or altered enzymatic activity of a rice-diverged glycosyltransferase (GT).

### CROSS-REFERENCE TO RELATED APPLICATIONS

This application claims priority to U.S. Provisional Patent Application Ser. No. 61/095,591, filed on Sep. 9, 2008, which is hereby incorporated by reference.

### STATEMENT OF GOVERNMENTAL SUPPORT

The invention described and claimed herein was made in part utilizing funds supplied by the U.S. Department of Energy grant DE-P502-06ER64304 to the Joint BioEnergy Institute. The government has certain rights in this invention.

### FIELD OF THE INVENTION

This invention relates generally to glycosyltransferases.

### BACKGROUND OF THE INVENTION

Biofuels have the potential to serve as an alternative energy source, relieving dependence on petroleum and reducing production of climate-changing greenhouse gasses. Sustainable biofuel production will rely on conversion of the large, untapped resource (several billion tons per year) of plant lignocellulosic biomass to sugars for use in fermentation of liquid fuels. Due to its abundance and high rate of production, lignocellulosic biomass has advantages as a biofuel feedstock compared with currently used starch and cane sugar; however, these advantages are overcome by the current expense of extracting sugars from lignocelluose. The source of lignocellulosic biomass is the plant cell wall, a complex and dynamic extracellular matrix that regulates cell growth, provides plants with mechanical support and protects against pathogens (Carpita, 1996). In addition to elucidating fundamental plant biology, understanding the function of genes involved in biosynthesis of plant cell wall constituents may provide avenues that facilitate use of lignocellulose for biofuel production.

Primary cell walls are composed of cellulose microfibrils embedded in a semi-structured matrix of non-cellulosic polysaccharides (Carpita et al., 2001; Somerville et al., 2004). As plant cells age and cease to grow, secondary cell walls are often deposited, in which the cellulose matrix becomes denser and typically crosslinked by a phenyl propanoid-derived lignin meshwork. There are two major classes of cell walls in plants, type I and type II, which differ in architecture, chemical composition and structures, and their associated biosynthetic processes (Carpita, 1996). Type I walls are found in dicotyledonous plants. In Type I primary walls, cellulose microfibrils are interwoven with xyloglucans and embedded in a matrix of pectin polysaccharides and glycoproteins. As Type I primary walls transition to secondary walls, a xylan polymer, glucuronoxylan accumulates in addition to mannans (Pauly and Keegstra, 2008). Type II walls are characteristic of Commelinoid monocots, including grasses such as rice and switchgrass. In such walls, glucuronoarabinoxylans and β1,3:1,4 mixed linkage glucan form the meshwork surrounding cellulose microfibrils. Though present, Type II walls possess a lower abundance of pectin polysaccharides, xyloglucan and structural proteins relative to Type I walls.

Glycosyltransferases (GTs; EC 2.4.x.y), which add sugar molecules onto acceptor molecules, are responsible for the synthesis of the branched and linear polysaccharides of both type I and II cell walls, among many other functions in biology, including signaling and metabolism (Coutinho et al., 2003). GTs have been hierarchically classified based on the following criteria: three-dimensional structure, catalytic reaction mechanism, and the their donor and acceptor substrates (Coutinho et al., 2003). At the tertiary structure level, GTs adopt one of the following two major folds: the GT-A (SpsA and SpsA-like) fold or the GT-B (B-GT and B-GT-like) fold (Bourne and Henrissat, 2001; Hu and Walker, 2002; Wimmerova et al., 2003). Recently a new GT fold, GT-C, was identified in the Pyrococcus furiosus Oligosaccharyltransferase (OST) (Igura et al., 2008). The fold class of a large number of GTs has not yet been determined and these are classified as “GT-U” for unknown fold. At the catalytic reaction level, glycosylation proceeds via two reaction mechanisms, inversion or retention of stereochemistry at the Cl position of the donor sugar. Beyond these general criteria, GTs have been traditionally grouped into families based on their activated sugar substrate (e.g., galactosyltransferases, sialyltransferases, etc.) and in many cases the acceptor group (e.g., protein, lipid, glycogen, etc.). However, sequence data has far outpaced our ability to identify biochemical activity of enzymes. This led the creation of the Carbohydrate-Active enZymes (CAZy; http://www.cazy.org/) database to build on the biochemical data by developing a hierarchical family classification scheme for grouping GTs at the primary sequence level (Campbell et al., 1997; Coutinho et al., 2003). As of February 2008, CAZy contained 33,359 GTs from organisms across all the kingdoms of life classified into 90 different GT families primarily based on amino acid sequence similarity.

While much remains to be learned, the last decade has seen a great expansion in our understanding of the GTs that synthesize the major constituents of Type I primary walls. Use of diverse plant species and the reference dicot, Arabidopsis (Arabidopsis thaliana), has led to the identification of many genes involved in synthesis of xyloglucan, mannan, and pectins (reviewed in (Farrokhi et al., 2006). In contrast, our depth of knowledge regarding synthesis of type II wall enriched polysaccharide components has lagged behind. The Cellulose synthase-like F (CslF) gene family has been shown to have a role in synthesis of mixed linkage glucan (Burton et al., 2006), but the synthesis of glucuronoarabinoxylan in grasses remains obscure. Progress may be possible based on emerging studies of glucuronoxylan in Arabidopsis secondary cell walls (Lee et al., 2007a; Lee et al., 2007b; Pena et al., 2007; York and O′Neill, 2008). However, the surprisingly complex view that those studies provide remains to be tested for grass primary cell walls.

There is now an opportunity to apply the genomic resources that have accumulated for grasses toward understanding the synthesis of Type II cell walls. Rice serves as a reference monocot species because of its small, sequenced genome (−389 Mb) and the availability of genetic and molecular resources, including indexed insertion mutants (IRGSP, 2005; Jung et al., 2008). Rice itself is also a potentially attractive biofuel feedstock source because it comprises a large portion (ca. 50%) of the world's agricultural residue. Due to a high level of genomic colinearity among grass species (Devos and Gale, 2000), information learned regarding rice cell walls is likely to apply to the cell walls of the other major cereal crops, maize (Zea mays) and wheat (Triticum aestivum), and potential dedicated energy crops, such as switchgrass (Panicum virgatum) and Miscanthus. Conversely, extensive cell wall biochemical and physiological studies on maize, wheat, and barley (Hordeum vulgare), are also likely to apply to rice.

One of the challenges to discovery of cell wall gene function in Arabidopsis has been genetic redundancy, such that single gene mutants have no measurable phenotype. For example, Richardson and Somerville reported that a number of single Cs1 gene mutants provide no phenotype (Richmond and Somerville, 2001). This challenge is likely to be exacerbated in grasses, which possess a larger gene complement compared with Arabidopsis. Estimates for the percent of the rice genome that consists of segmental duplication vary from 27 to 66%, depending on the method of detection used; however, consensus leans towards a higher value of ±50% (Ouyang et al., 2007; Yu et al., 2005). Thus, the rice genome encodes a large number of genes with redundant functions, creating a considerable challenge to the functional analysis of individual genes (Jung et al., 2008a; Jung et al., 2008b). This is also the case for GTs, for which we anticipate that the large number of members in some families, especially those associated with cell wall synthesis, will create difficulties in the functional analysis. Incorporating diverse systems biological datasets, including bioinformatic, genomic, gene expression, and proteomic data, can help inform rationale strategies for gene function discovery, even in large gene families. However, these approaches are hampered by current database formats that display only a single gene or field at a time, preventing simultaneous comparisons of multiple data sets and multi-gene families (Jung et al., 2008a, Jung et al., 2008b). Scattering of genomic data across multiple databases, exacerbated by different gene nomenclatures and data formats, creates additional challenges to integration. The field of phylogenomics, which merges phylogenetics and genomics and puts genomic data in a phylogenetic context, helps us to resolve these limitations. A successful application of phylogenomics for a family of genes for which redundancy poses enormous challenges is the Rice Kinase Database (http://rkd.ucdavis.edu/), which provides a template for the design of new phylogenomic databases (Dardick et al., 2007).

Toward identifying the functions of GTs in building the walls of diverse cell types throughout development, genomic and transcriptomics analyses of glycosyltransferase genes have been conducted for two dicot species, Arabidopsis and poplar (Populus trichocarpa) (Geisler-Lee et al., 2006; Henrissat et al., 2001). The Carbohydrate Active EnZymes (CAZy) database contains only GT family classification and sequence information for the rice enzymes and GTs from the other species it hosts. Yokoyama and Nishitani compared the numbers and phylogenetic relationships of known cell wall-related genes between rice and Arabidopsis soon after the publication of the rice genome (Yokoyama and Nishitani, 2004). However, that analysis focused only on genes, including six GT families, known at that time to be involved in cell wall synthesis and did not include other analyses. Mitchell et al. conducted a much more extensive comparison between grass and dicot GTs and other gene families (Mitchell et al., 2007). This effort examined expressed sequence tag (EST) abundance for orthologous gene groups from Arabidopsis and rice, leading to the identification of several gene families that are more abundantly expressed in grasses than dicots. These genes, including members of the GT47 and GT61 families, are good candidates for involvement in synthesis of glucuronoarabinoxylan and other type II wall-specific or enriched components (Mitchell et al., 2007).

### SUMMARY OF THE INVENTION

The present invention provides for a cell comprising a modified or altered enzymatic activity of a rice-diverged glycosyltransferase (GT). The rice-diverged GT of the present invention have an expression level at equal to higher than the expression level observed for the rice-diverged GT identified in Table 3. The rice-diverged GT have a high expression in vegetative above ground plant tissue. The enzymatic activity of the rice-diverged GT can be modified or altered in that the expression of the GT is modified or altered, or the GT has a modified or altered amino acid sequence as compared to the wild-type GT such that the enzymatic activity of the GT is modified, altered, or a combination thereof. In some embodiments of the invention, the cell is a plant cell.

The present invention also provides for a seed, plant tissue, plant part or a whole plant comprising a cell of the present invention. In some embodiments, the plant part is a leaf, leaf stalk, stem, root, or a combination thereof. In some embodiments, the whole plant includes, but is not limited to, a germinating seed.

The present invention also provides for a method of identifying or determining a rice-diverged GT of a plant or cell, comprising: the steps described herein in Example 1 of this present specification.

The present invention also provides for a method of identifying or determining a rice-diverged GT of a plant or cell, comprising: (a) providing the amino acid sequence of a monocot gene not known to be rice-diverged GT, and (b) identifying the gene as lacking a dicot ortholog. The method can further comprise: determining the expression level of the gene in vegetative above ground plant tissue. The method can further comprise: constructing a plant cell that is reduced or increased in the expression of the gene.

The present invention also provides for a method of constructing a cell of the present invention, comprising: modifying or altering the enzymatic activity of the rice-diverged GT.

The present invention also provides for a method of constructing a seed, plant tissue, plant part or a whole plant comprising a cell of the present invention, comprising: constructing a cell of the present invention.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows a screen shot of the Rice GT Database Tree viewer format. Checking each box and clicking the Submit button will display the selected data next to the phylogenetic tree.

FIG. 2 shows a screen shot of the topmost portion of the Rice GT Database phylogenetic tree. A subset of database content, including the TIGR gene model ID, CAZy family, corresponding RAP2 ID and a hyperlink to NCBI BLAST search are listed in spreadsheet format adjacent to the tree. This format allows for easy and flexible visualization of the data within the context of the tree. Data obtained in the spreadsheet can be searched using the browser search function.

FIG. 3 shows a screen shot of a Rice GT Database summary page Links to summary pages are provided from the TIGR model ID of each GT. Summary pages include all data in the database except the microarray data because of large amount of data. The Digital Northern data and MPSS expression data are represented in histogram format for easy comparison of rice GT expression patterns between different tissues.

FIG. 4 shows the hierarchical classification of rice GT families based on GT fold, reaction mechanism and known enzymatic activities. GT-A, GT-B, and GT-C, are the known GT folds. GT-U indicates that the GT fold is unknown. In each GT family, only one known enzymatic activity is shown on this figure for convenience.

FIG. 5 shows the distribution of rice, Arabidopsis and poplar GT gene models among different GT families. Number of GT gene models in each species is shown on the y-axis. GT families are listed along the x-axis.

FIG. 6 shows the distribution of GT gene models among different MPSS libraries and expression levels. Number of GT gene models in each tissue is shown on the y-axis. Tissues are listed along the x-axis and different expression levels are represented by different colors.

FIG. 7 shows the Affymetrix rice microarray expression profiles of rice (A) GT47 and (B) GT61 family members in different tissues/organs and during different developmental stages. The average log₂ signal values of rice GTs in various tissues/organs and developmental stages (listed at the top of heatmap) are presented with the same gene order in the phylogenetic tree. The color scale (representing log₂ signal values) is shown at the top.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a GT” includes a plurality of such GTs, and so forth.

In this specification and in the claims that follow, reference will be made to a number of terms that shall be defined to have the following meanings:

In describing the present invention, the term “GT” encompasses rice-diverged GT.

The terms “expression vector” or “vector” refer to a compound and/or composition that can be introduced into a cell by any suitable method, including but not limited to transduction, transformation, transfection, infection, electroporation, conjugation, and the like; thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a cell and replicated therein. Such expression vectors include plasmids, particularly those with restriction sites that have been well documented and that contain the operational elements preferred or required for transcription of the nucleic acid sequence. Such plasmids, as well as other expression vectors, are well known to those of ordinary skill in the art.

The term “operably linked” refers to a functional linkage between a nucleic acid expression control sequence (such as a promoter) and a second nucleic acid sequence, wherein the expression control sequence directs transcription of the nucleic acid corresponding to the second sequence.

The term “recombinant” refers to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

The term “sequence similarity” refers to the level of nucleic acid or amino acid sequence identity between two or more aligned sequences, when aligned using a sequence alignment program. For example, 70% sequence similarity means the same thing as 70% sequence identity determined by a defined algorithm. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at (ncbi.nlm.gov/BLAST/). See, also, Altschul, S. F. et al., 1990 and Altschul, S. F. et al., 1997.

The term “transgenic plant” refers to a plant that has incorporated exogenous nucleic acid sequences, i.e., nucleic acid sequences which are not present in the native (“untransformed”) plant or plant cell. Thus a plant having within its cells a heterologous polynucleotide is referred to herein as a “transgenic plant”. The heterologous polynucleotide can be either stably integrated into the genome, or can be extra-chromosomal. Typically, the polynucleotide of the present invention is stably integrated into the genome such that the polynucleotide is passed on to successive generations. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. “Transgenic” is used herein to include any cell, cell line, callus, tissue, plant part or plant, the genotype of which has been altered by the presence of heterologous nucleic acids including those transgenics initially so altered as well as those created by sexual crosses or asexual reproduction of the initial transgenics.

The term “expression” with respect to a protein or peptide, such as a GT, refers to the process by which the protein or peptide is produced based on the nucleic acid sequence of a gene. The process includes both transcription and translation. The term “expression” may also be used with respect to the generation of RNA from a DNA sequence.

The term “plant cell” refers to any cell derived from a plant, including undifferentiated tissue (e.g., callus) as well as plant seeds, pollen, progagules and embryos.

The term “mature plant” refers to a fully differentiated plant.

The terms “native” and “wild-type” relative to a given plant trait or phenotype refers to the form in which that trait or phenotype is found in the same variety of plant in nature.

The term “plant” includes reference to whole plants, plant organs (for example, leaves, stems, roots, etc.), seeds, and plant cells and progeny of same. Plant cell, as used herein includes, without limitation, seeds, suspension cultures, embryos, meristematic regions, callus tissue, leaves roots shoots, gametophytes, sporophytes, pollen, and microspores. The class of plants that can be used in the methods of the present invention is generally as broad as the class of higher plants amenable to transformation techniques, including both monocotyledonous and dicotyledonous plants.

The term “seed” is meant to encompass all seed components, including, for example, the coleoptile and leaves, radicle and coleorhiza, scutulum, starchy endosperm, aleurone layer, pericarp and/or testa, either during seed maturation and seed germination.

The term “promoter” is a promoter capable of initiating (promoting) transcription in cells, wherein if the cell is a plant cell, then the promoter is a plant promoter. Such promoters need not be of plant origin. For example, promoters derived from plant viruses, such as the CaMV 35S promoter, or from Agrobacterium tumefaciens such as the T-DNA promoters, can serve as plant promoters. An example of a plant promoter of plant origin is the maize ubiquitin-1 (ubi-1) promoter. Other suitable plant promoters include those known to persons of ordinary skill in the art. A plant promoter can direct expression of a nucleotide sequence in all or certain tissues of a plant, e.g., a constitutive promoter such as 35S or a broadly expressing promoter such as p326. Alternatively, a plant promoter can direct transcription of a nucleotide sequence in a specific tissue (tissue-specific promoters) or can be otherwise under more precise environmental control (inducible promoters).

The term “inducible promoter” refers to a promoter that is regulated by particular conditions, such as light, anaerobic conditions, temperature, chemical concentration, protein concentration, conditions in an organism, cell, or organelle. Stress-inducible promoters, for example, can be activated under conditions of stress, such as drought, high or low temperature, lack of appropriate nutrients. One example of an inducible promoter that can be utilized with the polynucleotides provided herein is PARSK1. This promoter is from an Arabidopsis gene encoding a serine-threonine kinase enzyme, and is induced by dehydration, ABA, and sodium chloride (Wang and Goodman (1995) Plant J. 8:37). Other examples of stress-inducible promoters include PT0633 and PT0688. These promoters may be inducible under conditions of drought.

These and other objects, advantages, and features of the invention will become apparent to those persons skilled in the art upon reading the details of the invention as more fully described below.

The present invention provides for a cell comprising a modified or altered enzymatic activity of a rice-diverged glycosyltransferase (GT). The rice-diverged GT of the present invention have an expression level at equal to higher than the expression level observed for the rice-diverged GT identified in Table 3. The rice-diverged GT have a high expression in vegetative above ground plant tissue. The enzymatic activity of the rice-diverged GT can be modified or altered in that the expression of the GT is modified or altered, or the GT has a modified or altered amino acid sequence as compared to the wild-type GT such that the enzymatic activity of the GT is modified, altered, or a combination thereof. In some embodiments of the invention, the cell is a plant cell.

The rice-diverged GT is a GT identified by the method taught in Example 1 of the present specification. The rice-diverged GT can also be a sole GT gene expressed in a plant or the GT that is most rice-diverged among all redundant GT genes in a plant. Such rice-diverged GT include, but is not limited to, the 33 GT genes described herein, such as Table 3. Such rice-diverged GT include the GT having an amino acid sequence comprising an amino acid sequence, or at least 70% amino acid sequence similarity to, chosen from SEQ ID NO:1-30, wherein the GT has a GT enzymatic activity. In some embodiments, the amino acid sequence similarity is at least 80%. In some embodiments, the amino acid sequence similarity is at least 90%. In some embodiments, the amino acid sequence similarity is at least 95%. In some embodiments, the amino acid sequence similarity is at least 99%.

In some embodiments, the GT is rice-diverged in above-ground vegetative tissues. In some embodiments, the GT is involved in directly or indirectly in cellulose or cell wall synthesis, including but not limited to synthesis of a type I or a type II wall-specific or enriched component, including but not limited to glucuronoarabinoxylan.

The following amino acid sequences (SEQ ID NOs:1-269) depict amino acids of identified known rice-diverged GTs. The amino acid sequences SEQ ID NOs:1-30 depict amino acids of identified known rice-diverged GTs that have a high expression in vegetative above ground plant tissue.

Amino acid sequence of LOC_Os02g28900.1|12002.m08034
(SEQ ID NO: 1)
MPTASPAHAVFFPYPVQGHVASALHLAKLLHARGGVRVTFVHSERNRRRVIRSHGEGALA
AGAPGFCFAAVPDGLPSDDDDDGPSDPRDLLFSIGACVPHLKKILDEAAASGAPATCVVS
DVDHVLLAAREMGLPAVAFWTTSACGLMAFLQCKELIDRGIIPLKDAEKLSNGYLDSTVV
DWVPGMPADMRLRDFFSFVRTTDTDDPVLAFVVSTMECLRTATSAVILNTFDALEGEVVA
AMSRILPPIYTVGPLPQLTAASHVVASGADPPDTPALSAASLCPEDGGCLEWLGRKRPCS
VLYVNFGSIVYLTSTQLVELAWGLADSGHDFLWVIRDDQAKVTGGDGPTGVLPAEFVEKT
KGKGYLTSWCPQEAVLRHDAIGAFLTHCGWNSVLEGISNGVPMLCYPMAADQQTNCRYAC
TEWRVGVEVGDDIEREEVARMVREVMEEEIKGKEVRQRATEWKERAAMAVVPSGTSWVNL
DRMVNEVFSPGNNM*
Amino acid sequence of LOC_Os04g25440.1|12004.m07672
(SEQ ID NO: 2)
MGSLPAAAEARPHAVMVPYPAQGHVTPMLTLAKLLYSRGFHVTFVNNEFNHRRLLRARGA
RALDGAPGFRFAAMDDGLPPSDADATQDVPALCHSVRTTWLPRFMSLLAKLDDEAAAAAA
ADGAARRVTCVVADSNMAFGIHAARELGLRCATLWTASACGFMGYYHYKHLLDRGLFPLK
SEADLSNGHLDTTVDWIPGMTGDLRLRDLPSFVRSTDRDDIMFNFFVHVTASMSLAEAVI
INTFDELDAPSSPLMGAMAALLPPIYTVGPLHLAARSNVPADSPVAGVGSNLWKEQGEAL
RWLDGRPPRSVVYVNFGSITVMSAEHLAEFAWGLAGSGYAFLWNLRPDLVKGDGGAAPAL
PPEFAAATRERSMLTTWCPQAEVLEHEAVGVFLTHSGWNSTLESIAGGVPMVCWPFFAEQ
QTNCRYKRTEWGIGAEIPDDVRRGEVEALIREAMDGEKGREMRRRVAELRESAVAAAKPG
GRSVHNIDRLIDEVLMA*
Amino acid sequence of LOC_Os11g04860.1|12011.m04684
(SEQ ID NO: 3)
MANAANQHTCDGPQPSAPTHFLIVAYGIQSHINPAQNLAHRLASIDASSVMCTLSIHASA
HRRMFSSLIASPDEETTDGIISYVPFSDGFDDISKLSILSGDERARSRCTSFESLSAIVS
QLAARGRPVTCIVCTMAMPPVLDVARKNGIPLVVFWNQPATVLAAYYHYYHGYRELFASH
ASDPSYEVVLPGMQPLCIRSLPSFLVDVTNDKLSSFVVEGFQELFEFMDREKPKVLVNTL
NVLEAATLTAVQPYFQEVFTIGHLVAGSAKERIHMFQRDKKNYMEWLDTHSERSVVYISF
GSILTYSKRQVDEILHGMQECEWPFLWVVRKDGREEDLSYLVDNIDDHHNGMVIEWCDQL
DVLSHPSVGCFVTQCGWNSTLEALELGVPMVAVPNWSDQPTIAYLVEKEWMVGTRVYRND
EGVIVGTELAKSVKIVMGDNEVATKIRERVNSFKHKIHEEAIRGETGQRSLQIFAKTIIE
SD*
Amino acid sequence of LOC_Os02g49332.1|12002.m33282
(SEQ ID NO: 4)
MAGSGGGVVSGGRQRGPPLFATEKPGRMAMAAYRVSAATVFAGVLLIWLYRATHLPPGGG
DGVRRWAWLGMLAAELWFGFYWVLTLSVRWCPVYRRTFKDRLAQSYSEDELPSVDIFVCT
ADPTAEPPMLVISTVLSVMAYDYLPEKLNIYLSDDAGSVLTFYVLCEASEFAKHWIPFCK
KYKVEPRSPAAYFAKVASPPDGCGPKEWFTMKELYKDMTDRVNSVVNSGRIPEVPRCHSR
GFSQWNENFTSSDHPSIVQILIDSNKQKAVDIDGNALPTLVYMAREKKPQKQHHFKAGSL
NALIRVSSVISNSPIIMNVDCDMYSNNSESIRDALCFFLDEEQGQDIGFVQYPQNFENVV
HNDIYGHPINVVNELDHPCLDGWGGMCYYGTGCFHRREALCGRIYSQEYKEDWTRVAGRT
EDANELEEMGRSLVTCTYEHNTIWGIEKGVRYGCPLEDVTTGLQIQCRGWRSVYYNPKRK
GFLGMTPTSLGQILVLYKRWTEGFLQISLSRYSPFLLGHGKIKLGLQMGYSVCGFWAVNS
FPTLYYVTIPSLCFLNGISLFPEKTSPWFIPFAYVMVAAYSCSLAESLQCGDSAVEWWNA
QRMWLIRRITSYLLATIDTFRRILGISESGFNLTVKVTDLQALERYKKGMMEFGSFSAMF
VILTTVALLNLACMVLGISRVLLQEGPGGLETLFLQAVLCVLIVAINSPVYEALFLRRDK
GSLPASVARVSICFVLPLCILSICK*
Amino acid sequence of LOC_Os07g36630.1|12007.m07914
(SEQ ID NO: 5)
MAANGGGGGAGGCSNGGGGGAVNGAAANGGGGGGGGSKGATTRRAKVSPMDRYWVPTDEK
EMAAAVADGGEDGRRPLLFRTFTVRGILLHPYRLLTLVRLVAIVLFFIWRIRHPYADGMF
FWWISVIGDFWFGVSWLLNQVAKLKPIRRVPDLNLLQQQFDLPDGNSNLPGLDVFINTVD
PINEPMIYTMNAILSILAADYPVDKHACYLSDDGGSIIHYDGLLETAKFAALWVPFCRKH
SIEPRAPESYFAVKSRPYAGSAPEDFLSDHRYMRREYDEFKVRLDALFTVIPKRSDAYNQ
AHAEEGVKATWMADGTEWPGTWIDPSENHKKGNHAGIVQVMLNHPSNQPQLGLPASTDSP
VDFSNVDVRLPMLVYIAREKRPGYDHQKKAGAMNVQLRVSALLTNAPFIINFDGDHYVNN
SKAFRAGICFMLDRREGDNTAFVQFPQRFDDVDPTDRYCNHNRVFFDATLLGLNGIQGPS
YVGTGCMFRRVALYGVDPPRWRPDDGNIVDSSKKFGNLDSFISSIPIAANQERSIISPPA
LEESILQELSDAMACAYEDGTDWGKDVGWVYNIATEDVVTGFRLHRTGWRSMYCRMEPDA
FRGTAPINLTERLYQILRWSGGSLEMFFSHNCPLLAGRRLNFMQRIAYINMTGYPVTSVF
LLFYLLFPVIWIFRGIFYIQKPFPTYVLYLVIVIFMSEMIGMVEIKWAGLTLLDWIRNEQ
FYIIGATAVYPLAVLHIVLKCFGLKGVSFKLTAKQVASSTSEKFAELYDVQWAPLLFPTI
VVIAVNICAIGAAIGKALFGGWSLMQMGDASLGLVFNVWILLLIYPFALGIMGRWSKRPY
ILFVLIVISFVIIALADIAIQAMRSGSVRLHFRRSGGANFPTSWGF*
Amino acid sequence of LOC_Os08g06380.1|12008.m04777
(SEQ ID NO: 6)
MAPAVAGGGGRRNNEGVNGNAAAPACVCGFPVCACAGAAAVASAASSADMDIVAAGQIGA
VNDESWVAVDLSDSDDAPAAGDVQGALDDRPVFRTEKIKGVLLHPYRVLIFVRLIAFTLF
VIWRIEHKNPDAMWLWVTSIAGEFWFGFSWLLDQLPKLNPINRVPDLAVLRRRFDHADGT
SSLPGLDIFVTTADPIKEPILSTANSILSILAADYPVDRNTCYLSDDSGMLLTYEAMAEA
AKFATLWVPFCRKHAIEPRGPESYFELKSHPYMGRAQEEFVNDRRRVRKEYDDFKARING
LEHDIKQRSDSYNAAAGVKDGEPRATWMADGSQWEGTWIEQSENHRKGDHAGIVLVLLNH
PSHARQLGPPASADNPLDFSGVDVRLPMLVYVAREKRPGCNHQKKAGAMNALTRASAVLS
NSPFILNLDCDHYINNSQALRAGICFMLGRDSDTVAFVQFPQRFEGVDPTDLYANHNRIF
FDGTLRALDGLQGPIYVGTGCLFRRITLYGFEPPRINVGGPCFPRLGGMFAKNRYQKPGF
EMTKPGAKPVAPPPAATVAKGKHGFLPMPKKAYGKSDAFADTIPRASHPSPYAAEAAVAA
DEAAIAEAVMVTAAAYEKKTGWGSDIGWVYGTVTEDVVTGYRMHIKGWRSRYCSIYPHAF
IGTAPINLTERLFQVLRWSTGSLEIFFSRNNPLFGSTFLHPLQRVAYINITTYPFTALFL
IFYTTVPALSFVTGHFIVQRPTTMFYVYLAIVLGTLLILAVLEVKWAGVTVFEWFRNGQF
WMTASCSAYLAAVLQVVTKVVFRRDISFKLTSKLPAGDEKKDPYADLYVVRWTWLMITPI
IIILVNIIGSAVAFAKVLDGEWTHWLKVAGGVFFNFWVLFHLYPFAKGILGKHGKTPVVV
LVWWAFTFVITAVLYINIPHIHGPGRHGAASPSHGHHSAHGTKKYDFTYAWP*
Amino acid sequence of LOC_Os02g51060.1|12002.m10140
(SEQ ID NO: 7)
MQGDLALRAGGDRLLVADTVAAVVESLVQAWRQVRMELLVPLLRGAVVACMVMSVIVLAE
KVFLGVVSAVVKLLRRRPARLYRCDPVVVEDDDEAGRASFPMVLVQIPMYNEKEVYQLSI
GAACRLTWPADRLIVQVLDDSTDAIVKELVRKECERWGKKGINVKYETRKDRAGYKAGNL
REGMRRGYVQGCEFVAMLDADFQPPPDFLLKTVPFLVHNPRLALVQTRWEFVNANDCLLT
RMQEMSMDYHFKVEQEAGSSLCNFFGYNGTAGVWRRQVIDESGGWEDRTTAEDMDLALRA
GLLGWEFVYVGSIKVKSELPSTLKAYRSQQHRWSCGPALLFKKMFWEILAAKKVSFWKKL
YMTYDFFIARRIISTFFTFFFFSVLLPMKVFFPEVQIPLWELILIPTAIILLHSVGTPRS
IHLIILWFLFENVMALHRLKATLIGFFEAGRANEWIVTQKLGNIQKLKSIVRVTKNCRFK
DRFHCLELFIGGFLLTSACYDYLYRDDIFYIFLLSQSIIYFAIGFEFMGVSVSS*
Amino acid sequence of LOC_Os03g15840.1|12003.m07021
(SEQ ID NO: 8)
MGQQAAEAQPLLLQGDQVDAEWGCRPHRIVLFVEPSPFAYISGYKNRFQNFIKHLREMGD
EMLVVTTHKGAPEEFHGAKVIGSWSFPCPLYQNVPLSLALSPRIFSAVAKFKPDIIHATS
PGVMVFGARFIAKMLSVPMVMSYHTHLPAYIPRYNLNWLLGPTWSLIRCLHRSADLTLVP
SVAIAEDFETAKVVSANRVRLWNKGVDSESFHPKFRKHEMRIKLSGGEPEKPLIIHVGRF
GREKNLDFLKRVMERLPGVRIAFVGDGPYRAELERMFTGMPAVFTGMLQGEELSQAYASG
DLFAMPSESETLGQVVLESMASGVPVVAARAGGIPDIIPKDKEGKTSFLFTPGDLDECVR
KIEQLLSSKVLRESIGRAAREEMEKCDWRAASKTIRNEHYCTATLYWRKKMGRTN*
Amino acid sequence of LOC_Os03g16140.1|12003.m07051
(SEQ ID NO: 9)
MARKQHIAIFTTASLPWMTGTAVNPLFRAAYLAKAGDWEVTLVVPWLSKGDQLLVYPNKM
KFSVPGEQEGYVRRWLEERIGLLPKFEIKFYPGKFSTEKRSILPAGDITQTVSDDKADIA
VLEEPEHLTWYHHGRRWKNKFRKVIGVVHTNYLEYVKRERNGYIHAFLLKHINSWVTDIY
CHKVIRLSAATQEVPRSIVCNVHGVNPKFIEIGKLKHQQISQREQAFFKGAYYIGKMVWS
KGYTELLQLLQKHQKELSGLKMELYGSGEDSDEVKASAEKLNLDVRVYPGRDHGDSIFHD
YKVFINPSTTDVVCTTTAEALAMGKIVICANHPSNEFFKRFPNCHMYNTEKEFVRLTMKA
LAEEPIPLSEELRHELSWEAATERFVRVADIAPIMSIKQHSPSPQYFMYISPDELKKNME
EASAFFHNAISGFETARCVFGAIPNTLQPDEQQCKELGWRLQE*
Amino acid sequence of LOC_Os11g05990.1|12011.m04795
(SEQ ID NO: 10)
MASYGVDTRPAAAAAGGGGAGAGAAGEGALSFLSRGLREDLRLIRARAGELETFLTAPVP
EPELLARLRRAYSSSAGTTRLDLSAIGKAFGTGVVGRGSRGARWGWEEVQEAEEWEPIRM
VKARLREMERRRQWQATDMLHKVKLSLKSMSFVPEASEEVPPLDLGELLAYFLKQSGPLF
DQLGIKRDVCDKLVESLCSKRKDHLAYNSFPASEPSAFSNDNAGDELDLRIASVVQSTGH
NYEGGFWNDGHKYETADKRHVAIVTTASLPWMTGTAVNPLFRAAYLAKSSKQDVTLVVPW
LCKSDQELVYPNSMTFSSPQEQEAYMRSWLEERVGFKTDFKISFYPGKFQKERRSIIPAG
DTSQFIPSKEADIAILEEPEHLNWYHHGKRWTDKFNHVVGVVHTNYLEYIKREKNGVIQA
FFVKHINNLVARAYCHKVLRLSGATQDLPKSMICNVHGVNPKFLEVGERIAAERESGQHS
FSKGAYFLGKMVWAKGYRELIDLYAKHKSDLEGIKLDIYGNGEDSHEVQSAAMKLNLNLN
FHKGRDHADDSLHGYKVFINPSISDVLCTATAEALAMGKFVVCADHPSNDFFRSFPNCLT
YKTSEDFVAKVKEAMARDPQPLTPEQRYNLSWEAATQRFMEHSELDKVLSSSNRDCTTST
SGCGKSGDNKMEKSASLPNMSDMVDGGLAFAHYCFTGNELLRLSTGAIPGTLNYNKQHSL
DLHLLPPQVQNPVYGW*
Amino acid sequence of LOC_Os03g11330.1|12003.m06584
(SEQ ID NO: 11)
MQLRISPSMRSITISSSNGVVDSMKVRVAPQPPPPPPPLALQGVVTPGAGRRGGGGGGGG
GGGGWWGAGWYWRAVAFPAVVALGCLLPFAFILAAVPALEADGSKCSSIDCLGRRIGPSF
LGRQGGDSMRLVQDLYRIFDQVNNEESPDDKRIPESFRDFLLEMKDSHYDARTFAVRLKA
TMENMDKEVKKLRLAEQLYKHYAATAIPKGIHCLSLRLTDEYSSNAHARKQLPPPELLPL
LSDNSFQHYILASDNILAASVVVSSTVRSSSVPHKVVFHVITDKKTYPGMHSWFALNSIS
PAIVEVKGVHQFDWLTRENVPVLEAIENHRGVRNHYHGDHAAVSSASDSPRVLASKLQAR
SPKYISLLNHLRIYLPELFPNLNKVVFLDDDIVIQRDLSPLWKINLEGKVNGAVETCRGE
DNWVMSKRFRTYFNFSHPVIARSLDPDECAWAYGMNIFDLAAWRKTNIRETYHFWLKENL
KSGLTLWKFGTLPPALIAFRGHLHGIDPSWHMLGLGYQENTDIEGVRRSAVIHYNGQCKP
WLDIAFKNLQPFWTKHVNYSNDFIRNCHILEPQYDKE*
Amino acid sequence of LOC_Os05g35200.1|12005.m07750
(SEQ ID NO: 12)
MGSLETRYRPAGAPSDDTTKRRTPKSRIYKDVENFGVLVLEKNSGCKFKTLRYLLLAITS
ATFLTLLTPTFYEHQLQSSRYVDVGWIWDKPSYDPRYVSSVDVQWEDVYKALENLNDGSQ
KLKVGLLNFNSTEYGSWAQLLPGSAVSIVRLEHAKDSITWDTLYPEWIDEEEETDIPACP
SLPDPNVRKGSHFDVIAVKLPCTRVGGWSRDVARLHLQLSAAKLAVASSKGNQKVHVLFV
TDCFPIPNLFPCKNLVKHEGNAWLYSPDLKALREKLRLPVGSCELAVPLKAKARLYSVDR
RREAYATILHSASEYVCGAISAAQSIRQAGSTRDLVILVDDTISDHHRKGLEAAGWKVRV
IQRIRNPKAERDAYNEWNYSKFRLWQLTDYDKIIFIDADLLILRNVDFLFAMPEITATGN
NATLFNSGVMVIEPSNCTFQLLMDHTNEITSYNGGDQGYLNEIFTWWHRIPKHMNFLKHF
WEGDDDSAKAKKTELFGADPPILYVLHYLGMKPWLCFRDYDCNWNIPLMREFASDVAHAR
WWKVHDNMPEKLQSYCLLRSKLKAGLEWERRQAEKANLEDGHWRRNITDPRLTICYEKFC
YWESMLLHWGEKNPTNNNPVPATISSS*
Amino acid sequence of LOC_Os06g12280.1|12006.m05945
(SEQ ID NO: 13)
MAGGRAFRPSAPRRAAFAALLTLLLLATLSFLLSSPPPTHASHRSSYLGASPPSRLAAIR
RHAADHAAVLAAYAAHARRLKEASAAQSLSFATMSSDLSALSSRLASHLSLPEDAVKPLE
KEARDRIKLARLLAADAKEGFDTQSKIQKLSDTVFAVGEHLARARRAGRMSSRIAAGSTP
KSLHCLAMRLLEARLAKPSAFADDPDPSPEFDDPSLYHYAVFSDNVLAVSVVVASAARAA
ADPSRHVFHVVTAPMYLPAFRVWFARRPPPLGVHVQLLAYSDFPFLNETSSPVLRQIEAG
KRDVALLDYLRFYLPDMFPALQRVVLLEDDVVVQKDLAGLWHLDLDGKVNGAVEMCFGGF
RRYSKYLNFTQAIVQERFDPGACAWAYGVNVYDLEAWRRDGCTELFHQYMEMNEDGVLWD
PTSVLPAGLMTFYGNTKPLDKSWHVMGLGYNPSISPEVIAGAAVIHFNGNMKPWLDVALN
QYKALWTKYVDTEMEFLTLCNFGL*
Amino acid sequence of LOC_Os02g54820.3|12002.m10510
(SEQ ID NO: 14)
MPSLSCHNLLDLVAAADDAAPSPASLRLPRVMSAASPASPTSPSTPAPARRVVVSHRLPL
RAAADAASPFGFSFTVDSDAVAYQLRSGLPPGAPVLHIGTLPPPATEAASDELCNYLLAN
FSCLPVYLPADLHRRFYHGFCKHYLWPLLHYLLPLTPSSLGGLPFDRALYHSFLSANRAF
ADRLTEVLSPDDDLVWIHDYHLLALPTFLRKRFPRAKVGFFLHSPFPSSEIFRTIPVRED
LLRALLNADLVGFHTFDYARHFLSACSRLLGLDYQSKRGYIGIEYYGRTVTVKILPVGID
MGQLRSVVSAPETGDLVRRLTESYKGRRLMVGVDDVDLFKGIGLKFLAMEQLLVEHPELR
GRAVLVQIANPARSEGRDIQEVQGEARAISARVNARFGTPGYTPIVLIDRGVSVHEKAAY
YAAAECCVVSAVRDGLNRIPYIYTVCRQESTGLDDAAKRSVIVLSEFVGCSPSLSGAIRV
NPWSVESMAEAMNAALRMPEPEQRLRHEKHYKYVSTHDVAYWAKSFDQDLQRACKDHFSR
RHWGIGFGMSFKVVALGPNFRRLSVDHIVPSYRKSDNRLILLDYDGTVMPEGSIDKAPSN
EVISVLNRLCEDPKNRVFIVSGRGKDELGRWFAPCEKLGIAAEHGYFTRWSRDSAWETCG
LAVDFDWKKTAEPVMRLYKEATDGSTIEDKESALVWHHDEADPDFGSCQAKELLDHLENV
LANEPVVVKRGQHIVEVNPQVSACHLPSFL*
Amino acid sequence of LOC_Os05g44210.1|12005.m08547
(SEQ ID NO: 15)
MPTPAPSASSSSSSCGGGGGGAGAASSYSSSPDDRMLRGECGRRHPFASSAAVGAGSPDA
MDTDSAEPSSAATSVADFGARSPFSPGAASPANMDDAGGASAAGHAARPPLAGPRSGFRR
LGLRGMKQRLLVVANRLPVSANRRGEDQWSLEISAGGLVSALLGVKDVDAKWIGWAGVNV
PDEVGQRALTRALAEKRCIPVFLDEEIVHQYYNGYCNNILWPLFHYLGLPQEDRLATTRN
FESQFNAYKRANQMFADVVYQHYKEGDVIWCHDYHLMFLPKCLKDHDINMKVGWFLHTPF
PSSEIYRTLPSRSELLRSVLCADLVGFHTYDYARHFVSACTRILGLEGTPEGVEDQGRLT
RVAAFPIGIDSERFKRALELPAVKRHITELTQRFDGRKVMLGVDRLDMIKGIPQKILAFE
KFLEENHEWNDKVVLLQIAVPTRTDVPEYQKLTSQVHEIVGRINGRFGTLTAVPIHHLDR
SLDFHALCALYAVTDVALVTSLRDGMNLVSYEYVACQGSKKGVLILSEFAGAAQSLGAGA
ILVNPWNITEVADSIKHALTMSSDEREKRHRHNYAHVTTHTAQDWAETFVCELNETVAEA
QLRTRQVPPDLPSQAAIQQYLHSKNRLLILGFNSTLTEPVESSGRRGGDQIKEMELKLHP
ELKGPLRALCEDEHTTVIVLSGSDRSVLDENFGEFNMWLAAEHGMFLRPTNGEWMTTMPE
HLNMDWVDSVKNVFEYFTERTPRSHFEHRETSFVWNYKYADVEFGRLQARDMLQHLWTGP
ISNAAVDVVQGSRSVEVRSVGVTKGAAIDRILGEIVHSKSMITPIDYVLCIGHFLGKVIM
QLIYMCISVSFSARCFFCL*
Amino acid sequence of LOC_Os08g34580.1|12008.m07447
(SEQ ID NO: 16)
MPSLPNSGDEGGAPPPTPPPPGARRVVVAHRLPLRADPNPGAPHGFDFSLDPHALPLQLS
HGVPRPVVFVGVLPSAVAEAVQASDELAADLLARFSCYLVFLPAKLHADFYDGFCKHYMW
PHLHYLLPLAPSYGRGGGLPFNGDLYRAFLTVNTHFAERVFELLNPDEDLVFVHDYHLWA
FPTFLRHKSPRARIGFFLHSPFPSSELFRAIPVREDLLRALLNADLVGFHTFDYARHFLS
ACSRVLGLSNRSRRGYIGIEYFGRTVVVKILSVGIDMGQLRAVLPLPETVAKANEIADKY
RGRQLMLGVDDMDLFKGIGLKLLAMERLLESRADLRGQVVLVQINNPARSLGRDVDEVRA
EVLAIRDRINARFGWAGYEPVVVIDGAMPMHDKVAFYTSADICIVNAVRDGLNRIPYFYT
VCRQEGPVPTAPAGKPRQSAIIVSEFVGCSPSLSGAIRVNPWNVDDVADAMNTALRMSDG
EKQLRQEKHYRYVSTHDVVYWAQSFDQDLQKACKDNSSMVILNFGLGMGFRVVALGPSFK
KLSPELIDQAYRQTGNRLILLDYDGTVMPQGLINKAPSEEVIRTLNELCSDPMNTVFVVS
GRGKDELAEWFAPCDEKLGISAEHGYFTRWSRDSPWESCKLVTHFNWKNIAGPVMKHYSD
ATDGSYIEVKETSLVWHYEEADPDFGSCQAKELQDHLQNVLANEPVFVKSGHQIVEVNPQ
GVGKGVAVRNLISTMGNRGSLPDFILCVGDDRSDEDMFEAMISPSPAFPETAQIFPCTVG
NKPSLAKYYLDDPADVVKMLQGLTDSPTQQQPRPPVSFENSLDD*.
Amino acid sequence of LOC_Os12g05550.1|12012.m04548
(SEQ ID NO: 17)
MKRRHLPPVLVLLLLSILSLSFRRRLLVLQGPPSSSSSSRHPVGDPLLRRLAADDGAGSS
QILAEAAALFANASISTFPSLGNHHRLLYLRMPYAFSPRAPPRPKTVARLRVPVDALPPD
GKLLASFRASLGSFLAGRRRRGRGGNVAGVMRDLAGVLGRRYRTCAVVGNSGVLLGSGRG
PQIDAHDLVIRLNNARVAGFAADVGVKTSLSFVNSNILHICAARNAITRAACGCHPYGGE
VPMAMYVCQPAHLLDALICNATATPSSPFPLLVTDARLDALCARIAKYYSLRRFVSATGE
PAANWTRRHDERYFHYSSGMQAVVMALGVCDEVSLFGFGKSPGAKHHYHTNQKKELDLHD
YEAEYDFYGDLQARPAAVPFLDDAHGFTVPPVRLHW*
Amino acid sequence of LOC_Os08g02370.1|12008.m04381
(SEQ ID NO: 18)
MFAPAAAARPHKQAPLARVPTRLVAALCTACFFLGVCVVNRYWAVPELPDCRTKVNSDNP
GAVMNQVSQTREVIIALDRTISEIEMRLAAARTMQARSQGLSPSDSGSDQGSTRARLFFV
MGIVTTFANRKRRDSIRQTWLPQGEHLQRLEKEKGVVIRFVIGRSANPSPDSEVERAIAA
EDKEYNDILRLDHVERNGSLPLKIQMFLSTALSIWDADFYVKVDDDVHVNIGITRSILAR
HRSKPRVYIGCMKSGPVVDKNESKYYEPDHWKFGTEGNNYFRHATRQLYAVTRDLATYIS
ANRHILHKYSNEDVSFGSWLIGLDVEHVDERSLCCGTPPDCEWKAQAGNPCAASFDWNCT
GICNPVERMEEVHRRCWEGHVADLQAQF*
Amino acid sequence of LOC_Os02g52560.1|12002.m10287
(SEQ ID NO: 19)
MDVKRARSPRAPGVDADDDKKRAAEWRGAVRPHMVLVGFLITLPVLVFVFGGRWGSFQTT
SAPNVGGRHVVPGGVTTTQKNEAPKNVSVPATATKSLPQPQDKLLGGLLSAAFEESSCQS
RYKSSLYRKKSPFPLSPYLVQKLRKYEAYHKKCGPGTKRYRKAIEQLKAGRNADNAECKY
VVWFPCNGLGNRMLTIASTFLYALISNRVLLMHVAAEQEGLFCEPFPGSSWVLPGDFPHN
NPQGLHIGAPESYVNMLKNNVVRNDDPGSVSASSLPPYVYLHVEQFRLKLSDNIFCDEDQ
LILNKFNWMILKSDSYFAPALFMTPMYEKELEKMFPQKESVFHHLGRYLFHPTNKVWGIV
SRYYEAYLARVDEKIGFQIRIFPEKPIKFENMYDQLTRCIREQRLLPELGTAEPANTTAE
AGKVKAVLIASLYSGYYEKIRGMYYENPTKTGEIVAVYQPSHEEQQQYTSNEHNQKALAE
IYLLSYCDKIAMSAWSTFGYVAYSFAGVKPWILLRPDWDKERSEVACVRSTSVEPCLHSP
PILSCRAKKEVDAATVKPYVRHCEDVGFGLKLFDS*
Amino acid sequence of LOC_Os07g49370.1|12007.m09146
(SEQ ID NO: 20)
MASAGGCKKKTGNSRSRSPRSPVVLRRAMLHSSLCFLVGLLAGLAAPSDWPAAAGAAVFL
RTLRASNVIFSRSSNRPQQPQLVVVVTTTEQSDDSERRAAGLTRTAHALRLVSPPLLWLV
VEEAPAEKHAAPPTARLLRRTGVVHRHLLMKQGDDDFSMQISMRREQQRNVALRHIEDHR
IAGVVLFGGLADIYDLRLLHHLRDIRTFGAWPVATVSAYERKVMVQGPLCINTSSSSVIT
RGWFDMDMDMAAGGERRAAADRPPPETLMEVGGFAFSSWMLWDPHRWDRFPLSDPDASQE
SVKFVQRVAVEEYNQSTTRGMPDSDCSQIMLWRIQTTL*
Amino acid sequence of LOC_Os01g70190.1|12001.m13067
(SEQ ID NO: 21)
MAMRLSSAAVALALLLAATALEDVARGQDTERIEGSAGDVLEDDPVGRLKVYVYELPTKY
NKKMVAKDSRCLSHMFAAEIFMHRFLLSSAIRTLNPEEADWFYTPVYTTCDLTPWGHPLP
FKSPRIMRSAIQFISSHWPYWNRTDGADHFFVVPHDFGACFHYQEEKAIERGILPLLRRA
TLVQTFGQKDHVCLKEGSITIPPYAPPQKMKTHLVPPETPRSIFVYFRGLFYDTANDPEG
GYYARGARASVWENFKNNPLFDISTDHPPTYYEDMQRSIFCLCPLGWAPWSPRLVEAVVF
GCIPVIIADDIVLPFADAIPWDEIGVFVAEDDVPKLDTILTSIPMDVILRKQRLLANPSM
KQAMLFPQPAQPGDAFHQILNGLGRKLPHPKSVYLDPGQKVLNWTQGPVGDLKPW*
Amino acid sequence of LOC_Os04g57510.2|12004.m10636
(SEQ ID NO: 22)
MWHVRKEIAPAILLVVDFGGWYKLDSNSASSNVSHMIQHTQVSLLKDVIVPYTHLLPTMH
LSENKDRPTLLYFKGAKHRHRGGLVREKLWDLMVNEPDVVMEEGYPNATGREQSIKGMRT
SEFCLHPAGDTPTSCRLFDAVASLCIPVIVSDEIELPFEGMIDYTEFAIFVSVNNSMRPK
WLTNYLRNVPRQQKDEFRRNMAHVQPIFEYDSIYPGRMASAAQDGAVNHIWKKIHQKLPM
IQEAVTREKRKPDGTSIPLRCHCT*
Amino acid sequence of LOC_Os02g58560.2|12002.m100475
(SEQ ID NO: 23)
MDVPTNLDARRRISFFANSLFMDMPSAPKVRHMLPFSVLTPYYKEDVLFSSQALEDQNED
GVSILFYLQKIYPDEWKHFLQRVDCNTEEELRETEQLEDELRLWASYRGQTLTRTAVALP
YSQRDDVLQTSFGASGFS*
Amino acid sequence of LOC_Os06g02260.1|12006.m04960
(SEQ ID NO: 24)
MLTFFFTTVGYYVCTMMTVLTVYIFLYGRVYLALSGLDYEISRQFRFLGNTALDAALNAQ
FLVQIGIFTAVPMIMGFILELGLLKAIFSFITMQLQFCSVFFTFSLGTRTHYFGRTILHG
GAKYHATGRGFVVRHIKFAENYRLYSRSHFVKALEVALLLIIYIAYGYTRGGSSSFILLT
ISSWFLVVSWLFAPYIFNPGSFEWQKTVEDFDDWTNWLLYKGGVGVKGENSWESWWDEEQ
AHIQTLRGRILETILSLRFLIFQYGIVYKLKIASHNTSLAVYGFSWIVLLVLVLLFKLFT
ATPKKSTALPTFVRFLQGLLAIGMIAGIALLIALTKFTIADLFASALAFVATGWCVLCLA
VTWKRLVKFVGLWDSVREIARMYDAGMGALIFVPIVFFSWFPFVSTFQSRFLFNQAFSRG
LEISLILAGNKANQEA*
Amino acid sequence of LOC_Os02g22380.1|12002.m07476
(SEQ ID NO: 25)
MTSTAYSRPSKLPGGGNGSDRRLPPRLMRGLTTKIEPKKLGVGLLAGCCLALLTYVSLAK
LFAIYSPVFASTANTSALMQNSPPSSPETGPIPPQETAAGAGNNDSTVDPVDLPEDKSLV
EAQPQEPGFPSAESQEPGLPAALSRKEDDAERAAAAAASEIKQSEKKNGVAAGGDTKIKC
DENGVDEGFPYARPSVCELYGDVRVSPKQKTIYVVNPSGAGGFDENGEKRLRPYARKDDF
LLPGVVEVTIKSVPSEAAAPKCTKQHAVPAVVFSVAGYTDNFFHDMTDAMIPLFLTTAHL
KGEVQILITNYKPWWVQKYTPLLRKLSNYDVINFDEDAGVHCFPQGYLGLYRDRDLIISP
HPTRNPRNYTMVDYNRFLRDALELRRDRPSVLGEEPGMRPRMLIISRAGTRKLLNLEEVA
AAATELGFNVTVAEAGADVPAFAALVNSADVLLAVHGAGLTNQIFLPAEAVVVQIVPWGN
MDWMATNFYGQPARDMQLRYVEYYVGEEETSLKHNYSRDHMVFKDPKALHAQGWQTLAAT
IMKQDVEVNLTRFRPILLQALDRLQQ*
Amino acid sequence of LOC_Os06g27560.1|12006.m07301
(SEQ ID NO: 26)
MSSTAYTRPSKPPGPAGERRPPRLAKELGRIEPKKLGIGLVAGCCLALLTYISFARLFAI
YSPVFESTSLVMKNAPPASTQQNPVLAQQQSKEEDEKDVGEDETDRKVPSFAETTEKNEE
EETVTKPSGDEAEATISCDENGVDEGFPYARPPVCELTGDIRISPKEKTMFFVNPSSAGA
FDGNGEKKIRPYARKDDFLLPGVVEVIIKSVSSPAIAPACTRTHNVPAVVFSVAGYTDNF
FHDNTDVMIPLFLTTSHLAGEVQFLITNFKPWWVHKFTPLLKKLSNYGVINFDKDDEVHC
FRRGHLGLYRDRDLIISPHPTRNPRNYSMVDYNRFLRRAFGLPRDSPAVLGDKTGAKPKM
LMIERKGTRKLLNLRDVAALCEDLGFAVTVAEAGADVRGFAEKVNAADVLLAVHGAGLTN
QIFLPTGAVLVQIVPWGKMDWMATNFYGQPARDMRLRYVEYYVSEEETTLKDKYPRDHYV
FKDPMAIHAQGWPALAEIVMKQDVTVNVTRFKPFLLKALDELQE*
Amino acid sequence of LOC_Os06g28124.1|12006.m07356
(SEQ ID NO: 27)
MVSQSKTPAPEPSTTKMPSAPPAVKGVAKTLAQHHKAVIGFLLGFFLVLLLYTFLSGQLV
SSEDAIVRAVTQQSTPAVHTDQDGRTTSPTSPTSTSSNTTQDNLEGKNTERSSQPAVNDE
ASDKMEEDLIRQDIDQAGTKNGTNHKPGAPRKPICDLLDPRYDICEISGDARTMGTNRTI
LYVPPVGERGLADDSHEWSIRDQSRKYLEYINKVTVRSLDAQAAPGCTSRHAVPAVVFAM
NGLTSNPWHDFSDVLIPLFITTRVYEGEVQFLVSDLQPWFVDKYRLILTNLSRYDIVDFN
QDSDVRCYPKITVGLRSHRDLGIDPARTQRNYTMLDFRLYIREVYSLPPAGVDIPFKESS
MQRRPRAMLINRGRTRKFVNFQEIAAAVVAAGFEVVPVEPRRDLSIEEFSRVVDSCDVLM
GAHGAGLTNFFFLRTNAVMLQVVPWGHMEHPSMVFYGGPAREMRLRDVEYSIAAEESTLY
DKYSKDHPAIRDPESIHKQGWQFGMKYYWIEQDIKLNVTRFAPTLQQVLQMLRG*
Amino acid sequence of LOC_Os03g40270.1|12003.m09110
(SEQ ID NO: 28)
MAGTVTVPSASVPSTPLLKDELDIVIPTIRNLDFLEMWRPFFQPYHLIIVQDGDPTKTIR
VPEGFDYELYNRNDINRILGPKASCISFKDSACRCFGYMVSKKKYVFTIDDDCFVAKDPS
GKDINALEQHIKNLLSPSTPFFFNTLYDPYREGADFVRGYPFSLREGAKTAVSHGLWLNI
PDYDAPTQMVKPRERNSRYVDAVMTVPKGTLFPMCGMNLAFDRDLIGPAMYFGLMGDGQP
IGRYDDMWAGWCMKVICDHLSLGVKTGLPYIWHSKASNPFVNLKKEYKGIFWQEDIIPFF
QNATIPKECDTVQKCYLSLAEQVREKLGKIDPYFVKLADAMVTWIEAWDELNPSTAAVEN
GKAK*
Amino acid sequence of LOC_Os03g63270.1|12003.m11195
(SEQ ID NO: 29)
MLGGGKMKGGETMGGGGGSGSSSISPLVSFVLGAAMATVCILFVMSASPGRRLADISAWS
NADDAPPLPLPLQDAAVDSNDSLAAAAAANVTVVAAPAPAPVQAPAPASPYGDLEEVLRR
AATKDRTVIMTQINLAWTKPGSLLDLFFESFRLGEGGVSRLLDHLVIVTMDPAAYEGCQA
VHRHCYFLRTTGVDYRSEKMFMSKDYLEMMWGRNKFQQTILELGYNFLFTDVDVMWFRDP
FRHISMGADIAISSDVFIGDPYSLGNFPNGGFLFVRSNDKTLDFYRSWQQGRWRFFGKHE
QDVFNLIKHEQQAKLGIAIQFLDTTYISGFCQLSKDLNKICTLHANCCVGLGAKMHDLRG
VLDVWRNYTAAPPDERRSGKFQWKLPGICIH*
Amino acid sequence of LOC_Os07g19444.1|12007.m06379
(SEQ ID NO: 30)
MTKSATSLLLGAALATVFFLLYTSVCRDLGDGPPKSSPPRWAHAQEQGTATVTPATRVVD
AEQGTGRPGRQEEEVVAPREEKQTKDEAASRSGHGGGSVEQQQNQRRIVMPTSQQKETPS
SPPQRQQQDLGELLRRAATPDKTVLMTAINEAWAAPGSFLDLFLESFRHGEGTEHLVRHL
LVVAMDGRAFERCNAVHQFCYWFRVDGMDFAAEQSYMKGDYLEMMWRRNRFQQTILELGF
SFLFTDVDILWFRSPFPHLSPDAQVVMSSDFFVGDPTSPGNYPNGGLLYVRSSASTVRFY
EHWQSSRARFPGKHEQFVFDRIVKEGVPPHVGATVRFLDTGHFGGFCQHGKDLGRVVTMH
ANCCVGLHNKLFDLRNVLDDWKTYKERVAAGNMDYFSWRVPGRCIH*
>LOC_Os01g08080.1
(SEQ ID NO: 31)
MAAELHFLVVPLIAQGHIIPMVEVARLLAARGARATVVTTPVNAARNGAAVEAARRDGLAVDLAEVAFPGPEFGVPE
GLENMDQLADADPGMYLSLQRAIWAMAARLERLVRALPRRPDCLVADYCNPWTAPVCDRLGIARVVMHCPSAYFLLA
THNLSKHGVYGRLALAAGDGELEPFEVPDFPVRAVVYTATFRRFFQWPGLEEEERDAVEAERTADGFVINTFRDIEG
AFVDGYAAALGRRAWAIGPTFGSISHLAAKQVIELARGVEASGRPFVWTIKEAKAAAAAVREWLDGEGYEERVKDRG
VLVRGWAPQVSILSHPATGGFLTHCGWNAALEAIARGVPALTWPTILDQFSSERLLVDVLGVGVRSGVTAPPMYLPA
EAEGVQVTGAGVEKAVAELMDGGADGVARRARARELAATARAAVEEGGSSHADLTDMIRHVGAQ
>LOC_Os01g43270.1
(SEQ ID NO: 32)
MAPAHAVTPHVVLLPSPGAGHVAPAAQLAARLATHHGCTATIVTYTNLSTARNSSALASLPTGVTATALPEVSLDDL
PADARIETRIFAVVRRTLPHLRELLLSFLGSSSPAGVTTLLTDMLCPAALAVAAELGIPRYVFFTSNLLCLTTLLYT
PELATTTACECRDLPEPVVLPGCVPLHGADLIDPVQNRANPVYQLMVELGLDYLLADGFLINTFDAMEHDTLVAFKE
LSDKGVYPPAYAVGPLVRSPTSEAANDVCIRWLDEQPDGSVLYVCLGSGGTLSVAQTAELAAGLEASGQRFLWVVRF
PSDKDVSASYFGTNDRGDNDDPMSYLPEGFVERTKGAGLAVPLWAPQVEVLNHRAVGGFLSHCGWNSTLEAASAGVP
TLAWPLFAEQKMNAVMLSSERVGLAALRVRPDDDRGVVTREEVASAVRELMAGKKGAAAWKKARELRAAAAVASAPG
GPQHQALAGMVGEWKGRG
>LOC_Os01g49230.1
(SEQ ID NO: 33)
MSQETPARRSLPHLLLVSAPLQGHVNPLLCLGGRLSSRGLLVTFTTVPHDGLKLKLQPNDDGAAMDVGSGRLRFEPL
RGGRLWAPADPRYRAPGDMQRHIQDAGPAALEGLIRRQANAGRPVSFIVANAFAPWAAGVARDMGVPRAMLWTQSCA
VLSLYYHHLYSLVAFPPAGAETGLPVPVPGLPALTVGELPALVYAPEPNVWRQALVADLVSLHDTLPWVLVNTFDEL
ERVAIEALRAHLPVVPVGPLFDTGSGAGEDDDCVAWLDAQPPRSVVFVAFGSVVVIGRDETAEVAEGLASTGHPFLW
VVRDDSRELHPHGESGGGGDKGKVVAWCEQRRVLAHPAVGCFVTHCGWNSTTEALAAGVPVVAYPAWSDQITNAKLL
ADVYGVGVRLPVRRRGHERAGGGGDAAEGAGVERQGERGGG
>LOC_Os01g53380.1
(SEQ ID NO: 34)
MRVEKAPFPEGFLRRTKGRGLVVMSWAPQRKVLEHSAVGGFVTHCGWNSMLEALTAGVPMLAWPLYAEQRMNKVFLV
EEMRLAVAVEGYDKGVVTAEEIQEKARWIMDSNGGRELRERSLAAMWEVKEALSDKGEFKIALLQLTSQWKNYNNS
>LOC_Os01g53420.1
(SEQ ID NO: 35)
MTANNSAVGNIDSHLYPLVVLHKHAENTSHNRAMRSRVVLYTWMVRGHLHPMTQLADRIANHGVPVTVAVADVPSSG
ESRKTVARLSAYYPSVSFQLLPPAAPARSGADTADPDADPFITLLADLRATNAALTAFVRSLPSVEALVIDFFCAYG
LDAAAELGVPAYLFFVSCASALASYLHIPVMRSAVSFGQMGRSLLRIPGVHPIPASDLPEVLLLDRDKDQYKATIAF
FEQLAKAKSVLVNTFEWLEPRAVKAIRDGIPRPGEPAPRLFCVGPLVGEERGGEEEKQECLRWLDAQPPRSVVFLCF
GSASSVPAEQLKEIAVGLERSKHSFLWAVRAPVAADADSTKRLEGRGEAALESLLPEGFLDRTWGRGLVLPSWAPQV
EVLRHPATGAFVTHCGWNSTLEAVTAGVPMVCWPMYAEQRMNKVFVVEEMKLGVVMDGYDDDGVVKAEEVETKVRLV
MESEQGKQIRERMALAKQMATRAMEIGGSSTASFTDFLGGLKIAMDKDN
>LOC_Os01g64910.1
(SEQ ID NO: 36)
MDKTIVLYPGLYVSHFVPMMQLADALLEHGYAVAVALIHVTMDEDATFAAAVARVAAAAKPSVTFHKLPRIHDPPAI
TTIVGYLEMVRRYNERLREFLRSGVRGRSGGIAAVVVDAPSIEALDVARELGIPAYSFFASTASALAVFLHLPWFRA
RAASFEELGDAPLIVPGVPPMPASHLMPELLEDPESETYRATVSMLRATLDADGILVNTFASLEPRAVGALGDPLFL
PATGGGEPRRRVPPVYCVGPLVVGHDDDDERKENTRHECLAWLDEQPDRSVVFLCFGGTGAVTHSAEQMREIAAGLE
NSGHRFMWVVRAPRGGGDDLDALLPDGFLERTRTSGHGLVVERWAPQADVLRHRSTGAFVTHCGWNSASEGITARVP
MLCWPLYAEQRMNKVFMVEEMGVGVEVAGWHWQRGELVMAEEIEGKIRLVMESEEGERLRSSVAAHGEAAAVAWRKD
GGAGAGSSRAALRRFLSDVGGRELRSVETLLLWAFHEIVVARIGLPLD
>LOC_Os01g66620.1
(SEQ ID NO: 37)
MAYLLGKPQHELVERQQIGWLAGPFAVIVDLLAPLFVDLLRRRPADAVVFDGVLPWAATAAARLRIPRYAFTGTGCF
ALPVQRALLLHAPQDRVASDDEPFLVPGLPDAVRLFRP
>LOC_Os02g11130.1
(SEQ ID NO: 38)
MTAPMTAESTTQPPSPQPHFVLAPLAAHGHVIPMVDLAGLLAAHGARASLVTTPLNATRLRGVADKAAREKLPLEIV
ELPFSPAVAGLPSDCQNADKLSEDAQLTPFLIAMRALDAPFEAYVRALERRPSCIISDWCNTWAAGVAWRIGIPRLF
FHGPSCFYSLCDLNAVVHGLHEQIVADDEQETTYVVPRMPVRVTVTKGTAPGFFNFPGYEALRDEAIEAMLAADGVV
VNTFLDLEAQFVACYEAALGKPVWTLGPLCLHNRDDEAMASCGTGSTDLRAITAWLDEQVTGSVVYVSFGSVLRKLP
KHLFEVGNGLEDSGKPFLWVVKESELVSSRPEVQEWLDEFMARTATRGLVVRGWAPQVTILSHRAVGGFLTHCGWNS
LLEAIARGVPVATWPHFADQFLNERLAVDVLGVGVPIGVTAPVSMLNEEYLTVDRGDVARVVSVLMDGGGEEAEERR
RKAKEYGEQARRAMAKGGSSYENVMRLIARFMQTGVEEH
>LOC_Os02g11670.1
(SEQ ID NO: 39)
MYRLDKRIYEERQETKLFNDMIQKVPKYLFEVGHGLEDSGKPFIWVVKVSEVATPEVQEWLSALEARVAGRGVVVRG
WAPQLAILSHRAVGGFVTHCGCNSILEDITHGVPVVTWPHISDQFLNERLAVDVLGVGVPEARLPVVTAVKINPYLY
RYLGSTTGRYLGSTTACGNCY
>LOC_Os02g11680.1
(SEQ ID NO: 40)
MAPTPDSVSATSPPPPLPPPHFVIVPFPAQGHTIPMVDLARLLAERGARASLVVTPVNAAHLRGVADHAARAKLPLE
IVEVSFSPSAADAGLPPGVENVDQITDYAHFRPFFDVMRHLAAPLEAYLRALPVPPSCVISDWSNPWTAGVASRVGV
PRLFFHGPSCFYSLCDLNAAAHGLQQQGDDDRILQLTMEAMRTADGAVVNTFKDLEDEFIACYEAALGKPVWTLGPF
CLYNRDADAMASRGNTLDVAQSAITTWLDGMDTDSVTYVNFGSLACKVPKYLFEVGHGLEDSGKPFICVVKESEVAT
PEVQEWLSALEARVAGRGVVAACGTIFGVIPFVSRRSLGIISGMTGAGVNFGAGLTQL
>LOC_Os02g11700.1
(SEQ ID NO: 41)
MAPTAELDTATSPPPPHFVIVPFPAQGHTIPMVDLARLLAERGVRASLVVTPVNAARLRGAADHAARAELPLEIVEV
PFPPSAADAGLPPGVENVDQITDYAHFRPFFDVMRELAAPLEAYLRALPAPPSCIISDWSNSWTAGVARRAGVPRLF
FHGPSCFYSLCDLNAAAHGLQQQGDDDRYVVPGMPVRVEVTKDTQPGFFNTPGWEDLRDAAMEAMRTADGGVVNTFL
DLEDEFIACFEAALAKPVWTLGPFCLYNRDADAMASRGNTPDVAQSVVTTWLDAMDTDSVIYVNFGSLARKVPKYLF
EVGHGLEDSGKPFIWVVKESEVAMPEVQEWLSALEARVAGRGVVVRGWAPQLAILSHRAVGGFVTHCGWNSILESIA
HGVPVLTWPHFTDQFLNERLAVNVLGVGVPVGATASVLLFGDEAAMQVGRADVARAVSKLMDGGEEAGERRRKAKEY
GEKAHRAMEKGGSSYESLTQLIRRFTLQEPKNSSSITVECSANRHI
>LOC_Os02g14540.1
(SEQ ID NO: 42)
MRRFVPQLRALVVGIGSTTAAIVCDFFGTPALALVAELGVPGYVFFPTSISFISVVRSVVELHDDAAVGEYRDLPDP
LVLPGCAPLRHDEIPDGFQDCADPNYAYVLEEGRRYGGADGFLVNSFPEMEPGAAEAFRRDAENGAFPPVYLVGPFV
RPNSNEDPDESACLEWLDHQPAGSVVYVSFGSGGALSVEQTAELAAGLEMSGHNFLWVVRMPSTGRLPYSMGAGHSN
PMNFLPEGFVERTSGRGLAVASWAPQVRVLAHPATAAFVSHCGWNSTLESVSSGVPMIAWPLYAEQKMNTVILTEVA
GVALRPVAHGGDGGVVSRKEVAAAVKELMDPGEKGSAVRRRARELQAAAAARAWSPDGASRRALEEVAGKWKNAVRE
DR
>LOC_Os02g14570.1
(SEQ ID NO: 43)
MAEAATGATDTSLPPPPPHVVLMASPGAGHLIPLAELARRLVSDHGFAVTVVTIASLSDPATDAAVLSSLPASVATA
VLPPVALDDLPADIGFGSVMFELVRRSVPHLRPLVVGSPAAAIVCDFFGTPALALAAELGVPGYVFFPTSISFISVV
RSVVELHDGAAAGEYRDLPDPLVLPGCAPLRHGDIPDGFRDSADPVYAYVLEEGRRYGGADGFLVNSFPEMEPGAAE
AFRRDGENGAFPPVYLVGPFVRPRSDEDADESACLEWLDRQPAGSVVYVSFGSGGALSVEQTRELAAGLEMSGHRFL
WVVRMPRKGGLLSSMGASYGNPMDFLPEGFVERTNGRGLAVASWAPQVRVLAHPATAAFVSHCGWNSALESVSSGVP
MIAWPLHAEQKMNAAILTEVAGVALPLSPVAPGGVVSREEVAAAVKELMDPGEKGSAARRRARELQAAAAARAWSPD
GASRRALEEVAGKWKNAVHEDR
>LOC_Os02g14590.1
(SEQ ID NO: 44)
MAELARRLVAFHGCAATLVTFSGLAASLDAHSAAVLASLPASSVAAVTLPEVTLDDVPADANFGTLIFELVRRSLPN
LRQFLRSIGGGVAALVSDFFCGVVLDLAVELGVPGYVFVPSNTASLAFMRRFVEVHDGAAPGEYRDLPDPLRLAGDV
TIRVADMPDGYLDRSNPVFWQLLEEVRRYRRADGFLVNSFAEMESTIVEEFKTAAEQGAFPPVYPVGPFVRPCSDEA
GELACLEWLDRQPAGSVVFVSFGSAGMLSVEQTRELAAGLEMSGHGFLWVVRMPSHDGESYDFATDHRNDDEEDRDG
GGHDDDPLAWLPDGFLERTSGRGLAVASWAPQVRVLSHPATAAFVSHCGWNSALESVSAGVPMVPWPLYAEQKVNAV
ILTEVAGVALRPAAARGGVDGVVTREEVAAAVEELMDPGEKGSAARRRAREMQAAAARARSPGGASHRELDEVAGKW
KQTNRAPYE
>LOC_Os02g14630.1
(SEQ ID NO: 45)
METFTADDQRDADAPRPPRVVLLASPGAGHLIPLAELARWLADHHGVAPTLVTFADLEHPDARSAVLSSLPATVATA
TLPAVPLDDLPADAGLERTLFEVVHRSLPNLRALLRSAASLAALVPDIFCAAALPVAAELGVPGYVFVPTSLAALSL
MRRTVELHDGAAAGEQRALPDPLELPGGVSLRNAEVPRGFRDSTTPVYGQLLATGRLYRRAAGFLANSFYELEPAAV
EEFKKAAERGTFPPAYPVGPFVRSSSDEAGESACLEWLDLQPAGSVVFVSFGSAGTLSVEQTRELAAGLEMSGHRFL
WVVRMPSFNGESFAFGKGAGDEDDHRVHDDPLAWLPDGFLERTSGRGLAVAAWAPQVRVLSHPATAAFVSHCGWNST
LESVAAGVPMIAWPLHAEQTVNAVVLEESVGVAVRPRSWEEDDVIGGAVVTREEIAAAVKEVMEGEKGRGMRRRARE
LQQAGGRVWSPEGSSRRALEEVAGKWKAAATATAHK
>LOC_Os02g14680.1
(SEQ ID NO: 46)
MEPFTSAAVEPAPPTADDQRDAPRPHVVLLASPGAGHLIPLAELARRLADHHGVAPTLVTFADLDNPDARSAVLSSL
PASVATATLPAVPLDDIPADAGLERMLFEVVHRSLPHLRVLLRSIGSTAALVPDFFCAAALSVAAELGVPGYIFFPT
SITALYLMRRTVELHDFAAAGEYHALPDPLELPGGVSLRTAEFPEAFRDSTAPVYGQLVETGRLYRGAAGFLANSFY
ELEPAAVEDSKKAAEKGTFPPAYPVGPFVRSSSDEAGESACLEWLDLQPAGSVVFVSFGSFGVLSVEQTRELAAGLE
MSGHRFLWVVRMPSLNDAHRNGGHDEDPLAWVPDGFLERTRGRGLAVAAWAPQVRVLSHPATAAFVSHCGWNSTLES
VATGVPMIAWPLHSEQRMNAVVLEESVGMALRPRAREEDVGGTVVRRGEIAVAVKEVMEGEKGHGVRRRARELQQAA
GRVWSPEGSSRRALEVVAGKWKAAAQK
>LOC_Os02g56010.1
(SEQ ID NO: 47)
MASGDGAAQTTEDSRRRATTSTSTPTCSAATSRSASRSSRPLAPSRAPGRPRHPPRPTRRAHPASPRASWAEAARLE
RRRRRRATGQPKRRWPPGSSGGGARGGSSGGDSDGHQGGPSGGGDDSGGRAFLRAVLSLLAAAHTLANNIAINRPLG
FIDHTSTRSDDSGEREIVGDGRRRRRRPGRGDVPLAGVRPHDPLPPAVQAPRGEGPRRLLPLHAAKPRQAPAGAGEP
FRSSPPPAAADADTVEGLPEGAESTADVTPDKDGLVKKACDGLAAPFAAFLAGRAKRPDWIVVDFCHHWLPPIADEH
CVPCAMFHIIPAAMNAMFGPRWANARYPRTAPEDFTVPPKWIPFPSTIAFRRREFGWIAGAFKPNASGLPDVERFWR
TEERCRLIINSSCHELEPPQLFDFLTGLFRKPTVPAGILPPTTNLVTDDDDDDDRSEVLQWLDGQPPKSVIYVALGS
EAPLSANDLHELALGLELAGVRFLWAIRSPTAGGVLPDGFEQRTRGRGVVWGRWVAQVRVLAHGAVGAFLTHCGWGS
TIEGVALGQPLVMLPLVVDQGIIARAMAERGVGVEIARDESDGSFDRDAVAAAVRRVAVGGEREAFASNANRIKDVV
GDQEREERYIDELVGYLRRYS
>LOC_Os03g11350.1
(SEQ ID NO: 48)
MQSPENAAPRVYFIPFPTPGHALPMCDLARLFASRGADATLVLTRANAARLGGAVARAAAAGSRIRVHALALPAEAA
GLTGGHESADDLPSRELAGPFAVAVDLLAPLFADLLRRRPADAVVFDGVLPWAATAAAELRVPRYAFTGTGCFALSV
QRALLLHAPQDGVASDDEPFLVPGLPDAVRLTKSRLAEATLPGAHSREFLNRMFDGERATTGWVVNSFADLEQRYIE
HYEKETGKPVFAVGPVCLVNGDGDDVMERGRGGEPCAATDAARALAWLDAKPARSVVYVCFGSLTRFPDEQVAELGA
GLAGSGVNFVWVVGGKNASAAPLLPDVVHAAVSSGRGHVIAGWAPQVAVLRHAAVGAFVTHCGWGAVTEAAAAGVPV
LAWPVFAEQFYNEALVVGLAGTGAGVGAERGYVWGGEESGGVVVCREKVAERVRAAMADEAMRRRAEEVGERARRAV
EVGGSSYDAVGALLEDVRRREMAADPRNVKEV
>LOC_Os03g24430.1
(SEQ ID NO: 49)
MAAAHFVFVPLMAQGHLIPAVDTALLLATHGAFCTVVATPATAARVRPTVDSARRSGLPVRLAEFPLDHAGAGLPEG
VDNMDNVPSEFMARYFAAVARLREPVERHLLLRADEGGAPPPTCVVADFCHPWASELAAGLAVPRLTFFSMCAFCLL
CQHNVERFGAYDGVADDNAPVVVPGLARRVEVTRAQAPGFFRDIPGWEKFADDLERARAESDGVVINTVLEMEPEYV
AGYAEARGMKLWTVGPVALYHRSTATLAARGNTAAIGADECLRWLDGKEPGSVVYVSFGSIVHPEEKQAVELGLGLE
ASGHPFIWVVRSPDRHGEAALAFLRELEARVAPAGRGLLIWGWAPQALILSHRAAGAFVTHCGWNSTLEAATAGLPV
VAWPHFTDQFLNAKMAVEVLGIGVGVGVEEPLVYQRVRKEIVVGRGTVEAAVRSAMDGGEEGEARRWRARALAAKAR
AAAREGGSSHANLLDLVERFRPRHVAASEAANGTTAPPPPPRQ
>LOC_Os03g46400.1
(SEQ ID NO: 50)
MHTTNGAPACCDANADTPPLHLIFVPFLSRSHFGPVTAMAAEADACHRGGRTAATIVTTRHFAAMAPASVPVRVAQF
GFPGGHNDFSLLPGEVSAAAFFAAAEEALAPALGAAVRGLLREGGSTATVTVVSDAVLHWAPRVARECGVLHVTFHT
IGAFAAAAMVAIHGHLHLREAMPDPFGVDEGFPLPVKLRGVQVNEEALVHLPLFRAAEAESFAVVFNSFAALEADFA
EYYRSLDGSPKKVFLVGPARAAVSKLSKGIAADGVDRDPILQWLDGQPAGSVLYACFGSTCGMGASQLTELAAGLRA
SGRPFLWVIPTTAAEVTEQEERASNHGMVVAGRWAPQADILAHRAVGGFLSHCGWNSILDAISAGVPLATWPLRAEQ
FLNEVFLVDVLRVGVRVREAAGNAAMEAVVPAEAVARAVGRLMGDDDAAARRARVDELGVAARTAVSDGGSSCGDWA
ELINQLKALQLTSSRDRRTDAVTRD
>LOC_Os03g48740.1
(SEQ ID NO: 51)
MAHVLVVPYPSQGHMNPMVQFARKLASKGVAVTVVTTRFIERTTSSSAGGGGLDACPGVRVEVISDGHDEGGVASAA
SLEEYLATLDAAGAASLAGLVAAEARGAGADRLPFTCVVYDTFAPWAGRVARGLGLPAVAFSTQSCAVSAVYHYVHE
GKLAVPAPEQEPATSRSAAFAGLPEMERRELPSFVLGDGPYPTLAVFALSQFADAGKDDWVLFNSFDELESEVLAGL
STQWKARAIGPCVPLPAGDGATGRFTYGANLLDPEDTCMQWLDTKPPSSVAYVSFGSFASLGAAQTEELARGLLAAG
RPFLWVVRATEEAQLPRHLLDAATASGDALVVRWSPQLDVLAHRATGCFVTHCGWNSTLEALGFGVPMVAMPLWTDQ
PTNALLVERAWGAGVRARRGDADADDAAGGTAAMFLRGDIERCVRAVMDGEEQEAARARARGEARRWSDAARAAVSP
GGSSDRSLDEFVEFLRGGSGADAGEKWKTLVWEGSEAAASEM
>LOC_Os03g53350.1
(SEQ ID NO: 52)
MADAIGGGGRRRRLRVFFLPFFAKGHLIPMTDLACRMAAAGPEEMDATMVVTPGNAALIATAVTRAAARGHPVGVLC
YPFPDVGMERGVECLGVAAAHDAWRVYRAVDLSQPIHEALLLEHRPDAIVADVPFWWATDIAAELGVPRLTFSPVGV
FPQLAMNNLVTVRAEIIRAGDAAPPVPVPGMPGKEISIPASELPNFLLRDDQLSVSWDRIRASQLAGFGVAVNTFVD
LEQTYCHEFSRVDARRAYFVGPVGMSSNTAARRGGDGNDECLRWLSTKPSRSVVYVSFGSWAYFSPRQVRELALGLE
ASNHPFLWVIRPEDSSGRWAPEGWEQRVAGRGMVVHGCAPQLAVLAHPSVGAFVSHCGWSSVLEAASAGVPVLAWPL
VFEQFINERLVTEVVAFGARVRGGGRRSAREGEPETVPAEAVARAVAGIMARGGDGDRARARARVLAERARAAVGEG
GSSWRDIHRLIDDLTEATASPEPQLQ
>LOC_Os03g59030.1
(SEQ ID NO: 53)
MGDGGGGGLDVVVFPWLAFGHMIPYLELSKRLAARGHDVTFVSTPRNVSRLPPVPAGLSARLRFVSLPMPPVDGLPE
GAESTADVPPGNDELIKKACDGLAAPFAAFMADLVAAGGRKPDWIIIDFAYHWLPPIAAEHNAAAIAFLGPRWANAA
HPRAPLDFTAPPRWFPPPSAMAYRRNEARWVVGAFRPNASGVSDIERMWRTIESCRFTIYRSCDEVEPGVLALLIDL
FRRPAVPAGILLTPPPDLAAADDDDVDGGSSADRAETLRWLDEQPTKSVIYVALGSEAPVTAKNLQELALGLELAGV
RFLWALRKPAAGTLSHASAADADELLPDGFEERTRGRGVVWTGWVPQVEVLAHAAVGAFLTHCGWGSTIESLVFGHP
LVMLPFVVDQGLVARAMAERGVGVEVAREDDDEGSFGRHDVAAAVRRVMVEDERKVFGENARKMKEAVGDQRRQEQY
FDELVERLHTGGGEINDEKYC
>LOC_Os03g59350.1
(SEQ ID NO: 54)
MAAATADGHGGRRRLRVFFLPFFARGHLIPMTDLACLMAAASTDAVEVEATMAVTPANAAAIAATVAGNAAVRVVCY
PFPDVGLARGVECLGAAAAHDTWRVYRAVDLSRPAHESLLRHHRPDAIVADVPFWWATGVAAELGVPRLTFNPVGVF
PQLAMNNLVAVRPDIVRGGADGPPVTVPGMPGGREITIPVSELPDFLVQDDHLSMSWDRIKASQLAGFGVVVNTFAA
LEAPYCDEFSRVDARRAYFVGPVSQPSRAAAAAVRRGGDGDVDCLRWLSTKPSQSVVYVCFGSWAHFSVTQTRELAL
GLEASNQPFLWVIRSDSGDGGGERWEPEGWERRMEGRGMVVRGWAPQLAVLAHPSVGAFVTHCGWNSVLEAAAAGVP
ALTWPLVFEQFINERLVTEVAAFGARVWEDGGGKRGVRAREAETVPAGVIARAVAGFMAGGGGRRERAAAMATALAE
SARVAVGENGSSWRDIRRLIQDLTDATASQP
>LOC_Os04g04240.1
(SEQ ID NO: 55)
MEAEVASALVGTCMPCNQFVLSYQIVDSMIWLGIRDMINEFRKKKLKLRPVTYLSGAQGSGNDIPHGYIWSPHLVPK
PKDWGLKIDVVGFCFLDLASNYVPPEPLIKWLEAGDKPIYVGFGSLPVQDPAKMTEVIVKALEITGQRGIINKGWGG
LGTLAEPKDFVYLLDNCPHDWLFLQCKAVLLIMSSFHGVHHGGAGTTAAGLKAACPTTIVPFFGDQPFWGDRVHARG
VGPLPIPVDQFSLRKLVDAINFMMEPKVKEKAVELAKAMESEDGVSGVVRAFLRHLPLRAEETTPQPTSSFLEFLGP
FSFYDIFHVMALQAIVALYMCQYHLEASLSVSSRVIILVDPTAEGIC
>LOC_Os04g12669.1
(SEQ ID NO: 56)
MPHLADQPTISKYMESLWGMGVRVWQEKSGGIQREEVERCIREVMDGDRKEDYRRSAARLMKKAKEAMHEGGRQCGA
HVSTCRISITFPKPSGAARLPATKEGGRAYRSGDDGGGVDAGARRGDEAQRGGGARRLRGGGGGFQIGGDAGDGKER
GKGEKVMRTNMLIECRKSRVFVASRLHELMSEAKRHSLYFCRLGKQRAQLTVFGHMIHPFAEEAGDLRSWAFFNWMV
LVAIGALSNAITTIVSYLSAKSTCLNLR
>LOC_Os04g12678.1
(SEQ ID NO: 57)
MGSMSTPPPAAVTAANATSNVGDDNRGGGRVLLLPFPAAQGHTNPMLQFGRRLAYHGLRPTLVTTRYVLSTTPPPGD
PFRVAAISDGFDDDAGGMAALPDYGEYHRSLEAHGARTLAELLVSEARAGRPARVLVFDPHLPWALRVARDAGVGAA
AFMPQPCAVDLIYGEVCAGRLALPVTPADVSGLYARGALGVELGHDDLPPFVATPELTPAFCEQSVAQFAGLEDADD
VLVNSFTDLEPKEAAYMEATWRAKTVGPLLPSFYLGDGRLPSNTAYGFNLFTSTVPCMEWLDKQPPRSVVFVSYGTF
SGYDAAKLEEVGNGLCNSGKPFLWVVRSNEEHKLSRELREKCGKRGLIVPFCPQLEVLSHKATG
>LOC_Os04g12710.1
(SEQ ID NO: 58)
MASMNDQHGGATAHVLLVPLPAQGHMNPMLQFGRRLAYHGLRPTLVATRYVLSRSPPPGDPFRVAAFSDGFDAGGMA
SCPDPVEYCRRLEAVGSETLARVIDAKARAGRAATVLVYDPHMAWVPRVARAAGVPTAAFLSQPCAVDAIYGEVWAG
RVPLPMEDGGDLRRRGVLSVDLATADLPPFVAAPELYPKYLDISIVRISPL
>LOC_Os04g12720.1
(SEQ ID NO: 59)
MGSMSTPAASATTSNIEDNNNGGQVLLLPFPAAQGHTNPMLQFGRRLAYHGLRPTLVTTQYVLSTTPPPGDPFRVAA
ISDGFDDASGMAALPDPGEYLRTLEAHGSPTLAELLLSEARAGRPARVLVYDPHLPWARRVARAAGVATVAFLSQPC
AVDLIYGEVCARRLALPVTPTDASGLYARGVLGVELGPDDVPPFVAAPELTPAFCEQSVEQFAGLEDDDDILVNSFT
DLEPKEAAYMESTWRGKTVGPLLPSFYLDDGRLRSNTAYGFNLFRSTVPCMEWLDKQPPRSVVLVSYGTISTFDVAK
LEELGNGLCNSGKPFLWVVRSNEEHKLSVQLRKKCEKRGLIVPFCPQLEAIVNGIPLVAMPHWADQPTISKYVESLW
GTGVRVQLDKSGSLQREEVERCIREVMDGDRKEDYRRNAARLMKKAKESMQEGGSSDKNIAEFAAKYSN
>LOC_Os04g20540.1
(SEQ ID NO: 60)
MSTPKKHVLFPFTSKGHIAGFLSLASRLHRILPHATITLVSTPRNVAALRAAAAAPFLDFHALRFDPAEHGLPPGGE
SQDEIFPPLLIPLYEAFETLQPAFDDFVASTAAAAARVVVISDVFVAWTVEVARRHGSQVPKYMLYQYGLPAAGAAN
DGSGGRADRRFLDRQLAHGNNTDAVLVNAVAEPEPAGLAMLRRTLRVLPVWPIGPLSRDRRDAATEPTDDTVLRWMD
TQPPGSVLYISFGTNSMIRPEHMLELAAALESSGRCFLWKIKPPEGDVAGLNGGATTPSSYNRWLAEGFEERVRILA
HPSTAAFLSHCGWSSVLESMAHGVPVIGWLLTAEQFHNVMVLEGLGVCVEVARGNTDETVVERRRVAEVVKMVMGET
AKADDMRRRVQEVRTMMVDAWKEEGGSSFEASQAFLEAMKLK
>LOC_Os04g24840.1
(SEQ ID NO: 61)
MATARGARGATGDATATRRGSGGAQLEAAMGRDTGLGVDVTSTKSGTYGYQTDLGTWYLQDKVLEHDAVGVFLTHSG
WNSTLESPASGVLMLSWLFFAEQQTNCRYKQTEWGVAMEIGGEAWRGEVAAMTLEAMEGEKGREMRQRAEEWKQKAV
QVTLLGGPWDTNLDRVIHEVLLSCKDKTLSVNASASAQILALYELQKQQHKFLEQQHNFQMPQNFQKQQHQSSAVAI
SFQQQQQQQILANSTIKAGSLIFPLATPGAEDEDATQDIPALCQSTMTNCLGHLLALLARLKEWKEKAVRVTMPSGP
GDTNLDRIIHEVLLSCKGENGSGSVGSHPSHS
>LOC_Os04g24850.1
(SEQ ID NO: 62)
MGATGDKPPHAVCVPYPSQGDITPTLHLAKLLHARGFHVTLVNTEFNHRRLLASRGAAALDGVPGFVFAAIPDGLPA
MSGEHEDATQDIPALCQSTMTNCLGHLLALLSRLNEPASGSPPVTCLVADGLMSFAYDAASACGFVGCRLYRELIDR
GLVPLRDAAQLTDGYLDTVVDGAAARGMCDGVQLRDYPSFIRTTDLGDVMLNFIMREAERLSLPDAVILNTFDDLER
PALDAMRAVLPPPVYAVGPLHLHVRRAVPTGSPLHGVGSNLWKEQDGLLEWLDGHRPSSVVYVSYGSIAVMTSEQLL
EFAWGLADSGYAFVWVVRPDLVKGGEGDAAALPPEFHAAVEGRGVLPAWCPQEKVLEHDAVGVFLTHSGWNSTLESL
AAGVPMLSWPFFAEQQTNCRYKRTEWGIGMEIGGNARRGEVAAMIREAMEGKKGREIRRRAQEWKEKAVRVTLPGGP
GDTNLDRVIHDVLLSCKDKISRVNGESV
>LOC_Os04g25490.1
(SEQ ID NO: 63)
MGSFPAAEETTATAAARPHAVMVPYPAQGHVTPMLKLAVLLHARGFHVTFVNNEFNHRRLLRARGAGALDGAPGFRF
AAIDDGLPPSDADATQDVPALCHSVRTTCLPRFKALLAKLDEEADADAGAGAGDARRVTCVVADSTMAFAILAAREL
GLRCATLWTASACGEADLSNGHLDTKMDWIPGMPADLRLRDLPSVVRSTDRDDIMFNFFIDVTATMPLASAVILNTF
DELDAPLMAAMSALLPPIYTVGPLHLTARNNLPADSPVAGVGSNLWKEQGEALRWLDGRPPRSVVYGSITVMSAEHL
LEFAWGLAGSGYAFLWNVRPDLVKGDAAALPPEFAAATGERSMLTTWCPQAEVLEHEAVGVFLTHSGWNSTLESIVG
DVPMVCWPFFAEQQTNCRYKRTEWGIGAEIPDDVRRGEVEALIREAMDGEKGREMRRRVAELRESAVASGQQGGRSM
QNLDRLIDEVLLA
>LOC_Os04g25820.1
(SEQ ID NO: 64)
MATARGARGATGDATATRRGSGGAQLEAATGRDTGLGVDVTSTKSGTYGYQTDLGTCGVLRAWCPQDKVLEHDAVGV
FLTHSGWNSTLESPASGVPMLSWLFFAEQQTNCRYKQTEWGVAMEIGGEAWRGEVAAMTLEAMEGEKGREMRQRAEE
WKHKAVQVTLLGGPWDTNLDRVIHEVLLSCKDKTLRVNASASAQILALYELQKQQHKFLEQQHNFQMPQNFQKQQHQ
SSAVAISFQQQQQQQILANSTIKAGSLIFPLATPGAEDEDATQDIPALCQSTMTNCLGHLLALLARLKEWKEKAVRV
TMPSGPGDTNLDRIIHEVLLSCKGENGSGSLHYEKRNAWRKRLALFAPWS
>LOC_Os04g44240.1
(SEQ ID NO: 65)
MDCHRNESQRELEMGTKPHFVVIPWLATSHMIPIVDIACLLAAHGAAVTVITTPANAQLVQSRVDRAGDQGASRITV
TTIPFPAAEAGLPEGCERVDHVPSPDMVPSFFDAAMQFGDAVAQHCRRLTGPRRLSCLIAGISHTWAHVLARELGAP
CFIFHGFCAFSLLCCEYLHAHRPHEAVSSPDELFDVPVLPPFECRLTRRQLPLQFLPSCPVEYRMREFREFELAADG
IVVNSFEELERDSAARLAAATGKKVFAFGPVSLCCSPALDDPRAASHDDAKRCMAWLDAKKARSVLYVSFGSAGRMP
PAQLMQLGVALVSCPWPVLWVIKGAGSLPGDVKEWLCENTDADGVADSQCLAVRGWAPQVAILSHRAVGGFVTHCGW
GSTLESVAAGVPMAAWPFTAEQFVNEKLIVDVLGIGVSIGVTKPTGGMLTAGGGGGEETAEVGTEQVKRALNSLMDG
GVEGEERAKKVHELKAKAHAALEKEGSSYMNLEKLILSAV
>LOC_Os04g44250.1
(SEQ ID NO: 66)
METATSKPHFVLVPWIGSISHILPMTDIGCLLASHGAPVTIITTPVNSPLVQSRVDRATPHGAGITVTTIPFPAAEA
GLPEGCERLDLIPSPAMVPGFFRASRGFGEAVARHCRRQDARPRRRPSCIIAGMCHTWALGVARELGVPCYVFHGFG
AFALLCIEYLFKQRRHEALPSADELVDIPVLPPFEFKVLGRQLPPHFVPSTSMGSGWMQELREFDMSVSGVVVNIFE
DLEHGSAALLAASAGKKVLAVGPVSLPHQPILDPRAASDDARRCMAWLDAKEARSVVYVSFGSAGRMPAAQLMQLGM
ALVSCPWPTLWVFNGADTLPGDVRDWLRENTDADGVAHAHSKCLVVRGWAPQVAILDHPAVGGFMTHCGWGSTLESV
AAGMPMVTWPFFAEQFINERLIVDVLGIGVSVGVTRPTENVLTAGKLGGAEAKVEIGADQVKKALARLMDEGEDMRR
KVHELKEKARAALEEGGSSYMNLEKLIHSSV
>LOC_Os05g08480.1
(SEQ ID NO: 67)
MALSSPSSPPIKSRKPASSEDSVAMAAAPLHFVLVPLPAQGHVIPMMDMARLIAGHGGGGARVTVVLTPVMAARHRA
AVAHAARSGLAVDVSVLEFPGPALGLAAGCESYDMVADMSLFKTFTDAVWRLAAPLEAFLRALPRRPDCVVADSCSP
WTAGVARRLGVPRLVFHGPSALYILAVHNLARHGVYDRVAGDLEPFDVPDLPAPRAVTTNRASSLGLFHWPGLESHR
QDTLDAEATADGLVFNTCAAFEEAFVRRYAEVLGGGARNVWAVGPLCLLDADAEATAARGNRAAVDAARVVSWLDAR
PPASVLYVSFGSIARLNPPQAAELAAGLEASHRPFIWVTKDTDADAAAAAGLDARVVADRGLVIRGWAPQVTILSHP
AVGGFLTHCGWNSTVESLSHGVPLLTWPHFGDQFLNECLAVDVLGAGVRAGVKVPVTHVDAVNSPVQVRSGEVASAV
EELMGDGAAAAARRARARELAAEARAAMADGGSSARDLADMVWHVARRRDMVVVDPPPPPSPGGIAGGHGKMVSPSV
ASEVA
>LOC_Os05g08490.1
(SEQ ID NO: 68)
MNSLDDVPKPHFVLIPFMAQGHTIPMIDMAHLLAKHGAMVSFITTPVNAARIQSTIDRARELNIPIRFVPLRLPCAE
VGLLDGCENVDEILEKDQVMKMTDAYGMLHKPLVLYLQEQSVPPSCIVSDLCQPWTGDVARELGIPRLMFNGFCAFA
SLCRYLIHQDKVFENVPDGDELVILPGFPHHLEVSKARSPGNFNSPGFEKFRTKILDEERRADSVVTNSFYELEPSY
VDSYQKMIGKRGIFFIYNFFY
>LOC_Os05g08510.1
(SEQ ID NO: 69)
MARTVFLQLEEIALGLEASKRPFLWVIKSDNMPSETDKLFLPEGFEERTRGRGLIIQGWAPQALILSHPSVGGFVTH
CGWNSKIEGVSAGLPMITWPHCAEQFLNEELIMNALKVGLAVGVQSITNRTMKAHEISVVKRDQIERAVVELMGDET
GAEERRARAKELKEKARKAIDEGSSYNNIVLKNFRRCILRPLSKEKVGKIVGRKGTWKGNQG
>LOC_Os05g12450.1
(SEQ ID NO: 70)
MGGGRGGAPATASARARRPHQPRVLLLCSPCLGHLIPFAELARRLVADHGLAATLLFASARSPPSEQYLAVAASVLA
EGVDLVALPAPAPADALPGDASVRERAAHAVARSVPRVRDVARSLAATAPLAALVVDMIGAPARAVAEELGVPFYMF
FTSPWMLLSLFLHLPSLDADAARAGGEHRDATEPIRLPGCVPIHAHDLPSSMLADRSSATYAGLLAMARDAARADGV
LVNTFRELEPAIGDGADGVKLPPVHAVGPLIWTRPVAMERDHECLSWLNQQPRGSVVYVSFGSGGTLTWQQTAELAL
GLELSQHRFIWAIKRPDQDTSSGAFFGTANSRGEEEGMDFLPEGFIERTRGVGLLVPSWAPQTSILGHASIGCFLTH
CGWNSTLESVSNGVPMIAWPLYAEQKMNAAMMEVQAKVAIRINVGNERFIMNEEIANTIKRVMKGEEAEMLKMRIGE
LNDKAVYALSRGCSILAQVTHVWKSTVG
>LOC_Os05g42020.1
(SEQ ID NO: 71)
MASTDRSKKLRVLLIPFFATSHIGPFTDLAVRLVTARPDAVEPTIAVTPANVSVVRSALERHGSAATSVVSIATYPF
PEVAGLPRGVENLSTAGADGWRIDVAATNEALTRPAQEALISGQSPDALITDAHFFWNAGLAEELGVPCVSFSVIGL
FSGLAMRFVTAAAANDDSDSAELTLAGFPGAELRFPKSELPDFLIRQGNLDGIDPNKIPQGQRMCHGLAVNAFLGME
QPYRERFLRDGLAKRVYLVGPLSLPQPPAEANAGEASCIGWLDSKPSRSVLYVCFGTFAPVSEEQLEELALGLEASG
EPFLWAVRADGWSPPAGWEERVGERGVLVRGWVPQTAILSHPATAAFLTHCGSSSLLEAVAAGVPLLTWPLVFDQFI
EERLVTDVLRIGERVWDGPRSVRHEEAMVVPAAAVARAVARFLEPGGAGDAARLRAQELAAEAHAAVAEGGSSYRDL
RRLVDDMVEARAAGGEAAAAPQPQ
>LOC_Os05g42040.1
(SEQ ID NO: 72)
MASAERSKKLRVLLMPFFATSHIGPCTDLAVRLAAARPDVVEPTLAVTPANVSVVRSALRLHGSAASTVVSIATYPF
PEAAGLPPGVENLSTAGDERWRVDAAAFDEAMTWPAQEALIKDQSPDVLITDFHFSWNVGIAEELAMPCVQLNVIGL
FSTLAVYLAAAVVNDSDSEELTVAGFPGPELRIPRSELPDFLTAHRNLDLVDNMRKLVQVNTRCHGFAVNSFLFLDK
PYCEKFMCNGFAKRGYYVGPLCLPQPPAVASVGEPTCISWLDSKPNRSVVYICFGTFAPVSEEQLHELALGLEASGK
PFLWAVRAADGWAPPAGWEERVGDRGLLVRDWVPQTAILAHSATAAFLTHCGWNSVLEGVTAGVPLLTWPLVFEQFI
TERLVMDVLRIGERVWDGARSVRYKEAALVPAAAVARAVARFLEPGGAGDAARIRAQDFAAEAHAAVAEGGSSYGDL
RRLIDDLVEARADAGESALQPL
>LOC_Os05g42060.1
(SEQ ID NO: 73)
MASAERSKKLRILFIPFFATSHIGPFTDLAVRLAAARPDIVEPTIAVTPANVSVVRSAVKRHGSVASSMVSIAKYPF
PDVAGLSPGVENLSTAGDEGWRIDNAAFNEALTRPPQEAVIREQSPDVLITDSHFSWIVYIAEGLGMACFRFCVIGF
FSILAMRLLAGAAADANGSDSESLTAAGFPGPKLQIPRSEVPDFLTRQQNFDKFDMRKLQQSQDRCHGIVVNSFLFL
DKPYCEKFVCNGFAKRGYHVGPLCLPKPPAVGNVGEPSCISWLDSKPSRSVVYICFGTFAPVSEEQLHELALGLEAS
GKPFLWAVRAADGWAPPAGWEERVGDRGLLVRDWVPQTAILAHSATAAFLTHCGWNSMLEGATAGVPLLTWPLVFEQ
FITERFVTDVLRIGERVWDGPRSVRYEEKAVVPAAAVARAVARFLEPGGTGDAARIRAQELAAEAHAAVAEGGSSYD
DLRRLIDDMVEARAAAGGVAPARQPQ
>LOC_Os05g42070.1
(SEQ ID NO: 74)
MASDGSSKKLRVVLIPFFATSHIGPFTDFAVRLAAAWPDAVEATLAVTPANVPVVRSLLERHGPAGAGSVAIATYPF
PAVDGLPAGVENLSKAAPGDAWRINAVADDEALMRPAQESLVRELRPDVIVTDAHFFWNAGLADELGVPCVQFYAIG
AFSTIAMAHLVGAVKEGAKEAAGKPFLWVVRTDMWAPPDGWKERVGDRGMVIRGWAPQKAILAHPSVGAFVTQCGWN
SVLEAVSAGVPVLTWPMVFEQLDFSHFGPFEKLISQIDP
>LOC_Os05g45140.1
(SEQ ID NO: 75)
MKKTMVLYPGLSVSHFLPMMQFADELIDRGYAITVALIDPVFQQHIAFPATVDRAISSKPAIRFHRLPRVELPPAIT
TKDNDFSLLGYLDLVRRHNECLHDFLCSMPPGGAHALVVDPLSVEALDVAKRLNVPGYVFHPGNASAFAIHLQLPLI
RAEGQPSFRELGDTPLELPGLPPIPVSYLYEELLEDPESEVYKAIVDLFHRDIQDSNGFLMNTFESLEARVVNALRD
ARRHGDPAALPPFYCVGPLIEKAGERRETAERHECLAWLDRL
>LOC_Os05g45150.1
(SEQ ID NO: 76)
MFQRDSRCHHGGPALPPFYCIRPLVEKADERRDRAERHECLAWLDRQPERSVVFLCFGSTGAGSHSVEQLREIAVGL
EKSGQRFLWVVRAPRVAIDDDDDSFNPRAEPDVDALLPAGFLERTTGRGVVVKLWAPQVDVLYHRATGAFVTHCGWN
SVLEGITAGVPMLCWPLHSEQKMNMVLMVEEMDIAVEMAGWKQGLVTAEELEAKVRLVMESEAGSQLRARVTAHKEG
AATAWADGGSSRSAFARRGKRKWEAPHCGAIARREDGEPEPKLCRPAAPTLPFPALLRHLPCSPRSPSAQPPLCRGL
DRHPLSCLSSLRSGPPSATTFIFTTSRAHLSATIVIGAAIPTANFAVGVHGYGSEDLVGGSGCTDLERIGECYGGVG
GDQGWQCGGGDERGGGMEREEKNRREGEEGLKKGSEPNIWDSLFRLDSVIESLLE
>LOC_Os05g45170.1
(SEQ ID NO: 77)
MKKTIVLYPGVAVSHFLPMMQLADELVDHGYAVAVALIDPAFQQHTAFPATVDRVVSSKPTVRFHRLPRVELPPATA
TDDGDFLLLGYLDLVRRHNECLHDFLCSMLPGGVHAFVVDSLSVEALDVGERLNVPGFVFHPANLGAFAIFLQLPSI
RAEGEPSFRELGDNPLELPGLPPMPASHLFSQFLEHPESQVYKAMMNVSRRMLPTWKRSKIFSENQREFGRRGEETE
RKTEM
>LOC_Os05g45180.1
(SEQ ID NO: 78)
MKKTMVLYPGLSVSHFLPMMKLADELVEHGYAVTVALIDDPLQKQIAFTATVDRVISSKPSICFHRLPRVDHLPAVT
TNDGEFYLPGYLDLVRRHNEPLHGFLSSHFRGGIQALVVDMMSVEALDIAERLKVPGYLFHPSNASLFAFFLQIPSI
CAESKRSFSELGDTPLEIPGLPPMPASHFIDNRPEEPPESEVYKAVMDLVRRYTNKCSNGFLVNTVDSLEARVVNTL
RHARRQGGRALPPFYCVGPLVNKAGERGERPERHECLAWLDRQPDRTVVFLCFGSTGIGNHSTEQLREIAVGLEKSG
HRFLWVVRAPVVSDDPDRPDLDALLPAGFLERTSGQGAVVKQWAPQVDVLHHRATGAFVTHCGWNSVLEGITAGVPM
LCWPLHSEQKMNKVLMVEEMGIAVEMVGWQQGLVTAEEVEAKVRLVMESEAGVELRARVTAHKEAAAVAWTDVGSSR
AAFTEFLSNADSRQTS
>LOC_Os05g45180.2
(SEQ ID NO: 79)
MKKTMVLYPGLSVSHFLPMMKLADELVEHGYAVTVALIDDPLQKQIAFTATVDRVISSKPSICFHRLPRVDHLPAVT
TNDGEFYLPGYLDLVRRHNEPLHGFLSSHFRGGIQALVVDMMSVEALDIAERLKVPGYLFHPSNASLFAFFLQIPSI
CAESKRSFSELGDTPLEIPGLPPMPASHFIDNRPEEPPESEVYKAVMDLVRRYTNKCSNGFLVNTVDSLEARVVNTL
RHARRQGGRALPPFYCVGPLVNKAGERGERPERHECLAWLDRQPDRTVVFLCFGSTGIGNHSTEQLREIAVGLEKSG
HRFLWVVRAPVVSDDPDRPDLDALLPAGFLERTSGQGAVVKQWAPQVDVLHHRATGAFVTHCGWNSVLEGITAGVPM
LCWPLHSEQKMNKVLMVEEMGIAVEMVGWQQGLVTAEEVEAKVRLVMESEAGVELRARVTAHKEAAAVAWTDVGSSR
AAFTEFLSNADSRQTS
>LOC_Os06g10860.1
(SEQ ID NO: 80)
MAVATPTSTISPAARGKSAAIDADECIQWLDSKDPSSVIYVSFGSIARTDPKQLIELGLGLEASAHPFIWMVKNAEL
YGDTAREFFPRFEISGVDTVNADPVARHGRWLRDALRVNSIMEVVATRLPMVTWPHSVDQLLNQKMAVEVLGIGVGV
GLDESVTEGHCGGEGGGGEGNREHT
>LOC_Os06g11710.1
(SEQ ID NO: 81)
MAPAPATLPNAAARRPHALLVPFPSSGFINPMFHFARLLRSAGFVVTFVNTERNHALMLSRGRKRDGDGIRYEAIPD
GLSPPERGAQDDYGFGLLHAVRANGPGHLRELIARLNTGRGGGAGDSPPPPVTCVVASELMSFALDVAAELGVAAYM
LWGTSACGLAVRELRRRGYVPLKETQVNAGAHNPLAPYEHL
>LOC_Os06g11720.1
(SEQ ID NO: 82)
MCDSPSSSSCSSLSLALAMGERMRRAAHAMLFPFPCSGHINPTLKLAELLHSRGVHVTFVNTEHNHERLLRRRGGGG
ALRGREGFRFEAVPDGLRDDERAAPDSTVRLYLSLRRSCGAPLVEVARRVASGGGVPPVTCVVLSGLVSFALDVAEE
LGVPAFVLWGTSACGFACTLRLRQLRQRGYTPLKDESYLTNGYLDTPIDWIAGVPTVRLGDVSSFVRTLDPTSFALR
VEEDEANSCARAQGLILNTFDDLESDVLDALRDEFPRVYTVGPLAADRANGGLSLWEEDAACMAWLDAQPAGSVLYV
SFGSLTVMSPEELAELAWGLADTRRTFLWVIRPGLIAGAGAGDHDVVTNALPDGFVAETKGRCFIAEWCAQEEVLRH
RAVGGFLTHSGWNSTTESICAGVPMICWPGFADQYINSRYVRDEWGIGLRLDEELRREQVAAHVEKLMGGGGGGGDR
GKEMRRNAARWKAAAEAATAKGGSSYGGLDKLVEQLRLGQ
>LOC_Os06g16000.1
(SEQ ID NO: 83)
MAKGHAMPLLHLTRLLLARGLASKVTFFTTPRDAPFIRASLAGAGAAAVVELPFPTDDGLNDGAAPPQSMDDELASP
SQLADVVAASAALRPAFAAAFARLEPRPDVLVHDGFLPWAELAAADAGGVPRLVSYGMSAFATYVAGAVTAHKPHAR
VGSPSEPFEVDGLPGLRLTRADLNPPIDEPEPTGPLWDLACETKASMDSSEGIIVNSFVELEPLCFDGWSRMSPVKL
WPVGPLCLASELGRNMDRDVSDWLDSRLAMDRPVLYVAFGSQADLSRTQLEEIALGLDQSGLDFLWVVRSKWFDSED
HFENRFGDKGKVYQGFIDQVGVLSHKSIKGFFSHCGWNSVLESISMGVPILAFPMAAEQKLNAKFVVDMLRVGLRVW
PQKREDDMENGLVAREEVQVMARELIFGEEGKWASTRVSELAVLSKKAMEIGGSSYKKLEEMVHEISELTRDKSM
>LOC_Os06g17110.1
(SEQ ID NO: 84)
MVIRGWAPQLAALRHRAVGWFVTHSGWISVVEAVAAGVAMLTWPMVADQFVNARLVVDELRAAVPVSWGGVAVPPSA
NEVARVLEATVLAADGGEVGARVEELAVEAAAATREGGSSWVEVDELVRELGGHMQR
>LOC_Os06g18790.1
(SEQ ID NO: 85)
MMSLCSYFPIYLDNKDAQADVGDVDVPGVRHLQRSWLPQPLLDLDMLFTKQFIENGREVVKTDGVLINTFDALEPVA
LAALRDGTVVRGFPPVFAVGPYSSLASEKKAADADQSSALAWLDQQPARSVVYVAFGNRCTVSNDQLREIAAGLEAS
GCRFLWILKTTVVDRDEAAAGGVRDVLGDGFMERVKGRGMVTKEWVDQEAVLGHPAVGLFLSHSGWNSVTEAAAAGV
PLLAWPRGGDHRVAATVVASSGVGVWMEQWSWDGEEWLVSGEEIGGKVKEMMADDAVRERAAKVGEEAAKAVAEGGT
SHTSMLEFVAKLKAA
>LOC_Os06g23560.1
(SEQ ID NO: 86)
MAAARRVVLFPSLGVGHLAPMLELAAVCIRHGLAVTVAVPDPATTAPAFSAALRKYASRLPSLSVHPLPPPPHPPAS
SGADAAAHPLLRMLAVLRAHAPALGDLLRGPHAARALVADMFSVYALDVAAELGVPGYLLFCTGATNLAVFLRLPRF
CAGSSGSLRELGDAPVSFPGVRPLPASHLPEEVLDRGTDISAAMLDAFDRMADARGILVNTFDALEGPGVAALRDGR
CLSNRATPPVYCVGPLITDGGAEEERHPCLAWLDAQPERSVVFLCFGSRGALSPEQVSEMATGLERSEQRFLWALRA
PAGTKPDAAMSLLPDGFLARTADRGVVVTASWVPQVAVLQHASTGAFVTHCGWNSTLEAVAAGVPMVCWPLDAEQWM
NKVFIVEEMKIGIEVRGYKPGALVQADIVDAILRRIMESDAQQGVLERVMAMKESAAAAWKEGGSSCTAFAEFLKDM
EEGNVAMAHSNQVET
>LOC_Os07g10160.1
(SEQ ID NO: 87)
MAASASSSSPLHIVMFPWLAFGHMIPFLELAKRLARRGLAVTFVSTPRNAARLGAIPPALSAHLRVVPLDLPAVDGL
PEGAESTADAPPEKVGLLKKAFDGLAAPFAGFVAEACAAGHGESTPTAAGFSRKPDWIILDFAQNWVWPIAEEHKIP
CAMFSIFPAAMVAFVGPRQENLAHPRTKTEHFMVQPPWIPFPSNVAYRRRHGAEWIAAVFRPNASGVSDADRFWEME
HACCRLIIHRSCPEAEPRLFPLLTELFAKPSVPAGLLMPPPPPAAGVDDDDDDVSMDDQHIAMAMRWLDEQPERSVI
YVALGSEAPLTVGHVRELALGLELAGVRFLWALRAPPSASSVNRDKCAADADLLLPDGFRSRVAAARGGLVCARWVP
QLRILAHRATGGFLTHCGWSSIFESLRFALPLVMLPLFADQGLGVQALPAREIGVEVACNDDGSFRRDAIAAAVRQV
MVEEKGKALSRKAEELRDVLGDEGRQEMYLDELVGYLQRYK
>LOC_Os07g10190.1
(SEQ ID NO: 88)
MAATSDSTPAAAAAAAASSSSSPLHIVVFPWLAFGHMIPFLELSKRLASRGHAVTFVTTPRNAARLGATPPAPLSSS
SRLRVVPLDLPAVDGLPEGAESTADVPPEKVGLLKKAFDGLAAPFARFVAEACAAGDGEAVTAAAGFLRKPDWIIPD
FAHSWIWPIAEEHKIPYATFLIVPAALVAILGPRRENLTHPRTTAEDYMVQPPWIPFPSNIAYRRRHEAEWMVAAFR
ANASGVSDMDRFWESEQHPNCRLIIYRTCPEIEPRLFPLLTELYTKPAIPSGLLVPPALDDNDIGVYNRSDRSFVAV
MQWLDKQPNKSVIYVSLGTEAPITADHMHELAFGLELAGVRFLWALRRPSGINCHDDMLLPSGFETRVAARGLVCTE
WVPQVRMLAHGAVGVFLTHCGWGSTVESFHYGQPLVMLPFIADQGLIAQAVAATGVGVEVARNYDDGSFYRDDVAAA
IQRVMVEEEGKELAHKAIELCGILGDRVQQEMYLYELIGYLQCYK
>LOC_Os07g10230.1
(SEQ ID NO: 89)
MKQSGSLLHSSLTPLECPMLTVCWKWNAPAAALSSCPEAEPRLFPLLNKLFARPAVPASLLLPADIVHDEDAPNTTS
NQSFVSAIQWLDKQPNGSVIYVALGSEAPITTNHVRELALGLELSGVRFLWALRPPSGINSQTGTFLPSGFESRVAT
RGIVCTEWVPQVRVLAHGAIGAFLTHCGWGSTVESFCFGHPLVMLPFVADQGLIAQAMAARGIGVEVARNYDDGSFY
RDDVAAAVRRVMVEEEGKVLARKAKEVHSILGDRAREEQYLDEFVGYLQRYK
>LOC_Os07g10240.1
(SEQ ID NO: 90)
MAINGVAGAGAGDVDVDVDVDASAPPPPLHLVMFPWLAFGHLIPFLQLAKRLAARGHAAVTFLATPRNASRLAALPP
ELAAYVRVVSLPLPVLDGLPEGAESTADVPPEKVELLKKAFDGLAAPFAAFLADACAAGDREGRPDPFSRRPDWVVV
DFAHGWLPPIADEHRVPCAFFSIYSAAALAFLGPKAAHDAHPRTEPEDFMSPPPWITFPSTIAFRRHEAAWVAAAAY
RPNASGVSDIDRMWQLHQRCHLIVYRSCPDVEGAQLCGLLDELYHKPVVPAGLLLPPDAAGDDDDGHRPDLMRWLDE
QPARSVVYVALGTEAPVTADNVRELALGLELAGARFLWALRDAGERLPEGYKARVAGRSVVEAGWVPQVRVLAHAAV
GAFLTHCGWGSTVESLRFGGLPLVMLPFIADQGLIARAMADRGLGVEVARDDDGDGSFRGEDVAAAVRRVMAEEEGK
VFARNAREMQEALGDGERQDRYVDELAERLRRRRSLS
>LOC_Os07g13780.1
(SEQ ID NO: 91)
MASASVVGGGARHGGERRRRVLVFPLPFQGHTNPMLQLAGALHGRGGLCVTVLHTRFNALDPSRHPELAFVEVADGI
PPDVAARGRVAEIILAMNAAMEATEDESGAASPSNIREVLASVVAAGEGQPSVACLVIDSHLLAVQKAAAGLGIPTL
VLRTGSAACLRCYLAYDMLLQKAICLPKVRTKQSHIFIHPRLKL
>LOC_Os07g13810.1
(SEQ ID NO: 92)
MATQEREPERQPHAGRRVALFPLPFQGHLSPMLQLADLLRARGLAVTVLHTRSNAPDPARHRHGPDLAFLPIHEAAL
PEEATSPGADIVAQLLALNAACEAPFRDALASLLPGVACAVVDGQWYAALGAAARLGVPALALRTDSAATFRSMLAF
PRLRDAGFIPIQGERLDEAVPELEPLRVRDLIRVDGCETEALCGFIARVADAMRDSASGVVVNTFDAIEASELGKIE
AELSKPTFAVGPLHKLTTARTAAEQYRHFVRLYGPDRACLAWLDAHPPRSVLYVSLGSVACIDHDMFDEMAWGLAAS
GVPFLWVNRPGSVRGCMPALPYGVDVSRGKIVPWAPQRDVLAHPAIGGFWTHCGWNSTLESVCEGVPMLARPCFADQ
TVNARYVTHQWGVGLELGEVFDRDRVAVAVRKLMVGEEGAAMRETARRLKIQANQCVAATLAIDNLVKYICSL
>LOC_Os07g13940.1
(SEQ ID NO: 93)
MTGARRHCRRVVMFPFPFRSHIAPMLQLAELLRGRGLAVTVVRTTFNAPDAARHPELIFVPIHERLPDAATDPGTDL
VEQMLALNAACEAPFREALRRVWYWYAALTAAAEVGVAALALRTDNAAALHCMLSYSRLRYSGYLPIKGKLFPESRD
EVLPPVEPLRGRDLIRVDGGDAERVREFIARVDNAMRTAAMGFVINTFRAIEKPVLRNIRRHLPRIPAFAIGPMHRL
LGAPEEHGLHAPDSGCVAWLHAHSPRSVLYVSLGSVARIDREVFDEMALGLAGSGVPFLWVIRPGFVTGIVSDALPL
TEPLTAVVDNGMGKPCFGDQTVNARYVTHQWGVGLELGEVFDRDRVAEAVRKLMVGEEGAAMRDKARGLKAKASKSV
EDDGASNAAIDRLVRYMVSF
>LOC_Os07g42970.1
(SEQ ID NO: 94)
MASPAASKPHVVLIPYPAQGHVTFVHTEFNRARLLRSRGAAAVAGADGLPPPGQPAELDATQDIWAICEATRRTGPG
HVRALVERLGREAAAGGVPPVSFVVADGAMGFAVHVTKEMGIPTYLFFTHSACGLLAYLNFDQLVKRGYVPLKYESC
LTNGYLDTRLDWVAGMIAGVRLRDLPTFIRTTDPDDVMLNITMKQCELDAPAADGILLNTFDGLERAALDAIRARLP
NTIAREDGRCAAWLDAHADAAVVYANFGSITVMGRAQVGEFARGLAAAGAPFLWVIRPDMVRDAGDGDGEPLLPEGF
EEEVVASGSGRGLMVGWCDQEAVLGHRATGAFLSHCGWNSTVESLAAGVPMLCWPFFSEQVTNCRYACEEWGVGVEM
ARDAGRREVEAAVREVMGGGEKAAAMRRKAAAAVAPGGSSRRNLESLFAEIAGGVQPIGLCQFIRGNCDIVGVKNGN
EDKSILEIDKVTTVASLSTGTLPTTESTEPLNLGKFRTGASLSTDIHIAKWDICRFGFMGRAASV
>LOC_Os08g07170.1
(SEQ ID NO: 95)
MARPHAVVVPYPGSGNINPALQLAKLLHGHGIYITFVNTEHNHRRALAAEGAAAVRGRDGFQFETIPDGLLDADRDA
ADYDLGLSVATSHRCAAPLRDLVARLNGAAAGSADGGGGAPPVTCMVLTALMSFALDVARGLGLPTMVLWGGSAASL
MAHMRIRELRERGYIPLKASGSDQFFRLLLKPEETMSVEKSVQYNQASDQFIFMNNNGLKNDK
>LOC_Os08g07180.1
(SEQ ID NO: 96)
MARPHAVVVPYPGSGNINPALQLAKLLHGHGVYITFVNTEHNHRRIVAAEGAGAVRGRDGFRFEAIPDGMADADHDI
GNYDLALSAATSNRCAAPLRELLARLDDGGAGAPPVTCVVVTALMSFALYVARELGLPTMVLWGSSAAALVTQMRTR
ELRERGYIPLKGNEIKDDRDRTV
>LOC_Os08g07200.1
(SEQ ID NO: 97)
MSSSLSLLLLPPSLSLPSLFLSRGAIGRQGQRRRQRARAGAASGKQRRQRAAETWSWRGSPRAAEMQEWHDVVDDDG
GAQPLADESLLTNGHLDTTIIDWIPGMPPISLGDISSFVRTTDADDFGLRFNEDEANNCTMAGALVLNTFDGLEADV
LAALRAEYPRIFTVGPLGNLLLNAAADDVAGLSLWKQDTECLAWLDAQEMGAVVYVNFGSLTVLTPQQLAEFAWGLA
ATGRPFLWVIRENLVVPGDGGGDALLPTGFAAATEGRRCVATWCPQDRVLRHRAVGCFVTHSGWNSTCEGVAAGVPM
VCWPVFADQYTNCKYACEAWGVGVRLDAEVRREQVAGHVELAMESEEMRRAAARWKAQAEAAARRGGSSYENLQSMV
EVINSFSSKA
>LOC_Os08g07270.1
(SEQ ID NO: 98)
MPPIKLGDMSSFVRTTDPDDFGLRFNEEEANNCTKANALILNTFDELEADVLAALRAEYARIYTIGPLGTLLNHAAD
AIGGGLSLWKQDTECLAWLDTQQPRSAVENLVPGGPNALPPEFVVETDGRRCLATWCSQEQVLRHPAVGCFLTHSGW
NSKCESVASGVPMVCWPVFADQYINRKYACESWDVGLRLDEEVRREQVTAQVKQVMESEEMRQDAARWKAKAEQAAR
LGGSSYKNLQSVVEVIRSFASDSKKAEA
>LOC_Os08g15330.1
(SEQ ID NO: 99)
MTHIWWASVMEGVSSGVPMVCRPFFGNQKMNALLVSHVWGFGMAFDRVMTCDGVATVVVSLVGGKDGCRMRARAQEL
QAKVATMFIEPNGNCRKNFARLVEIICAS
>LOC_Os09g21170.1
(SEQ ID NO: 100)
MSALRPRLEASLAAARPRVGLLVADALLYWAHDAAAGLGVPTVAFYATSMFAHVIRDVILRDNPAAALVAGGAGATF
AVPEFPHVRLTLTDIPVPFNDPSPAGPLIEMDAKMANAIAAHYIEHWDCHHVGHRAWPVGPLCLARQPCRAAGDSAA
AIKPSWMRWLDEMAAAGRAVLYVALGTLNAEPHAQLRELAGGLEASGVDFLW
>LOC_Os09g30980.1
(SEQ ID NO: 101)
MKKTVVLYPGLAVGHFNPMMVLADVFLDHGYAVAVALINPSVKDDDAAFTAAVARAVSSKSSATVSFHMLPRIPDPP
SLAFDDDKFFTNYFDLVRRYDEHLHDFLCSVQGLHAVVVDASCGFAIQAVRKLGVPAYELYPCDAGALAVNIQIPSL
LAGFKKLGGGEEGSAPLELLGVPPMSASHVTDLFGRSLSELISKDPEATTVAAGARVMAEFDGILINTFVSLEERAL
RALADPRCCPDGVVLPPVYAVGPLVDKAAAGAGDETSRRHESLVWLDGQPDRSIVFLCFGSIGGNHAEQQLREIAAG
LDKSGHRFLWVVRRAPSTEHLDALLPEGFLARTSGRGLVVNTWVPQPSVLRHRATAAFVTHCGWNSVLEGITAGVPM
LCWPMYAEQRINKVLMVDDMGVGVEMEGWLEGWVTAEEVEAKVRLVVESEHGRKLRERVEAHRDGAAMAWKDGGSSR
VAFARLMTELDNAQR
>LOC_Os10g07970.1
(SEQ ID NO: 102)
MMPRRPTRRRASPRPTLPSRSASCRRRPRPARTPARTVSGAASTRSGSPTRCSWSSSARCRPLSTRSCSTCSASTRS
TSRPSSPSPHTSSSPPRQAPSPSSSTSRITTPTGRHSGRWDKESETTKIRLYQFKRMMEGKGVLVNSFDWLEPKALK
ALAAGVCVPDKPTPSVYCVGPLVDTGNKVGSGAERRHACLVWLDAQPRRSVVFLSFGSQGALPAAQLKEIARGLESS
GHRFLWVVRSPPEEQATSPEPDLERLLPAGFLERTKGTGMVAKNWAPQAEVVQHEAVGVFVTHCGWNSTLEAIMSAL
PMICWPLYAEQAMNKVIMVEEMKIAVPLDGYEEGGLVKAEEVEAKVRLVMETEEGRKLREKLVETRDMALDAVKEGG
SSEVAFDEFMRDLEKSSLENGVCS
>LOC_Os10g09990.1
(SEQ ID NO: 103)
MAAASAAKELHFLLVPLVAQGHIIPMVDLARLLAGRGARVTVVTTPVNAARNRAAVEGARRGGLAVELAEITFTGPE
FGLPEGVENMDQLVDIAMYLAFFKAVWNMEAALEAYVRALPRRPDCVVADACNPWTAAVCERLAIPRLVLHCPSVYF
LLAIHCLAKHGVYDRVADQLEPFEVPGFPVRAVVNTATCRGFFQWPGAEKLARDVVDGEATADGLLLNTFRDVEGVF
VDAYASALGLRAWAIGPTCAARLDDADSSASRGNRAVVDAARIVSWLDARPPASVLYVSFGSLTHLRATQAIELARG
LEESGWPFVWAIKEATAAAVSEWLDGEGYEERVSDRGLLVRGWAPQVTILSHPAAGGFLTHCGWNATLEAISHGVPA
LTWPNFSDQFSSEQLLVDVLRVGVRSGVTVPPMFLPAEAEGVQLTSDGVVKAVTELMDGGDEGTARRARAKELAAKA
RAAMEEGGSSHADLTDVIGYVSEFSAKKRQERDAGETAQQPPPSPAELGDISGDKVEADPALSVQS
>LOC_Os10g12120.1
(SEQ ID NO: 104)
MLAVCRHLVAADAALSVTVVVTEEWHALLESAGVPAALPDRISFATIPNVIPSEHGRGADHIGFIVAVHTRMAAAVE
WLLDRLLLEQKWRPDAIVADTYLAWGVAVGARRGIPVCSLWTMAATFFWALYHFNLWPPVDGSESEQELSCRSLEQY
VPGLSSVRLSDIKTFRASWERPMKIAEEALVNVRKAQCILFTSFHELEPEIINRIAETVPCPIYPIGPSIPHLPRNG
DDPGKIGNDDHHSWLDARQENSVLYVSFGSYVTSESNHKN
>LOC_Os10g17489.1
(SEQ ID NO: 105)
MAAAAAAAADHDAAPRAHALILPYPAQGHVIPLMELAYCLIDRGFAVTFVNTEHNHRRVVAAAAGAGGVQAPGSRAR
RLRLVAVADGMGDGDDRDNLVRLNAVMEEAIPPQLEPILDGAGGEGQLGKVTCVVVDVGMSWALDAVKRRGLPGAAL
WAASAAVLAVLLGAQKLIRDGVIDDDGAPLKLENNSFRLSEFTPPMDATFLAWNFMGNRDAERMVFHYLTSSARAAA
AKADILLCNSFVELEPAIFTLKSPATILPIGPLRTGQRFAHQVEVVGHFWQTNDDTCLSFLDEQPYGSVVYVAFGSL
TIMSPGQLKELALGLEASGHPFLWVVRPGLAGNLPTSFLDATMGQGKGIVVEWAPQEQVLAHPAVGCFVTHCGWNST
VESIRNGVPMLCWPYFTDQFTNQIYICDIWRIGLKMVQTCGEGIVTKEIMVERLKELLLDEGIKERVQRLKEFAETN
MSEEGESTSNLNAVVELMTRPMS
>LOC_Os10g30570.1
(SEQ ID NO: 106)
MGQQQPQDAVAANGNGGGKRPHAVVIPYPLQGHVIPAVHLALRLAARGFAVTFVNTESVHRQITSSGGGHGVGGGDD
IFAGAGGGAMIRYELVSDGFPLGFDRSRNHDQYMEGVLHVLPAHVDELLRRVVGDGDAAAATCLVADTFFVWPATLA
RKLGVPYVSFWTEPAIIFSLYYHMDLLTKNGHFNCKAAPSSSSLPSPILPHASILDADSIPRILSLTGDSMEVDEVM
GSSESEKSARRDAVTVARLMLS
>LOC_Os04g35030.1
(SEQ ID NO: 107)
MAAASGEKEEEEKKLQERAPIRRTAWMLANFVVLFLLLALLVRRATAADAEERGVGGAAWRVAFACEAWFAFVWLLN
MNAKWSPARFDTYPENLAGRCGAAHRPRKSSCISGHLDLMRRQCALMQDRRAAGGRHVRDDGGPGARAAGGDGEQGA
LAARRRLLPGRRRRRRRRRLACYVSDDGCSPVTYYALREAAGFARTWVPFCRRHGVAVRAPFRYFASAPEFGPADRK
FLDDWTFMKSEYDKLVRRIEDADETTLLRQGGGEFAEFMDAKRTNHRAIVKVIWDNNSKNRIGEEGGFPHLIYVSRE
KSPGHHHHYKAGAMNALTRVSAVMTNAPIMLNVDCDMFANDPQVVLHAMCLLLGFDDEISSGFVQVPQSFYGDLKDD
PFGNKLEVIYKKLLGGVAGI
>LOC_Os06g39970.1
(SEQ ID NO: 108)
MDGESPEIMPVECPDPEPASSESGDDHDIPEPLSSRLSVPSGELNLYRAAVALRLVLLAAFFRYRVTRPVADAHALW
VTSVACELWLAASWLIAQLPKLSPANRVTYLDRLASRYEKGGEASRLAGVDVFVAAADAAREPPLATANTVLSVLAA
DYPAGGVACYVHDDGADMLVFESLFEAAGFARRWIPFCRRHGVEPRAPELYFARGVDYLRDRAAPSFVKDRRAMKRE
YEEFKVRMNHLAARARKVPEEGWIMSDGTPWPGNNSRDHPAMIQVLLGHPGDRDVDGGELPRLFYVSREKRPGFRHH
GKAGAMNALLRVSAVLTNGAYVLNLDCDHCVNNSSALREAMCFMMDPVAGNRTCFVQFALRDSGGGDSVFFDIEMKC
LDGIQGPVYVGSGCCFSRKALYGFEPAAAADDGDDMDTAADWRRMCCFGRGKRMNAMRRSMSAVPLLDSEDDSDEQE
EEEAAGRRRRLRAYRAALERHFGQSPAFIASAFEEQGRRRGGDGGSPDATVAPARSLLKEAIHVVSCAFEERTRWGK
EIGWMYGGGVATGFRMHARGWSSAYCSPARPAFRRYARASPADVLAGASRRAVAAMGILLSRRHSPVWAGRRLGLLQ
RLGYVARASYPLASLPLTVYCALPAVCLLTGKSTFPSDVSYYDGVLLILLLFSVAASVALELRWSRVPLRAWWRDEK
LWMVTATSASLAAVFQGILSACTGIDVAFSTETAASPPKRPAAGNDDGEEEAALASEITMRWTNLLVAPTSVVVANL
AGVVAAVAYGVDHGYYQSWGALGAKLALAGWVVAHLQGFLRGLLAPRDRAPPTIAVLWSVVFVSVASLLWVHAASFS
APTAAPTTEQPIL
>LOC_Os07g36610.1
(SEQ ID NO: 109)
MALSPAAAGRTGRNNNNDAGLADPLLPAGGGGGGGKDKYWVPADEEEEICRGEDGGRPPAPPLLYRTFKVSGVLLHP
YRLLTLVRLIAVVLFLAWRLKHRDSDAMWLWWISIAGDFWFGVTWLLNQASKLNPVKRVPDLSLLRRRFDDGGLPGI
DVFINTVDPVDEPMLYTMNSILSILATDYPADRHAAYLSDDGASLAHYEGLIETARFAALWVPFCRKHRVEPRAPES
YFAAKAAPYAGPALPEEFFGDRRLVRREYEEFKARLDALFTDIPQRSEASVGNANTKGAKATLMADGTPWPGTWTEP
AENHKKGQHAGIVKVMLSHPGEEPQLGMPASSGHPLDFSAVDVRLPILVYIAREKRPGYDHQKKAGAMNAQLRVSAL
LSNAPFIFNFDGDHYINNSQAFRAALCFMLDCRHGDDTAFVQFPQRFDDVDPTDRYCNHNRVFFDATLLGLNGVQGP
SYVGTGCMFRRVALYGADPPRWRPEDDDAKALGCPGRYGNSMPFINTIPAAASQERSIASPAAASLDETAAMAEVEE
VMTCAYEDGTEWGDGVGWVYDIATEDVVTGFRLHRKGWRSMYCAMEPDAFRGTAPINLTERLYQILRWSGGSLEMFF
SRNCPLLAGCRLRPMQRVAYANMTAYPVSALFMVVYDLLPVIWLSHHGEFHIQKPFSTYVAYLVAVIAMIEVIGLVE
IKWAGLTLLDWWRNEQFYMIGATGVYLAAVLHIVLKRLLGLKGVRFKLTAKQLAGGARERFAELYDVHWSPLLAPTV
VVMAVNVTAIGAAAGKAVVGGWTPAQVAGASAGLVFNVWVLVLLYPFALGIMGRWSKRPCALFALLVAACAAVAAGF
VAVHAVLAAGSAAPSWLGWSRGATAILPSSWRLKRGF
>LOC_Os07g36680.1
(SEQ ID NO: 110)
MSMAGDVWFGFSWVLNQLPKLSPIKRFPDLAALADRHSDELPGVDVFVTTVDPVDEPILYTVNTILSILAADYPVDS
SRRKSLAKAISEKAANEVEVASASPQMAAHFGYRRESQMGRESVVYIGGTQETGGRPPEKLESILGCYFFSFFLTDI
YGSHVCHVRQNHYLNSEGLDLLRFSKMEEVLYPVLQLRETLWEDDMGHRGERTGGIRGGRGVGQAASTRR
>LOC_Os07g36690.1
(SEQ ID NO: 111)
MAATAASTMSAAAAVTRRINAALRVDATSGDVAAGADGQNGRRSPVAKRVNDGGGGKDDVWVAVDEKDVCGARGGDG
AARPPLFRTYKVKGSILHPYRFLILLRLIAIVAFFAWRVRHKNRDGVWLWTMSMVGDVWFGFSWVLNQLPKLSPIKR
VPDLAALADRHSGDLPGVDVFVTTVDPVDEPILYTVNTILSILAADYPVDRYACYLSDDGGTLVHYEAMVEVAKFAE
LWVPFCRKHCVEPRSPENYFAMKTQAYKGGVPGELMSDHRRVRREYEEFKVRIDSLSSTIRQRSDVYNAKHAGENAT
WMADGTHWPGTWFEPADNHQRGKHAGIVQVLLNHPSCKPRLGLAASAENPVDFSGVDVRLPMLVYISREKRPGYNHQ
KKAGAMNVMLRVSALLSNAPFVINFDGDHYVNNSQAFRAPMCFMLDGRGRGGENTAFVQFPQRFDDVDPTDRYANHN
RVFFDGTMLSLNGLQGPSYLGTGTMFRRVALYGVEPPRWGAAASQIKAMDIANKFGSSTSFVGTMLDGANQERSITP
LAVLDESVAGDLAALTACAYEDGTSWGRDVGWVYNIATEDVVTGFRMHRQGWRSVYASVEPAAFRGTAPINLTERLY
QILRWSGGSLEMFFSHSNALLAGRRLHPLQRVAYLNMSTYPIVTVFIFFYNLFPVMWLISEQYYIQRPFGEYLLYLV
AVIAMIHVIGMFEVKWAGITLLDWCRNEQFYMIGSTGVYPTAVLYMALKLVTGKGIYFRLTSKQTTASSGDKFADLY
TVRWVPLLIPTIVIIVVNVAAVGVAVGKAAAWGPLTEPGWLAVLGMVFNVWILVLLYPFALGVMGQWGKRPAVLFVA
MAMAVAAVAAMYVAFGAPYQAELSGGAASLGKAAASLTGPSG
>LOC_Os07g36700.1
(SEQ ID NO: 112)
MSAAAAVTSWTNGCWSPAATRVNDGGKDDVWVAVDEADVSGARGSDGGGRPPLFQTYKVKGSILHPYRFLILARLIA
IVAFFAWRIRHKNRDGAWLWTMSMVGDVWFGFSWVLNQLPKQSPIKRVPDIAALADRHSGDLPGVDVFVTTVDPVDE
PILYTVNTILSILAADYPVDRYACYLSDDGGTLVHYEAMVEVAKFAELWVPFCRKHCVEPRSPENYFAMKTQAYKGG
VPGELMSDHRRVRREYEEFKVRIDSLSSTIRQRSDVYNAKHAGENATWMADGTHWPGTWFEPADNHQRGKHAGIVQV
LLNHPSCKPRLGLAASAENPVDFSGVDVRLPMLVYISREKRPGYNHQKKAGAMNVMLRVSALLSNAPFVINFDGDHY
VNNSQAFRAPMCFMLDGRGRGGENTAFVQFPQRFDDVDPTDRYANHNRVFFDGTMLSLNGLQGPSYLGTGTMFRRVA
LYGVEPPRWGAAASQIKAMDIANKFGSSTSFVGTMLDGANQERSITPLAVLDESVAGDLAALTACAYEDGTSWGRDV
GWVYNIATEDVVTGFRMHRQGWRSVYASVEPAAFRGTAPINLTERLYQILRWSGGSLEMFFSHSNALLAGRRLHPLQ
RVAYLNMSTYPIVTVFIFFYNLFPVMWLISEQYYIQRPFGEYLLYLVAVIAMIHVIGMFEVKWAGITLLDWCRNEQF
YMIGSTGVYPTAVLYMALKLVTGKGIYFRLTSKQTAASSGDKFADLYTVRWVPLLIPTIVIMVVNVAAVGVAVGKAA
AWGPLTEPGWLAVLGMVFNVWILVLLYPFALGVMGQWGKRPAVLFVAMAMAVAAVAAMYVAFGAPYQAELSGVAASL
GKVAAASLTGPSG
>LOC_Os07g36740.1
(SEQ ID NO: 113)
MSAAAVTRRINAGGLRVEVTNGNGAAGVYVAAAAAPCSPAAKRVNDGGGKDDVWVAVDEADVSGPSGGDGVRPTLFR
TYKVKGSILHPYRFLILVRLIAIVAFFAWRVRHKNRDGAWLWTMSMAGDVWFGFSWALNQLPKLNPIKRVADLAALA
DRQQHGTSGGGELPGVDVFVTTVDPVDEPILYTVNSILSILAADYPVDRYACYLSDDGGTLVHYEAMVEVAKFAELW
VPFCRKHCVEPRAPESYFAMKTQAYRGGVAGELMSDRRRVRREYEEFKVRIDSLFSTIRKRSDAYNRAKDGKDDGEN
ATWMADGTHWPGTWFEPAENHRKGQHAGIVQVLLNHPTSKPRFGVAASVDNPLDFSGVDVRLPMLVYISREKRPGYN
HQKKAGAMNALLRVSALLSNAPFIINFDCDHYVNNSQAFRAPMCFMLDRRGGGDDVAFVQFPQRFDDVDPTDRYANH
NRVFFDGTTLSLNGLQGPSYLGTGTMFRRAALYGLEPPRWGAAGSQIKAMDNANKFGASSTLVSSMLDGANQERSIT
PPVAIDGSVARDLAAVTACGYDLGTSWGRDAGWVYDIATEDVATGFRMHQQGWRSVYTSMEPAAFRGTAPINLTERL
YQILRWSGGSLEMFFSHSNALLAGRRLHPLQRIAYLNMSTYPIVTVFIFFYNLFPVMWLISEQYYIQQPFGEYLLYL
VAIIAMIHVIGMFEVKWSGITVLDWCRNEQFYMIGSTGVYPTAVLYMALKLFTGKGIHFRLTSKQTTASSGDKFADL
YTVRWVPLLIPTIVVLAVNVGAVGVAVGKAAAWGLLTEQGRFAVLGMVFNVWILALLYPFALGIMGQRGKRPAVLFV
ATVMAVAAVAIMYAAFGAPYQAGLSGVAASLGKAASLTGPSG
>LOC_Os07g36750.1
(SEQ ID NO: 114)
MASPASVAGGGEDSNGCSSLIDPLLVSRTSSIGGAERKAAGGGGGGAKGKHWAAADKGERRAAKECGGEDGRRPLLF
RSYRVKGSLLHPYRALIFARLIAVLLFFGWRIRHNNSDIMWFWTMSVAGDVWFGFSWLLNQLPKFNPVKTIPDLTAL
RQYCDLADGSYRLPGIDVFVTTADPIDEPVLYTMNCVLSILAADYPVDRSACYLSDDSGALILYEALVETAKFATLW
VPFCRKHCIEPRSPESYFELEAPSYTGSAPEEFKNDSRIVHLEYDEFKVRLEALPETIRKRSDVYNSMKTDQGAPNA
TWMANGTQWPGTWIEPIENHRKGHHAGIVKVVLDHPIRGHNLSLKDSTGNNLNFNATDVRIPMLVYVSRGKNPNYDH
NKKAGALNAQLRASALLSNAQFIINFDCDHYINNSQAFRAAICFMLDQREGDNTAFVQFPQRFDNVDPKDRYGNHNR
VFFDGTMLALNGLQGPSYLGTGCMFRRLALYGIDPPHWRQDNITPEASKFGNSILLLESVLEALNQDRFATPSPVND
IFVNELEMVVSASFDKETDWGKGVGYIYDIATEDIVTGFRIHGQGWRSMYCTMEHDAFCGTAPINLTERLHQIVRWS
GGSLEMFFSHNNPLIGGRRLQPLQRVSYLNMTIYPVTSLFILLYAISPVMWLIPDEVYIQRPFTRYVVYLLVIILMI
HMIGWLEIKWAGITWLDYWRNEQFFMIGSTSAYPTAVLHMVVNLLTKKGIHFRVTSKQTTADTNDKFADLYEMRWVP
MLIPTMVVLVANIGAIGVAIGKTAVYMGVWTIAQKRHAAMGLLFNMWVMFLLYPFALAIMGRWAKRSIILVVLLPII
FVIVALVYVATHILLANIIPF
>LOC_Os10g20260.1
(SEQ ID NO: 115)
MPPSAGLATESLPAATCPAKKDAYAAAASPESETKLAAGDERAPLVRTTRISTTTIKLYRLTIFVRIAIFVLFFKWR
ITYAARAISSTDAGGIGMSKAATFWTASIAGELWFAFMWVLDQLPKTMPVRRAVDVTALNDDTLLPAMDVFVTTADP
DKEPPLATANTVLSILAAGYPAGKVTCYVSDDAGAEVTRGAVVEAARFAALWVPFCRKHGVEPRNPEAYFNGGEGGG
GGGKARVVARGSYKGRAWPELVRDRRRVRREYEEMRLRIDALQAADARRRRCGAADDHAGVVQVLIDSAGSAPQLGV
ADGSKLIDLASVDVRLPALVYVCREKRRGRAHHRKAGAMNALLRASAVLSNAPFILNLDCDHYVNNSQALRAGICFM
IERRGGGAEDAGDVAFVQFPQRFDGVDPGDRYANHNRVFFDCTELGLDGLQGPIYVGTGCLFRRVALYGVDPPRWRS
PGGGVAADPAKFGESAPFLASVRAEQSHSRDDGDAIAEASALVSCAYEDGTAWGRDVGWVYGTVTEDVATGFCMHRR
GWRSAYYAAAPDAFRGTAPINLADRLHQVLRWAAGSLEIFFSRNNALLAGGRRRLHPLQRAAYLNTTVYPFTSLFLM
AYCLFPAIPLIAGGGGWNAAPTPTYVAFLAALMVTLAAVAVLETRWSGIALGEWWRNEQFWMVSATSAYLAAVAQVA
LKVATGKEISFKLTSKHLASSATPVAGKDRQYAELYAVRWTALMAPTAAALAVNVASMAAAGGGGRWWWWDAPSAAA
AAAAALPVAFNVWVVVHLYPFALGLMGRRSKAVRPILFLFAVVAYLAVRFLCLLLQFHTA
>LOC_Os10g42750.1
(SEQ ID NO: 116)
MASKGILKNGGKPPTAPSSAAPTVVFGRRTDSGRFISYSRDDLDSEISSVDFQDYHVHIPMTPDNQPMDPAAGDEQQ
YVSSSLFTGGFNSVTRAHVMEKQASSARATVSACMVQGCGSKIMRNGRGADILPCECDFKICVDCFTDAVKGGGGVC
PGCKEPYKHAEWEEVVSASNHDAINRALSLPHGHGHGPKMERRLSLVKQNGGAPGEFDHNRWLFETKGTYGYGNAIW
PEDDGVAGHPKELMSKPWRPLTRKLRIQAAVISPYRLLVLIRLVALGLFLMWRIKHQNEDAIWLWGMSIVCELWFAL
SWVLDQLPKLCPINRATDLSVLKDKFETPTPSNPTGKSDLPGIDIFVSTADPEKEPVLVTANTILSILAADYPVDKL
ACYVSDDGGALLTFEAMAEAASFANLWVPFCRKHEIEPRNPDSYFNLKRDPFKNKVKGDFVKDRRRVKREYDEFKVR
VNGLPDAIRRRSDAYHAREEIQAMNLQREKMKAGGDEQQLEPIKIPKATWMADGTHWPGTWLQASPEHARGDHAGII
QVMLKPPSPSPSSSGGDMEKRVDLSGVDTRLPMLVYVSREKRPGYDHNKKAGAMNALVRASAIMSNGPFILNLDCDH
YVYNSKAFREGMCFMMDRGGDRLCYVQFPQRFEGIDPSDRYANHNTVFFDVNMRALDGLQGPVYVGTGCLFRRIALY
GFDPPRSKDHTTPWSCCLPRRRRTRSQPQPQEEEEETMALRMDMDGAMNMASFPKKFGNSSFLIDSIPVAEFQGRPL
ADHPSVKNGRPPGALTIPRETLDASIVAEAISVVSCWYEEKTEWGTRVGWIYGSVTEDVVTGYRMHNRGWKSVYCVT
HRDAFRGTAPINLTDRLHQVLRWATGSVEIFFSRNNALFASSKMKVLQRIAYLNVGIYPFTSVFLIVYCFLPALSLF
SGQFIVQTLNVTFLTYLLIITITLCLLAMLEIKWSGIALEEWWRNEQFWLIGGTSAHLAAVLQGLLKVIAGIEISFT
LTSKQLGDDVDDEFAELYAVKWTSLMIPPLTIIMINLVAIAVGFSRTIYSTIPQWSKLLGGVFFSFWVLAHLYPFAK
GLMGRRGRTPTIVYVWSGLVAITISLLWIAIKPPSAQANSQLGGSFSFP
>LOC_Os12g29300.1
(SEQ ID NO: 117)
MDVFVTTADPDGIAALDDDALLPAMDVFVTTADPDKEPPLATANTVLSIYPRRGLPRRQVVQVLIDSAGSVPQLGVA
DGSKLIDVASVDVCLPALVYVCREKRRGHAHHRKAGAMNAPFILDLDCDHYVNNSQALRAGICFMIERGGGGAAEDA
VAVAFVQFPQRVDGVDPSDRYANHNRVFFDCTELGLDGLQGPIYVGTGCLFRRVALYSVDLPRWRPRRSLGCRLLGE
DERLWSRLKQMVI
>LOC_Os03g07350.1
(SEQ ID NO: 118)
MEGQWGRWRLAAAAAASSSGDQIAAAWAVVRARAVAPVLQFAVWACMAMSVMLVLEVAYMSLVSLVAVKLLRRVPER
RYKWEPITTGSGGVGGGDGEDEEAATGGREAAAFPMVLVQIPMYNEKEVYKLSIGAACALTWPPDRIIIQVLDDSTD
PAIKDLVELECKDWARKEINIKYEIRDNRKGYKAGALKKGMEHIYTQQCDFVAIFDADFQPESDFLLKTIPFLVHNP
KIGLVQTRWEFVNYDVCLMTRIQKMSLDYHFKVEQESGSSMHSFFGFNGTAGVWRVSAINEAGGWKDRTTVEDMDLA
VRASLKGWQFLYVGDIRVKSELPSTFKAYRHQQHRWTCGAANLFRKMATEIAKNKGVSVWKKLHLLYSFFFVRRVVA
PILTFLFYCVVIPLSVMVPEVSIPVWGMVYIPTAITIMNAIRNPGSIHLMPFWILFENVMAMHRMRAALTGLLETMN
VNQWVVTEKVGDHVKDKLEVPLLEPLKPTDCVERIYIPELMVAFYLLVCASYDLVLGAKHYYLYIYLQAFAFIALGF
GFAGTSTPCS
>LOC_Os03g26044.1
(SEQ ID NO: 119)
MEAGEAAGAVLFLLAAAVSLLAAVSTGALDFTYLVTVVGEGSSTSPGSGGGAWWREAWVGARSRAVAPALQVGVWAC
MVMSVMLVVEATYNSAVSVAARLVGWRPERWFKWEPLGGGAGAGDEEKGEAAAAAYPMVMVQIPMYNELEVYKLSIG
AVCGLKWPKERLIIQVLDDSTDAFIKNLVELECEDWASKGLNIKYATRSGRKGFKAGALKKGMEWDYAKQCEYVAIF
DADFQPEPDFLLRTVPFLMHNQNVALVQARWVFVNDRVSLLTRIQKTFLDYHFKAEQEAGSATFAFFSFNGTAGVWR
TEAINDAGGWKDRTTVEDMDLAVRATLKGWKFIYLGDLRVKSELPSTYKAYCRQQFRWSCGGANLFRKMIWDVLVAK
KVSSLKKIYILYSFFLVRRVVAPAVAFILYNVIIPVSVMIPELFLPIWGVAYIPTALLIVTAIRNPENLHTVPLWIL
FESVMSMHRLRAAVAGLLQLQEFNQWIVTKKVGNNAFDENNETPLLQKSRKRLINRVNLPEIGLSVFLIFCASYNLV
FHGKNSFYINLYLQGLAFFLLGLNCVGTLPDHCCF
>LOC_Os03g56060.1
(SEQ ID NO: 120)
MVLVQIPMCNEKEVYQQSIAAVCNLDWPRSNFLVQVLDDSDDPTTQTLIREEVLKWQQNGARIVYRHRVLRDGYKAG
NLKSAMSCSYVKDYEFVAIFDADFQPNPDFLKRTVPHFKDNDELGLVQARWSFVNKDENLLTRLQNINLCFHFEVEQ
QVNGIFLNFFGFNGTAGVWRIKALDDSGGWMERTTVEDMDIAVRAHLRGWKFIFLNDVECQCELPESYEAYRKQQHR
WHSGPMQLFRLCLPDIIKCKIVFWKKANLIFLFFLLRKLILPFYSFTLFCIILPMTMFVPEAELPDWVVCYIPALMS
LLNILPSPKSFPFIIPYLLFENTMSVTKFNAMISGLFQLGNAYEWVVTKKSGRSSEGDLISLAPKELKHQKTESAPN
LDAIAKEQSAPRKDVKKKHNRIYKKELALSLLLLTAAARSLLSKQGIHFYFLLFQGISFLLVGLDLIGEQIE
>LOC_Os03g60700.2
(SEQ ID NO: 121)
MAAAGWPLSSSVADLLPASLSLTLLLASLVHPLPPSAPFLLRLLALLIPSPRPSRAQVVVVVLAAAAFFFEHIRKIG
CTHSLERTEVSAAFFEDPNSLNKVRCPSIYDPAEKYISLIIPAYNEEHRLPEALTETLNYLKQRSAVEKSFTYEVLI
VDDGSTDHTSKVAFEFVRKHKIDNVRVLLLGRNHGKGEAVRKGMLHSRGELLLMLDADGATKVTDLEKLEAQVCHKL
NQNMFYKVLLCIL
>LOC_Os03g60939.2
(SEQ ID NO: 122)
MADDAGGGRREYSIIVPTYNERLNVALIVYLIFKHLPDVNFEIIVVDDGSPDGTQDIVKQLQQIYGENRVLLRARPR
KLGLGTAYLHGLKHASGDFVVIMDADLSHHPKYLPSFIRKQKETGADVVTGTRYVQNGGVHGWNLMRKLTSRGANVL
AQTLLQPGASDLTGSFRCYINGMS
>LOC_Os06g12460.1
(SEQ ID NO: 123)
MAMAGADGPTAGAAAAVRWRGGESLLLLLLRWPSSAELVAAWGAARASAVAPALAAASAACLALSAMLLADAVLMAA
ACFARRRPDRRYRATPLGAGAGADDDDDDEEAGRVAYPMVLVQIPMYNEREVYKLSIGAACGLSWPSDRLIVQVLDD
STDPTVKTWYDRLRKTLVQQAHPAQADMDVHQSTKRKNKELMTRVPILECDSNHGLASIISSYLIAVGLVELECKSW
GNKGKNVKYEVRNTRKGYKAGALKEGLLRDYVQQCNYVAIFDADFQPEPDFLLRTIPYLVRNPQIGLVQAHWEFVNT
SECLMTRIQKMTLHYHFKVEQEGGSSTFAFFGFNGTAGVWRISALEEAGGWKDRTTVEDMDLAVRAGLKGWKFVYLA
DVKVKSELPSNLKTYRHQQHRWTCGAANLFRKVGAEILFTKEVPFWWKFYLLYSFFFVRKVVAHVVPFMLYCVVIPF
SVLIPEVTVPVWGVVYVPTTITLLHAIRNTSSIHFIPFWILFENVMSFHRTKAMFIGLLELGGVNEWVVTEKLGNGS
NTKPASQILERPPCRFWDRWTMSEILFSIFLFFCATYNLAYGGDYYFVYIYLQAIAFLVVGIGFCGTISSNS
>LOC_Os07g03260.1
(SEQ ID NO: 124)
MAPWSGFWAASRPALAAAAAGGTPVVVKMDNPNWSISEIDADGGEFLAGGRRRGRGKNAKQITWVLLLKAHRAAGCL
AWLASAAVALGAAARRRVAAGRTDDADAETPAPRSRLYAFIRASLLLSVFLLAVELAAHANGRGRVLAASVDSFHSS
WVRFRAAYVAPPLQLLADACVVLFLVQSADRLVQCLGCLYIHLNRIKPKPISSPAAAAAALPDLEDPDAGDYYPMVL
VQIPMCNEKEVYQQSIAAVCNLDWPRSNILVQVLDDSDDPITQSLIKEEVEKWRQNGARIVYRHRVLREGYKAGNLK
SAMSCSYVKDYEYVAIFDADFQPYPDFLKRTVPHFKDNEELGLVQARWSFVNKDENLLTRLQNINLCFHFEVEQQVN
GIFINFFGFNGTAGVWRIKALEDSGGWMERTTVEDMDIAVRAHLNGWKFVFLNDVECQCELPESYEAYRKQQHRWHS
GPMQLFRLCLPDIIRCKIAFWKKANLIFLFFLLRKLILPFYSFTLFCIILPMTMFIPEAELPDWVVCYIPALMSFLN
ILPAPKSFPFIIPYLLFENTMSVTKFNAMISGLFQLGSAYEWVVTKKSGRSSEGDLIALAPKELKQQKILDLTAIKE
QSMLKQSSPRNEAKKKYNRIYKKELALSLLLLTAAARSLLSKQGIHFYFLMFQGLSFLLVGLDLIGEDVK
>LOC_Os07g43710.1
(SEQ ID NO: 125)
MVEAGEIGGAAVFALAAAAALSAASSLGAVDFRRPLAAVGGGGAFEWDGVVPWLIGVLGGGDEAAAGGVSVGVAAWY
EVWVRVRGGVIAPTLQVAVWVCMVMSVMLVVEATFNSAVSLGVKAIGWRPEWRFKWEPLAGADEEKGRGEYPMVMVQ
IPMYNELEVYKLSIGAACELKWPKDKLIVQVLDDSTDPFIKNLVELECESWASKGVNIKYVTRSSRKGFKAGALKKG
MECDYTKQCEYIAIFDADFQPEPNFLLRTVPFLMHNPNVALVQARWAFVNDTTSLLTRVQKMFFDYHFKVEQEAGSA
TFAFFSFNGTAGVWRTTAINEAGGWKDRTTVEDMDLAVRASLNGWKFIYVGDIRVKSELPSTYGAYCRQQFRWACGG
ANLFRKIAMDVLVAKDISLLKKFYMLYSFFLVRRVVAPMVACVLYNIIVPLSVMIPELFIPIWGVAYIPMALLIITT
IRNPRNLHIMPFWILFESVMTVLRMRAALTGLMELSGFNKWTVTKKIGSSVEDTQVPLLPKTRKRLRDRINLPEIGF
SVFLIFCASYNLIFHGKTSYYFNLYLQGLAFLLLGFNFTGNFACCQ
>LOC_Os08g33740.1
(SEQ ID NO: 126)
MSSSGGGGVAEEVARLWGELPVRVVWAAVAAQWAAAAAAARAAVVVPAVRALVAVSLAMTVMILAEKLFVAAVCLAV
RAFRLRPDRRYKWLPIGAAAAAASSEDDEESGLVAAAAAFPMVLVQIPMFNEREVYKLSIGAACSLDWPSDRVVIQV
LDDSTDLVVKVFIVIYFTDISSRIIRSTSSLVIKDLVEKECQKWQGKGVNIKYEVRGNRKGYKAGALKEGLKHDYVK
ECEYIAMFDADFQPESDFLLRTVPFLVHNSEIALVQTRWKFVNANECLLTRFQEMSLDYHFKYEQEAGSSVYSFFGF
NGTAGVWRIAAIDDAGGWKDRTTVEDMDLAVRATLQGWKFVYVGDVKVKSELPSTFKAYRFQQHRWSCGPANLFKKM
MVEILENKKVSFWNKIHLWYDFFFVGKIAAHTVTFIYYCFVIPVSVWLPEIEIPLWGVVYVPTVITLCKAVGTPSSF
HLVILWVLFENVMSLHRIKAAVTGILEAGRVNEWVVTEKLGDANKTKPDTNGSDAVKVIDVELTTPLIPKLKKRRTR
FWDKYHYSEIFVGICIILSGFYDVLYAKKGYYIFLFIQGLAFLIVGFDYIGVCPP
>LOC_Os09g26770.1
(SEQ ID NO: 127)
MASLRAATGLPFSPRPACCRPPSSPGSRRGFVFPPRFAPGVFLFFPLDSAGGGGVARRRAYPRIEATARHGARKENP
KVRNRRLQKKFNGTATKPRLSVFCSNRQLYAMLVDDHNKKILFYGSTLQKAICGDPPCGAVEAAGRIGEELIRACKE
LDITEISSYDRNGFARGEKMMAFEVPDLVELECIDWARKEINIKYEIRDNRKGYKAGALKKGMEHIYTQQCDFVAIF
DADFQPESDFLLKIIPFLVHNPKIGLVQTRWEFVNYDVCLMTRIQKMSLDYHFKVEQESGSSMHSFFGFNGTAAVWR
VSATINEAGGWKDHTTVEDMDLAVRLLRVNSQVPSKPTDIGSIDGLVGVSVWKKLHLLYSFFFVRRVVAPILTFLFY
RVVIPLSVMVPEISIPVWGMILFENVMAMHRMRAALTGLLETMNVNQWVVTEKVGDHVKDKLEVPLLEPLKPTDCVE
RIYIPELVVAFYLLEGFSGRNATGVNRSKDV
>LOC_Os09g26770.2
(SEQ ID NO: 128)
MASLRAATGLPFSPRPACCRPPSSPGSRRGFVFPPRFAPGVFLFFPLDSAGGGGVARRRAYPRIEATARHGARKENP
KVRNRRLQKKFNGTATKPRLSVFCSNRQLYAMLVDDHNKKILFYGSTLQKAICGDPPCGAVEAAGRIGEELIRACKE
LDITEISSYDRNGFARGEKMMAFEVPDLVELECIDWARKEINIKYEIRDNRKGYKAGALKKGMEHIYTQQCDFVAIF
DADFQPESDFLLKIIPFLVHNPKIGLVQTRWEFVNYDVCLMTRIQKMSLDYHFKVEQESGSSMHSFFGFNGTAAVWR
VSATINEAGGWKDHTTVEDMDLAVRLLRVNSQVPSKPTDIGSIDGLVGVSVWKKLHLLYSFFFVRRVVAPILTFLFY
RVVIPLSVMVPEISIPVWGMILFENVMAMHRMRAALTGLLETMNVNQWVVTEKVGDHVKDKLEVPLLEPLKPTDCVE
RIYIPELVVAFYLLEGFSGRNATGVNRSKDV
>LOC_Os09g39920.1
(SEQ ID NO: 129)
MMSGGLAWAWRAVRCGVVLPTLQLAVYVCVAMSIMLFLERLYMALVVAALWLIRRRRRRSNRREQDDDGAENDQLLQ
DPEAANSPMVLVQIPMFNEKQVYRLSIGAACGMTWPSDKLVIQVLDDSTDPAIREMVEGECGRWAGKGVSIRYENRR
NRSGYKAGAMREGLRKAYARECELVAIFDADFQPDADFLLRTVPVLVADPGVALVQARWRFVNADECLLTRIQEMSL
DYHFRVEQEVGSACHGFFGFNGTAGVWRVRALEEAGGWKERTTVEDMDLAVRASLRGWRFVYVGHVGVRNELPSTLR
AYRYQQHRWSCGPANLFRKIFLEAPPPACPPGRSSTSSTISSSSASSSPTSSPSPSTASSSPPASSPAPTTSASPST
SPSTSPPPSPSSTPPAPRAPAISSSSGSSSRTSCPCTGPRPRSSACSRPPAPTSGSSPTSEATPTPSTSSQLIPPPG
LGGRPPPAPAAQASSIMTSMSPRSSWGPACSTAPSTTSPTAATASTSTCSSSRPPPSSSASATSGPSSYYYSYSTCI
HV
>LOC_Os10g26630.1
(SEQ ID NO: 130)
MASSSSSSLPAAWAAAVRAWAVAPALRAAVWACLAMSAMLVAEAAWMGLASLAAAAARRLRGYGYRWEPMAAPPDVE
APAPAPAEFPMVLVQIPMYNEKEVYKLSIGAACALTWPPDRIIIQVLDDSTDPFVKFSLVQELVELECKEWASKKIN
IKYEVRNNRKGYKAGALRKGMEHTYAQLCDFVAIFDADFEPESDFLLKTMPYLLHNPKIALVQTRWEFVNYNVCLMT
RIQKMSLDYHFKVEQESGSFMHAFFGFNGTAGVWRVSAINQSGGWKDRTTVEDMDLAVRASLKGWEFLYVGDIRVKS
ELPSTFQAYRHQQHRWTCGAANLFRKMAWEIITNKEVSMWKKYHLLYSFFFVRRAIAPILTFLFYCIVIPLSAMVPE
VTIPVWGLVYIPTAITIMNAIRNPGSVHLMPFWILFENVMAMHRMRAALSGLLETARANDWVVTEKVGDQVKDELDV
PLLEPLKPTECAERIYIPELLLALYLLICASYDFVLGNHKYYIYIYLQAVAFTVMGFGFVGTRTPCS
>LOC_Os01g04920.2
(SEQ ID NO: 131)
MEYPPQFPTPQLHTPISSSSSSSSSPRLYTRRVELLLLLFLAPPQHRRLEAHANAGSEEKDCFFFFFCVCAVFLGFL
AMVIGAEIKDEMEEAPPLLLDEAARPRRVALFVEPSPFAYISGYKNRFQNFIKHLREMGDEVIVVTNHEGVPQEFHG
AKVIGSWSFPCPMYGKVPLSLALSPRIISEVAKFKPDIIHASSPGIMVFGALAIAKLLGVPLVMSYHTHVPVYIPRY
TFSWLVEPMWQVIRFLHRAADLTLVPSVAISKDFETAHVISANRIRLWNKGVDSASFHPKFRSHEMRVRLRTLWA
>LOC_Os01g04920.3
(SEQ ID NO: 132)
MEYPPQFPTPQLHTPISSSSSSSSSPRLYTRRVELLLLLFLAPPQHRRLEAHANAGSEEKDCFFFFFCVCAVFLGFL
AMVIGAEIKDEMEEAPPLLLDEAARPRRVALFVEPSPFAYISGYKNRFQNFIKHLREMGDEVIVVTNHEGVPQEFHG
AKVIGSWSFPCPMYGKVPLSLALSPRIISEVAKFKPDIIHASSPGIMVFGALAIAKLLGVPLVMSYHTHVPV
>LOC_Os02g09170.1
(SEQ ID NO: 133)
MLRHFSALAPSPLLFLLFLPFPWLRLHSSAHSSPPPRSRRDLHGGGGGMAGNDNWINSYLDAILDAGKAAIGGDRPS
LLLRERGHFSPARYFVEEVITGYDETDLYKTWLRANAMRSPQERNTRLENMTWRIWNLARKKKEFEKEEACRLLKRQ
PEAEKLRTDTNADMSEDLFEGEKGEDAGDPSVAYGDSTTGSSPKTSSIDKLYIVLISLHGLVRGENMELGRDSDTGG
QVKYVVELAKALSSSPGVYRVDLLTRQILAPNFDRSYGEPTEMLVSTSFKNSKQEKGENSGAYIIRIPFGPKDKYLA
KEHLWPFIQEFVDGALGHIVRMSKTIGEEIGCGHPVWPAVIHGHYASAGIAAALLSGSLNIPMAFTGHFLGKDKLEG
LLKQGRHSREQINMTYKIMCRIEAEELSLDASEIVIASTRQEIEEQWNLYDGFEVILARKLRARVKRGANCYGRYMP
RMVIIPPGVEFGHIIHDFEMDGEEENPCPASEDPPIWSQIMRFFTNPRKPMILAVARPYPEKNITSLVKAFGECRPL
RELANLTLIMGNREAISKMNNMSAAVLTSVLTLIDEYDLYGQVAYPKHHKHSEVPDIYRLAARTKGAFVNVAYFEQF
GVTLIEAAMNGLPIIATKNGAPVEINQVLNNGLLVDPHDQNAIADALYKLLSDKQLWSRCRENGLKNIHQFSWPEHC
KNYLSRILTLGPRSPAIGGKQEQKAPISGRKHIIVISVDSVNKEDLVRIIRNTIEVTRTEKMSGSTGFVLSTSLTIS
EIRSLLVSAGMLPTVFDAFICNSGSNIYYPLYSGDTPSSSQVTPAIDQNHQAHIEYRWGGEGLRKYLVKWATSVVER
KGRIERQIIFEDPEHSSTYCLAFRVVNPNHLPPLKELRKLMRIQSLRCNALYNHSATRLSVVPIHASRSQALRYLCI
RWGIELPNVAVLVGESGDSDYEELLGGLHRTVILKGEFNIPANRIHTVRRYPLQDVVALDSSNIIGIEGYSTDDMKS
ALQQIGVLTQ
>LOC_Os02g09170.2
(SEQ ID NO: 134)
MLRHFSALAPSPLLFLLFLPFPWLRLHSSAHSSPPPRSRRDLHGGGGGMAGNDNWINSYLDAILDAGKAAIGGDRPS
LLLRERGHFSPARYFVEEVITGYDETDLYKTWLRANAMRSPQERNTRLENMTWRIWNLARKKKEFEKEEACRLLKRQ
PEAEKLRTDTNADMSEDLFEGEKGEDAGDPSVAYGDSTTGSSPKTSSIDKLYIVLISLHGLVRGENMELGRDSDTGG
QVKYVVELAKALSSSPGVYRVDLLTRQILAPNFDRSYGEPTEMLVSTSFKNSKQEKGENSGAYIIRIPFGPKDKYLA
KEHLWPFIQEFVDGALGHIVRMSKTIGEEIGCGHPVWPAVIHGHYASAGIAAALLSGSLNIPMAFTGHFLGKDKLEG
LLKQGRHSREQINMTYKIMCRIEAEELSLDASEIVIASTRQEIEEQWNLYDGFEVILARKLRARVKRGANCYGRYMP
RMVIIPPGVEFGHIIHDFEMDGEEENPCPASEDPPIWSQIMRFFTNPRKPMILAVARPYPEKNITSLVKAFGECRPL
RELANLTLIMGNREAISKMNNMSAAVLTSVLTLIDEYDLYGQVAYPKHHKHSEVPDIYRLAARTKGAFVNVAYFEQF
GVTLIEAAMNGLPIIATKNGAPVEINQVLNNGLLVDPHDQNAIADALYKLLSDKQLWSRCRENGLKNIHQFSWPEHC
KNYLSRILTLGPRSPAIGGKQEQKAPISGRKHIIVISVDSVNKEDLVRIIRNTIEVTRTEKMSGSTGFVLSTSLTIS
EIRSLLVSAGMLPTVFDAFICNSGSNIYYPLYSGDTPSSSQVTPAIDQNHQAHIEYRWGGEGLRKYLVKWATSVVER
KGRIERQIIFEDPEHSSTYCLAFRVVNPNHLPPLKELRKLMRIQSLRCNALYNHSATRLSVVPIHASRSQALRYLCI
RWGIELPNVAVLVGESGDSDYEELLGGLHRTVILKGEFNIPANRIHTVRRYPLQDVVALDSSNIIGIEGYSTDDMKS
ALQQIGVLTQ
>LOC_Os02g09170.3
(SEQ ID NO: 135)
MAPRERDAEPAGEEHAAGEHDVEDLEPREEEEGDMSEDLFEGEKGEDAGDPSVAYGDSTTGSSPKTSSIDKLYIVLI
SLHGLVRGENMELGRDSDTGGQVKYVVELAKALSSSPGVYRVDLLTRQILAPNFDRSYGEPTEMLVSTSFKNSKQEK
GENSGAYIIRIPFGPKDKYLAKEHLWPFIQEFVDGALGHIVRMSKTIGEEIGCGHPVWPAVIHGHYASAGIAAALLS
GSLNIPMAFTGHFLGKDKLEGLLKQGRHSREQINMTYKIMCRIEAEELSLDASEIVIASTRQEIEEQWNLYDGFEVI
LARKLRARVKRGANCYGRYMPRMVIIPPGVEFGHIIHDFEMDGEEENPCPASEDPPIWSQIMRFFTNPRKPMILAVA
RPYPEKNITSLVKAFGECRPLRELANLTLIMGNREAISKMNNMSAAVLTSVLTLIDEYDLYGQVAYPKHHKHSEVPD
IYRLAARTKGAFVNVAYFEQFGVTLIEAAMNGLPIIATKNGAPVEINQVLNNGLLVDPHDQNAIADALYKLLSDKQL
WSRCRENGLKNIHQFSWPEHCKNYLSRILTLGPRSPAIGGKQEQKAPISGRKHIIVISVDSVNKEDLVRIIRNTIEV
TRTEKMSGSTGFVLSTSLTISEIRSLLVSAGMLPTVFDAFICNSGSNIYYPLYSGDTPSSSQVTPAIDQNHQAHIEY
RWGGEGLRKYLVKWATSVVERKGRIERQIIFEDPEHSSTYCLAFRVVNPNHLPPLKELRKLMRIQSLRCNALYNHSA
TRLSVVPIHASRSQALRYLCIRWGIELPNVAVLVGESGDSDYEELLGGLHRTVILKGEFNIPANRIHTVRRYPLQDV
VALDSSNIIGIEGYSTDDMKSALQQIGVLTQ
>LOC_Os06g43630.1
(SEQ ID NO: 136)
MYGNDNWINSYLDAILDAGKGAAASASASAVGGGGGAGDRPSLLLRERGHFSPARYFVEEVITGYDETDLYKTWLRA
NAMRSPQEKNTRLENMTWRIWNLARKKKELEKEEANRLLKRRLETERPRVETTSDMSEDLFEGEKGEDAGDPSVAYG
DSTTGNTPRISSVDKLYIVLISLHGLVRGENMELGRDSDTGGQVKYVVELAKALSSCPGVYRVDLFTRQILAPNFDR
SYGEPVEPLASTSFKNFKQERGENSGAYIIRIPFGPKDKYLAKEHLWPFIQEFVDGALSHIVKMSRAIGEEISCGHP
AWPAVIHGHYASAGVAAALLSGALNVPMVFTGHFLGKDKLEELLKQGRQTREQINMTYKIMCRIEAEELALDASEIV
IASTRQEIEEQWNLYDGFEVILARKLRARVKRGANCYGRYMPRMVIIPPGVEFGHMIHDFDMDGEEDGPSPASEDPS
IWSEIMRFFTNPRKPMILAVARPYPEKNITTLVKAFGECRPLRELANLTLIMGNREAISKMHNMSAAVLTSVLTLID
EYDLYGQVAYPKRHKHSEVPDIYRLAVRTKGAFVNVPYFEQFGVTLIEAAMHGLPVIATKNGAPVEIHQVLDNGLLV
DPHDQHAIADALYKLLSEKQLWSKCRENGLKNIHQFSWPEHCKNYLSRISTLGPRHPAFASNEDRIKAPIKGRKHVT
VIAVDSVSKEDLIRIVRNSIEAARKENLSGSTGFVLSTSLTIGEIHSLLMSAGMLPTDFDAFICNSGSDLYYPSCTG
DTPSNSRVTFALDRSYQSHIEYHWGGEGLRKYLVKWASSVVERRGRIEKQVIFEDPEHSSTYCLAFKVVNPNHLPPL
KELQKLMRIQSLRCHALYNHGATRLSVIPIHASRSKALRYLSVRWGIELQNVVVLVGETGDSDYEELFGGLHKTVIL
KGEFNTSANRIHSVRRYPLQDVVALDSPNIIGIEGYGTDDMRSALKQLDIRAQ
>LOC_Os08g34000.1
(SEQ ID NO: 137)
MGPTCQSLFLSFSVHLLPPAPPGCGAATRRGSCTEAEAENDPFDVIHSESVAMFHCWARDVPNLVVSWHGISLEALH
SRIYQDLTRGEDERMSPASNHSLAQSVYRVLSEVHFFRSYVHHVAISDTTGEMLRDVYQIPNRRVHVILNGVDEAQF
EPDAALGRAFREDLRLPKGANLVLGVSGRLVKGADLVLVAVGQISLSLP
>LOC_Os06g04200.1
(SEQ ID NO: 138)
MSALTTSQLATSATGFGIADRSAPSSLLRHGFQGLKPRSPAGGDATSLSVTTSARATPKQQRSVQRGSRRFPSVVVY
ATGAGMNVVFVGAEMAPWSKTGGLGDVLGGLPPAMAANGHRVMVISPRYDQYKDAWDTSVVAEIKVADRYERVRFFH
CYKRGVDRVFIDHPSFLEKVWGKTGEKIYGPDTGVDYKDNQMRFSLLCQAALEAPRILNLNNNPYFKGTYGEDVVFV
CNDWHTGPLASYLKNNYQPNGIYRNAKVAFCIHNISYQGRFAFEDYPELNLSERFRSSFDFIDGYDTPVEGRKINWM
KAGILEADRVLTVSPYYAEELISGIARGCELDNIMRLTGITGIVNGMDVSEWDPSKDKYITAKYDATTAIEAKALNK
EALQAEAGLPVDRKIPLIAFIGRLEEQKGPDVMAAAIPELMQEDVQIVLLGTGKKKFEKLLKSMEEKYPGKVRAVVK
FNAPLAHLIMAGADVLAVPSRFEPCGLIQLQGMRYGTPCACASTGGLVDTVIEGKTGFHMGRLSVDCKVVEPSDVKK
VAATLKRAIKVVGTPAYEEMVRNCMNQDLSWKGPAKNWENVLLGLGVAGSAPGIEGDEIAPLAKENVAAP
>LOC_Os06g04200.2
(SEQ ID NO: 139)
MSALTTSQLATSATGFGIADRSAPSSLLRHGFQGLKPRSPAGGDATSLSVTTSARATPKQQRSVQRGSRRFPSVVVY
ATGAGMNVVFVGAEMAPWSKTGGLGDVLGGLPPAMAANGHRVMVISPRYDQYKDAWDTSVVAEIKVADRYERVRFFH
CYKRGVDRVFIDHPSFLEKVWGKTGEKIYGPDTGVDYKDNQMRFSLLCQAALEAPRILNLNNNPYFKGTYGEDVVFV
CNDWHTGPLASYLKNNYQPNGIYRNAKVAFCIHNISYQGRFAFEDYPELNLSERFRSSFDFIDGYDTPVEGRKINWM
KAGILEADRVLTVSPYYAEELISGIARGCELDNIMRLTGITGIVNGMDVSEWDPSKDKYITAKYDATTAIEAKALNK
EALQAEAGLPVDRKIPLIAFIGRLEEQKGPDVMAAAIPELMQEDVQIVLLGTGKKKFEKLLKSMEEKYPGKVRAVVK
FNAPLAHLIMAGADVLAVPSRFEPCGLIQLQGMRYGTPCACASTGGLVDTVIEGKTGFHMGRLSVDCKVVEPSDVKK
VAATLKRAIKVVGTPAYEEMVRNCMNQDLSWKGPAKNWENVLLGLGVAGSAPGIEGDEIAPLAKENVAAP
>LOC_Os06g04200.3
(SEQ ID NO: 140)
MSALTTSQLATSATGFGIADRSAPSSLLRHGFQGLKPRSPAGGDATSLSVTTSARATPKQQRSVQRGSRRFPSVVVY
ATGAGMNVVFVGAEMAPWSKTGGLGDVLGGLPPAMAANGHRVMVISPRYDQYKDAWDTSVVAEIKVADRYERVRFFH
CYKRGVDRVFIDHPSFLEKVWGKTGEKIYGPDTGVDYKDNQMRFSLLCQAALEAPRILNLNNNPYFKGTYGEDVVFV
CNDWHTGPLASYLKNNYQPNGIYRNAKVAFCIHNISYQGRFAFEDYPELNLSERFRSSFDFIDGYDTPVEGRKINWM
KAGILEADRVLTVSPYYAEELISGIARGCELDNIMRLTGITGIVNGMDVSEWDPSKDKYITAKYDATTAIEAKALNK
EALQAEAGLPVDRKIPLIAFIGRLEEQKGPDVMAAAIPELMQEDVQIVLLGTGKKKFEKLLKSMEEKYPGKVRAVVK
FNAPLAHLIMAGADVLAVPSRFEPCGLIQLQGMRYGTPCACASTGGLVDTVIEGKTGFHMGRLSVDCKVVEPSDVKK
VAATLKRAIKVVGTPAYEEMVRNCMNQDLSWKGPAKNWENVLLGLGVAGSAPGIEGDEIAPLAKENVAAP
>LOC_Os06g04200.4
(SEQ ID NO: 141)
MSALTTSQLATSATGFGIADRSAPSSLLRHGFQGLKPRSPAGGDATSLSVTTSARATPKQQRSVQRGSRRFPSVVVY
ATGAGMNVVFVGAEMAPWSKTGGLGDVLGGLPPAMAANGHRVMVISPRYDQYKDAWDTSVVAEIKVADRYERVRFFH
CYKRGVDRVFIDHPSFLEKVWGKTGEKIYGPDTGVDYKDNQMRFSLLCQAALEAPRILNLNNNPYFKGTYGEDVVFV
CNDWHTGPLASYLKNNYQPNGIYRNAKVAFCIHNISYQGRFAFEDYPELNLSERFRSSFDFIDGYDTPVEGRKINWM
KAGILEADRVLTVSPYYAEELISGIARGCELDNIMRLTGITGIVNGMDVSEWDPSKDKYITAKYDATTAIEAKALNK
EALQAEAGLPVDRKIPLIAFIGRLEEQKGPDVMAAAIPELMQEDVQIVLLGTGKKKFEKLLKSMEEKYPGKVRAVVK
FNAPLAHLIMAGADVLAVPSRFEPCGLIQLQGMRYGTPCACASTGGLVDTVIEGKTGFHMGRLSVDCKVVEPSDVKK
VAATLKRAIKVVGTPAYEEMVRNCMNQDLSWKGPAKNWENVLLGLGVAGSAPGIEGDEIAPLAKENVAAP
>LOC_Os01g52710.1
(SEQ ID NO: 142)
MKVYITSAAPLAGEATKAMASPPSPPPHQHQQAATRRGCRSAVVTGLLAGVLLFRAALLTIEAGASLCPSTTGCLDW
RAGLGDWLYGGSGDAMEEFMKEWRRGRREASLLDPVVVEAAPDSLDGLMAEMDTMLASYDRLDMEAVVLKIMAMLLK
MDRKVKSSRIRALFNRHLASLGIPKSMHCLTLRLAEEFAVNSAARSPVPLPEHAPRLADASYLHVTIVTDNVLAAAV
AVASAVRSSAEPARLVFHVVTDKKSYVPMHSWFALHPVSPAVVEVKGLHQFDWRDGGAIASVMRTIEEVQRSSMEYH
QCDASVVREYRRLEASKPSTFSLLNYLKIHLPEFFPELGRVILLDDDVVVRKDLTGLWEQHLGENIIGAVGGHNPGE
DGVVCIEKTLGDHLNFTDPEVSNVLESARCAWSWGVNVVNLDAWRRTNVTDTYQLWLEKNRESGFRLWKMGSLPPAL
IAFDGRVQAVEPRWHLRGLGWHTPDGEQLQRSAVLHFSGPRKPWLEVAFPELRELWLGHLNRSDSFLQGCGVVE
>LOC_Os02g35020.1
(SEQ ID NO: 143)
MVKTNRFINSLSKRGYIGSYHYEKDAKYRPFSALLPEGSNPKMLYVKLVLIILMCGSFVSLLNSPSIHHNDDHHTES
SAGVPRVSYEPDDTRYVSDVTVDWPKISKAMQLVAGAEHGGGARVALLNFDDGEVQQWRTALPQTAAAVARLERAGS
NVTWEHLYPEWIDEEELYHAPTCPDLPEPAVDADGDGEEVAVFDVVAVKLPCRRGGGWSKDVARLHLQLAAARLAAT
RGRGGAAAHVLVVSASRCFPIPNLFRCRDEVAPRDGDVWLYRPDADALRRDLALPVGSCRLAMPFSALAAPHVAAAS
APPPRREAYATILHSEELYACGALVAAQSIRMASASGAPSEPERDMVALVDETISARHRGALEAAGWKVRAIRRVRN
PRAAADAYNEWNYSKFWLWSLTEYDRVVFLDADLLVQRPMSPLFAMPEVSATANHGTLFNSGVMVVEPCGCTLRLLM
DHIADIDSYNGGDQGYLNEVFSWWHRLPSHANFMKHFWEGDSGERLAAARRAVLAAEPAVALAVHFVGMKPWFCFRD
YDCNWNSPQLRQFASDEAHARWWRAHDAMPAALQGFCLLDERQKALLRWDAAEARAANFSDGHWRVPIADPRRNICA
TAAGDGEAAAACVEREIENRRVEGNRVTTSYAKLIDNF
>LOC_Os02g51130.1
(SEQ ID NO: 144)
MAGGGGGGRASAQRRAALAALITLLLLASLAFLLSATGTASAPNSAPFRLAAIRRHAEDHAAVLAAYAAQARKLSAA
SASQTESFLSISGHLSSLSSRISLSTVALLEKETRGQIKRARSLAGAAKEAFDTQSKIQKLSDTVFAVDQQLLRARR
AGLLNSRIAAGSTPKSLHCLVMRLLEARLANASAIPDDPPVPPPQFTDPALYHYAIFSDNVLAVSVVVASAARAAAE
PARHVFHVVTAPMYLPAFRVWFARRPPPLGTHVQLLAVSDFPFLNASASPVIRQIEDGNRDVPLLDYLRFYLPEMFP
ALRRVVLLEDDVVVQRDLAGLWRVDLGGKVNAALETCFGGFRRYGKHINFSDPAVQERFNPRACAWSYGLNVFDLQA
WRRDQCTQRFHQLMEMNENGTLWDPASVLPAGLMTFYGNTRPLDKSWHVMGLGYNPHIRPEDIKGAAVIHFNGNMKP
WLDVAFNQYKHLWTKYVDTEMEFLTLCNFGL
>LOC_Os06g49810.1
(SEQ ID NO: 145)
MAGARAMAFVALCALAAFPVAVTGAQVDPLYSSKQVLDWSSQANIKLQNFSLTEEDGLQLLVRPEEVTHRKLRERTR
IKKKIEPVQQDDEALVKLENAGIERSKAVDSAVLGKYSIWRRENENEKADSKVRLMRDQMIMARIYSVLAKSRDKLD
LHQDLLSRLKESQRSLGEATADAELPKSASERVKVMGQLLAKARDQLYDCKAITQRLRAMLQSADEQVRSLKKQSTF
LSQLAAKTIPNGIHCLSMRLTIDYYLLSPEKRKFPKSENLENPDLYHYALFSDNVLAASVVVNSTIMNAKEPEKHVF
HLVTDKLNFGAMNMWFLLNPPGDATIHVENVDDFKWLNSSYCPVLKQLESVAMKEYYFKADRPKTLSAGSSNLKYRN
PKYLSMLNHLRFYLPQVYPKLNKILFLDDDIVVQKDLTGLWEVDLNGNVNGAVETCGESFHRFDKYLNFSNPNIAQN
FDPNACGWAYGMNMFDLEEWKKKDITGIYHKWQNMNENRLLWKLGTLPPGLLTFYKLTHPLDKSWHVLGLGYNPSIE
RSEIDNAAVIHYNGNMKPWLEIAMSKYRPYWTKYINYEHTYVRGCKISQ
>LOC_Os08g38740.1
(SEQ ID NO: 146)
MATMASAAAASASARRWRWRWKWRTRDAVLALLIASVLAPPLLLYGGAPIAPFSGPILMGSAASGLDLSNLIARKEV
RERLNALKQDAFAAVKEPIQTVASDAAALKAGLIQHIVDQSSGIDRGTKDNGMVASVNKKGGVEFTKENGLIDDGKL
RENKVRAMRNSSGLNITLNKVKGSYAVSTEEYAFHQTIPPLTDLMFGTFPPALLDHTADRPPEKTTDTTSEDSDIRA
ISNNTSHSTASPDSTIRVLRDQLKRARTYIGFLSSRGNHGFIKDLRRRMRDIQQALSGATNDKQLPKKYYLSHRYTK
FFTVGISDDDLCLVSGVHGRIREMELTLTKIKQVHENCAAIISKLQATLHSTEEQMQAHKQEANYVTQIAAKALPKR
LNCLAMRLTNEYYSSSSSNKHFPYEEKLEDPKLQHYALFSDNVLGAAVVVNSTIIHAKTPENHVFHIVTDKLNYAAM
RMWFLENSQGKAAIEVQNIEDFTWLNSSYSPVLKQLESQFMINYYFKTQQDKRDNNPKFQNPKYLSILNHLRFYLPE
IFPKLNKVLFLDDDIVVQQDLSALWSIDLKGKVNGAIQTCGETFHRFDRYLNFSNPLIAKNFERRACGWAYGMNMFD
LSEWRKRNITDVYHYWQEQNEHRLLWKLGTLPAGLVTFWNQTFPLDHKWHLLGLGYKPNVNQKDIEGAAVIHYNGNR
KPWLEIAMAKYRKYWSKYVNFDNVFIRQCNIHP
>LOC_Os09g30280.1
(SEQ ID NO: 147)
MVAVARGRRCRGVVLLLLLSSVLAPLVLYGGSPVSVSTLPDSTVASGVLDRDGEYNLVVAASDVSLTKDLTIERLGE
HKNRVLSATEDWQVVEAASKNPAFEKSDASVSRKDPGSGDANVVITEGNGAAQSGRDGVIWEVVSRDRGSDGFTQPW
EINGGEERDGERVDRVKLGVSVEEQNDGTGETGVNNIAGTHTSGNLNSSLEKERSTGRLSEQVTKAIEKESYTPTTN
SNSALPTSVSAGHSTTSPDATIRTIKDQLTRATTYLSLVASRGNHGFARELRARMRDIQRVLGDATSGGQLPQNVLS
KIRAMEQTLGKGKRILDSCSGALNRLRATLHSTEERLQSHKKETNYLAQVAAKSLPKGLHCLPLRLTNEYYYTNSNN
KKFPHIEKLEDPKLYHYALFSDNVLAAAVVVNSTIIHAKKPADHVFHIVTDRLNYAAMKMWFLANPLGEAAIQVQNI
EEFTWLNSTYSPVMKQLESQSMIDYYFKSGQARRDENPKFRNPKYLSMLNHLRFYLPEIFPKLSKVLFLDDDTVVQQ
DLSAIWSIDLKGKVNGAVETCGETFHRFDKYLNFSNPLIASNFDPRACGWAYGMNVFDLSEWRRQKITDVYHNWQRL
NENRILWKLGTLPAGLVTFWNRTFPLHHSWHQLGLGYNPNINEKDIRRASVIHYNGNLKPWLEIGLSRYRKYWSKYV
DFDQVFLRDCNINP
>LOC_Os04g35270.1
(SEQ ID NO: 148)
MSSPPSSGSERSLASLVSAAAHSVKLNRAYLLAPAVAAGLLAAVLLSSLLDFSAFSASPRPAFPPPTAGAPANASAL
SAPPRAPVRTALDTLGTRPREPFTALRDAYARWDAAVGCAAFAEKHRSRSSPPPGPAALQDPEAAPCGSLRLPHVAL
AVRGVTWVPDILDGVYQCRCGLTCLWSRNEEALADTPDVVLYEIWPPPDTRKQGEPLRAFMDIEPTRKRSGHEDIFI
GYHADDDVQVTYAGKFFRITHNYHVATHKRDDVLVYWSSSRCFEHRNKIARELFRHLPAHSFGRCENNVGGGDKALE
LYPDCARDGHGAAEWWDHLHCAMSHYKFVLAIENTIADSYSTEKLYYALEAGSVPIYFGAPNARDLAPPGSYIDGAA
FASAEELAAYVREVAGDPAAYAEFHAWRRCGVLGGYGRNRLVSLDTLPCRLCERASRMGGRHAPAPNATVS
>LOC_Os01g56570.1
(SEQ ID NO: 149)
MHLSGARLRIPTWCTMPHGSWLLQTCSPSAALASLAVVTTSLLIIGYASSSFFLGAPAYEYDDVVEAAAAVPRRGPG
YPPVLAYYISGGHGDSVRMTRLLKAVYHPRNRYLLHLDAGAGAYERARLAGYARSERAFLEYGNVHVVGKGDPVDGR
GPSAVAAVLRGAAVLLRVGAEWDWLVTLGASDYPLVTPDDLLYAFSSVRRGLSFIDHRMDSGGAEAVVVDQNLLQST
NAEISFSSGQRAKPDAFELFRGSPRPILSRDFVEYCVVAPDNLPRTLLLYFSNSLSPMEFYFQTVMANSAQFRNSTV
NHNLRHTVAQDGGAPTSQGADGQQASRYDAMVGSGAAFAGAFGDDDDALLQRIDEEVLRRPLDGVTPGEWCVADGEE
GTDNECSVGGDIDVVRHGAKGRKLATLVVDLVGA
>LOC_Os03g05180.1
(SEQ ID NO: 150)
MLMYYTNMPLPHRKYFQTVLCNSPEFNRTVVNHDLHYSKWDSSSKKEPLLLTLDDVENMTQSGVAFGTRFSMDDPVL
NHIDEEILHRQPEEPAPGGWCIGVGDASPCSVSVDLQACLINSRAASTSAQTLTPRRLRPTLPEKTLKHSSSDVVPA
TRRSWSSIPSSTNSATYRLSDRIMATASSMVYRRIRGTTMDGRLYSLRTCTIVFASAASVTLAAARLARSSSGSMNR
SICAARPRKMLSTLPFSAMIVLSTLVAPAATPLSGSRHSSPA
>LOC_Os03g48560.1
(SEQ ID NO: 151)
MDTHAVPRAAATVDLRWLLSVAAGAVFALLLLLAASPPFPLRPASLFTTTSPRRALPPLFVESSSTLSAPPPTPPPS
PPRFAYLISGSAGDAPMMRRCLLALYHPRNSYTLHLDAEAPDDDRAGLAAFVAAHPALSAAANVRVIRKANLVTYRG
PTMVTTTLHAAAAFLWGRGGGRGADWDWFINLSASDYPLVTQDDLMHVFSKLPRDLNFIDHTSDIGWKAFARAMPMI
VDPALYMKTKGELFWIPERRSLPTAFKLFTGSAWMVLSRPFVEYLIWGWDNLPRTVLMYYANFISSPEGYFHTVACN
AGEFRNTTVNSDLHFISWDNPPMQHPHYLADADWGPMLASGAPFARKFRRDDSVLDRIDADLLSRRPGMVAPGAWCG
AAAAADGDSNSTTTGGAVDPCGVAGGGGEAVRPGPGAERLQRLVASLLSEENFRPRQCKVVEAN
>LOC_Os04g23580.1
(SEQ ID NO: 152)
MRPPARSPPRLAAAAAALATSAALLLICGTWPASASGFGAYTASSSARRASSTGGADAPPPSFAYLISGTGGEAARV
VRLLRAVYHPRNRYLLHLDAAAGAEERAELAAAVRGVRAWRERANVDVVGEGYAVDRAGPSALAAALHGAAVLLRVA
ADWDWFVTLSSSDYPLVTQDDLLYAFSSVPRDLNFIDHTSDLGWKEHERFEKLIVDPSLYMDRNSEILPATEPRQMP
DAFKIFTVNYKFLLRTQSVLKHERRTNNDDGSPWVILSRNFTEHCVHGWDNLPRKLLMYFANTAYSMESYFQTVICN
SSKFRNTTVNGDLRYFVWDDPPGLEPLVLDESHFDDMVNSSAAFARRFVDDSPVLKKIDKEILNRSSAVCASFSRRR
GMDVDSCSKWGDVNVLQPARAGEQLRRFISEISQTRGCS
>LOC_Os06g40060.2
(SEQ ID NO: 153)
MMLTHQFIEYCIWGWDNLPRTVLMYYANFLSSPEGYFHTVICNVPEFRNTTVNHDLHFISWDNPPKQHPHYLTLNDF
DGMVNSNAPFARKFGREDPVLDKIDQELLGRQPDGFVAGGWMDLLNTTTVKGSFTVERVQDLRPGPGADRLKKLVTG
LLTQEGFDDKHCL
>LOC_Os09g25890.1
(SEQ ID NO: 154)
MPHHRHLTPSPSHEEHETPNPSLTPPPMQLAALASDEPPPPPPEQSPRRIVVAHRLPLNATPDPGSPFGFAFSLSAD
AHALQLSHGLGLAHVVFVGTLPAEAARALRRSDELDRHLLGCFSCLPVFLPPRAHDEFYAGFCKHYLWPRLHYLLPH
APAANGYLHFDAGLYRSYASANRSFAARVVEVLSPDDGDLVFVHDYHLWLLPSFLRRGCPRCRVGFFLHSPFPSAEV
FRSIPVREDLLRALLNADLVGFHTYDYARHFLSACSRLLGLAYTSRHGRVGINYHGRTVLIKFLSVGVDMGLLRTAM
ASPEAAAKFREITEVEYKGRVLMVGVDDVDIFKGVRLKLLAMESLLETYPALRGRVVLVQIHNPTRCGGRDVERVRG
ETAKIQARINARFGGPGYQPVVVVDRAVPMAEKVAYYAAAECCVVSAVRDGLNRIPYFYTVCREEGPVDAKGAAGGQ
PRHSAIVLSEFVGCSPSLSGAIRVNPWNIEAMAEAMHGALTMNVAEKQARHVKHYTYLKLHDVIVWARSFAADLQLA
CKDRSTMRTIGMGIGPSYRVVAVDAAFKKLPPELVNLSYRAAAAAAAGGGGGRLILLDYDGTLEPTGAFDNAPSDAV
IVILDELCSDPNNVVFIVSGRSKDDLERWLAPCANLGIAAEHGYFIRWSRDAPWETMASKQLAAAMEWKAAAKNVMR
HYAEATDGSYIEAKETGMVWRYEDADPRLAPLQAKELLDHLATVLASEPVAVRSGYKIVEVIPQGVSKGVAAECIVS
AMAARRGGALGFVLCVGDDRSDEDMFGALASLCGGGKNGGASSSTTTTTALLAAAQVFACTVGNKPSMASYYLNDKE
EVVDMLHGLAFSSPSSRLRAAAAPRRPADFDIKSLLRCE
>LOC_Os09g25890.2
(SEQ ID NO: 155)
MPHHRHLTPSPSHEEHETPNPSLTPPPMQLAALASDEPPPPPPEQSPRRIVVAHRLPLNATPDPGSPFGFAFSLSAD
AHALQLSHGLGLAHVVFVGTLPAEAARALRRSDELDRHLLGCFSCLPVFLPPRAHDEFYAGFCKHYLWPRLHYLLPH
APAANGYLHFDAGLYRSYASANRSFAARVVEVLSPDDGDLVFVHDYHLWLLPSFLRRGCPRCRVGFFLHSPFPSAEV
FRSIPVREDLLRALLNADLVGFHTYDYARHFLSACSRLLGLAYTSRHGRVGINYHGRTVLIKFLSVGVDMGLLRTAM
ASPEAAAKFREITEVEYKGRVLMVGVDDVDIFKGVRLKLLAMESLLETYPALRGRVVLVQIHNPTRCGGRDVERVRG
ETAKIQARINARFGGPGYQPVVVVDRAVPMAEKVAYYAAAECCVVSAVRDGLNRIPYFYTVCREEGPVDAKGAAGGQ
PRHSAIVLSEFVGCSPSLSGAIRVNPWNIEAMAEAMHGALTMNVAEKQARHVKHYTYLKLHDVIVWARSFAADLQLA
CKDRSTMRTIGMGIGPSYRVVAVDAAFKKLPPELVNLSYRAAAAAAAGGGGGRLILLDYDGTLEPTGAFDNAPSDAV
IVILDELCSDPNNVVFIVSGRSKDDLERWLAPCANLGIAAEHGYFIRWSRDAPWETMASKQLAAAMEWKAAAKNVMR
HYAEATDGSYIEAKETGMVWRYEDADPRLAPLQAKELLDHLATVLASEPVAVRSGYKIVEGVSKGVAAECIVSAMAA
RRGGALGFVLCVGDDRSDEDMFGALASLCGGGKNGGASSSTTTTTALLAAAQVFACTVGNKPSMASYYLNDKEEVVD
MLHGLAFSSPSSRLRAAAAPRRPADFDIKSLLRCE
>LOC_Os02g44510.2
(SEQ ID NO: 156)
MILSVLKQTQRPVKFWFIKNYLSPQFKDVIPHMAQEYGFEYELVTYKWPTWLHKQKEKQRIIWAYKILFLDVIFPLS
LRKVIFVDADQIVRADMGELYDMNLKGRPLAYTPFCDNNKEMDGYRFWKQGFWKDHLRGRPYHISALYVVDLAKFRQ
TASGDTLRVFYETLSKDPNSLSNLDQDLPNYAQHTVPIFSLPQEWLWCESWCGNATKARAKTIDLCNNPMTKEPKLQ
GAKRIVPEWVDLDSEARQFTARILGDNPESPGTTSPPSDTPKSDDKGAKHDEL
>LOC_Os02g44510.3
(SEQ ID NO: 157)
MILSVLKQTQRPVKFWFIKNYLSPQFKDVIPHMAQEYGFEYELVTYKWPTWLHKQKEKQRIIWAYKILFLDVIFPLS
LRKVIFVDADQIVRADMGELYDMNLKGRPLAYTPFCDNNKEMDGYRFWKQGFWKDHLRGRPYHISALYVVDLAKFRQ
TASGDTLRVFYETLSKDPNSLSNLDQDLPNYAQHTVPIFSLPQEWLWCESWCGNATKARAKTIDLCNNPMTKEPKLQ
GAKRIVPEWVDLDSEARQFTARILGDNPESPGTTSPPSDTPKSDDKGAKHDEL
>LOC_Os07g23740.1
(SEQ ID NO: 158)
MRGASGGGEGFVGASSVSNNISLPNEGTSPRGTDNAECSETSSDRSNSESIKPEECAMPSSIFDKKISIKKKLRLLS
RMAILKDDGTVEVDIPTNAEAASLDLSSNDYCNEAFSGEPLASSDFQHRPPMQIVMLIVGTRGDVQPFIAIGKRLQI
YGHRVRLATHANFKDFVVTAGLEFYPLGGDPKLLAGYMVKNKGFLPATPSEIPIQRKEIKEIIFSLLPACKDPDTDT
GAPFNVNAIIANPAAYGHVHVAEALKVPIHIIFTMPWTPTCEFPHPFSRVKQPAGYRLSYQIVDSFVWLGIRDIIND
LRKRKLKLRPVTYLSSAHAYSNDIPHAYIWSPYLVPKPKDWGPKIDVVGFCFLDLASNYKPPEPLLKWLESGEKPIY
IGFGSLPIPEPDKLTRIIVEALEITGQRGIINKGWGGLGNLEEPKEFVYVIDNIPHDWLFLQCKAVVHHGGAGTTAA
SLKAACPTTIVPFFGDQFFWGNMVHARGLGAPPVPVEQLQLHLLVDAIKFMMDPKVKERAVELAKAIESEDGVDGAV
KAFLKHLPQPRSLEKPQPAPPSSTFMQPFLLPVKRCFGIAT
>LOC_Os08g20420.1
(SEQ ID NO: 159)
MSDTGGGHRASAEALRDAFRLEFGDAYQVFVRDLGKEYGGWPLNDMERSYKFMIRHVRLWKVAFHGTSPRWVHGMYL
AALAYFYANEVVAGIMRYNPDIIISVHPLMQHIPLWVLKWQSLHPKVPFVTVITDLNTCHPTWFHHGVTRCYCPSAE
VAKRALLRGLEPSQIRVYGLPIRPSFCRAVLDKDELRKELDMDPDLPAVLLMGGGEGMGPVEETARALSDELYDRRR
RRPVGQIVVICGRNQVLRSTLQSSRWNVPVKIRGFEKQMEKWMGACDCIITKAGPGTIAEALIRGLPIILNDFIPGQ
EVGNVPYVVDNGAGVFSKDPREAARQVARWFTTHTNELRRYSLNALKLAQPEAVFDIVKDIHKLQQQPATVTRIPYS
LTSSFSYSI
>LOC_Os08g20420.2
(SEQ ID NO: 160)
MSDTGGGHRASAEALRDAFRLEFGDAYQVFVRDLGKEYGGWPLNDMERSYKFMIRHVRLWKVAFHGTSPRWVHGMYL
AALAYFYANEVVAGIMRYNPDIIISVHPLMQHIPLWVLKWQSLHPKVPFVTVITDLNTCHPTWFHHGVTRCYCPSAE
VAKRALLRGLEPSQIRVYGLPIRPSFCRAVLDKDELRKELDMDPDLPAVLLMGGGEGMGPVEETARALSDELYDRRR
RRPVGQIVVICGRNQVLRSTLQSSRWNVPVKIRGFEKQMEKWMGACDCIITKAGPGTIAEALIRGLPIILNDFIPGQ
VCADATILNSLE
>LOC_Os04g42760.1
(SEQ ID NO: 161)
MKRRHWSHPSCGLLLLVAVFCLLLVFRCSQLRHSGDGAAAAAPDGGAGRNDGDDVDERLVELAAVDPAAMAVLQAAK
RLLEGNLARAPERHRDVALRGLREWVGKQERFDPGVMSELVELIKRPIDRYNGDGGGGGEGEGRRYASCAVVGNSGI
LLAAEHGELIDGHELVVRLNNAPAGDGRYARHVGARTGLAFLNSNVLSQCAVPRRGACFCRAYGEGVPILTYMCNAA
HFVEHAVCNNASSSSSGAADATAAAPVIVTDPRLDALCARIVKYYSLRRFARETGRPAEEWARRHEEGMFHYSSGMQ
AVVAAAGVCDRVSVFGFGKDASARHHYHTLQRRELDLHDYEAEYEFYRDLESRPEAIPFLRQRNSGFRLPPVSFYR
>LOC_Os02g06840.1
(SEQ ID NO: 162)
MSWRKGGGGDGGVSRRWAVLLCLGSFCLGLLFTNRMWTLPEANEIARPNGNGDEGNTLVAAECGPKKVQHPDYKDIL
RVQDTHHGVQTLDKTIASLETELSAARSLQESLLNGSPVAEEFKLSESIGRRKYLMVIGINTAFSSRKRRDSIRYTW
MPQGEKRKKLEEEKGIIIRFVIGHSAISGGIVDRAIEAEDRKHGDFMRIDHVEGYLALSGKTKTYFATAVSLWDADF
YVKVDDDVHVNIATLGQILSNHALKPRVYIGCMKSGPVLTEKGVRYYEPEHWKFGEPGNKYFRHATGQLYAISKDLA
TYISINRHVLHKYINEDVSLGSWFIGLDVEHIDDRRLCCGTPPDCEWKAQAGNTCAASFDWRCSGICNSEGRIWEVH
NKCAEGEKALWNATF
>LOC_Os02g06840.2
(SEQ ID NO: 163)
MSWRKGGGGDGGVSRRWAVLLCLGSFCLGLLFTNRMWTLPEANEIARPNGNGDEGNTLVAAECGPKKVQHPDYKDIL
RVQDTHHGVQTLDKTIASLETELSAARSLQESLLNGSPVAEEFKLSESIGRRKYLMVIGINTAFSSRKRRDSIRYTW
MPQGEKRKKLEEEKGIIIRFVIGHSAISGGIVDRAIEAEDRKHGDFMRIDHVEGYLALSGKTKTYFATAVSLWDADF
YVKVDDDVHVNIATLGQILSNHALKPRVYIGCMKSGPVLTEKGVRYYEPEHWKFGEPGNKYFRHATGQLYAISKDLA
TYISINRHVLHKYINEDVSLGSWFIGLDVEHIDDRRLCCGTPPDCEWKAQAGNTCAASFDWRCSGICNSEGRIWEVH
NKCAEGEKALWNATF
>LOC_Os02g36770.1
(SEQ ID NO: 164)
MAHAADTAIMLVFVFRLLAFTLTILLSPLMWVTKRLGITVLIVLFPLLIVHHLIVNSPVSGPSRYQVIHSNLLGWLS
DSLGNSVAQNPDNTPVEVIPADASASNSSDSGNSSLEGFQWLNTWNHMKQLTNISDGLPHANEAIDNARTAWENLTI
SVHNSTSKQIKKERQCPYSIHRMNASKPDTGDFTIDIPCGLIVGSSVTIIGTPGSLSGNFRIDLVGTELPGGSGKPI
VLHYDVRLTSDELTGGPVIVQNAFTASNGWGYEDRCPCSNCNNATQVDDLERCNSMVGREEKRAINSKQHLNAKKDE
HPSTYFPFKQGHLAISTLRIGLEGIHMTVDGKHVTSFPYKAGLEAWFVTEVGVSGDFKLVSAIASGLPTSEDLENSF
DLAMLKSSPIPEGKDVDLLIGIFSTANNFKRRMAIRRTWMQYDAVREGAVVVRFFVGLHTNLIVNKELWNEARTYGD
IQVLPFVDYYSLITWKTLAICIYGTGAVSAKYLMKTDDDAFVRVDEIHSSVKQLNVSHGLLYGRINSDSGPHRNPES
KWYISPEEWPEEKYPPWAHGPGYVVSQDIAKEINSWYETSHLKMFKLEDVAMGIWIAEMKKGGLPVQYKTDERINSD
GCNDGCIVAHYQEPRHMLCMWEKLLRTNQATCCN
>LOC_Os02g54390.1
(SEQ ID NO: 165)
MAMKRLSFSLFLLPFLLLAFVYSLFFPGYFSILPSLAARCSNSVAATPANATGPAVDLRVLLGVVTRAEMYERRALL
RLAYALQPAPARAVVDVRFFVCSLAREEDAVLVSLEIIAHGDVVVLNCTENMDDGKTHSYFSSLPALFADAPYDYVG
KIDDDSYYRLASLADTLRDKPRRDLYHGFPAPCHADPRSQFMSGMGYIVSWDVAAWVAATEALRGDVKGPEDEVFGR
WLRRGGKGSNRYGEETRMYDYLDGGMREGVNCFRHALVADTVVVHKLKDRLKWARTLKFFNATQGLKPSKLYHVDL
>LOC_Os02g54420.1
(SEQ ID NO: 166)
MAMKKSFSLLFFLPFLLLAIIYFVIFPNEFRLQSSLAACGDSAPATAADAVAKAAPDIRVLLGVLTRADKYERRALV
RLAYALQPAPARAVVHVRFVVCNLTAEEDAALVGLEIAAYGDIIVLDCTENMDNGKTYTYFSAVPRLFAGEPYDYVG
KTDDDTYYRLGALADALRDKPRRDAYYGFLTPCHADPRTQYMSGMGYVVSWDVAAWVAATPELQNDLKGPEDKLFGR
WLRWGGRGRNVFGAEPRMYDYLDGGMRHGPTCFRHLLQADTVAVHKLKDNLKWARTLNFFNATEGHKASPLFHVDH
>LOC_Os02g54450.1
(SEQ ID NO: 167)
MAANFSACLVPVAVLALFYLVIFPNDLSQLKSALAPCDAASKSVAAAAAADDDVDFRMFFGILTRPDFYERRALLRM
AYALQPPPRRAAIDVRFVMCSLDKEEDAVLVAMEIITHGDILVLNCTENMNDGKTYDYFSALPRLFPAGAEPRYDFA
GKIDDDTYYRLGALADTLRRKPRRDMYHGFLNPCHIDPAWQYMSGMGYIVSWDVAEWIAASPELRGREIGYEDDVFG
RWLRGAGKGKNRFGEEPRMYDYLDREMYGADVNCFRHELIADTVAVHKLKDRLKWARTLRFFNATDGLKPSKMYHVD
LTPRI
>LOC_Os03g48610.1
(SEQ ID NO: 168)
MRRPRRAAAGCGCGRRLRPLLMLLPFAALLSVATFSLHSPVGLVVPAAVTVATSTDTDTDTASSHHHHHGLVGDAVS
GIDIRALNATPPLHAAAVRAFRSGGRLLREAFLPGAAPPPAVGGGPDPSPPRCPPFVALSGAELRGAGDALALPCGL
GLGSHVTVVGSPRRVAANAVAQFAVEVRGGGDGDGDEAARILHFNPRLRGDWSGRPVIEQNTRFRGQWGPALRCEGW
RSRPDEETVDGLVKCEQWGGNYGSKLNELKKMWFLNRVAGQRNRGSMDWPYPFVEDELFVLTLSTGLEGYHVQVDGR
HVASFPYRVGYSLEDAAILSVNGDVDIQSIVAGSLPMAYPRNAQRNLELLTELKAPPLPEEPIELFIGILSAGSHFT
ERMAVRRSWMSSVRNSSGAMARFFVALNGRKKVNEDLKKEANFFGDIVIVPFADSYDLVVLKTVAICEYATRVISAK
YIMKCDDDTFVRLDSVMADVRKIPYGKSFYLGNINYYHRPLREGKWAVSFEEWPREAYPPYANGPGYIVSSDIANFV
VSEMEKGRLNLFKMEDVSMGMWVGQFVDTVKAVDYIHSLRFCQFGCVDDYLTAHYQSPGQMACLWDKLAQGRPQCCN
PR
>LOC_Os03g58900.1
(SEQ ID NO: 169)
MPPPKRACRLALLAAGGAYLLFLLLFELPSVSISVSTASPAAAAAATTHRPRRRELEAASSSSSSSSSPLRPLKTAF
PSRRSPLAVSSIRFRRRNSSSIDASAASAFAAARPLMHHLLSSFSSPSPSSSPSPSPSTSDSCPSTISVPTHRLTSG
GGGGNGGGVTVELPCGMGVGSHVTVVARPRPARPESEPRIAERRGGEAAVMVSQFMVELLGTKAVQGEEPPRILHFN
PRIRGDFSGRPVIELNTCYRMQWAQPQRCEGWASQPHEETVDGQLKCERWIRDDNSKSEESNAQLWLNRLIGRGNEV
AADRPYPFEEGKLFALTVTAGLDGYHVNVDGRHVASFPYRTGYSLEDATGLSLKGDLDIESILAGHLPNSHPSFAPQ
RYLEMSEQWKAPPLPTEPVELFIGILSAANHFAERMAVRKSWMIDIRKSSNVVARFFVALNGEKEINEELKKEAEFF
SDIVIVPFMDSYDLVVLKTIAIAEYGVRIVPAKYIMKCDDDTFVRIDSVLDQVKKVEREGSMYIGNINYYHRPLRSG
KWSVSYEEWQEEVYPPYANGPGYVISSDIAQYIVSEFDNQTLRLFKMEDVSMGMWVEKFNSTRQPVKYSHDVKFFQS
GCFDGYYTAHYQSPQQMICLWRKLQFGSAQCCNMR
>LOC_Os05g11060.1
(SEQ ID NO: 170)
MPLHHHRHHHHSAAVAVAVADDDDEAKPRRPYSTFASPRAPTSAFSAAFSTHRLLVLFSVACLLVAAASLAFAFSAR
AATLQPPPLAAVAEATAKVAFRCGRAEDTLRAFLASSSGNYSSAAEGREREKVLAVVGVHTEIGSAARRAALRATWF
PPKPEGIVSLEHGTGLSFRFVVGRTKDKEKMADLQKEVDMYHDFLFVDAEEDTKPPQKMLAFFKAAYDMFDADFYVK
ADDAIYLRPDRLAALLAKDRLHQRTYIGCMKKGPVVNDPNMKWYESSWELLGNEYFSHASGLLYALSSEVVGSLAAT
NNDSLRMFDYEDVTIGSWMLAMNVKHEDNRAMCDSACTPTSIAVWDSKKCSNSCNTTEIVKALHNTTLCSKSPTLPP
EVEDE
>LOC_Os05g11060.2
(SEQ ID NO: 171)
MPLHHHRHHHHSAAVAVAVADDDDEAKPRRPYSTFASPRAPTSAFSAAFSTHRLLVLFSVACLLVAAASLAFAFSAR
AATLQPPPLAAVAEATAKVAFRCGRAEDTLRAFLASSSGNYSSAAEGREREKVLAVVGVHTEIGSAARRAALRATWF
PPKPEGIVSLEHGTGLSFRFVVGRTKDKEKMADLQKEVDMYHDFLFVDAEEDTKPPQKMLAFFKAAYDMFDADFYVK
ADDAIYLRPDRLAALLAKDRLHQRTYIGCMKKGPVVNDPNMKWYESSWELLGNEYFSHASGLLYALSSEVVGSLAAT
NNDSLRMFDYEDVTIGSWMLAMNVKHEDNRAMCDSACTPTSIAVWDSKKCSNSCNTTEIVKALHNTTLCSKSPTLPP
EVEDE
>LOC_Os05g47880.1
(SEQ ID NO: 172)
MSSSSSLYKQLGLGAGSPVSASHLLLLVLGAGFLALTVFVVHPNEFRIQSFFSGGCGRPGTDAATAAVAASPVKNVS
GGASDAAAATTAARSPDNDVRVLIGIQTLPSKYERRNLLRTIYSLQAREQPSLAGSVDVRFVFCNVTSPVDAVLVSL
EAIRHGDIIVLDCAENMDNGKTYTFFSTVARAFNSSDGEGSGSGSPPPPRYDYVMKADDDTYLRLAALVESLRGAAR
RDAYYGLQMPCDRENFYPFPPFMSGMGYALSWDLVQWVATAEESRRDHVGPEDMWTGRWLNLASKAKNRYDMSPRMY
NYRGASPPSCFRRDFAPDTIAVHMLKDAARWAETLRYFNATAALRPSHL
>LOC_Os06g09270.1
(SEQ ID NO: 173)
MSYLQKPSYYTISLVVVLLLPFTILFASFLLPFSAYLRGPPPIAAGSVVAGGCRHGAADGGGGGGGGGGVRPEISIL
VGVHTMAKKHSRRHLVRMAYAVQQTAALRGAARVDVRFALCARPMPQEHRAFVALEARAYGDVMLIDCDESPDKGKT
YDYFAGLPAMLSSGGGGGGGGEGRPYDYVMKVDDDTYLRLDELAETLRRAPREDMYYGAGLPFLDKESPPFMLGMGY
VLSWDLVEWIAGSDMAKALAIGAEDVTTGTWLNMGNKAKNRVNIFPRMYDFKGVKPEDFLEDTIGVHQLKQDLRWAQ
TLEHFNVTCLDPSSKMTNSLLS
>LOC_Os06g46570.1
(SEQ ID NO: 174)
MSWRRGDGGVARRWVLLLCTGSFFLGLLFTDRMWTLPEVTEVARPNGRREKEDELTAGDCNSAKVNVKRDYREILQT
QDTHHAVWTLDKTIAKLETELSAARTLQESFLNGSPVSEGHKGSDSTGRQKYLMVIGINTAFSSRQRRDSIRNTWMP
QGIKRRKLEEEKGIVIRFVIGHSAISGGIVERAIKAEERKHGDFMRIDHVEGYLELSGKTKTYFATAVSLWDADFYV
KVDDDVHVNIATLGQILSNHVKKPRVYIGCMKSGPVLSDKDVRYYEPEHWKFGDQYFRHATGQLYAISKDLATYISI
NKRVLHKYINEDVSLGAWFIGLDVEHIDERRLCCGTPPDCEWKAQAGNTCAVSFDWKCSGICDSVENMQWVHNRCGE
SEKSLWISSF
>LOC_Os06g46570.2
(SEQ ID NO: 175)
MSWRRGDGGVARRWVLLLCTGSFFLGLLFTDRMWTLPEVTEVARPNGRREKEDELTAGDCNSAKVNVKRDYREILQT
QDTHHAVWTLDKTIAKLETELSAARTLQESFLNGSPVSEGHKGSDSTGRQKYLMVIGINTAFSSRQRRDSIRNTWMP
QGIKRRKLEEEKGIVIRFVIGHSAISGGIVERAIKAEERKHGDFMRIDHVEGYLELSGKTKTYFATAVSLWDADFYV
KVDDDVHVNIATLGQILSNHVKKPRVYIGCMKSGPVLSDKDVRYYEPEHWKFGDQYFRHATGQLYAISKDLATYISI
NK
>LOC_Os07g09670.1
(SEQ ID NO: 176)
MPPPPRKRLGRAALLLAAAAYLAFLLLFELPSLDLFPSSDAAAGAAMPTHRPRRRELEASSSSSAFASPVLRRPATA
VSPAPASAAAAAAGALPIFSSLLLLPRPNATATPFDGTAAEAFAAARPHLDHLRTAAAAAAEEASSSSTAPTCPTSI
SVHADGLPGDGVRTVELPCGLAVGSHVTVVARPRAARPEYDPKIAERKSGQEPLMVSQFMVELVGTKAVDGEAPPRI
LHFNPRIRGDYSGKPVIEMNSCYRMQWGQSQRCEGYASRPADETVDGQLKCEKWIRDDDKKSEESKMKWWVKRLIGR
PKDVHISWPYPFAEGKLFVLTLTAGLEGYHVNVDGRHVTSFPYRTGYTLEDATGLSLNGDIDIESIFASSLPNSHPS
FAPERYLEMSEQWRAPPLPTEPVELFIGILSAASHFAERMAVRKSWMMYTRKSTNIVARFFVALNGKKEVNAELKRE
AEFFQDIVIVPFMDSYDLVVLKTIAIAEYGVRVIPAKYIMKCDDDTFVRIDSVLDQVKKVRSDKSVYVGSMNYFHRP
LRSGKWAVTYEEWPEEAYPNYANGPGYVISADIARYIVSEFDNQTLRLFKMEDVNMGMWVEKFNNTLRPVEYRHDVR
FYQSGCFDGYFTAHYQSPQHMICLWRKLQSGSSRCCNVR
>LOC_Os07g09670.2
(SEQ ID NO: 177)
MVSQFMVELVGTKAVDGEAPPRILHFNPRIRGDYSGKPVIEMNSCYRMQWGQSQRCEGYASRPADETVDGQLKCEKW
IRDDDKKSEESKMKWWVKRLIGRPKDVHISWPYPFAEGKLFVLTLTAGLEGYHVNVDGRHVTSFPYRTGYTLEDATG
LSLNGDIDIESIFASSLPNSHPSFAPERYLEMSEQWRAPPLPTEPVELFIGILSAASHFAERMAVRKSWMMYTRKST
NIVARFFVALNGKKEVNAELKREAEFFQDIVIVPFMDSYDLVVLKTIAIAEYGVRVIPAKYIMKCDDDTFVRIDSVL
DQVKKVRSDKSVYVGSMNYFHRPLRSGKWAVTYEEWPEEAYPNYANGPGYVISADIARYIVSEFDNQTLRLFKMEDV
NMGMWVEKFNNTLRPVEYRHDVRFYQSGCFDGYFTAHYQSPQHMICLWRKLQSGSSRCCNVR
>LOC_Os09g26300.1
(SEQ ID NO: 178)
MKTASSSSSSSHGFPATASLCTPYLLLVPLGLLAVVLVVPSLGSSHVRSDGLGVLCHAGPSTADGYLVTPGGDAASA
AAAAAETKAVVRPELRLLVGVLTTPKRYERRNIVRLAYALQPAVPPGVAQVDVRFVFCRVADPVDAQLVVLEAARHG
DILVLNCTENMNDGKTHEYLSSVPRMFASSPYDYVMKTDDDTYLRVAALVDELRHKPRDDVYLGYGFAVGDDPMQFM
HGMGYVVSWDVATWVSTNEDILRYNDTHGPEDLLVGKWLNIGRRGKNRYSLRPRMYDLNWDMDNFRPDTVLVHMLKD
NRRWAAAFRYFNVTAGLQPSNLYHFP
>LOC_Os09g26310.1
(SEQ ID NO: 179)
MAMKAPASSNSYLLLAPLALLLLAAVVFLLPSLNGARVGSDGGLGVLCARRSAGAEDYTVAAPAAPKEEEKPELSLL
VGVLTMPKRYERRDIVRLAYALQPAAARARVDVRFVFCRVADPVDAQLVALEAARHGDVVVLGGCEENMNHGKTHAY
LSSVPRLFASSPYDYVMKTDDDTYLRVAALADELRGKPRDDVYLGYGYAMGGQPMPFMHGMGYVVSWDVATWVSTAE
EILARNDTEGPEDLMVGKWLNLAGRGRNRYDLKPRMYDLSWDMDNFRPDTVAVHMLKDNRRWAAAFSYFNVTAGINL
HHLSP
>LOC_Os09g26320.1
(SEQ ID NO: 180)
MALSSLVVLSVSGCLSAPRSRPVVDNTNNDGGLGAETTAAREPEFRLLVGVLTTPSRYERRGILRLAYALQPAPGAQ
VDVRFVLCDVTDAADAVLVAAEAARHGDILVLDGCSTENMNDGKTHAYLSSVPRLFAPCPYDYVMKADDDTYLRVAA
LADELRGKPRRTSTSAGATPSATTRCRSCTAWATSCPGTSRAGCPPTRTSGRNRYNLKPRMYDINWDMDEFRPNTIA
VHRLKNNRRWAAVFRHFNVTVGIKPSTAARPHN
>LOC_Os12g16480.1
(SEQ ID NO: 181)
MCNQCLQGPYPLHTQALPQSYLDMSTVWQSSPLPNEPVDIFIGILSSGNHFAERMGVRKTWMSAVRNSPNVVARFFV
ALVHVVSARYVMKCDDDTFVRLDSIITEVNKVQSGRGLYIGNINFHHRSLRHGKWAVTYEEWPEEVYPPYANGPGYV
ISSDIAGAIVSEFRDRKLRVLSYSFLSGSATDWDESDGRRAAFVQVRSSVVAPRRQFNACWDWHLPDADDSGCVPAP
DPSLILGK
>LOC_Os12g41956.1
(SEQ ID NO: 182)
MKRARSSEVFLGGRGRARRRVAPLLAAVAFVYLLFVSFKLSGLAGIADPAAVTRPASGGAGEVVMPRRLEDPAPRAR
GDGDGVAVAGYGRITGEILRRRWEAGGRGRRRWGRGGNFSELERMADEAWELGGKAWEEACAFTGDVDSILSRDGGG
ETKCPASINIGGGDGETVAFLPCGLAVGSAVTVVGTARAARAEYVEALERRGEGNGTVMVAQFAVELRGLRAVEGEE
PPRILHLNPRLRGDWSHRPVLEMNTCFRMQWGKAHRCDGNPSKDDDQVDGLIKCEKWDRRDSVDSKETKTGSWLNRF
IGRAKKPEMRWPYPFSEGKMFVLTIQAGIEGYHVSVGGRHVASFPHRMGFSLEDATGLAVTGGVDVHSIYATSLPKV
HPSFSLQQVLEMSDRWKARPVPEEPIQVFIGIISATNHFAERMAIRKSWMQFPAIQLGNVVARFFVALSHRKEINAA
LKTEADYFGDVVILPFIDRYELVVLKTVAICEFGVQNVTAEYIMKCDDDTFVRLDVVLKQISVYNRTMPLYMGNLNL
LHRPLRHGKWAVTYEEWPEFVYPPYANGPGYVISIDIARDIVSRHANHSLRLFKMEDVSMGMWVEDFNTTAPVQYIH
SWRFCQFGCVHNYFTAHYQSPWQMLCLWNKLSSGRAHCCNYR
>LOC_Os02g31210.1
(SEQ ID NO: 183)
MSPLSLSFPSTLLPLSFFAWMWMALRLRRDREEGRRGRRLAGEERCGEEGRCDGRRHRCLLSRGWPRRSSPSTVGLD
AAQQSAPVHTPLHAPPRRGQAARRYSTDPVLVRPDDHARWEQGAEVTAVAVVVVLVEFDHAKTYYIGAPSESVEQDV
MHSYSMAFGGGGFAISYPAA
>LOC_Os03g16290.1
(SEQ ID NO: 184)
MVHLYPAAVPPHELQTPLRTFRAWSGSPAGPFTVNTRPEATPNATALPCHRKPIMFYLDRVTAMSTSTTNWTLTEYV
PEVLSGERCNTTGFDAATKVQMIQVIALKMNPAIWKRAPRRQCCKMQNANEGDKLIVKIHECKPDEATTSV
>LOC_Os06g19820.1
(SEQ ID NO: 185)
MATGGRRGRAVPLSKSFSRRLRHGRTGFGGSGHARGMMQLHVASAPSRCRRCWPTGGEETDGARWERRSGGRRYGGS
DDGDDAGAGEQGGGGGGEMSMVRYWRRSSVGAGSGSTAVDGGHGIASRPNAGASAGIARPRAISPTAVMNCNAPRAE
GQCGGDGVTDGCHRGGSGEAAEEGEIEDDGAVSAGVAQQSMEMPMRTFLNWYRCADYTAYVFNTRPLACQPCQMPQV
YYMRQSRLDRRRNTTVTEYERHRVAPVNCGWRIPDLATLLDRVIVLKKPDPDLWKRVIKHTP
>LOC_Os11g02650.1
(SEQ ID NO: 186)
MSACPVSLYFFPLFSPFPFSLSLFSLGALGRPAPPADGWGGDRRCEVGEEVRRPATWRRRRRGSRGTRRQRRRDVHG
EVSWGFAVVVTRTFLNWYRCADYTAYAFNTWPVACQPCQTPQVYYMQQSRLDRRRNTTVTVYERRRVVPAKCGWRIR
DPAALLDRVIVLKKPDPDL
>LOC_Os03g19310.1
(SEQ ID NO: 187)
MSKLQDRHGGEAAADVGRRARHQRLLLSFPVFPIVLLLLAPCTIFFFTSGDVPLPRIRIEYARRDAPTITAVAADTS
PPPPSPPSSSPPPLSFPPPPPPPSSPPPPALPVVDDHSDTQRSLRRLRQLTDSPYTLGPAVTGYDARRAEWLRDHTE
FPASVGRGRPRVLMVTGSAPRRCKDPEGDHLLLRALKNKVDYCRVHGFDIFYSNTVLDAEMSGFWTKLPLLRALMLA
HPETELLWWVDSDVVFTDMLFEPPWGRYRRHNLVIHGWDGAVYGAKTWLGLNAGSFIIRNCQWSLDLLDAWAPMGPP
GPVRDMYGKIFAETLTNRPPYEADDQSALVFLLVTQRHRWGAKVFLENSYNLHGFWADIVDRYEEMRRQWRHPGLGD
DRWPLITHFVGCKPCGGDDASYDGERCRRGMDRAFNFADDQILELYGFAHESLDTMAVRRVRNDTGRPLDADNQELG
RLLHPTFKARKKKTSPAARPM
>LOC_Os03g19330.1
(SEQ ID NO: 188)
MEKHGGKVTSDRRAGRRQHGQRCSASDAAPLVVVVILIVAALFLILGPTGSSSFTVPRIRVVFNEPVHVAVAAPPPP
PPPAQMQAGANASSEEDSGLPPPRQLTDPPYSLGRTILGYDARRSAWLAAHPEFPARVAPAGRPRVLVVTGSAPARC
PDPDGDHLLLRAFKNKVDYCRIHGLDVFYNTAFLDAEMSGFWAKLPLLRMLMVAHPEAELIWWVDSDAVFTDMLFEI
PWERYAVHNLVLHGWEAKVFDEKSWIGVNTGSFLIRNCQWSLDLLDAWAPMGPRGPVRDRYGELFAEELSGRPPFEA
DDQSALIYLLVTQRQRWGDKVFIESSYDLNGFWEGIVDKYEELRRAGRDDGRWPFVTHFVGCKPCRRYADSYPAERC
RRGMERAFNFADDQILKLYGFAHESLNTTAVRRVRNETGEPLDAGDEELGRLLHPTFRAARPT
>LOC_Os12g05380.1
(SEQ ID NO: 189)
MAVTGGGRPAVRQQAARGKQMQRTFNNVKITLICGFITLLVLRGTVGINLLTYGVGGGGGSDAVAAAEEARVVEDIE
RILREIRSDTDDDDDDEEEEPLGVDASTTTTTNSTTTTATAARRRSSNHTYTLGPKVTRWNAKRRQWLSRNPGFPSR
DARGKPRILLVTGSQPAPCDDAAGDHYLLKATKNKIDYCRIHGIEIVHSMAHLDRELAGYWAKLPLLRRLMLSHPEV
EWVWWMDSDALFTDMAFELPLARYDTSNLVIHGYPELLFAKRSWIALNTGSFLLRNCQWSLELLDAWAPMGPKGRVR
DEAGKVLTASLTGRPAFEADDQSALIHILLTQKERWMEKVYVEDKYFLHGFWAGLVDKYEEMMERHHPGLGDERWPF
VTHFVGCKPCGGYGDYPRERCLGGMERAFNFADNQVLRLYGFRHRSLASARVRRVANRTDNPLVNKEAALKMDAKIES
>LOC_Os02g17534.1
(SEQ ID NO: 190)
MGSAGGGGWDDDDDGDEQCATPPPPRSFSPMMMTEAGMKLVTPPWRRWRRWRGGCAESGRAVRAACVAAAVVLAVVV
LSYYARWGGDQDEMPTSLFTTRGSEGATSANLTDDQLLGGLLTAAFSPQSCRSRYEFAGYHKRKPPHKPSPYLVAKL
RSHEALQKRCGPGTAPYDKALRQLKSGDGAAAADGDDDCRYLVSISYNRGLGNRIIAIVSAFLYAVLTERALLVAPY
NGDVAALFCEPFPGTTWLLPDGGRRFPLLHLRDLDGKSKESLGALLKSNGIVSVAAGVNGSTSSSWSGRPPPPYVYL
HLDGGADYHDKLFYCDEQQRLLRGVPWLLMKTDSYLVPGLFLVPSLRGELERMFPEKDAVFHHLSRYLLHPANAVWH
AITAYHRDHLAGAGHLVGIQIRVYHEETPPVSQVVLDQVLSCARRENLLPAAGNTSSSDQAVLVTSLSSWYYEKIRD
ELGGGGGGVHQPSHEGLQRMGDTAHDMRALSEMYLLSTCDALLTTGFSTFGYVAQGLAGLRPWIMPRRPWWEKEAAT
AVPDPPCARVATPEPCFHSPSYYECAARRNYDDIGKVVPYVRRCEDVSWGIQLVNGSSQSQW
>LOC_Os02g17600.1
(SEQ ID NO: 191)
MEASLDQLPDQLSRGLEDAPPSNSNLTGDQLLGDLLSAAFSWQSCRSRHEALQVRCGPGTAPYEKALRQPKSGDGAI
AADGDDDDCRYVVSIVYDRGLGNRVIPIISAFLYAVLTERALLVAPYNGDVDALFCEPFPGTTWIHPGGRRFPLRRL
RDLDGKSRESLGTLLKSNAVSVDAGGNGTSSWSGRPPPYVYLHLDGGADYHDKLFYCDEQQRLLRGTPWLLMKTDSY
LVPGLFLVPSLRGELERMFPEKDAVFHHLSRYLLHPANAVWHAITAYHRDHLAGAGHLVGIQIRVYHEETPPVSQVV
LDQVLSCARREKLIPFPTAGTTTNTSSSDQAVLVTSLNSWYSDRIRDELGGGGGVHQPSHEGWQRMGDTAHDMRALS
EMYLLST
>LOC_Os02g25630.1
(SEQ ID NO: 192)
MRTTTRRRCDDCCSRLPTPLAKMLAWSVYGLPKWNFTNHINQRAKSQKLPYNMISGAGATCRGRGLDDEAEVEARDV
VERLDAAEEGRVWGRDGGAVEHGGVDLDLLILGAAGGVEAHPRGRHRRPLLTVERRGSREEGGEKVSNQITPPFSSP
SSLLPYLICEQFPGSTWTLPEGDFPFSGIRGFNACTRESLGNALRRGNALPETHYRHGCTCTCSTTYFNRNGNEPRF
FCDDGLDALWRVDWMVLLSDNYFVLGLFLVSRIERVLPRMFPCHDAAFHLLGRYLLHPRNVRTSCPVCSRSSTLPLP
ESSRAARPGRRKPVLVVSLHGAYSERIKDLYYEQDIAGRESMSVFQPTHLDRQQSGEKLHNQEEEEEYDKWGQGYF
>LOC_Os02g52590.1
(SEQ ID NO: 193)
MKSSKVQLHRAAAAASPSHGAPEDTPETSTRHDDDRLLGGLLSPAFDEHSCRSRYTSSLYRRRSPFRPSTYLVERLR
RYEARHKRCGPGSALFQEAVEHLRSGRNAARSECQYVVWTPFNGLGNRMLALASTFLYALLTDRVLLVHAPPEFDGL
FCEPFPGSSWTLPADFLITDFDGVFTMWSPTSYKNMRQAGTISNATAEQSLPAYVFLDLIQSFTDAAFCDDDQQVLA
KFNWMVIKSDVYFAAMLFLMPAYERELTQLFPEKEAVFHHLARYLFHPSNDVWGIVHRFYEAYLARADELVGLQVRV
FPEMPIPFDNMYEQIIRCSEQEGLLPKLGQTVVVTAANGSSVVAPSTKLTSILVTSLFPDYYDRIRGVYHARPTETG
EYVAVHQPSHEREQRTEARGHNQRALAEIYLLSFCDRVVTSAVSTFGYIAHGLAGVRPWVLLRPPSPVARAEPACVR
SETVEPCLQALPRRMCGAAEGSDIGALVPHIRHCEDVQKGIKLFS
>LOC_Os03g50800.1
(SEQ ID NO: 194)
MCPLSLSPPSSSFSLLATPAFSLPWQAARAGAAAGGDGGERRRRGSGGGRHWIGDGGLGNRILAAASAFLYAVLTAR
VLLVDTSNEMDELFSEPFPGTAWLLLRDFPLVLMRKTHTGLEGS
>LOC_Os04g37640.1
(SEQ ID NO: 195)
MSPSIRMAPAPSSSLATAGHGKTKSGRSSSSAVRPALLATAVSVMVVLLMAVLFGARWTPSGGHGGGADTSWVSAGA
RVVLNAVSSQQGADPVVKVAQPHDRLLGGLLSPDFNDTSCLSRYRASLYRRRSLHVLSSHLVSALRRYESLHRLCGP
GTSAYERAVARLRSPSSSNTTSDAPSECRYLVWTPHAGLGNRMLSITSAFLYALLTGRVLLFHRSGDDMKDLFCEPF
PGATWVLPEKDFPIRGMERFGIRTRESLGNALGRGEGGRDPPPPWMYVHLRHDYTRPGASDRLFFCDDGQDALRRVG
WVVVLLSDNYFVPGLFLIPRYERELSRMFPRRDAVFHHLGRYLFHPSNTVWGMVMRYHGSYLAKAEERVGVQVRTFSW
APISTDELYGQIVSCAQGENILPRVRESSSGSDNATAIPGSGRQQQQRPARRKAVLVVSLHGEYYERIRDMYYEHGA
AGGDAVSVFQPTHLGGQRSEERMHNQKALAEMMLLSFSDVALTSAASTFGYVSHGLAGLRPWVLMVPVRKKAPNPPC
RLAATVEPCFHTPPHYDCQARTKGDNGKTVRHVRHCEDLKDGVQLVD
>LOC_Os04g37650.1
(SEQ ID NO: 196)
MSPRVRMSVARWLPSSPAHGKTKSRRSSSAVRPTLLVIAVTVIAVLLVAVVFGGAGRWTLSGGGDTSWVSAGARVVI
NAVSGQQRDGDDPVAAAVEPRNDRLLGGLLSPDFDDSSCLSRYRAGLYRRQSPHAVSPHLVASLRRYESIHRRCGPG
TSAYERAVERLRSPPPSNTSDAECRYLVWTPLEGLGNRMLTLTSAFLYALLTDRVLLFHHPAGEGLRDLFCEPFPGS
TWTLPEGDFPFSGMQGFNARTRESLGNALRRGEGAAKDHPPPPPPWMYVHLRHDYNRNANDPRFFCDDGQDALRRVG
WVVLLSDNYFVPGLFLVPRFERALSRMLPRRDAAFHHLGRYLLHPSNTVWGMVARYHASYMACANERVGIQVRSFYW
ARISTDELYGQIMSCAHGENILPRVTQQGPNFTAAGDQPQPAARPGRRKAVLVVSLHGAYSERIKDLYYEHGAAGGE
SVSVFQPTHLDRQRSGEQLHNQKALAEMMLLSFSDVVVTSAASTFGYVGHGLAGLRPWVLMSPLDKKVPDPPCRLAA
TIEPCFHNPPNYDCRTRAKGDTGKIVRHIRHCEDFENGVQLVD
>LOC_Os06g10920.1
(SEQ ID NO: 197)
MATRGKKLGGVAGGGGAAVRVVGVVCVMAVPLFALLVLGGWASASTVWQSAARLTAVTAGFTNASKPSATGDAATGA
DELFGGLLAAGGCFDRGACLSRHESPRYYKSSPFSPSPYLLQKLRDYEARHRRCGPGTPGYAKSDEQLRSGHSSEVM
ECNYLVGLPYNGLGNRMLSLVASFLYALLTDRVFLVHFPDDFADHFCEPFPGGEGETATTWVLPPDFPVADLWRLGV
HSNQSYGNLLAAKKITGDPARETPVSVPPYVYLHLAHDLRGDDERFYCNDDQLVLAKVNWLLLQSDLYFVPSLYAIP
EFQDELRWMFPEKESVTHLLARYLLHPSNSVWGMVMRYHHAYLAPAAEMIGVQIRMFSWASIPVDDMYKQVMACSSQ
ERILPDTDGGDAPAPARTNTSGGGATTAILVASLQVEYYERLKGKYYEHAATASGGGRRWVGVFQPSHEEKQEMGKR
AHNQKALAEIYLLSFADVLLTSGMSTFGYMSSALAGLRPAMLLTAFNHKVPRTPCVRAVSMEPCFHKPPPAAATCQG
KLAVSENVTRHIKRCEDLAGGIKLFD
>LOC_Os06g10980.1
(SEQ ID NO: 198)
MDIDKLGEAAAAHPPEAEKRRGVAAPGAATVLVLVALPLMLVSYFFGDLAADTVVRLHRFKESSLSSSSPAAAADRL
LGGLLSPEFDEASCLSRYEASSRWKPSPFRVSPYLVERLRRYEANHRRCGPGTARYRDAVARLRSGDGDGDAECRYV
VWLPIQGLGNRMLSLVSTFLYALLTGRVVLVHEPPEMEGLFCEPFPGTSWLLPPDFPYKGGFSAASNESYVNMLKNG
VVRHDGDGGALPPYVYLHLEQIHLRLQNHTFCEEDHRVLDRFNWMVLRSDSYFAVALFLVPAYRAELDRMFPAKGSV
FHHLGRYLFHPGNRAWGIVERFYDGYLAGADERLGIQVRIVPQMAVPFDVMYEQILRCIREHGLLPQVTSTSESAGG
RPPPPPTATATKVKAVLVVSLKREYYDKLHGAYYTNATASGEVVAVYQPSHDGDQHTEARAHNERALAEIYLLSFSD
AVVTTAWSTFGYVAHALAGVRPWQLAPLDWGKMRADVACARPASVEPCLHSPPPLVCRARRDRDPAAHLPFLRHCED
VPAGLKLFD
>LOC_Os08g24750.1
(SEQ ID NO: 199)
MEMSGAGAGGVPTKLEHDDAAAVAAEREPCGGGAPRREEKERWRRVLVVGCLVALLLFAFFVLGRESASEVLQIASS
KLSAMNGGFTTKNPSHGGGAAKHADELLGGLLAPGMDRRSCRSRYQAAHYYKHFPYAPSPHLLDKLRAYEARHRRCA
PGTPLYNRSVEQLRSGRSAGGVECNYVVWLPFDGLGNRMLSMVSGFLYALLTDRVLLVDLPHDSSDLFCEPFPGATW
LLPPDFPVANLFGLGPRPEQSYTTLLNKKKITAVVNNDDDPASKNATAALPPPPAYVYLSLGYQMADKLFFCGDDQR
ALAKVNWLLLYSDLYFVPSLYSVAEFNGELRRLFPAKESACHLLARYLLHPTNAVWGMVTRYYNSYLAQASRRIGVQ
IRMFNFASIPVDDLYNQILTCSRQEHVLPETTTDNDNDDDLATAYDSNSSNGSGGGNYSAILIASLYPDYYERIRAT
YYEHATRGRVRVGVFQPTHEERQATQRLFHNQKALAEILLLGFSDELVTSGMSTFGYVGSSLAGVRPTILMPAHGHR
VPAPPCRRAVSMEPCNLTPPRVGEAECREMAAVVDKEDVARHVKVCEDFDRGVKFFD
>LOC_Os09g28460.1
(SEQ ID NO: 200)
MDSKPTRPHRRPPPLPSKTSGVWPVALLVVLCFAALPLFLALSRARPTLSDVSQMGVTVTVHDEDPAGTPPESSPAN
RDRLLGGLLSPDIGESACLSRYKSSLHRKPSPHSPSPYLVSRLRKYEALHRKCGPGTLFYKKSLMQLTSAYSMGLVE
CTYLVWTPCGGSHLGDRMLSMASAFLYALLTHRVFVVHVTDDMAGLFCEPFPAASWELPAGFLVHNLTQLGRGSEHS
YANLLGAKKIKTDDPAGVRSESLPSYAYVHLEHDYQQSDQLFFCDDDQTVLAKVNWLILRSNLYFTPGLFLVPQFED
ELRWMFPARDTVFHHIGRYLFHPSNKVWELITRYHTSYMAKFEENIGIQITTFAGSKVSSEEYFKQIVACTSQEKIL
PEIDPNATSSANEAALATTASKAVLVSSAQPSEYAEKLKAMYYEHATVTGEPVSVLQPAGAGKQAPNQKALVEMFLQ
SYCDVSVVSGRSTVGYVGHGLAGVKPWLLLTPTNRTASANPPCIQTTSMEPCFHAPPSYDCRAKKDGDLGAVLRHVR
HCEDVGDGLKLYD
>LOC_Os10g03650.1
(SEQ ID NO: 201)
MAEVGVIPVLPEPGPTTISAPRHTAADARGAPETESYNRAVQRLKDGSGKGSATEADARCGCSRATSRWCRSYANFS
ADSAESYGNMMKNKVLGTDGSDGDMPAAQMPAFAYLHLNHDYGDDDKMFFCDDDQRLVMRTDTYIVPSLFLVTTFQD
ELDALFPERGAVFHYLGRYLFPQANHTAVLQRVPRAGVAAAGRRPDCGSQALFCSSAAAAEDDTLTCAKPWRDILQI
LMSWLSINYFLESYVSLNRAKAALKGSYNPRGQEGWIWRSIESSLNQVAKEPKRRMGKDTGCARRGNRSRRCMEIRG
GEGGEGD
>LOC_Os02g28830.1
(SEQ ID NO: 202)
MGNALKDAGRVEEAINCYRSCLALQANHPQALTNLGNIYMEWNLISAAASFYKAAISVTSGLSSPLNNLAVIYKQQG
NYADAITCYTEVLRVDPTAADALVNRGNTFKEIGRVNEAIQDYIQAATIRPTMAEAHANLASAYKDSGHVETAIVSY
KQALRLRPDFPEATCNLLHTLQCVCDWENRNAMFRDVEEIIRKQIKMSVLPSVQPFHAIAYPIDPMLALEISCKYAA
HCSLIASRFGLPSFVHPPPVPVKAEGKHCRLRVGYVSSDFGNHPLSHLMGSVFGMHDRDNVEVFCYALSQNDGTEWR
QRIQSEAEHFVDVSAMTSDMIARIINQDKIQILINLNGYTKGARNEIFALQPAPIQVSYMGFPGTTGAAYIDYLVTD
EFVSPTCYSHIYSEKLVHLPHCYFVNDYKQKNRDCLDPVCPHKRSDYGLPEDKFIFACFNQLYKMDPEIFDTWCNIL
KRVPNSALWLLRFPAAGETRVRAHAAARGVRPDQIIFTDVAMKNEHIRRSSLADLFLDTPLCNAHTTGTDILWAGLP
MITLPLEKMATRVAGSLCLATGLGEEMIVSSMKEYEDRAVDLALNPAKLQALTNKLKEVRMTCPLFDTARWVRNLER
AYYKMWNLYCSGRHREPFKVIEDDNEFPYDR
>LOC_Os01g05400.1
(SEQ ID NO: 203)
MATPGPDELLKSHHILAKRREIRKREMEGVVVFADENSILRTELFDEVQKVKSVGAMPVGVLGEDEGTNEMFLQAPP
GCLPLTAGCYASSPVIASGEPEFAPAPFCRCPSDPRPPPPPSPSIRSLLPRADGDGYQEGNTWLSSSLCRIESLESL
STPLKNENDDSIFSLPTNIAVWAYDEGLLW
>LOC_Os01g06450.1
(SEQ ID NO: 204)
MDSEERSKKRLRLWSRAVVHFSLCFAIGVFAALLPLAATGATSIDSIRASFRPTVAATPPVPELDLLLIVTVTRPDD
DDDDGMSQEASLTRLGHTLRLVEPPLLWIVVGAENTTATARAVNALRGTRVMFRHLTYAAENFTGPAGDEVDYQMNV
ALSHIQLHRLPGVVHFAAASSVYDLRFFQQLRQTRGIAAWPIATVSSADQTVKLEGPTCNSSQITGWYSKDSSSNIT
ETTWDSSSNTTQTTWDSSSNKTQTTTLAALDTNASKQNSSSGPPEINMHAVGFKSSMLWDSERFTRRDNSSTGINQD
LIQAVRQMMINDEDKKRGIPSDCSDSQIMLWHLDMPRHTPKIEQATPEKESLTKGDEEESHDMTLDNVVPKTEEHET
LEKENLMKGDEKGSHDMMLDNVVAKIEEQETPEKENLTKGEEKESHDMMLDNVVAKIEEQETPEKENLTKGDEKESH
DMMLDNVVAKIDEQETTEKESLTKGDEKESHDMMLDNVVAKIEEQETPEKESLTKGDEKETHDMMLDNVVAKIEEQE
TPEEGKTKEG
>LOC_Os04g01280.1
(SEQ ID NO: 205)
MASIRRPHSPAKQQHLLRHGHLGPFASSSPPSSPLRHSSSSSSPRSAAHHHHHLLAAAGHTSFRRPLPRFAAFFLLG
SFLGLLHFLSHLPRPLGPIPNPNSHHRHRDPFPILQHPHPPSTPHSNHKLLIVVTPTRARPSQAYYLTRMAHTLRLL
HDSPLLWIVVQAGNPTPEAAAALRRTAVLHRYVGCCHNINASAPDFRPHQINAALDIVDNHRLDGVLYFADEEGVYS
LHLFHHLRQIRRFATWPVPEISQHTNEVVLQGPVCKQGQVVGWHTTHDGNKLRRFHLAMSGFAFNSTMLWDPKLRSH
LAWNSIRHPEMVKESLQGSAFVEQLVEDESQMEGIPADCSQIMNWHVPFGSESVVYPKGWRVATDLDVIIPLK
>LOC_Os04g58040.1
(SEQ ID NO: 206)
MGDEEEKGSRSRRMLIVVTTTRSDGGVRQRRNAALAHVEKHRLFSVVHFAHASGVYDAYFFDEIRQIERCHGRSPPP
GASLASCVCVVVVEGNDPAAVGSQEIAVPYRYTRYSSTCSRRKLGHGDVEEDEAEPSSPVGRHHIRARDWVSGRRET
PPQPRRETAAVLLSSSSSPIHLPRRDDAAAAIPNPHVCSVRACYATRAAASNSLPITGSIGLGDWEQFDVDHVHRAS
SELSMVAWSFTLPHHAGQCLVAQGLSAGLVLMHARL
>LOC_Os01g16460.1
(SEQ ID NO: 207)
MGAAAGSDERQMRPVVYVPSLFLVRARQSLWSAVAATGDRQRGGSNVDDRRLGEEARGVEGKEMGRPASRARRPASR
AGMPARRLASSAAGEEAAGARRCHADAGWRNTEIQPASMESNVDLRCQQTGAPAGECTSTSSLRGWYRLYSRSMDVC
KLVVNDGFGPALPSGGALPERDVYDTDQYMLALIYHTRMRRYECLTGERMARKKIRDAWSKLSPPPPDLSDTHDTRR
HRSRRAPSPSPASKLPPPPPLPPRPGGLVPSSAARLA
>LOC_Os01g70180.1
(SEQ ID NO: 208)
MGTRPCAGVASAVAAAVAVLLLAVSCFAAAATTTQKHGRMSGKGGDVLEDDPTGKLKVFVYEMPRKYNLNLLAKDSR
CLQHMFAAEIFMHQFLLSSPVRTLDPEEADWFYTPAYTTCDLTPQGFPLPFRAPRIMRSAVRYVAATWPYWNRTDGA
DHFFLAPHDFGACFHYQEERAIERGILPVLRRATLVQTFGQRHHPCLQPGSITVPPYADPRKMEAHRISPATPRSIF
VYFRGLFYDMGNDPEGGYYARGARASVWENFKDNPLFDISTEHPATYYEDMQRAIFCLCPLGWAPWSPRLVEAVVFG
CIPVIIADDIVLPFADAIPWGEISVFVAEEDVPRLDTILASVPLDEVIRKQRLLASPAMKQAVLFHQPARPGDAFHQ
ILNGLARKLPHPKGVFLEPGEKGIDWDQGLENDLKPW
>LOC_Os01g70180.2
(SEQ ID NO: 209)
MDVDVDCAGKGGDVLEDDPTGKLKVFVYEMPRKYNLNLLAKDSRCLQHMFAAEIFMHQFLLSSPVRTLDPEEADWFY
TPAYTTCDLTPQGFPLPFRAPRIMRSAVRYVAATWPYWNRTDGADHFFLAPHDFGACFHYQEERAIERGILPVLRRA
TLVQTFGQRHHPCLQPGSITVPPYADPRKMEAHRISPATPRSIFVYFRGLFYDMGNDPEGGYYARGARASVWENFKD
NPLFDISTEHPATYYEDMQRAIFCLCPLGWAPWSPRLVEAVVFGCIPVIIADDIVLPFADAIPWGEISVFVAEEDVP
RLDTILASVPLDEVIRKQRLLASPAMKQAVLFHQPARPGDAFHQILNGLARKLPHPKGVFLEPGEKGIDWDQGLEND
LKPW
>LOC_Os02g32110.1
(SEQ ID NO: 210)
MVGARAGRVPAAAAAAAAVLIVAACVFSSLAGAAAAAEVVGGAAQGNTERISGSAGDVLEDNPVGRLKVFVYDLPSK
YNKRIVAKDPRCLNHMFAAEIFMHRFLLSSAVRTLNPEQADWFYAPVYTTCDLTHAGLPLPFKSPRMMRSAIQFLSR
KWPFWNRTDGADHFFVVPHDFGACFHYQEEKAIERGILPLLRRATLVQTFGQKNHVCLKEGSITIPPYAPPQKMQAH
LIPPDTPRSIFVYFRGLFYDNGNDPEGGYYARGARASLWENFKNNPLFDISTEHPATYYEDMQRSVFCLCPLGWAPW
SPRLVEAVVFGCIPVIIADDIVLPFADAIPWDEIGVFVDEEDVPRLDSILTSIPIDDILRKQRLLANPSMKQAMLFP
QPAQPRDAFHQILNGLARKLPHPDSVYLKPGEKHLNWTAGPVADLKPWK
>LOC_Os03g05060.1
(SEQ ID NO: 211)
MRFSVVISSWILNRQLIKSAPKGGGRQQRRAKGMEKVAVGLLPPLRFIAVLAVVSWTSFIYCHFSLLSGGLLLGHGG
GDDGADPCRGRYIYVHDLPRRFNDDILRDCRKTRDHWPDMCGFVSNAGLGRPLVDRADGVLTGEAGWYGTHQFALDA
IFHNRMKQYECLTNQSAVADAVFVPFYAGFDFVRYHWGYDNATRDAASVDLTQWLMRRPEWRRMGGRDHFLVAGRTG
WDFRRDTNINPNWGTNLLVMPGGRDMSVLVLESSLLNGSDYAVPYPTYFHPRSDADVFRWQDRVRGMQRRWLMAFVG
APRPDDPKNIRAQIIAQCNATSACSQLGCAFGSSQCHSPGNIMRLFQKATFCLQPPGDSYTRRSVFDSMVAGCIPVF
FHNATAYLQYAWHLPREHAKYSVFISEHDVRAGNVSIEATLRAIPAATVERMREEVIRLIPSVIYADPRSKLETVRD
AFDVAVEGIIDRIAMTRGGYARSWLRPKQSRQALDARRRRLS
>LOC_Os03g05060.2
(SEQ ID NO: 212)
MEKVAVGLLPPLRFIAVLAVVSWTSFIYCHFSLLSGGLLLGHGGGDDGADPCRGRYIYVHDLPRRFNDDILRDCRKT
RDHWPDMCGFVSNAGLGRPLVDRADGVLTGEAGWYGTHQFALDAIFHNRMKQYECLTNQSAVADAVFVPFYAGFDFV
RYHWGYDNATRDAASVDLTQWLMRRPEWRRMGGRDHFLVAGRTGWDFRRDTNINPNWGTNLLVMPGGRDMSVLVLES
SLLNGSDYAVPYPTYFHPRSDADVFRWQDRVRGMQRRWLMAFVGAPRPDDPKNIRAQIIAQCNATSACSQLGCAFGS
SQCHSPGNIMRLFQKATFCLQPPGDSYTRRSVFDSMVAGCIPVFFHNATAYLQYAWHLPREHAKYSVFISEHDVRAG
NVSIEATLRAIPAATVERMREEVIRLIPSVIYADPRSKLETVRDAFDVAVEGIIDRIAMTRGGYARSWLRPKQSRQA
LDARRRRLS
>LOC_Os03g05070.1
(SEQ ID NO: 213)
MERTGAHGGKRLLPRLLFLAALSVTPWLLIFCLHFSVFDGAPPVSSPAARQSLVAVVSEGGEDSQRFLLEQEEQLRR
LPSARDVTTTTAAAVAGDAHACEGRYVYIHDLPPRFNDDILRNCREWYQWINMCVYLSNGGLGEPVDNADGAFADEG
WYATDHFGLDVIFHSRIKQYECLTDDSSRAAAVFVPFYAGFDVVQHLWGSNASVKDAASLELVDWLTRRPEWRSMGG
RDHFVMSGRTAWDHQRQTDSDSEWGNKFLRLPAVQNMTVLFVEKTPWTEHDFAVPYPTYFHPAKDAEIFQWQQRMRG
MKREWLFTFAGGTRPGDPNSIRHHLIRQCGASSLCNLIQCRKGEKKCLIPSTFMRVFQGTRFCLQPPGDTYTRRSAF
DAMLAGCVPVFFHPASAYTQYKWHLPDVHETYSVFIAEEDIRSGNVSVEETLRRIPPDVAEKMTETVISLVPRLLYA
DPRSKLETVKDAVDLTVEAVIERVKKLRKEMHGAGASSRLSTALGANTNGGFQSS
>LOC_Os03g08420.1
(SEQ ID NO: 214)
MYELPPRFNAEIVRDCRLYSRSMDVCKLVMNDGFGPAALPSGGALPERDVYDTDQYMLALIYHARMRRYECLTGESM
ARKKTRDARSKLAPPPPDLSDTTLAAIGADVRPRLLPHRSRRCRFLPHLAPGASSPAAPPGLPEKRREERKECKCAL
LGEEGIEINVGVEVVIDGVLNEGVDVLAIPEEGREKEGDNEGGDWGSGERHATARKEQLEEEGSVGRGLKLREALND
NMRRSIIYHLLTANLMLVNLIISSIWLVGS
>LOC_Os04g32670.1
(SEQ ID NO: 215)
MGSRTVGWWLLAAAVVLAAAAADSGEAERAAEQHSERISGSAGDVLEDNPVGRLKVFIYDLPRKYNKKMVNKDPRCL
NHMFAAEIFMHRFLLSSAVRTLNPKEADWFYTPVYTTCDLTPAGLPLPFKSPRVMRSAIQYISHKWPFWNRTDGADH
FFVVPHDFGACFHYQEEKAIERGILPLLQRATLVQTFGQENHVCLKEGSITIPPYAPPQKMQAHLIPPDTPRSIFVY
FRGLFYDTGNDPEGGYYARGARASLWENFKNNPLFDISTDHPPTYYEDMQRAVFCLCPLGWAPWSPRLVEAVVFGCI
PVIIADDIVLPFADAIPWEEIGVFVEEKDVPKLDTILTSMPIDDILRKQRLLANPSMKQAMLFPQPAQPRDAFHQIL
NGLARKLPHPEGVYLQPSDKRLNWTAGPVGDLKAW
>LOC_Os04g54100.1
(SEQ ID NO: 216)
MAAVAAATCDDTVGECDVDDEEEVEEMALMGAAGAAAGETGCRTLQRRLLDYLAARPEWRRSGGRDHVVLAHHPNGM
LDARYKLWPCVFVLCDFGRYPPSVVGLDKDVIAPYRHVVPNFAGQRLRRLR
>LOC_Os06g43160.1
(SEQ ID NO: 217)
MERSFRVFVYPDGDPGTFYQTPRKLTGKYASEGYFFQNIRESRFRTDDLEKAHLFFVPISPHKMRGKVPSSLLLVTY
AWLILHIRSYDRSILFLDLYWWCPLCSSFRGHWGVGADHFFVTCHDVGVRAFEGLPFIIKNSIRVVCSPSYNAGYIP
HKDVALPQILQPFALPAGGNDIENRTILGFWAGHRNSKIRVILARIWENDTELAISNNRINRAIGNLVYQKHFFRTK
FCVCPGGSQVNSARISDSIHYGCMPVILSDYYDLRFSGILNWRKFAVVLKESDVYELKSILKSLSQKEFVSLHKSLV
QVQKHFEWHSPPVPYDAFHMIMYELWLRHHVIKY
>LOC_Os06g46690.1
(SEQ ID NO: 218)
MASKNSCACHGVVVTLASCLLLVAAAVSVSVLAAHVAVGRVWSPAGAAAAAGHHHSLSPAWVPSPSSRHAHHARELV
NRRVQVGRMEAGLVQARVSIRRASRTRSCTPDDGGGFIPRGAVYRDAYAFHQSYIEMEKRFKVWTYREGEPPVVQKG
GAAFAGNDGIEGHLIAELDSSGGGGRHRARHPGEAHAFFLPISVASIAGYVYRRDMIDFWDPQLRLVAGYVDGLAAM
YPFWNRSRGADHFLVSCHQWAPILSAAKAELRGNAIRVMCDADMSDGFDPATDVALPPVVASARATPPQGRVASERT
VLAFFAAGGGGGGAVREALLARWEGRDDRVVVYGRLPAGVDHGELMRRARFCLCPCGGGEGAAAASRRVVEAITAGC
VPVLVDDGGYSPPFSDVLDWARFSVAVPAERVGEIKDILGGVSDRRYGVLRRRVLRVRRHFRLNRPPAKRFDVVNMV
IHSIWLRRLNLSLPY
>LOC_Os07g09050.3
(SEQ ID NO: 219)
MRASSFSSLMLPCSHGHGGGRATASTCAAAAAACLALVALVILVVSMDPRAQASSWFFLSSSSSSSSSSSSTLVRPA
ASSHAASLRKPSSWGGGNGGGGGGEHLLVTSSSFGSGGGARGSWSRNSTSKEVLFQGGGGGGGDEMTSTAAAPTPAL
IIGSSSGDGVSPSRVAVTAAAAEPTPALAPAPAPEWGVGDAASGDDIIQVMPQAQRRRDVKLELLELGLAKARATIR
EAIYLECVPVVIGDDYTLPFADVLNWAAFSVRVAVGDIPRLKEILAAVSPRQYIRMQRRVRAVRRHFMVSDGAPRRF
DVFHMILHSIWLRRLNVRVIARED
>LOC_Os10g10080.1
(SEQ ID NO: 220)
MGTRRRSARARARPPLAMPLAVLLLFACSSGVAAAAAQGIERIKDDPVGKLKVYVYELPPKYNKNIVAKDSRCLSHM
FATEIFMHRFLLSSAIRTSNPDEADWFYTPVYTTCDLTPWGHPLTTKSPRMMRSAIKFISKYWPYWNRTEGADHFFV
VPHDFAACFYFQEAKAIERGILPVLRRATLVQTFGQKNHACLKDGSITVPPYTPAHKIRAHLVPPETPRSIFVYFRG
LFYDTSNDPEGGYYARGARASVWENFKNNPMFDISTDHPQTYYEDMQRAVFCLCPLGWAPWSPRLVEAVVFGCIPVI
IADDIVLPFSDAIPWEEIAVFVAEDDVPQLDTILTSIPTEVILRKQAMLAEPSMKQTMLFPQPAEPGDGFHQVMNAL
ARKLPHGRDVFLKPGQKVLNWTEGTREDLKPW
>LOC_Os10g10080.2
(SEQ ID NO: 221)
MPLAVLLLFACSSGVAAAAAQGIERIKEDDPVGKLKVYVYELPPKYNKNIVAKDSRCLSHMFATEIFMHRFLLSSAI
RTSNPDEADWFYTPVYTTCDLTPWGHPLTTKSPRMMRSAIKFISKYWPYWNRTEGADHFFVVPHDFAACFYFQEAKA
IERGILPVLRRATLVQTFGQKNHACLKDGSITVPPYTPAHKIRAHLVPPETPRSIFVYFRGLFYDTSNDPEGGYYAR
GARASVWENFKNNPMFDISTDHPQTYYEDMQRAVFCLCPLGWAPWSPRLVEAVVFGCIPVIIADDIVLPFSDAIPWE
EIAVFVAEDDVPQLDTILTSIPTEVILRKQAMLAEPSMKQTMLFPQPAEPGDGFHQVMNALARKLPHGRDVFLKPGQ
KVLNWTEGTREDLKPW
>LOC_Os10g10080.3
(SEQ ID NO: 222)
MFATEIFMHRFLLSSAIRTSNPDEADWFYTPVYTTCDLTPWGHPLTTKSPRMMRSAIKFISKYWPYWNRTEGADHFF
VVPHDFAACFYFQEAKAIERGILPVLRRATLVQTFGQKNHACLKDGSITVPPYTPAHKIRAHLVPPETPRSIFVYFR
GLFYDTSNDPEGGYYARGARASVWENFKNNPMFDISTDHPQTYYEDMQRAVFCLCPLGWAPWSPRLVEAVVFGCIPV
IIADDIVLPFSDAIPWEEIAVFVAEDDVPQLDTILTSIPTEVILRKQAMLAEPSMKQTMLFPQPAEPGDGFHQVMNA
LARKLPHGRDVFLKPGQKVLNWTEGTREDLKPW
>LOC_Os10g32080.1
(SEQ ID NO: 223)
MAASVVSDKSSGGASLLRPSRVLFLAVLSTAFWSVIFYAHHSAVQGNATMASVLLRPSSFSRPLLTSFRLIGGGLDR
CAGRRVYMYELPPRFNAELVRDCRLYSRSMDVCKLVVNDGFGPALPGGGALPERDVYDTDQYMLALIYHARMRRYEC
LTGDAAAADAVFVPFYAGFDAAMNLMKSDLAARDALPRQLAEWLVRRPEWRAMGGRDHFMVAARPVWDFYRGGDDGW
GNALLTYPAIRNTTVLTVEANPWRGIDFGVPFPSHFHPTSDADVLRWQDRMRRRGRRWLWAFAGAPRPGSTKTVRAQ
IIEQCTASPSCTHFGSSPGHYNSPGRIMELLESAAFCVQPRGDSYTRKSTFDSMLAGCIPVFLHPASAYTQYTWHLP
RDYRSYSVFVPHTDVVAGGRNASIEAALRRIPAATVARMREEVIRLIPRITYRDPAATLVTFRDAFDVAVDAVLDRV
ARRRRAAAEGREYVDVFDGHDSWKHNLLDDGQTQIGPHEFDPYL
>LOC_Os10g32110.1
(SEQ ID NO: 224)
MAILSAVFWFLVFSLLSGMPGGGDLSSVLFRPSSLSLPLLNSFTFDQNPSPEQQPPPAPAPAEDRCAGRYIYMYDMP
ARFNEELLRDCRALRPWTAEGMCRYVANGGMGEPMGGDGGGIFSERGWFDTDQFVLDIIFHGRMKRYGCLTGDPAAA
AAVFVPFYGSCDLGRHIFHRNASVKDALSEDLVGWLTRRSEWRAMGGRDHFFVAGRTTWDFRRERDEGWEWGSKLLN
YPAVQNMTAILVEASPWSRNNLAVPYPTYFHPETAADVAAWQRRVRAAARPWLFSFAGGPRKGNGTIRADIIRQCGA
SSRCNLFHCHGAAASGCNAPGAVMRVFESSRFCLEPRGDTMTRRSTFDAILAGCIPVFFHPGSAYTQYTLHLPPERG
GWSVLIPHADVTGRNVSIEETLAAISPEKVRSMREEVIRLIPTVVYADTRSSRVDFRDAFDVAVDAVVGRVARRRRD
EPDARR
>LOC_Os10g32160.1
(SEQ ID NO: 225)
MKRHNAAEVPVPVSYGGERDEKTGSKVDKMGGGAARRWRSGGCCSRLWLVLVVFATVTMLLRHRYDSGLGHGAAAVV
RIEPVHRKVKPADRGGARPSFSDSGSAKPVTVDHKSATTDSSTGTESDGGGEPSSASSSLPAAAHPFSRALAAAGDK
GDRCGGRYVYVQELPPRFNTDMVKNCVALFPWKDMCKFTANGGFGPPMSGGGGMFQETGWYNSDKYTVDIIFHERMR
RYECLTDDPSLAAAVYVPFFAGLEVWRHLWGFNATARDAMALEVVDIITSRPEWRAMGGRDHFFTAGLITWDFRRLA
DGDAGWGSKLFSLPAIKNMTALVVEASPWHLNDAAIPFPTAFHPASDEAVFVWQDKVRRLERPWLFSFAGAARPGSA
KSIRSELITQCRASSACSLMECRDGPSNKCGSAASYMRLFQSSTFCLQPQGDSYTRKSAFDAMLAGCIPVFFHPGTA
YVQYTWHLPRNHADYSVYISEDDVRRNASIEERLRRIAPAAVERMRETVISLIPTVVYAQPSSRLDTMKDAFDVAVD
AIVDKVTRLRRDIVDGRGEEEKLEMYSWKYPLLREGQKVEDPHEWDSLFAFA
>LOC_Os10g32170.1
(SEQ ID NO: 226)
MKRHNTAEVPVPVSYGGKVEKTMGGAKQGRGGGGGCCSRLWFMVVLSATVTLLVRHCYDSGVIGHGAAAGGVVRIEP
VHRGLYHTRKASPVDRGGGGGGTSFSGHSPSPPDAGGSAKPESPHDSGVKAPSELTTVEHTKQPSEPASTGTESDDG
GKPSSASSSSLPAAAHPFARALAAAGDKGDRCGGRYVYVQELPPRFNTDMVKNCATLFPWTDMCAFTANGGFGPQMS
GGDGGVFQETGWYNSDQYTVDIIFHDRIRRYECLTDDPSLAAAVYVPFFAGLEVARHLWGFNVTTRDAMALEVVDII
TSRSEWRAMGGRDHFFTAGRTTWDFRRLNDGDAGWGSKLFSLPAIKNMTALVVEASPWHLNDAAIPFPTAFHPASDE
AVFVWQDKVRRLERPWLFSFAGAARPGSAKSIRSELIAQCRASSVCSLMECADGPSNKCGSPASYMRLFQSSTFCLQ
PQGDSYTRKSAFDAMLAGCIPVFFHPGTAYVQYTWHLPRNHADYSVYISEDDVRRNASIEERLRRIAPAAVERMRET
VISLIPTVVYAQPSSRLDTMKDAFDVAVDAIVDKVTRLRRDIVDGRGEEEKLEMYSWKYPLLREGQKVEDPHEWDPL
FAFG
>LOC_Os12g12290.1
(SEQ ID NO: 227)
MEANYPKYVLYGLLIVGSWLLSCLLHFQVFHLSLFPYPSYLLSRRVVLPLALNARFLPPRPDVAGDDDGGIVRRRSS
SPAKAAAEASCDGRYVYVLEVPRRFQMLTECVEGPKVFDDPYHVCVVMSNSGLGPVIPPAAAGNATVDGDIIPNTGW
YNTDQYALEVIFHNRMRRYECLTSDMAAATAVYVAFYPALELNRHKCGSSATERNEPPREFLRWLTSQPSWAALGGR
DHFMVAARTTWMFRRGGAGDSLGCGNGFLSRPESGNMTVLTYESNIWERRDFAVPYPSYFHPSSAREVSAWQATARA
ARRPWLFAFAGARRANGTLAIRDHIIDECTASPPGRCGMLDCSHGLEGSITCRSPRRLVALFASARFCLQPPGDSFM
RRSSIDTVLAGCIPVFFHEASTFKKQYQWHERDADADNDNATVDRRRYSVVIDPDDVVEGRVRIEEVLRRFSDDEVA
AMREEVIRMIPRFVYKDPRVRFEGDMRDAFDITFDEIMARMRRIKNGEILGWKLDGDDDVVAKDS
>LOC_Os12g16230.1
(SEQ ID NO: 228)
MPLTLFFFLLSFLPLTRVAFFAAGGGAMREVLLTRWEGRDDQVLLYGLLPAGVDHGELMGRARFCLCPTGDDEGAAA
ASRRVVEAITVGCCAVDSAVSFLRRRHR
>LOC_Os12g38450.1
(SEQ ID NO: 229)
MDGSHGSAAALRATGRCLAPLIIPASCVVWVLFFFPSPSPDVAVRRDGFLPAVTLPVQRAGDTPPPPPIIDASPPPP
STSPPPPPPRRGRPARRDRCAGRYVYMHELPSRFNSDLLRDCRTLSEWTDMCRHVANGGIGPRLPPAARGGVLPATG
WYDTNQFTLEVIFHARMRRYGCLTADASRAAAVYVPYYPGLDVGRYLWGFSNGVRDLLAEDLAEWLRGTPAWAAHGG
RDHFLVGGRIAWDFRREDGGGEGSQWGSRLLLLPEAMNMTALVIEASPWHRRTDVAVPYPTYFHPWRPSDVSSWQRD
ARRARRPWLFAFAGAGRGNGDDHDRHHGGGVVRDRVIAQCARSRRCGLLRCGARGRRDDCYDPGNVMRLFKSAAFCL
QPRGDSYTRRSVFDAILAGCVPVFFHPGSAYTQYRWHLPRDHAAYSVFVPEDGVRNGTVRLEDVLRRVSAARVAAMR
EQVIRMIPTVVYRDPRAPSARGFTDAIDVAVDGVIERVRRIKQGLPPGGDDDDDHRWDAYFDTQ
>LOC_Os01g34880.1
(SEQ ID NO: 230)
MREGNVTHHEYMQVGKGRDVGMNQISSFEAKVANGNGEQTLSRDIYRLGRRFDFYRMLSFYFTTVGFYFSSMVTVLT
VYVFLYGRLYLVMSGLERSILLDPRIEQNIKPLENALASQSFFQLGLLLVLPMVMEVGLEKGFRTALGEFVIMQLQL
ASVFFTFQLGTKTHYYGRTILHGGAKYRPTGRGFVVYHAKFADNYRMYSRSHFVKGLELLILLVVYLVYGSSYRSSS
MYLFVTFSIWFLVASWLFAPFIFNPSCFEWQKTVDDWTDWRKWMGNRGGIGMSVDQSWEAWWISEQEHLRKTSIRSL
LLEIILSLRFLIYQYGIVYHLNIARRSKSILVYGLSWLVMLSVLVVLKMVSIGRQKFGTDLQLMFRILKGLLFLGFV
SVMAVLFVVCNLTISDVFASILGFMPTGWCILLIGQACSPLVKKAMLWDSIMELGRSYENLMGLVLFLPIGLLSWFP
FVSEFQTRLLFNQAFSRGLQISRILAGQKDIGEE
>LOC_Os01g34890.1
(SEQ ID NO: 231)
MPMGSRLGASPEMIERNMSLMLLLIVEKTGPFDIYAKEVEKEKASFSHYNILPLNISGQRQPVMEIPEIKAAVDLLR
KIDGLPMPRLDPVSAEKETDVPTVRDLFDWLWLTFGFQKGNVENQKEHLILLLANIDMRKGANAYQSDRHNHVMHSD
TVRSLMRKIFENYISWCRYLHLESNIKIPNDASTQQPEILYIGLYLLIWGEASNVRFMPECICYIFHHSHQYKNTII
PMCLFMEHVRQDFDPPFRREGSDDAFLQLVIQPIYSVMKQEAAMNKRGRTSHSKWRNYDDLNEYFWSKRCFKQLKWP
MDSAADFFAVPLKIKTEEHHDRVITRRRIPKTNFVEVRTFLHLFRSFDRMWAFFILAFQAMVIVAWSPSGLPSAIFD
PTVFRNVLTIFITAAFLNFLQATLEIILNWKAWRSLECSQMIRYILKFVVAVAWLIILPTTYMSSIQNSTGLIKFFS
SWIGNLQSESIYNFAVALYMLPNIFSALFFIFLPFRRVLERSNSRIIRFFLWWTQPKLYVARGMYEDTCSLLKYTLF
WILLLICKLAFSFYVEIYPLVGPTRTIMFLGRGQYAWHEFFPYLQHNLGVVITVWAPIVMVYFMDTQIWYAIFSTIC
GGVNGAFSRLGEIRTLGMLRSRFEAIPIAFGKHLVPGHDSQPKRHEHEEDKINKFSDIWNAFIHSLREEDLISNRER
NLLIVPSSMGDTTVFQWPPFLLASKIPIALDMANSVKKRDEELRKRINQDPYTYYAVVECYQTLFSILDSLIVEQSD
KKVVDRIHDRIEDSIRRQSLVKEFRLDELPQLSAKFDKLLNLLLRTDEDIEPIKTQIANLLQDIMEIITQDIMKNGQ
GILKDENRNNQLFANINLDSVKDKTWKEKCVRLQLLLTTKESAIYVPTNLDARRRITFFANSLFMKMPKAPQPFCFC
ISVLTPYFKEEVLFSAEDLYKKNEDGISILFYLRKIYPDEWKNFLERIEFQPTDEESLKTKMDEIRPWASYRGQTLT
RTVKLEHRRTVESSQQGWASFDMARAIADIKFTYVVSCQVYGMQKTSKDPKDKACYLNILNLMLMYPSLRVAYIDEV
EAPAGNGTTEKTYYSVLVKGGEKYDEEIYRIKLPGKPTDIGEGKPENQNHAIVFTRGEALQAIDMNQDNYLEEAFKM
RNVLEEFESEKYGKRKPTILGLREHIFTGSVSSLAWFMSNQETSFVTIGQRVLANPLNFYGPSFIDRHH
>LOC_Os01g34930.1
(SEQ ID NO: 232)
MCIMQISFVLCSKLMVVLIKGFNSTLRQGNVTHHEYIQLGKGRDVGMNQISNFEAKVANGNGEQTLCRDIYRLGHRF
DFYRMLSLYFTTVGFYFNSMVAVLTVYVFLYGRLYLVLSGLEKSILQDPQIKNIKPFENALATQSIFQLGMLLVLPM
MIEVGLEKGFGRALGEFVIMQLQLASVFFTFHLGTKTHYYGRTILHGGAKYRGTGRGFVVRHAKFAENYRMYSRSHF
VKALELLILLVVYLAYGISYRSSSLYLYVTISIWFLVFCWLFAPFVFNPSCFEWHKTVDDWTDWWHWMSNRGGIGLA
PEQSWEAWWISEHDHLRNGTIRSLLLEFVLSLRFLIYQYGIVYHLHIVHGNRSFMVYALSWLVIAIVLVSLKVVSMG
REKFITNFQLVFRILKGIVFIVLISLVVILFVVFNLTVSDVGASILAFIPTGWFILQIAQLCGPLFRRLVTEPLCAL
FCSCCTGGTACKGRCCARFRLRSRDVLRKIGPWDSIQEMARMYEYTMGILIFFPIAVLSWFPFVSEFQTRLLFNQAF
SRGLQISRILTGQNGSGSKRD
>LOC_Os01g48200.1
(SEQ ID NO: 233)
MFEAKVASGNGEQTLSRDVYRLGHRLDFFRMLSFFYTTIGFYFNTMMVVLTVYAFVWGRFYLALSGLEAFISSNTNS
TNNAALGAVLNQQFVIQLGIFTALPMIIENSLEHGFLTAVWDFIKMQLQFASVFYTFSMGTKTHYYGRTILHGGAKY
RATGRGFVVEHKKFAENYRLYARSHFIKAIELGVILTLYASYGSSSGNTLVYILLTISSWFLVLSWILAPFIFNPSG
LDWLKNFNDFEDFLNWIWFRGGISVKSDQSWEKWWEEETDHLRTTGLFGSILEIILDLRFFFFQYAIVYRLHIAGTS
KSILVYLLSWACVLLAFVALVTVAYFRDKYSAKKHIRYRLVQAIIVGATVAAIVLLLEFTKFQFIDTFTSLLAFLPT
GWGIISIALVFKPYLRRSEMVWRSVVTLARLYDIMFGVIVMAPVAVLSWLPGLQEMQTRILFNEAFSRGLHISQIIT
GKKSHGV
>LOC_Os02g14900.1
(SEQ ID NO: 234)
MEVEIVEQRWRPTPTPLPPPPPPLPPPPAASAASSSGAGDAAAVASAANQFDSEKLPQTLVSEIRPFLRVANQIEHE
SPRVAYLCRFHAFEKAHMMDPRSTGRGVRQFKTALLQRLEQDEKSTFTKRMAKSDSQEIRLFYEKKEKADERELLPV
LAEVLRAVQIGTGKEKQKRIASETFADKSALFRYNILPLYPGSTKQPIMLLPEEKKGNVANQREHLILLLANMHARL
NPKSSSETMLDDRAVDELLAKTFENYLTWCKFLGRKSNIWLPSVKQEIQQHKLLYISLYLLIWGEASNLRLMPECLC
YIFHHESLKNKNGVSDHSTWRNYDDLNEFFWSADCFKLGWPMRLNNDFFFTSNKNKNSRLPIVPPVQQTEQQINQLR
TSQQTDQQNTQLRTSQQTEQRNTQLRTPNGSSSFQNMLNPEAPGQTQQQTTSDTSQQKWLGKTNFVEVRSFWHIFRS
FDRMWTLLVLGLQFFRDIYLENLQYVSLVVSVKSNAGAILAVWAPIILVYFMDTQIWYSVFCTIFGEMDLMTMPMSL
EHRSGSIRWPMFLLAKKFSEAVDMVANFTGKSTRLFCIIKKDNYMLCAINDFYELTKSILRHLVIGDVEKSFSSACP
CEYYYDVLQILSRVIAAIYTEIEKSIQNASLLVDFKMDHLPSLVAKFDRLAELLYTNKQELRYEVTILLQDIIDILV
QDMLVDAQSVLGLINSSETLISDDDGTFEYYKPELFASISSISNIRFPFPENGPLKEQVKRLYLLLNTKEKVVEVPS
NLEARRRISFFATSLFMDMPSAPKVSNEWRNFLERLGPKVTQEEIRYWASFHGQTLSRTVRGMMYYRKALRLQAFLD
RTNDQELCKGPAANGRQTKNMHQSLSTELDALADMKFSYVISCQKFGEQKSSGNPHAQDIIDLMTRYPALRVAYIEE
KEIIVDNRPHKVYSSVLIKAENNLDQEIYRIKLPGPPLIGEGKPENQNHAIIFTRGEALQTIDMNQDNYLEEAYKMR
NVLQEFVRHPRGKAPTILGLREHIFTGSVSSLAGFMSYQETSFVTIGQRFLADPLRVRFHYGHPDIFDRMFHLTRGG
ISKASKTINLSEDVFAGYNSILRRGHITYNEYIQVGKGRDVGLNQISKFEAKVANGNSEQTLSRDIHRLGRRFDFFR
MLSCYFTTVGFYFNSLISVVGVYVFLYGQLYLVLSGLQRALLIEAETQNMKSLETALVSQSFLQLGLLTGLPMVMEL
GLEKGFRVALSDFILMQLQLASVFFTFSLGTKAHYYGRTILHGGAKYRPTGRKFVAFHASFTENYQLYSRSHFVKGF
ELVFLLIIYHIFRRSYVSTVVHVMITYSTWFMAVTWLFAPFLFNPAGFAWRKIVEDWADWTIWMRNQGGIGVQPEKS
WESWWNAENAHLRHSVLSSRILEVLLSLRFFMYQYGLVYHLKISQDNKNFLVYLLSWVVIIAIVGLVKLVNCASRRL
SSKHQLVFRLIKLLIFLSVMTSLILLSCLCQLSIMDLIICCLAFIPTGWGLLLIVQVLRPKIEYYAIWEPIQVIAHA
YDYGMGSLLFFPIAALAWMPVISAIQTRVLFNRAFSRQLQIQPFIAGKTKRR
>LOC_Os03g02756.1
(SEQ ID NO: 235)
MNQDNYFEEALKMRNLLEEFYQNHGKHKPSILGVREHVFTGSVSSLASFMSNQETSFVTLGQRVLANPLKVRMHYGH
PDVFDRIFHITRGGISKASRVINISEDIYAGFNSTLRLGNITHHEYIQVGKGRDVGLNQIALFEGKVAGGNGEQVLS
RDIYRLGQLFDFFRMLSFYVTTIGFYFCTMLTVWTVYIFLYGKTYLALSGVGESIQNRVDILQNTALNAALNTQFLF
QIGVFTAIPMILGFILEFGVLTAFVSFITMQFQLCSVFFTFSLGTRTHYFGRTILHGGAKYRATGRGFVVRHIKFAE
NYRLYSRSHFVKGLEVALLLVIFLAYGFNNGGAVGYILLSISSWFMAVSWLFAPYIFNPSGFEWQKVVEDFRDWTNW
LFYRGGIGVKGEESWEAWWDEELAHIHNVGGRILETVLSLRFFIFQYGVVYHMDASESSKALLIYWISWAVLGGLFV
LLLVFGLNPKAMVHFQLFLRLIKSIALLMVLAGLVVAVVFTSLSVKDVFAAILAFVPTGWGVLSIAVAWKPIVKKLG
LWKTVRSLARLYDAGTGMIIFVPIAIFSWFPFISTFQTRLLFNQAFSRGLEISLILAGNNPNAGV
>LOC_Os03g02756.2
(SEQ ID NO: 236)
MNQDNYFEEALKMRNLLEEFYQNHGKHKPSILGVREHVFTGSVSSLASFMSNQETSFVTLGQRVLANPLKVRMHYGH
PDVFDRIFHITRGGISKASRVINISEDIYAGFNSTLRLGNITHHEYIQVGKGRDVGLNQIALFEGKVAGGNGEQVLS
RDIYRLGQLFDFFRMLSFYVTTIGFYFCTMLTVWTVYIFLYGKTYLALSGVGESIQNRVDILQNTALNAALNTQFLF
QIGVFTAIPMILGFILEFGVLTAFVSFITMQFQLCSVFFTFSLGTRTHYFGRTILHGGAKYRATGRGFVVRHIKFAE
NYRLYSRSHFVKGLEVALLLVIFLAYGFNNGGAVGYILLSISSWFMAVSWLFAPYIFNPSGFEWQKVVEDFRDWTNW
LFYRGGIGVKGEESWEAWWDEELAHIHNVGGRILETVLSLRFFIFQYGVVYHMDASESSKALLIYWISWAVLGGLFV
LLLVFGLNPKAMVHFQLFLRLIKSIALLMVLAGLVVAVVFTSLSVKDVFAAILAFVPTGWGVLSVSFYRNINVHLRQ
DGAKVNSVLKIVPA
>LOC_Os01g07720.2
(SEQ ID NO: 237)
MTFLHAAMVLIMYHKWYLGLVIFSAAVSIKMNVLLFAPSLLLLMLKAMSIKGVFFALLGAAALQVLLGMPFLLSHPV
EYISRAFNLGRVFIHFWSVNFKFVPEKFFVSKELAVALLVLHLTTLLVFAHYKWLKHEGGLFHFLHSRFKDATSIGQ
LIFAKPKLSTLNKEHIVTVMFVGNFIGIVCARSLHYQFYSWYFYSLPFLLWKTRFPTFVRVILFLAVELCWNIYPST
AYSSLLLLFIHISILFGLWSSPAEYPYANGKK
>LOC_Os01g07720.3
(SEQ ID NO: 238)
MTFLHAAMVLIMYHKWYLGLVIFSAAVSIKMNVLLFAPSLLLLMLKAMSIKGVFFALLGAAALQVLLGMPFLLSHPV
EYISRAFNLGRVFIHFWSVNFKFVPEKFFVSKELAVALLVLHLTTLLVFAHYKWLKHEGGLFHFLHSRFKDATSIGQ
LIFAKPKLSTLNKEHIVTVMFVGNFIGIVCARSLHYQFYS
>LOC_Os01g07720.4
(SEQ ID NO: 239)
MTFLHAAMVLIMYHKWYLGLVIFSAAVSIKMNVLLFAPSLLLLMLKAMSIKGVFFALLGAAALQVLLGMPFLLSHPV
EYISRAFNLGRVFIHFWSVNFKFVPEKFFVSKELAVALLVLHLTTLLVFAHYKWLKHEGGLFHFLHSRFKDATSIGQ
LIFAKPKLSTLNKEHIVTVMFVGNFIGIVCARSLHYQFYS
>LOC_Os01g02910.1
(SEQ ID NO: 240)
MKAAARERKPRHSNGRAAAAAAKNLSKVEPGRHLAVVRLFPACLLALLICLCVVKFFSSLSSQSQRIGTRSRMVSSW
EGSASTNVPRIPVAPLIMGRVDEDISTRSPELGVVIVFVLAVSRVVTFLVETCKGLGFCHWKPLSYPMLILKGKQGS
VFKNENFKNGTDSENKSRSERQVAISTENDPPPGKEESLTKSPQTAVSESEVPKPKSKISCDDKSKDEGFPYARPIV
CHLSGDVRVSPATSSVTLTMPLQQGEAAARRIRPYARRDDFLLPLVREVAITSAASEGDAPSCNVSHGVPAVIFSIG
GYTGNFFHDMADVLVPLYLTTFHFKGKVQLFVANYKQWWIQKYKPVLRRLSHRAVVDFDSDGDVHCFDHVIVGLVRD
RDLILGQHPTRNPKGYTMVDFTRFLRHAYGLRRDKPMVLGETSGKKPRMLIISRRRTRKLLNLRQVAAMARELGFEV
VVSEAGVGGGSGGVKRFASAVNSCDVLVGVHGAGLTNQAFLPRGGVVVQIVPWGRMEWMATNFYGAPAAAMELRYVE
YHVAAEESSLARRYPREHAVFRDPMAIHGQGWKALADIVMTQDVKLNLRRFRPTLLRVLDLLQD
>LOC_Os01g02920.1
(SEQ ID NO: 241)
MGGDHGKLMKSLKGAAQKYLGVGFLLGFFLVLLTYFTVSEQFAIAAPNAIRKTSPGHASPTIPPPVEEKRPQLPPII
EQRQAPKAEHEHAAVVQEKTPSAEEIEIQKETEEDHTKEKPTDDVTTTVEESAPAKKPACDIQGPWASDVCSIDGDV
RIHGAAHDVVIPPPIEGGGSNPNPREWRVVPYSRKHMGGLKEVAVREVASAAEAPACDVRSPVPALVFAMGGLTGNY
WHDFSDVLIPLYLQARRFDGEVQLVVENIQMWYVGKYKRVLDRLSRHDIVDMDRDDKVRCFPGAVVGIRMHKEFSID
PARDPTGHSMPEFTKFLRDTFSLPRDAPVSLVDNAAAVRPRLMIISRRHPRKLMNVEEVVRAAERIGFEVVIGDPPF
NVDVGEFAKEVNRADVLMGVHGAGLTNSVFLPTGAVLIQVVPYGKMEHIGKVDFGDPAEDMRLKYMAYSAGVEESTL
VETLGRDHPAVRDPESVHRSGWGKVAEYYLGKQDIRLDLARFEPLLRDAMDYLKHQ
>LOC_Os01g02930.1
(SEQ ID NO: 242)
MGSEVKPAKLGLRRHLNAGFFAGFLLVLLTYVIVSQQFAMETPTAVTSRAPRIDENESVTKARVE
TEKKREQEWQRPKDTSGAVSAEEFSKRDSTNAKPIENGKVVCGSNGFYSDTCDVDGDVRINGTALSVTLVPASRRSE
RRREWKIQPYPRRTVSGIAEVTVTRQQDRAAAPACTVTHGVPGVVFALGGLTGNYWHDFSDVLVPLFVASRRYGGEV
QFLVSNIQPWWLGKYEAVVRRLSRYDAVDLDRDTEVRCFRRVAVGLRMHKEFSVKPELAPGGQRLTMADFAAFLRDT
YALPRAAAAGARRPRLVVIRRAHYRKIVNMDEVVRAAEAAGFEAAVMSPRFDEPVEEVARKVNAFDAMVGVHGAGLT
NAVFLPAGAVVIQVVPYGRLERMARADFGEPVADMGLRYMEYSVAADESTLLEMLGPEHQVVKDPEAVHRSGWDKVA
EYYLGKQDVRINVARFAATLAAAFDHLRPSHS
>LOC_Os01g02940.1
(SEQ ID NO: 243)
MEGGGKAGYSYGGHHHHQDAKLLKNLSRVEPRRFGLGLVAGFLIVTCAYFSTAKFDAIHIAMISSPAKNAAGFMNAS
SDGSNQQQLDLDRDAMSREGSKAQVLDTDGDDKISSLGPDLGHNASALEGKKKDETFAKDSGDASVSASTDEALAKD
DDAIVGAVLPPLSSEEPTNITQDSVLEDEELKVQETAPATTNPSPEKSSNNGSSPSVVPSDPATLPVQQIPPTQEAK
DPPAQQIPAVPEAKVPPVQQIPTFPVVKTDSEAAPRRKEWKPLCDLWSNRRIDWCELDGDVRVAGANGTVSLVAPPG
PADERTFRAESWHIKPYPRKADPNAMRHVRVLTVQSLPAPAASAAAPACTERHDVPGLVFSDRGYTGNYFHAYTDVI
LPLFLTARQYSGEVKLLVSDFQMWWLGKFLPVFKAVSNYDLINLDDDRRVHCFRHVQVGLTCHADFSIDPSRAPNGY
SMVDFTRFMRATYRLPRDAPFPASGEQQPRRPWRPRLLVIARARTRRFVNADEIVRGAERAGFEVVVSEGEHEVAPF
AELANTCDAMVGVHGAGLTNMVFLPTGGVVIQVVPLGGLEFVAGYFRGPSRDMGLRYLEYRITPEESTLIDQYPRDH
PIFTDPDGVKSKGWNSLKEAYLDKQDVRLDMKRFRPILKKAIAHLRKNSGNNNTTHN
>LOC_Os01g02940.2
(SEQ ID NO: 244)
MEGGGKAGYSYGGHHHHQDAKLLKNLSRVEPRRFGLGLVAGFLIVTCAYFSTAKFDAIHIAMISSPAKNAAGFMNAS
SDGSNQQQLDLDRDAMSREGSKAQVLDTDGDDKISSLGPDLGHNASALEGKKKDETFAKDSGDASVSASTDEALAKD
DDAIVGAVLPPLSSEEPTNITQDSVLEDEELKVQETAPATTNPSPEKSSNNGSSPSVVPSDPATLPVQQIPPTQEAK
DPPAQQIPAVPEAKVPPVQQIPTFPVVKTDSEAAPRRKEWKPLCDLWSNRRIDWCELDGDVRVAGANGTVSLVAPPG
PADERTFRAESWHIKPYPRKADPNAMRHVRVLTVQSLPAPAASAAAPACTERHDVPGLVFSDRGYTGNYFHAYTDVI
LPLFLTARQYSGEVKLLVSDFQMWWLGKFLPVFKAVSNYDLINLDDDRRVHCFRHVQVGLTCHADFSIDPSRAPNGY
SMVDFTRFMRATYRLPRDAPFPASGEQQPRRPWRPRLLVIARARTRRFVNADEIVRGAERAGFEVVVSEGEHEVAPF
AELANTCDAMVGVHGAGLTNMVFLPTGGVVIQVVPLGGLEFVAGYFRGPSRDMGLRYLEYRITPEESTLIDQYPRDH
PIFTDPDGVKSKGWNSLKEAYLDKQDVRLDMKRFRPILKKAIAHLRKNSGNNNTTHN
>LOC_Os01g02940.3
(SEQ ID NO: 245)
MEGGGKAGYSYGGHHHHQDAKLLKNLSRVEPRRFGLGLVAGFLIVTCAYFSTAKFDAIHIAMISSPAKNAAGFMNAS
SDGSNQQQLDLDRDAMSREGSKAQVLDTDGDDKISSLGPDLGHNASALEGKKKDETFAKDSGDASVSASTDEALAKD
DDAIVGAVLPPLSSEEPTNITQDSVLEDEELKVQETAPATTNPSPEKSSNNGSSPSVVPSDPATLPVQQIPPTQEAK
DPPAQQIPAVPEAKVPPVQQIPTFPVVKTDSEAAPRRKEWKPLCDLWSNRRIDWCELDGDVRVAGANGTVSLVAPPG
PADERTFRAESWHIKPYPRKADPNAMRHVRVLTVQSLPAPAASAAAPACTERHDVPGLVFSDRGYTGNYFHAYTDVI
LPLFLTARQYSGEVKLLVSDFQMWWLGKFLPVFKAVSNYDLINLDDDRRVHCFRHVQVGLTCHADFSIDPSRAPNGY
SMVDFTRFMRATYRLPRDAPFPASGEQQPRRPWRPRLLVIARARTRRFVNADEIVRGAERAGFEVVVSEGEHEVAPF
AELANTCDAMVGVHGAGLTNMVFLPTGGVVIQVVPLGGLEFVAGYFRGPSRDMGLRYLEYRITPEESTLIDQYPRDH
PIFTDPDGVKSKGWNSLKEAYLDKQDVRLDMKRFRPILKKAIAHLRKNSGNNNTTHN
>LOC_Os01g02940.4
(SEQ ID NO: 246)
MEGGGKAGYSYGGHHHHQDAKLLKNLSRVEPRRFGLGLVAGFLIVTCAYFSTAKFDAIHIAMISSPAKNAAGFMNAS
SDGSNQQQLDLDRDAMSREGSKAQVLDTDGDDKISSLGPDLGHNASALEGKKKDETFAKDSGDASVSASTDEALAKD
DDAIVGAVLPPLSSEEPTNITQDSVLEDEELKVQETAPATTNPSPEKSSNNGSSPSVVPSDPATLPVQQIPPTQEAK
DPPAQQIPAVPEAKVPPVQQIPTFPVVKTDSEAAPRRKEWKPLCDLWSNRRIDWCELDGDVRVAGANGTVSLVAPPG
PADERTFRAESWHIKPYPRKADPNAMRHVRVLTVQSLPAPAASAAAPACTERHDVPGLVFSDRGYTGNYFHAYTDVI
LPLFLTARQYSGEVKLLVSDFQMWWLGKFLPVFKAVSNYDLINLDDDRRVHCFRHVQVGLTCHADFSIDPSRAPNGY
SMVDFTRFMRATYRLPRDAPFPASGEQQPRRPWRPRLLVIARARTRRFVNADEIVRGAERAGFEVVVSEGEHEVAPF
AELANTCDAMVGVHGAGLTNMVFLPTGGVVIQVVPLGGLEFVAGYFRGPSRDMGLRYLEYRITPEESTLIDQYPRDH
PIFTDPDGVKSKGWNSLKEAYLDKQDVRLDMKRFRPILKKAIAHLRKNSGNNNTTHN
>LOC_Os01g02940.5
(SEQ ID NO: 247)
MEGGGKAGYSYGGHHHHQDAKLLKNLSRVEPRRFGLGLVAGFLIVTCAYFSTAKFDAIHIAMISSPAKNAAGFMNAS
SDGSNQQQLDLDRDAMSREGSKAQVLDTDGDDKISSLGPDLGHNASALEGKKKDETFAKDSGDASVSASTDEALAKD
DDAIVGAVLPPLSSEEPTNITQDSVLEDEELKVQETAPATTNPSPEKSSNNGSSPSVVPSDPATLPVQQIPPTQEAK
DPPAQQIPAVPEAKVPPVQQIPTFPVVKTEAAPRRKEWKPLCDLWSNRRIDWCELDGDVRVAGANGTVSLVAPPGPA
DERTFRAESWHIKPYPRKADPNAMRHVRVLTVQSLPAPAASAAAPACTERHDVPGLVFSDRGYTGNYFHAYTDVILP
LFLTARQYSGEVKLLVSDFQMWWLGKFLPVFKAVSNYDLINLDDDRRVHCFRHVQVGLTCHADFSIDPSRAPNGYSM
VDFTRFMRATYRLPRDAPFPASGEQQPRRPWRPRLLVIARARTRRFVNADEIVRGAERAGFEVVVSEGEHEVAPFAE
LANTCDAMVGVHGAGLTNMVFLPTGGVVIQVVPLGGLEFVAGYFRGPSRDMGLRYLEYRITPEESTLIDQYPRDHPI
FTDPDGVKSKGWNSLKEAYLDKQDVRLDMKRFRPILKKAIAHLRKNSGNNNTTHN
>LOC_Os01g02940.6
(SEQ ID NO: 248)
MEGGGKAGYSYGGHHHHQDAKLLKNLSRVEPRRFGLGLVAGFLIVTCAYFSTAKFDAIHIAMISSPAKNAAGFMNAS
SDGSNQQQLDLDRDAMSREGSKAQVLDTDGDDKISSLGPDLGHNASALEGKKKDETFAKDSGDASVSASTDEALAKD
DDAIVGAVLPPLSSEEPTNITQDSVLEDEELKVQETAPATTNPSPEKSSNNGSSPSVVPSDPATLPVQQIPPTQEAK
DPPAQQIPAVPEAKVPPVQQIPTFPVVKTEAAPRRKEWKPLCDLWSNRRIDWCELDGDVRVAGANGTVSLVAPPGPA
DERTFRAESWHIKPYPRKADPNAMRHVRVLTVQSLPAPAASAAAPACTERHDVPGLVFSDRGYTGNYFHAYTDVILP
LFLTARQYSGEVKLLVSDFQMWWLGKFLPVFKAVSNYDLINLDDDRRVHCFRHVQVGLTCHADFSIDPSRAPNGYSM
VDFTRFMRATYRLPRDAPFPASGEQQPRRPWRPRLLVIARARTRRFVNADEIVRGAERAGFEVVVSEGEHEVAPFAE
LANTCDAMVGVHGAGLTNMVFLPTGGVVIQVVPLGGLEFVAGYFRGPSRDMGLRYLEYRITPEESTLIDQYPRDHPI
FTDPDGVKSKGWNSLKEAYLDKQDVRLDMKRFRPILKKAIAHLRKNSGNNNTTHN
>LOC_Os02g22190.1
(SEQ ID NO: 249)
MGSPKMAKSAMKQGIWRRRIGAPFAAVLVAAVLAVVVFSGQFAKGPNASSQFAPVQVDNTLRPTRDKPVSADQDLER
TVSSKLEGEDTEQIRLEDGQSPNKEAAIEEQKPSQAAAIDQDDNTLNPGLKQASGDERSAGGSDSLSKESPPQSQEG
DGGTAESGAEPYIKCTAQSDIKICDLSNPRFDICELCGDARTIGQSSTVVYVPQNRASNGEEWIIRAQSRKHLPWIK
KVTIKSVNSSEPEPICTSKHHIPAIVFALGGLTANVWHDFSDVLVPLFLTARQFNRDVQLIITNNQPWFIKKYSAIF
SRLTRHEIIDFDSDGQIRCYPHVIVGLRSHRDLGIDPSSSPQNYTMVDFRLFVREAYGLPAAEVDIPYKADKDDPDK
KPRIMLIDRGKSRRFVNVAHVVQGLDWFGFEVVKADPKIDSNLDEFVRLVDSCDAIMGVHGAGLTNMVFLRSGGVVV
HIVPYGIKFMADGFYGAPARDMGLRHVEYSISPEESTLLEKYGWNHTVINDPETIRKGGWEKVAEFYMSKQDIVLNM
TRFGPSLLNAIEFIM
>LOC_Os04g12010.1
(SEQ ID NO: 250)
MRRGESKAATRGANRSSSSSSWKSRYIGYGLVLGFVLVLLYLMVNAQFSNSPNAYLGPATTSKTESIPATTYQGNQA
WQEDGSRGLEEGHREEVASTHTERSTGQRQEKDDESEKQRTEKNSIEEQLGNDRSSNYWEEGRQSEKKDTIEFSEFG
GGTDDFNNVANTKPICDTSFGKYDICVLDGDTRAQGGGGAGAAVVTLVSPRAAPREWKIKPYSRKYLDGLKPVTVRS
VPNPEDAPPCTTRLNVPAMVIELGGLTGNYWHDFTDVLVPLFIGARRFGGEVQLLVVNLLPFWVDKYRRIFSQISRH
DIVDLEKDDDRGVVRCYPHVVVGYGSRKEFTIDPSLDDTGGGYTMVNFTEFLRQSYSLPRDRPIKLGTNHGARPRMM
ILERTNSRKLMNLPEVAAAARAAGFEVTVAGGRPTSTYDEFAREVNSYDVMVGVHGAGLTNCVFLPTGAVLLQIVPY
GRLESIAQTDFGEPARDMGLRYIEYDIAADESSLMDVFGKDHPMIKDPVAVHLSGWGNVAEWYLGKQDVRVNIERFR
PFLTQALEHLQ
>LOC_Os04g12010.2
(SEQ ID NO: 251)
MRRGESKAATRGANRSSSSSSWKSRYIGYGLVLGFVLVLLYLMVNAQFSNSPNAYLGPATTSKTESIPATTYQGNQA
WQDGSRGLEEGHREEVASTHTERSTGQRQEKDDESEKQRTEKNSIEEQLGNDRSSNYWEEGRQSEKKDTIEFSEFGG
GTDDFNNVANTKPICDTSFGKYDICVLDGDTRAQGGGGAGAAVVTLVSPRAAPREWKIKPYSRKYLDGLKPVTVRSV
PNPEDAPPCTTRLNVPAMVIELGGLTGNYWHDFTDVLVPLFIGARRFGGEVQLLVVNLLPFWVDKYRRIFSQISRHD
IVDLEKDDDRGVVRCYPHVVVGYGSRKEFTIDPSLDDTGGGYTMVNFTEFLRQSYSLPRDRPIKLGTNHGARPRMMI
LERTNSRKLMNLPEVAAAARAAGFEVTVAGGRPTSTYDEFAREVNSYDVMVGVHGAGLTNCVFLPTGAVLLQIVPYG
RLESIAQTDFGEPARDMGLRYIEYDIAADESSLMDVFGKDHPMIKDPVAVHLSGWGNVAEWYLGKQDVRVNIERFRP
FLTQALEHLQ
>LOC_Os06g13710.1
(SEQ ID NO: 252)
MKAAVGNKKSKGTFCAFCHPSLLLLIVAIQFLMIYSPTLDQYMVMLTTDEFIPEPHLRCDFSDNKSDVYEMEGAIRI
LSRELEVFLVAPRLASISGRSGVNTTGLDANATRWKIQPYTHKGESRVMPSITEVTLRLVTVDEAPPCDEWHDVPVI
VYSNGGYCSN
>LOC_Os06g20570.1
(SEQ ID NO: 253)
MKGRHERIKKGWSGSAAVWLLLVPLFVLIVLKTDFLPQVARLGDTSFTKVADEMVQKVSSLGLDRARWQQQQTLDVA
KLEDSVVGTSDELTGHVDANNEDSNQPNQQILAMSRSKDSRLINSDVAAAKTSHLSCNFSSAHMDTCAMDGDIRIHG
RSGVVYVVASSDYRPENATAVIRPYPRKWEQATMERVRQITIRSTAPPGAAVADTDGGGAIIPLRCTVARDMPAVVF
STGGYSVNFFHTMNDILLPLYITAREHGGRVQLLAANYDRRWTAKYQHALAALSMYPVVDLDADAAVRCFPSARVGV
ESHRVLGIDTPLTGSNGYTMVGFLAFLRSAYSLPRHAVTRTTPRRPRVVMVLRRKSRALTNEAEVVAAVAEAGFEVV
AAGPEEAGDVAGFAATVNSCDVMVGVHGAGLTNMVFLPRNGTVVQIIPWGGMKWPCWYDYGEPVPAMGLRYVEYEVA
ANETTLRERYPMDHPVFADPVSIHRKGFNHLWSTFLNGQNLTLDVNRFKAVMAEVYTSITAAPV
>LOC_Os06g49300.1
(SEQ ID NO: 254)
MKAAVRSKKSKGSFCHPPLLLLIVAIQFLVIYSPTLDQYMVMLTTGKPGFPSMLIDGRRSFKQVDEFIPEPHLRCDF
RDNRSDVCEMEGAIRILGRTSEVFLVAPSLASISGGGGGVNATGVDANATRWKIQPYTRKGESRVMPGITEVTVRLV
TADEAPPCDEWHDVPAIVYSNGGYCGNYYHDFNDNIIPLFITSRHLAGEVQLLVTQKQRWWFGKYREIVEGLTKYEP
VDLDAEQRVRCYRRATVGLHSHKDLSIDPRRAPNNYSMVDFKRFLMWRYALPREHAIRMEEEDKSKKPRLLVINRRS
RRRFVNLDEIVAAAEGVGFEVAAAELDAHIPAAASAVNSYDAMVAVHGSGLTNLVFLPMNAVVIQVVPLGRMEGLAM
DEYGVPPRDMNMRYLQYNITAEESTLSEVYPRAHPVFLDPLPIHKQSWSLVKDIYLGQQDVRLDVRRFRPVLLKALH
LLR
>LOC_Os08g34680.1
(SEQ ID NO: 255)
MEGAIRILGRELEVFLVAPRLASISGRSGVNTTGLDANATRWKIQPYTHKGESRVMPAITEVTLRLVTVDEAPPCDE
WHDVPVIVYSNGGYCSN
>LOC_Os11g36700.1
(SEQ ID NO: 256)
MRAALAVLVARRRHHLLRPLEFQHRRLLHGQRRADGPVVPVAPAVPQAAADGERHRGGEDTSVHAQVGGAHHEQGRG
GAAPDGSSRHDAPLLVMTAGGYTGNLFHAFSDGFVPAWLTVQHLRRRVVLGVLLYNPWWAGTYGEIISGLLDYHVVD
LLHDKRKHCFPGAIIGTRFHGILSVNPARLRDNKTIVDFHDLLADVYETAGDTVVVDVPQPAPRRPRLGIVSCRGKR
VIENQAAVARLARTVGFDVDILETADGLQLPASYASVSACDVLVGVHSADLTKLLFLRPGAALV
>LOC_Os03g48010.1
(SEQ ID NO: 257)
MASSPRPAATAAAHRRGLIQRPPSAQAYLSAAAALLVLAAVAFSRAGHRFPHPPATRRCRPDAEGSWSAGVFLGDSP
FSLEPIEHWGISKADGAAWPVANPVVTCAEVEDAGFPSSFVAKPFLFLQGDAIYMFFETKNPITSQGDIAAAVSEDA
GVTWQQLGVVLDEEWHLSYPYVFTYKNKVYMMPESSKNGDIRLYRALDFPLKWELEKVLLEKPLVDSVIINFQGSYW
LLGTDLSSYGAKRNREISIWYSNSPLSPWIPHKQNLIHNTGKMLSTRNGGRPFIYNGNLYRVGKGQGGGSGHGIQVF
KVEILKSNEYKEVEVPFVINKQLKGRNAWNGARSHHLDVQQLPSGKPWIGVMDGDRVPSGDSVHRLTIGYMIYGVVL
ILVLVTGGLIGTINCSLPLRWSLPHTEKRSGLFNVEQRFFLYHKLSSLISNLNKLGSLICGRINYRTCKGRVYVVVV
MLILVVLTCVGTHYIYGGNGAEEPYPIKGKYSQFTLLTMTYDARLWNLKMFVEHYSNCASVRDIVVVWNKGQPPAQG
ELKSVVPVRIRVEDRNSLNNRFNIDSEIKTKAVMELDDDIMMTCDDLERGFKVWREHPDRIIGYYPRLSEGSPLEYR
NERYARQQGGYNMVLTGAAFMDHGLAFKKYWSKEAEVGRQIVDSFFNCEDILLNFLFANASLTSTVEYVKPAWAIDM
SKFSGVAISRNTQAHYHVRSKCLAKFSEIYGNLTAKRFFNSRGDGWDV
>LOC_Os05g44360.2
(SEQ ID NO: 258)
MAAAILAMVPSYISRSVAGSYDNEAVAIFALIFTFYLYVKTLNTGSLFYATLNALSYFYMVCSWGGYTFIINLIPIH
VLLCIVTGRYSSRLYIAYAPLVILGTLLAALVPVVGFNAVMTSEHFASFLVFIILHVVALVYYIKGLLTPRLFKVAM
TLVITVGLAVCFAVIAILIALVASSPTKGWSGRSLSLLDPTYASKYIPIIASVSEHQPPTWPSYFMDINVLAFLIPA
GIISCFLPLSDASSFVVLYLVTAVYFSGVMVRLMLVLAPAACILSGIALSEAFDVLTRSVKYQLSKLFDDSPAASGD
SSAESSSASTVSTNSAKNETRPEKTETAPKEKPSKKNRKKEKEVAESVPVKPKKEKKLLVLPMEASVLGILLLIVLG
GFYVVHCVWAAAEAYSAPSIVLTSRSRDGLHVFDDFREAYAWLSHNTDVDDKVASWWDYGYQTTAMANRTVIVDNNT
WNNTHIATVGTAMSSPEKAAWEIFNSLDVKYVLVVFGGLVGYPSDDINKFLWMVRIGGGVFPHIKEPDYLRDGNYRV
DAQGTPTMLNCLMYKLCYYRFDKQNSIDNV
>LOC_Os01g69140.1
(SEQ ID NO: 259)
MMMGGQQSALNQLVSFLLGVSAAAVLIFFFSSAGGGWSTTTDLSSWANGTVAATAKETNLTSTAAHVEEKANLTNSQ
AAAAEAAKEEEEKELEKLLAAVADEHKNIIMTSVNEAWAAPGSLLDLFLEGFRAGEGIARFVDHLLIVALDDGAFRR
CRDVHPHCYRLAVAGRNFTDEKVFMSEDYLDLVWSKVKLQQRILELGYNFLFTDVDILWFRDPFEQMSMAAHMVTSS
DFFVGGAYNPANFPNTGFLYVRSSRRAVGVMEAWRAARASYPGRHEQQVLNEIKRELVERRGVRIQFLDTAHVAGFC
SNTRDFATLYTMHANCCVGLGAKLHDLRNLLEEWRAYRRMPDEQRRQGPVRWKVPGICIH
>LOC_Os01g69160.1
(SEQ ID NO: 260)
MEIKGNLRRFFVFLFELWLAATLVLVLLCVLANTGGSPEMPAAAEVCNCSQIGIASSRISEEVTGTSGNSNESSFAD
LAELLPKVATDDRTVIITSVNEAFARPNSLLVLFRESFAAGEKIAHLLDHVLVVAVDPAAFHHCRAVHPHCYHLKVD
TMNLSSANNFMSEAYVELVWTKLSLQQRVLELGYNFLFTDVDILWFRDPFRHIGVYADMTTSCDVFNGDGDDLSNWP
NTGFYHVKSTNRTVEMLRRWRAARARYPPNHEQNIFNYIKHELAAGLGVRVRFLDTAVFGGFCQLFRNDMARACTMH
ANCCVGLGNKLHDLRSALDQWANYTSPAPPEGRKKKSGGGGGDRRAGWSVPAKCGTPDKRG
>LOC_Os01g69174.1
(SEQ ID NO: 261)
MYFPPGLLALGNMSGHYYHHLTSFLLGAVLPTVLLFFLASDRVSERLPTISSLGNGALVIGGRATAREGGDLTGVDG
SAPAPAEKEKFPGLAELLPEVAMEDKTVIITSVNDAWAAPGSLLDLFRDSFHNGDGIAHLLDHVLVVAVDAGGFRRC
KAVHPHCYLLDVPGHGGLRRLLRVPPRRRQGVHGARQLLRRAGEQGARPQERARRLEELHGRPDVAGEEGCQQVQVD
VPGQVQGVVETALTMKSGNGQRLIILYIG
>LOC_Os01g69190.1
(SEQ ID NO: 262)
MGLGLGGGGGMAMINRNHVVSFLAGAALPTLLLFFLASDRVSEKLAIVSSWGSGGSSSAAAADHDLRGAGGDAAPPP
AQQEKFPGLPELLPKVAMEDRTVIITSVNEAWAAPGSLLDLYRDSFKNGEGIAHLLDHVLVVAVDPAGFRRCKAVHP
HCYLLHVKSINLTSATRFMSREYLELVWTKLSLQQRVLELGYNFLFTDCDMVLFRDPFRHIAVYADMSTSSDDYSAA
RAPLDNPLNTGLYYVKATSQSVEMLRYWQAARPRFPGAHDQAVFGHIKHELVAKLRARIEPLDTLYFGGFCEYHDDL
ARAVTMHADCCVGLDTKVHDLTDIAADWKNYTGMSPEERKKGGFKWTYPTRCRNSIGWRKPVHP
>LOC_Os01g69200.1
(SEQ ID NO: 263)
MRGSAGMASSKNGLSPVVVFLLGAASATALIVFVFTSTASPAWPTPEATPATRQEKKAAAVACAPRAKGIDSETRRA
ARTNQTGGGDDDDEFARMVRRAAMEDRTVIMTSVNEAWAAPGSLMDSFLESFRVGENISHFVEHIVVVAMDEGALRR
CRAIHPHCYLLLPEVAGLDLSGAKSYMTKDYLDLVWSKLKLQQRASMIVGETRGVDDEEHDARWHWQDVDLAWFRNP
MVHITAAADITTSSDFYFGDPDDLGNYPNTGFIYFKATPRNARAMAYWHAARRRFPGEHDQFVFNEIKRELAAGAGE
GGGVGVRIRFIDTAAVSGFCQLGRDLNRIATVHMTCCIGLENKLHDLRNVIRDWRRYVARPRWERQMGKIGWTFEGG
KCIH
>LOC_Os03g03730.1
(SEQ ID NO: 264)
MANGTVILTTLNSAWAEPGSVVDVFLESFRIGDETRWLLDHLVMVSLDLTAHRRCLQIHRHCFALTTDDGFDFSGEK
NFMTDGYLKMMWRRIDFLGHVLAKGYSFIFTDTDIVWFRNPLPHLHHDGDFQIACDHFTGDPDDLSNSPNGGFAYVR
STSATAAFYRYWYAARERHPGLHDQDVLNLIKRDAYVARLGVRIRFLSTDLFAGLCEHGRNLSTVCTMHANCCVGLR
RKVDDLGLMLQDWRRFMATPGSDRHSVTWSVPRNCR
>LOC_Os03g63280.1
(SEQ ID NO: 265)
MAPKVAVTEATGRQAASFVLGCVATLTVMLLFQYQAPPDYGRAARSPVQFSTSRDQLLLHCGGNGTAPPPPVIARGG
EEANITGKPPTTATAVAEEQPPTKPPATSTASSPTHHIPATSTDLEEEGGEFRGLAAAVARAATDDRTVIITCVNHA
FAAPDSLLDIFLQGFRVGDGTPELLRHVLVVAMDPTALTRCRAVHPHCYLYTMPGLDVDFTSEKFFASKDYLELVWS
KLKLQRRILQLGYNFLFTDVDIVWLRNPFKHVAVYADMAISSDVFFGDPDNIDNFPNTGFFYVKPSARTIAMTKEWH
EARSSHPGLNEQPVFNHIKKKLVKKLKLKVQYLDTAYIGGFCSYGKDLSKICTMHANCCIGLQSKISDLKGVLADWK
NYTRLPPWAKPNARWTVPGKCIH
>LOC_Os08g41800.1
(SEQ ID NO: 266)
MACKVRGDMGKLIPVISFFLGAALTAAFVIATMDINWRLSALASWNNNDSPPAVTDEMKALSELTEVLRNASMDDRT
VIMTSINRAYAAPGSLLDLFLESFRLGEGTEPLLKHVLIVAMDPAALARCRQVHPHCYLLRRPEGAVDYSDEKRFMS
KDYLDMMWGRNLFQQTILQLGFNFLFTDIDIMWFRNPLRHIAITSDIAVANDYYNGDPESLRNRPNGGFLYVRAARR
TVDFYRRWRDARRRFPPGTNEQHVLERAQAELSRRADVRMQFLDTAHCGGFCQLSRDMARVCTLHANCCTGLANKVH
DLAAVLRDWRNYTAAPPAARRRGGFGWTTPGKCIR
Os01g70200.1
(SEQ ID NO: 267)

MRRWVLAIAILAAAVCFFLGAQAQEVRQGHQTERISGSAGDVLEDDPVGRLKVYVYDLPSKYNKKLLKKDPRCLNHM
FAAEIFMHRFLLSSAVRTFNPEEADWFYTPVYTTCDLTPSGLPLPFKSPRMMRSAIELIATNWPYWNRSEGADHFFV
TPHDFGACFHYQEEKAIGRGILPLLQRATLVQTFGQKNHVCLKDGSITIPPYAPPQKMQAHLIPPDTPRSIFVYFRG
LFYDTSNDPEGGYYARGARASVWENFKNNPLFDISTDHPPTYYEDMQRSVFCLCPLGWAPWSPRLVEAVVFGCIPVI
IADDIVLPFADAIPWEEIGVFVAEEDVPKLDSILTSIPTDVILRKQRLLANPSMKQAMLFPQPAQAGDAFHQILNGL
ARKLPHGENVFLKPGERALNWTAGPVGDLKPW

Os06g23420.1
(SEQ ID NO: 268)
MRRWVLAIAILAAAVCFFLGAQAQEVRQGHQTERISGSAGDVLEDDPVGRLKVYVYDLPSKYNKKLLKKDPRCLNHM
FAAEIFMHRFLLSSAVRTFNPEEADWFYTPVYTTCDLTPSGLPLPFKSPRMMRSAIELIATNWPYWNRSEGADHFFV
TPHDFGACFHYQEEKAIGRGILPLLQRATLVQTFGQKNHVCLKDGSITIPPYAPPQKMQAHLIPPDTPRSIFVYFRG
LFYDTSNDPEGGYYARGARASVWENFKNNPLFDISTDHPPTYYEDMQRSVFCLCPLGWAPWSPRLVEAVVFGCIPVI
IADDIVLPFADAIPWEEIGVFVAEEDVPKLDSILTSIPTDVILRKQRLLANPSMKQAMLFPQPAQAGDAFHQILNGL
ARKLPHGENVFLKPGERALNWTAGPVGDLKPW
Os06g23420.2
(SEQ ID NO: 269)
MRRWVLAIAILAAAVCFFLGAQAQEVRQGHQTERISGSAGDVLEDDPVGRLKVYVYDLPSKYNKKLLKKDPRCLNHM
FAAEIFMHRFLLSSAVRTFNPEEADWFYTPVYTTCDLTPSGLPLPFKSPRMMRSAIELIATNWPYWNRSEGADHFFV
TPHDFGACFHYQEEKAIGRGILPLLQRATLVQTFGQKNHVCLKDGSITIPPYAPPQKMQAHLIPPDTPRSIFVYFRG
LFYDTSNDPEGGYYARGARASVWENFKNNPLFDISTDHPPTYYEDMQRSVFCLCPLGWAPWSPRLVEAVVFGCIPVI
IADDIVLPFADAIPWEEIGVFVAEEDVPKLDSILTSIPTDVILRKQRLLANPSMKQAMLFPQPAQAGDAFHQILNGL
ARKLPHGENVFLKPGERALNWTAGPVGDLKPW

In some embodiments, the rice-diverged GT is naturally occurring or not naturally occurring, and comprises a GT enzymatic activity and an amino acid sequence that comprises an equal to or more than 70% sequence similarity with an amino acid sequence chosen from the group consisting of SEQ ID NOs:1-269 or SEQ ID NOs: 1-30. In some embodiments, the rice-diverged GT comprises an amino acid sequence that comprises an equal to or more than 80% sequence similarity with an amino acid sequence chosen from the group consisting of SEQ ID NOs:1-269 or SEQ ID NOs: 1-30. In some embodiments, the rice-diverged GT comprises an amino acid sequence that comprises an equal to or more than 90% sequence similarity with an amino acid sequence chosen from the group consisting of SEQ ID NOs:1-269 or SEQ ID NOs: 1-30. In some embodiments, the rice-diverged GT comprises an amino acid sequence that comprises an equal to or more than 95% sequence similarity with an amino acid sequence chosen from the group consisting of SEQ ID NOs:1-269 or SEQ ID NOs: 1-30. In some embodiments, the rice-diverged GT comprises an amino acid sequence that comprises an equal to or more than 99% sequence similarity with an amino acid sequence chosen from the group consisting of SEQ ID NOs:1-269 or SEQ ID NOs: 1-30.

In some embodiments, where the expression of the GT is modified or altered, the expression of the GT is completely shut down, essentially shut down, or reduced as compared to the expression of the GT in the wild-type cell. The expression of the GT is modified or altered as compared to the expression of the GT in a wild-type cell, wherein the GT is negative to the wild-type cell. In some embodiments, where the expression of the GT is modified or altered, the expression of the GT is increased as compared to the wild-type GT. The increase can be at least twice, at least thrice, at least five times, at least ten times, or at least twenty times that of the expression of the GT in the wild-type cell. In some embodiments, the expression of the GT is increased due to the increased expression of the GT from a promoter, or the presence of a plurality of a nucleic acid encoding the GT, such as, having multiple copies of nucleic acid encoding the GT, or a combination thereof.

In some embodiments, where the enzymatic activity of the GT is modified or altered, the GT has a modified or altered amino acid sequence as compared to the wild-type GT such that the enzymatic activity of the GT is modified altered, or a combination of both. The modification or alteration can be an insertion, deletion, substitution, or a frame-shift mutation. In some embodiments, the enzymatic activity of the GT is decreased as compared to the wild-type GT. In some embodiments, the GT is knocked out or absent.

In some embodiments, the cell is a recombinant cell. The GT in the cell can be on or integrated into the cell genome or chromosome, and/or on a stably introduced replicon. In some embodiments, the replicon is capable of stable replication and/or transmission in the cell. Suitable replicons and vectors can include, for example, origins of replication, and/or markers. A marker gene can confer a selectable phenotype on a plant cell. For example, a marker can confer, biocide resistance, such as resistance to an antibiotic (e.g., kanamycin, G418, bleomycin, or hygromycin), or an herbicide (e.g., chlorosulfuron or phosphinothricin).

In some embodiments, the cell, seed, tissue, organ, plant part or plant is transgenic.

The enzymatic activity is an enzymatic activity of GT (EC 2.4.x.y) and/or the formation of a glycosidic bind through transfer of one or more sugars from an activated donor molecule to an acceptor molecule. The GT is a GT of a plant. In some embodiments, the GT is modified or altered in a plant, wherein the plant is a monocot or a dicot. In some embodiments, the monocot is a grass. In some embodiments the plant is a woody plant such as Eucalyptus, cottonwood, alder, Douglas fir, Hemlock, pine or spruce. In some embodiments, the plant is a leguminous plant, including, but not limited to, alfalfa, clover, lucerne, birdsfoot trefoil, Stylosanthes, Lotononis bainessii, and sainfoin. In some embodiments, the plant is a forage grass, including, but not limited to, bahiagrass, bermudagrass, dallisgrass, pangolagrass, big bluestem, indiangrass, switchgrass, smooth bromegrass, orchardgrass, timothy, Kentucky bluegrass or tall fescue.

The present invention also provides for a seed, plant tissue, organ, plant part or a whole plant comprising a cell of the present invention. In some embodiments, the plant part is a leaf, leaf stalk, stem, root, or a combination thereof. In some embodiments, the whole plant includes, but is not limited to, a germinating seed. In some embodiments, the whole plant is a mature plant.

The present invention also provides for a method of identifying or determining a rice-diverged GT of a plant or cell, comprising: the steps described herein in Example 1 of this present specification.

The present invention also provides for a method of constructing a cell of the present invention, comprising: modifying or altering the enzymatic activity of the GT. In some embodiments of the invention, the method further comprises identifying or determining the GT, wherein the identifying or determining comprises the steps described herein in Example 1 of this present specification.

In some embodiments of the invention, the method of constructing a cell of the present invention comprises modifying or altering the expression of the GT. In some embodiments of the invention, the method further comprises identifying or determining the rice-diverged GT, wherein the identifying or determining comprises the steps described herein in Example 1 of the present specification.

In some embodiments, modifying or altering the expression of the GT comprises increasing or decreasing the expression of the GT as compared to the expression of the GT in a wild-type cell.

In some embodiments, the GT is native to the cell. In other embodiments, the GT is heterologous to the cell. In some embodiments, the expression of the GT is increased via one or more of the following: increased copies of ORFs encoding the GT, increased transcription of an ORF encoding the GT, increased translation of a messenger RNA or transcript encoding the GT, and/or increased post-translational processing of the GT. In yet further embodiments, the cell comprises more than one rice-diverged GT, wherein a first rice-diverged GT is native or heterologous to the cell and a second rice-diverged GT is heterologous to the cell and is different from the first rice-diverged GT. Increased transcription can result from the ORF encoding the GT operably linked with a promoter with a higher expression when compared to the wild-type promoter, the addition of one or more activator or enhancing nucleotide sequences capable of increasing the transcription of the ORF encoding the GT, or the deletion or inactivation of one or more repressor sequences capable of decreasing or reducing the transcription of the ORF encoding the GT, or a combination thereof.

In some embodiments, the expression of the GT is decreased or reduced. In some embodiments, the expression of the GT is shut down or knocked out, including but not limited to the deletion of all or part of the ORF encoding the GT, or one or more promoter which initiated transcription of the ORF encoding the GT. In some embodiments, the expression of the GT is decreased or reduced via one or more of the following: decreased or reduced copies of ORFs encoding the GT, decreased or reduced transcription of an ORF encoding the GT, decreased or reduced translation of a messenger RNA or transcript encoding the GT, and/or decreased or reduced post-translational processing of the GT. Decreased or reduced transcription can result from the ORF encoding the GT operably linked with a promoter with a lower expression when compared to the wild-type promoter or knocking out the wild-type promoter, the deletion or inactivation of one or more activator or enhancing nucleotide sequences capable of increasing the transcription of the ORF encoding the GT, or the addition of one or more repressor sequences capable of decreasing or reducing the transcription of the ORF encoding the GT, or a combination thereof.

In some embodiments of the invention, the GT is located on the cell genome, chromosome, integrated into the chromosome, or located on a replicon, such as an expression vector or vector. In some embodiments the expression vector or vector are capable of stable maintenance and replication in the cell.

In some embodiments of the invention, the method of constructing a cell of the present invention comprises modifying or altering the amino acid of the GT such that the enzymatic activity of the GT is modified or altered. In some embodiments of the invention, the method further comprises identifying or determining the rice-diverged GT, wherein the identifying or determining comprises the steps described herein in Example 1 of this present specification.

The present invention also provides for a method of constructing a seed, plant tissue, plant part, or whole plant comprising a cell of the present invention, comprising: constructing a cell of the present invention. In some embodiments of the invention, the method further comprises identifying or determining the rice-diverged GT, wherein the identifying or determining comprises the steps described herein in Example 1 of this present specification.

In some embodiments of the invention, the seed, plant tissue, organ, plant part, or whole plant comprises modified or altered cellulose or cell wall, as compared to the corresponding wild-type seed, plant tissue, plant part, or whole plant, wherein the modified or altered cellulose is caused wholly or in part by the modified or altered enzymatic activity of the rice-diverged GT. In some embodiments of the invention, the cellulose is less structurally rigid, more prone to mechanical breakdown, more prone to breakdown or enzymatic digestion, or a combination thereof.

Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.)

Methods of genetically modifying or altering the cells and plants of the present invention are well-known to those of ordinary skill in the art. Plant cells or tissues can be transformed with expression constructs (heterologous nucleic acid constructs, e.g., plasmid DNA into which the gene of interest has been inserted) using a variety of standard techniques. Effective introduction of vectors in order to facilitate plant gene expression is an important aspect of the invention. In some embodiment, the vector sequences are stably integrated into the cell genome, so that the introduced constructs are passed onto successive plant generations. The skilled artisan will recognize that a wide variety of transformation techniques exist in the art, and new techniques are continually becoming available. Any technique that is suitable for the target host plant may be employed within the scope of the present invention. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present invention.

### REFERENCES CITED HEREIN

1. Affymetrix. Affymetrix Microarray Suite User Guide. (2001): Affymetrix.
2. Altschul S F, Gish W, Miller W, Myers E W, Lipman D J. Basic local alignment search tool. Journal of molecular biology (1990) 215:403-410.
3. An S, et al. Generation and analysis of end sequence database for T-DNA tagging lines in rice. Plant physiology (2003) 133:2040-2047.
4. Bourne Y, Henrissat B. Glycoside hydrolases and glycosyltransferases: families and functional modules. Current opinion in structural biology (2001) 11:593-600.
5. Brenner S, et al. Gene expression analysis by massively parallel signature sequencing (MPSS) on microbead arrays. Nature biotechnology (2000) 18:630-634.
6. Burton R A, et al. Cellulose synthase-like Cs1F genes mediate the synthesis of cell wall (1,3;1,4)-beta-D-glucans. Science (New York, N.Y. (2006) 311:1940-1942.
7. Campbell J A, Davies G J, Bulone V, Henrissat B. A classification of nucleotide-diphospho-sugar glycosyltransferases based on amino acid sequence similarities. The Biochemical journal (1997) 326 (Pt 3):929-939.
8. Carpita N C. Structure and Biogenesis of the Cell Walls of Grasses. Annual review of plant physiology and plant molecular biology (1996) 47:445-476.
9. Carpita N C, et al. Cell wall architecture of the elongating maize coleoptile. Plant physiology (2001) 127:551-565.
10. Chen F, Mackey A J, Vermunt J K, Roos D S. Assessing performance of orthology detection strategies applied to eukaryotic genomes. PLoS ONE (2007) 2:e383.
11. Coutinho P M, Deleury E, Davies G J, Henrissat B. An evolving hierarchical family classification for glycosyltransferases. Journal of molecular biology (2003) 328:307-317.
12. Dardick C, Chen J, Richter T, Ouyang S, Ronald P. The rice kinase database. A phylogenomic database for the rice kinome. Plant physiology (2007) 143:579-586.
13. Devos K M, Gale M D. Genome relationships: the grass model in current research. The Plant cell (2000) 12:637-646.
14. Drakakaki G, Zabotina 0, Delgado I, Robert S, Keegstra K, Raikhel N. Arabidopsis reversibly glycosylated polypeptides 1 and 2 are essential for pollen development. Plant physiology (2006) 142:1480-1492.
15. Droc G, et al. OryGenesDB: a database for rice reverse genetics. Nucleic acids research (2006) 34:D736-740.
16. Egelund J, et al. Molecular characterization of two Arabidopsis thaliana glycosyltransferase mutants, rra1 and rra2, which have a reduced residual arabinose content in a polymer tightly associated with the cellulosic wall residue. Plant molecular biology (2007) 64:439-451.
17. Egelund J, et al. Arabidopsis thaliana RGXT1 and RGXT2 encode Golgi-localized (1,3)-alpha-D-xylosyltransferases involved in the synthesis of pectic rhamnogalacturonan-II. The Plant cell (2006) 18:2593-2607.
18. Egelund J, Skjot M, Geshi N, Ulvskov P, Petersen B L. A complementary bioinformatics approach to identify potential plant cell wall glycosyltransferase-encoding genes. Plant physiology (2004) 136:2609-2620.
19. Farrokhi N, et al. Plant cell wall biosynthesis: genetic, biochemical and functional genomics approaches to the identification of key genes. Plant biotechnology journal (2006) 4:145-167.
20. Finn R D, et al. Pfam: clans, web tools and services. Nucleic acids research (2006) 34:D247-251.
21. Geisler-Lee J, et al. Poplar carbohydrate-active enzymes. Gene identification and expression analyses. Plant physiology (2006) 140:946-962.
22. Haas B J, et al. Improving the Arabidopsis genome annotation using maximal transcript alignment assemblies. Nucleic acids research (2003) 31:5654-5666.
23. Hazen S P, Scott-Craig J S, Walton J D. Cellulose Synthase-Like Genes of Rice. Plant Physiol. (2002) 128:336-340.
24. Henrissat B, Coutinho P M, Davies G J. A census of carbohydrate-active enzymes in the genome of Arabidopsis thaliana. Plant molecular biology (2001) 47:55-72.
25. Hong Z, Zhang Z, Olson J M, Verma D P. A novel UDP-glucose transferase is part of the callose synthase complex and interacts with phragmoplastin at the forming cell plate. The Plant cell (2001) 13:769-779.
26. Hu Y, Walker S. Remarkable structural similarities between diverse glycosyltransferases. Chemistry & biology (2002) 9:1287-1296.
27. Igura M, et al. Structure-guided identification of a new catalytic motif of oligosaccharyltransferase. The EMBO journal (2008) 27:234-243.
28. IRGSP. The map-based sequence of the rice genome. Nature (2005) 436:793-800.
29. Jain M, et al. F-box proteins in rice. Genome-wide analysis, classification, temporal and spatial gene expression during panicle and seed development, and regulation by light and abiotic stress. Plant physiology (2007) 143:1467-1483.
30. Jeong D H, et al. Generation of a flanking sequence-tag database for activation-tagging lines in japonica rice. Plant J (2006) 45:123-132.
31. Jung K H, An G, Ronald P C. Towards a better bowl of rice: assigning function to tens of thousands of rice genes. Nature reviews (2008a) 9:91-101.
32. Jung K, Phetsom J, Lee J W, Chris Dardick, Patrick Canlas, Peijian Cao, Xia-Xu, Young-Su Seo, Shu Ouyang, Kyungsook An, Yun-Ja Cho, Geun Cheol Lee, Yoosook Lee, Gynheung An, and Pamela C. Ronald. 2008. Identification and Functional Analysis of Light-Responsive Unique Genes and Paralogous Gene Family Members in Rice. PLoS Genetics, (2008b) 4(8): e1000164
33. Kolesnik T, et al. Establishing an efficient Ac/Ds tagging system in rice: large-scale analysis of Ds flanking sequences. Plant J (2004) 37:301-314.
34. Larkin M A, et al. Clustal W and Clustal X version 2.0. Bioinformatics (Oxford, England) (2007) 23:2947-2948.
35. Lee C, O′Neill M A, Tsumuraya Y, Darvill A G, Ye Z H. The irregular xylem9 mutant is deficient in xylan xylosyltransferase activity. Plant & cell physiology (2007a) 48:1624-1634.
36. Lee C, Zhong R, Richardson E A, Himmelsbach D S, McPhail B T, Ye Z H. The PARVUS gene is expressed in cells undergoing secondary wall thickening and is essential for glucuronoxylan biosynthesis. Plant & cell physiology (2007b) 48:1659-1672.
37. Liepman A H, Nairn C J, Willats W G, Sorensen I, Roberts A W, Keegstra K. Functional genomic analysis supports conservation of function among cellulose synthase-like a gene family members and suggests diverse roles of mannans in plants. Plant physiology (2007) 143:1881-1893.
38. Lin H, et al. Characterization of paralogous protein families in rice. BMC plant biology (2008) 8:18.
39. Miki D, Itoh R, Shimamoto K. RNA silencing of single and multiple members in a gene family of rice. Plant physiology (2005) 138:1903-1913.
40. Mitchell R A, Dupree P, Shewry P R. A novel bioinformatics approach identifies candidate genes for the synthesis and feruloylation of arabinoxylan. Plant physiology (2007) 144:43-53.
41. Miyao A, et al. Target site specificity of the Tos17 retrotransposon shows a preference for insertion within genes and against insertion in retrotransposon-rich regions of the genome. The Plant cell (2003) 15:1771-1780.
42. Mulder N J, et al. New developments in the InterPro database. Nucleic acids research (2007) 35:D224-228.
43. O'Reilly M K, Zhang G, Imperiali B. In vitro evidence for the dual function of Alg2 and Alg11: essential mannosyltransferases in N-linked glycoprotein biosynthesis. Biochemistry (2006) 45:9593-9603.
44. Ouyang S, et al. The TIGR Rice Genome Annotation Resource: improvements and new features. Nucleic acids research (2007) 35:D883-887.
45. Pauly M, Keegstra K. Cell-wall carbohydrates and their modification as a resource for biofuels. Plant J (2008) 54:559-568.
46. Pena M J, et al. Arabidopsis irregular xylem8 and irregular xylem9: implications for the complexity of glucuronoxylan biosynthesis. The Plant cell (2007) 19:549-563.
47. Perrin R M, et al. Xyloglucan fucosyltransferase, an enzyme involved in plant cell wall biosynthesis. Science (New York, N.Y. (1999) 284:1976-1979.
48. Persson S, et al. The Arabidopsis irregular xylem8 mutant is deficient in glucuronoxylan and homogalacturonan, which are essential for secondary cell wall integrity. The Plant cell (2007) 19:237-255.
49. Remm M, Storm C E, Sonnhammer E L. Automatic clustering of orthologs and in-paralogs from pairwise species comparisons. Journal of molecular biology (2001) 314:1041-1052.
50. Richmond T A, Somerville C R. Integrative approaches to determining Csl function. Plant molecular biology (2001) 47:131-143.
51. Somerville C, et al. Toward a systems approach to understanding plant cell walls. Science (New York, N.Y. (2004) 306:2206-2211.
52. Tuskan G A, et al. The genome of black cottonwood, Populus trichocarpa (Torr. & Gray). Science (New York, N.Y. (2006) 313:1596-1604.
53. Wall D P, Fraser H B, Hirsh A E. Detecting putative orthologs. Bioinformatics (Oxford, England) (2003) 19:1710-1711.
54. Wimmerova M, Engelsen S B, Bettler E, Breton C, Imberty A. Combining fold recognition and exploratory data analysis for searching for glycosyltransferases in the genome of Mycobacterium tuberculosis. Biochimie (2003) 85:691-700.
55. Wrabl J O, Grishin N V. Homology between O-linked GlcNAc transferases and proteins of the glycogen phosphorylase superfamily. Journal of molecular biology (2001) 314:365-374.
56. Yokoyama R, Nishitani K. Genomic basis for cell-wall diversity in plants. A comparative approach to gene families in rice and Arabidopsis. Plant & cell physiology (2004) 45:1111-1121.
57. York W S, O'Neill M A. Biochemical control of xylan biosynthesis—which end is up? Curr Opin Plant Biol (2008) 11:258-265.
58. Young N D, et al. Sequencing the genespaces of Medicago truncatula and Lotus japonicus. Plant physiology (2005) 137:1174-1181.
59. Yu J, et al. The Genomes of Oryza sativa: a history of duplications. PLoS biology (2005) 3:e38.
60. Yuan Q, et al. The institute for genomic research Osa1 rice genome annotation database. Plant physiology (2005) 138:18-26.
61. Zhang J, et al. RMD: a rice mutant database for functional analysis of the rice genome. Nucleic acids research (2006) 34:D745-748.
62. Zhang J Z. Evolution by gene duplication: an update. Trends in Ecology & Evolution (2003) 18:292-298.

Each of the references described above are herein incorporated by reference as though each is individually and specifically indicated as being herein incorporated by reference.

The invention having been described, the following examples are offered to illustrate the subject invention by way of illustration, not by way of limitation.

### EXAMPLE 1

### Construction of a Rice Glycosyltransferases Phylogenomic Database and Identification of Rice-Diverged Glycosyltransferases

With completion of rice (Oryza sativa ssp. japonica) genome sequencing and deposition of a large number of GTs in the CAZy database, we now have the opportunity to identify all the rice GTs and analyze them on a whole genome scale (IRGSP, 2005). In this study, we identified 609 rice GT loci (769 gene models) and executed a set of genome-scale analyses on these GTs. We used the data to identify GTs that have diverged significantly compared with dicot GTs and that may contribute to the synthesis of Type II specific cell wall components or be responsible for more subtle divergences in cell wall structure and function (e.g., different functions throughout development in type I vs. type II walls). We also report construction of a phylogenomic database for rice GTs (http://ricephylogenomics.ucdavis.edu/cellwalls/gt/), which provides a logical format to integrate, host and display diverse sets of functional genomic information in a phylogenetic context, thereby facilitating plant cell wall research. Using the database, we identified 33 rice-diverged GT genes (45 gene models) that are rice-diverged in vegetative, above-ground tissues and are strong candidate genes for further functional analysis toward understanding and manipulating grass cell walls for biofuel production.

Glycosyltransferases (GTs; EC 2.4.x.y) constitute a large group of enzymes that form glycosidic bonds through transfer of sugars from activated donor molecules to acceptor molecules. GTs are critical to the biosynthesis of plant cell walls. Based on the Carbohydrate-Active enZymes (CAZy) database and sequence similarity searches, we have identified 609 potential GT genes (loci) corresponding to 769 transcripts (gene models) in rice (Oryza sativa), the reference monocotyledonous species. Based upon their domain composition and sequence similarity, these rice GTs are classified into 40 CAZy families plus an additional unknown class. We found that two Pfam domains of unknown function, PF04577 and PF04646, are associated with GT families GT61 and GT31, respectively. We created a phylogenomic Rice GT Database (http://ricephylogenomics.ucdavis.edu/cellwalls/gt/) to facilitate functional analysis of this important and large gene family. Through the database, several classes of functional genomic data, including mutant lines and gene expression data, can be displayed for each rice GT in the context of a phylogenetic tree allowing for comparative analysis both within and between GT families. Comprehensive digital expression analysis of public gene expression data, revealed that most rice GTs are expressed. Based on analysis with Inparanoid, we identified 282 “rice-diverged” GTs that lack orthologs in sequenced dicots (Arabidopsis thaliana, Populus tricocarpa, Medicago truncatula and Ricinus communis). Combining these analyses, we have identified 33 rice-diverged GT genes (45 gene models) that are rice-diverged in above-ground, vegetative tissues and thus are good targets for functional examination toward understanding and manipulating grass cell wall qualities. This list of 33 genes and the GT database will facilitate the study of cell wall synthesis in rice and other plants.

### Methods

### Identification of Rice GTs and Database Construction

We searched the CAZy database (http://www.cazy.org/) and downloaded all the rice GTs hosted in this database (Campbell et al., 1997; Coutinho et al., 2003). Because genes in CAZy are associated with different kinds of gene names, including RAP2 (Rice Annotation Project Version 2) IDs, NCBI IDs, common names and TIGR IDs, all identifiers were converted to TIGR Version 5 IDs using the RAP ID Converter (http://rapdb.dna.affrc.go.jp/tools/converter) and NCBI BLAST Version 2.2.17 searches (Altschul et al., 1990). Arabidopsis GTs from CAZy and identified by fold recognition (Egelund et al., 2004), were also used to scan all the annotated proteins in the rice (Oryza sativa ssp. japonica) genome at TIGR (Version 5) (Ouyang et al., 2007; Yuan et al., 2005), to find the corresponding rice homologs (i.e., homolog search). In addition, the GT-related domains from the Pfam database (http://pfam.sanger.ac.uk/) were used to search the rice genome to identify putative GTs containing GT-related domains using HMMER 2.3.2 (i.e., domain search) (Finn et al., 2006). Finally, the GTs identified by previous steps were used to search the corresponding paralogs using the TIGR Paralog Family Classification database (i.e., paralog search) (Lin et al., 2008). After assembling the initial putative rice GT list, the Pfam and Interpro databases (http://www.cbi.ac.uk/interpro/) were used to check if the candidates have GT related domains (Finn et al., 2006; Mulder et al., 2007). Except as mentioned in the Results, genes lacking a GT-related domain and not annotated as GT-related genes in the TIGR annotation database, were deleted from the current list. Additionally, 5 TE-related candidates were also discarded. A phylogenomic database was then constructed with ASP.NET and MSSQL, run on a Windows 2003 server. The http address is http://ricephylogenomics.ucdavis.edu/cellwalls/gt/.

### Phylogenetic Analysis

For each GT family with more than three members the corresponding GT domain sequences were extracted according to the Pfam and Interpro domain assignments. We aligned GT domain sequences in these families using Clustal W version 2.0 with default options (Larkin et al., 2007). The alignments were then corrected manually using the alignment editor software BioEdit Version 7.0.09 (http://www.mbio.ncsu.edu/BioEdit/bioedit.hlml). The unrooted, phylogenetic tree was constructed with the neighbor-joining method executed in PHYLIP version 3.67 (http://evolution.genetics.washington.edu/phylip.html) using only the domain sequences. Bootstrapping can provide an estimate of the confidence for each branch point, so 1000 bootstraps were adopted to infer the statistical support for the tree.

### Orthology Detection in Dicots

Inparanoid Version 2.0 was adopted to evaluate the orthology relationships among rice and sequenced, annotated dicots on the whole-genome scale (Remm et al., 2001). The Arabidopsis genome sequences were downloaded from the Arabidopsis Information Resource (TAIR8, http://www.arabidopsis.org/), P. trichocarpa from the DoE Joint Genome Institute and Poplar Genome Consortium annotation v1.1 (http://genome.jgi-psf.org/Poptr1_{—}1/) (Tuskan et al., 2006), M. truncatula from the Medicago Genome Sequence Consortium (MGSC) Mt2.0 release (http://www.medicago.org/) (Young et al., 2005), and R. communis from the TIGR Castor bean Database (http://castorbean.tigr.org/).

### Digital Expression Analysis (EST, MPSS and Microarray)

EST Analysis. The TIGR Digital Northern search page (http://www.tigr.org/tdb/e2k1/osa1/dnav.shtml) provides the number of ESTs from several different rice tissues mapped onto TIGR gene models and was used for digital expression analysis of rice GTs (Jung et al., submitted). Each of the TIGR locus IDs corresponding to all rice GT gene models was searched to find availability of corresponding EST evidence. The EST evidence was determined using the PASA program which utilizes a number of alignment programs to maximally align transcripts to the genome (Haas et al., 2003). The minimal alignment allowed by the PASA program is 95% identity over 90% length of the transcript.

MPSS Analysis. Expression evidence from MPSS tags was determined from the rice MPSS project (http://mpss.udel.edu/rice/) mapped onto the TIGR rice gene models. We used only the sense strand signatures (Classes 1, 2, 5 and 7) which have only one hit on the rice pseudomolecules and show a perfect match (100% identity over 100% of the length of the tag) in the analysis. The normalized abundance (tags per million, TPM) of these signatures for a given gene in a given library represents a quantitative estimate of expression level of that gene. MPSS expression data for 17 by signatures from 18 libraries representing 12 different tissues/organs of rice were used. The description of these libraries is: NCA, 35 days callus; NCL, 14 days young leaves stressed in 4 C cold for 24 h; NCR, 14 days young roots stressed in 4° C. cold for 24 h; NDL, 14 days young leaves stressed in drought for 5 days; NDR, 14 days young roots stressed in drought for 5 days; NGD, 10 days germinating seedlings grown in dark; NGS, 3 days germinating seed; NIP, 90 days immature panicle; NL4, 60 days mature leaves (combination of replicates); NME, 60 days crown vegetative meristematic tissue; NPO, mature pollen; NR2, 60 days mature roots (combination of replicates); NSL, 14 days young leaves stressed in 250 mM NaCl for 24 h; NSO, ovary and mature stigma; NSR, 14 days young roots stressed in 250 mM NaCl for 24 h; NST, 60 days stem; NYL, 14 days young leaves; NYR, 14 days young roots.

Microarray Analysis. The raw data for rice Affymetrix microarray experiment designed to profile the expression pattern of rice reproductive development was downloaded from the NCBI Gene Expression Omnibus (GEO, http://www.ncbi.nlm.nih.gov/geo/) (Jain et al., 2007). The GEO accession number is GSE6893. Then MAS 5.0 method provided by the R package, affy, for the Affymetrix rice array was used to conduct background correction, normalization, probe specific background correction, probe summarization and convert probe level data to expression values (Affymetrix, 2001). The trimmed mean target intensity of each array was arbitrarily set to 500. These data were then log₂ transformed. The rice Multi-platform Microarray Search tool (http://www.ricearray.org/matrix.search.shtml) was used assign the corresponding Affymetrix probe sets for rice GTs. We only included unique probe sets that match unique rice locus in the analysis (Jung et al., submitted). If several unique probe sets were available for a single rice GT gene model, we selected the probe set with the highest expression. The heatmap was generated by the TIGR MultiExperiment Viewer v4.1 (MeV, http://www.tm4.org/mev.html).

Identification of Rice GT Gene-indexed Mutant Lines and Relating Rice Functional Genomic Databases. Several rice mutant line libraries are available, including the National Institute of Agrobiological Sciences (NIAS) Tos17 Insertion Mutant Database (Miyao et al., 2003); the UCD Rice Transposon Flanking Sequence Tag Database with Ds Knockout (KO) lines (Kolesnik et al., 2004); the Oryza Tag Line (OTL) Database with Tos17 and T-DNA KO lines; the Rice Mutant Database (RMD) with T-DNA KO lines (Zhang et al., 2006); the Taiwan Rice Insertional Mutants Database (TRIM) with T-DNA KO lines; and the Postech Rice T-DNA Insertion Sequence Database with T-DNA KO and Activation (AC) lines (An et al., 2003; Jeong et al., 2006). The OryGenesDB database (http://orygenesdb.cirad.fr/index.html) was used to map flanking sequence tags (FSTs) from the different mutant libraries onto rice GTs (Droc et al., 2006). The flanking sequences have been placed in the TIGR Version 5 pseudomolecules by finding the highest hit based on an e⁻¹⁰ cut-off. The mapped insertions were then assigned to rice GT genes based on the insertion map locations relative to the TIGR genome annotations. In the OryGenesDB database, a gene was defined as beginning 800 by 5′ of the initiation codon and to the end of the 3′-UTR, where known. The Postech activation lines were obtained from the Postech Rice T-DNA Insertion Sequence Database (http://141.223.132.44/pfg/index.php) (Jeong et al., 2006).

### Results and Discussion

Identification of Rice GTs. Glycosyltransferases from across the kingdoms of life have previously been identified based upon domain compositions, sequence similarity and function. The CAZy database is a comprehensive database for carbohydrate enzymes that degrade, modify, or create glycosidic bonds. CAZy classifies GTs into different families primarily based on amino acid sequence similarities (Campbell et al., 1997; Coutinho et al., 2003). As of February 2008, there were 90 GT families and 33,359 entries in the CAZy database. We identified a total of 548 rice GT genes (loci) from this database. We then converted the Rice Annotation Project and other various identifiers associated with the rice GTs from CAZy into The Institute of Genomic Research (TIGR) Version 5 Locus Identifiers (IDs) for convenience in the further analysis. Not all GTs are included in the CAZy database (Egelund et al., 2004). Thus, we took advantage of the availability of the complete rice genome sequence to identify GT genes not included in the CAZy database through homolog, domain and paralog searches to identify additional “non-CAZy” rice GTs (IRGSP, 2005). Searching of the rice genome with the known GTs led to the identification of an additional 34 GTs by homolog search, 12 GTs by domain search and 15 GTs by paralog search (see Materials and Methods).

In total, 609 rice GT genes were identified in our analysis and classified into 40 CAZy families and an additional unknown class. One hundred and seven of these GT genes are predicted to code for 160 additional alternative splicing isoforms, resulting in a total of 769 GT transcripts (gene models) encoded in the rice genome. The 609 rice GT loci were found to be distributed randomly on all the 12 rice chromosomes with the maximum number present on the largest chromosome 1 (85) and minimum on chromosome 11 (16). BLASTP searching with these 769 GT proteins in the FGENESH-annotated proteins of Oryza sativa ssp. indica genome available at BGI Rise Rice Genome Database (http://rise.genomics.org.cn/rice) revealed that nearly all of these proteins ( 767/769 with E value<e⁻²⁰) are conserved in both rice subspecies (Yu et al., 2005). A domain search of rice GT proteins in Pfam and Interpro databases identified at least one GT related domain for each GT family except for four families. Although there is no GT related domain annotated in GT41 and GT65, they were retained because they were obtained from the CAZy database. That database also provides no GT-related domain for these two families. Furthermore, genes in these families are annotated as glycosyltransferase genes in the TIGR annotation database. GT77 also does not have a GT-related domain and the members are annotated as regulatory proteins rather than GT proteins in the TIGR database. However, rice members of this GT family have very high sequence similarities with Arabidopsis GT77 proteins, RGXT1 (At4g01770) and RGXT2 (At4g01750). RGXT1 and RGXT2 were identified via a fold recognition method and then experimentally validated (Egelund et al., 2006; Egelund et al., 2004). We therefore included rice GT77 proteins in the list. GT61 proteins and some members of the GT31 family also do not possess domains that are annotated as GT-related. Rather, we found that two Pfam domains of unknown function, PF04577 and PF04646, are associated with these rice GT families, respectively. All 39 GT61 family members contain Pfam domain PF04577. Fourteen out of 58 GT31 members contain PF04646 domain; whereas, the other GT31 members contain a galactosyl transferase domain, PF01762. Although the function of these two domains (PF04577 and PF04646) is unknown in the current Pfam database, they should be considered as GT related domains according to this observation.

Database Construction and Navigation. Though the GT section of the CAZy database is reasonably comprehensive in scope, it lacks depth of information on each GT, limiting further functional and reverse genetic analyses of this large gene family. Several kinds of functional genomic data are now available, such as expression data from expressed sequence tags (ESTs), massively parallel signature sequencing (MPSS) and oligonucleotide microarrays. However, these data are scattered in different databases and are not easily integrated for comparison between and within different rice GT families. To resolve this problem, we created a publicly accessible, phylogenomic database, the Rice GT Database (http://ricephylogenomics.ucdavis.edu/cellwalls/gt/) to integrate, host and display functional genomic data for rice GTs in a phylogenetic context. As listed in Table 1, eight types of functional genomic data were gathered for rice GTs and are available in this database, including sequence and ortholog information, mutant availability, protein topology predictions, and gene expression data. Further information about the development and content of the Rice GT database are provided in subsequent sections.

TABLE 1
Data available in the Rice GT Database.

| Data Type | Description |
|---|---|
| Sequence | TIGR and RAP annotations, CAZy families, GT domains and NCBI BLAST links |
| Information | |
| Sequence Quality | FL-cDNA/EST evidence, BAC/PAC and PASA status |
| Orthologs in Dicots | Orthologs identified in four selected dicot species using Inparaniod2 |
| Mutants | Knockout and Activation mutant lines from several mutant libraries |
| Topology | Predicted protein topology (such as transmembrane domain) and subcellular localization |
| MPSS Data | MPSS data determining the representation of transcripts within mRNA and regulatory |
| | small RNA |
| Digital Northern | Number of EST evidences within different rice tissues/organs from TIGR database |
| Data | |
| Microarray Data | There are three kinds of microarray platforms available in the database until now: |
| | Affymetrix, NSF 20K and BGI/Yale. Several hundreds slides are presented and heatmaps |
| | are also provided for easy visualization. |

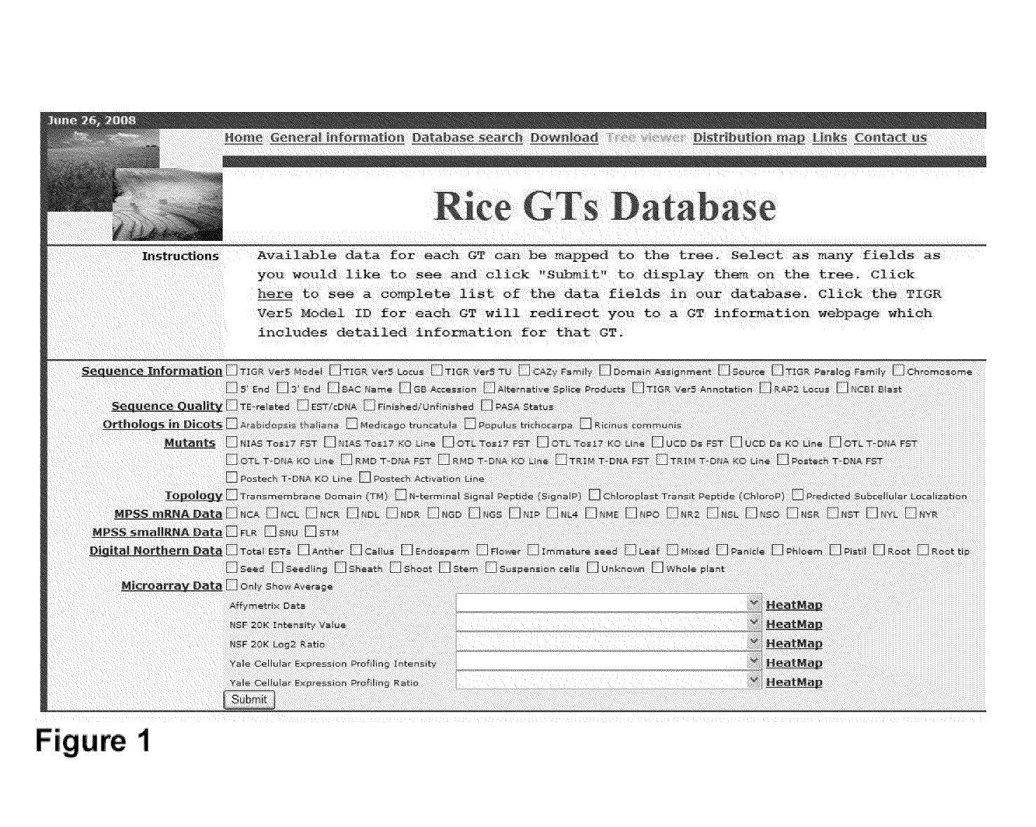

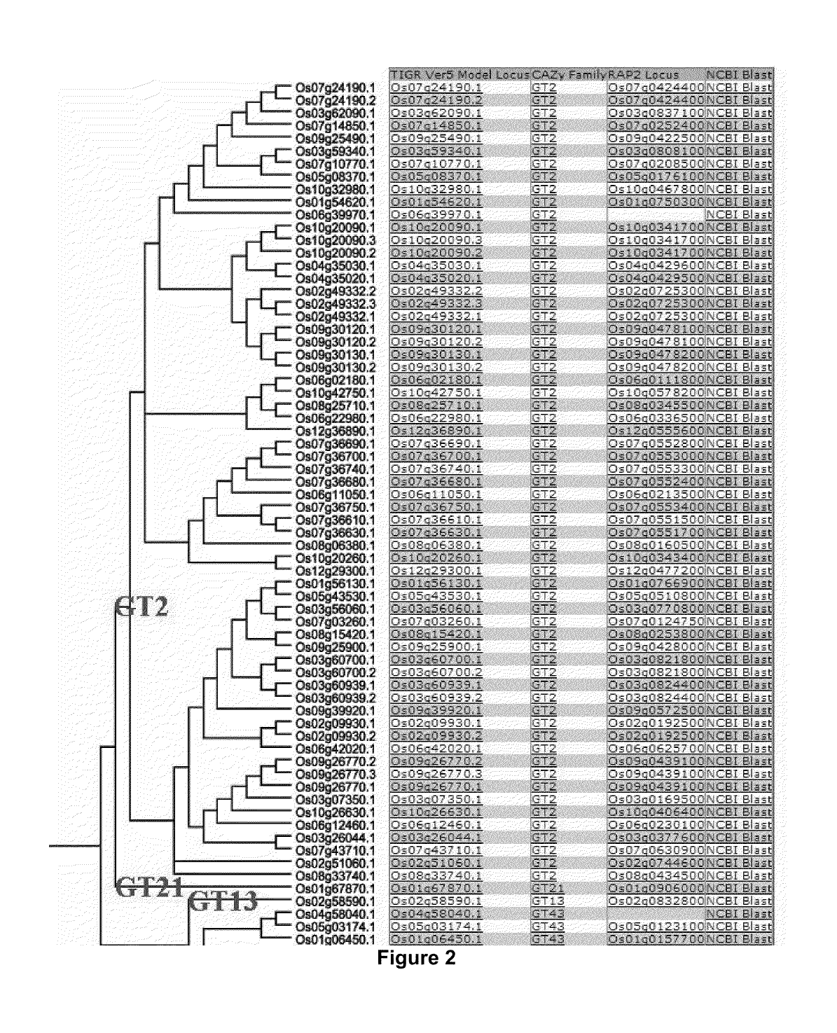

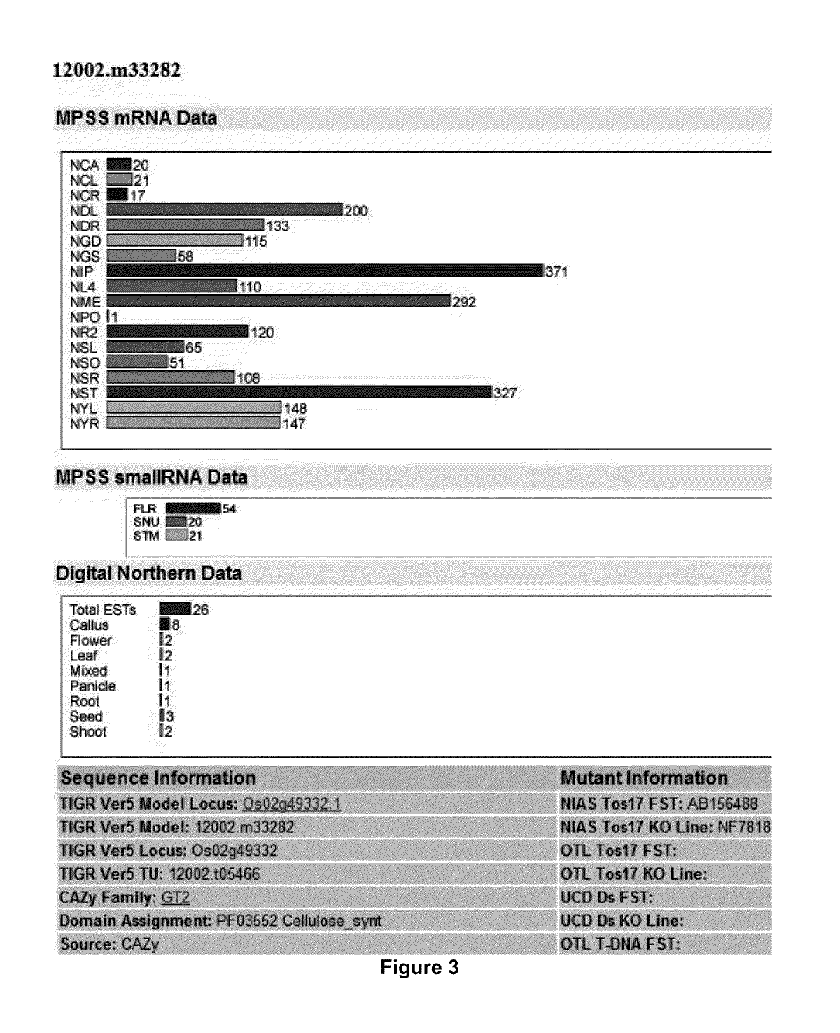

To assist in use of the Rice GT Database, links to the database information, database search, chromosome distribution map and phylogenetic tree viewer are provided on the home page. To aid comparisons within and between GT clades, most data is available in the context of a phylogenetic tree of rice GTs. In the tree viewer page, functional genomic fields can be selected by checking each box (FIG. 1). Pressing the submit button will display the selected data adjacent to the GT phylogenetic the tree (FIG. 2). The spreadsheet format allows all data or user-defined subsets of data to be readily transferred into any database or software, such as Excel, for further analysis. Once displayed, the spreadsheet can be searched for a particular locus ID or other field with the user's browser search function. Clicking on a gene model ID (12XXX.mXXXX) link brings up a summary webpage for that gene model showing all of the available data except for the microarray data, but including histogram representations of expression patterns from Digital Northern and MPSS data (FIG. 3) Links to the TIGR rice database, Rice Annotation Project Database (RAP-DB), CAZy database and NCBI BLAST search, are given for easy navigation. These links allow for simple navigation between all data display formats as well as complementary databases. Mutant line identification numbers are given as hyperlinks to the corresponding library when phenotypic information is available for that mutant. For display of microarray data, users may toggle between displaying numerical values for each replicate or averages for each sample. With separate links, we have also built red-green heatmaps for the easy examination of each microarray dataset. The chromosome distribution map is color coded according to the different CAZy families and rice GT loci are represented as colored boxes. Mousing over each box generates a pop up showing the ID of each rice GT locus. Clicking on the box directs the user back to the Tree viewer page, with the selected rice GT at the top of the view window. A search function is also available, enabling users to search the database with a locus ID or BLAST search.

Phylogenetic Analysis. Phylogenetic trees display a sorting of genes into groups based on sequence similarity and are particularly valuable when studying large gene families (Jung et al., 2008). Sensitive sequence-similarity detection methods such as hydrophobic cluster analysis or PSI-BLAST have revealed only very low sequence similarities between some GT families (Campbell et al., 1997; Wrabl and Grishin, 2001). These distant similarities, presumably a result of evolutionary divergence, make it difficult to construct a single phylogenetic tree using all the rice GTs from different GT families. Rather we adopted the hierarchical classification approach presented by Countinho et al. to build an assembled whole phylogentic tree (Coutinho et al., 2003).

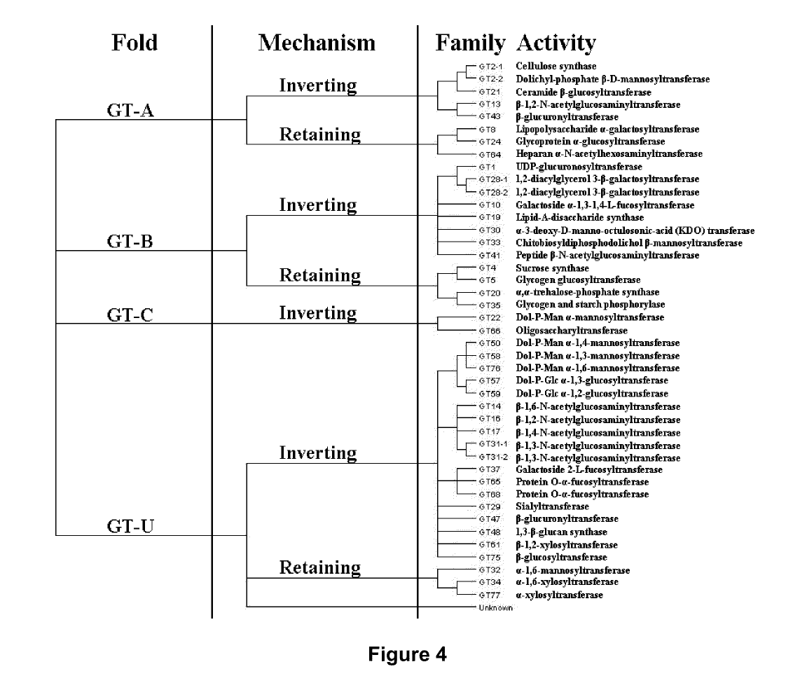

The forty GT families are hierarchically classified based on their GT fold type, reaction mechanism and known activities. This classification is shown in FIG. 4. There are two different GT domains in GT2, GT28 and GT31, so these GT families were divided into two subfamilies according to GT domain. Then the unmated phylogenetic trees were constructed in each GT family or subfamily with more than three members based on GT domain sequences and neighbor-joining method. For GT families with less than three members, the phylogenetic relationships among their members were determined manually. There is no GT related domain in GT77, thus the whole protein sequences were used for the phylogenetic analysis in this family. Finally, all the trees were assembled into a whole phylogenetic tree according to the family hierarchical classification.

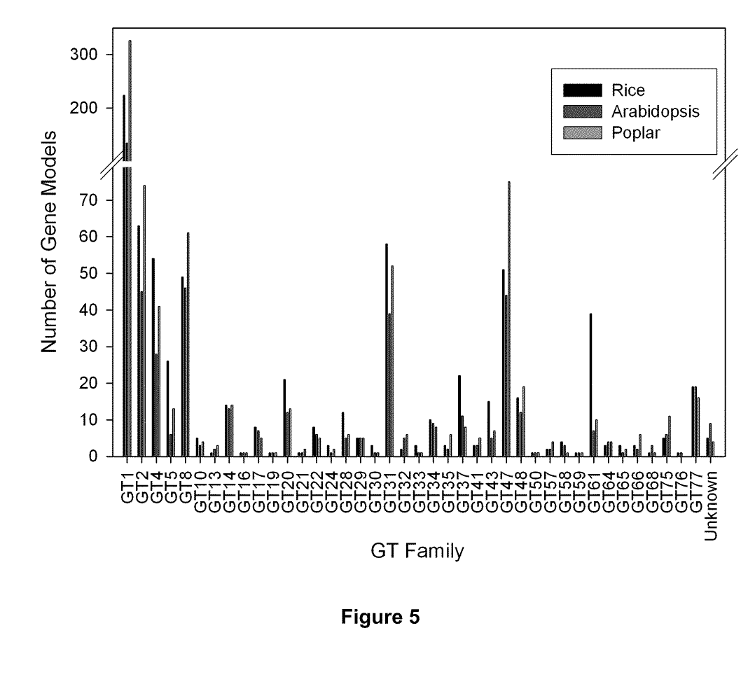

Interspecies Comparison Identifying Rice-Diverged GTs. In principle, the difference between type I and type II cell wall polysaccharide content might be reflected by qualitative difference in the GT content among reference plant species. To test this hypothesis we compared the distribution of GT gene models between rice and the two dicots for which GTs have been comprehensively annotated, Arabidopsis and poplar (FIG. 5). In Arabidopsis, there are 452 GT genes (507 gene models) based on the content of the CAZy database and fold recognition (Egelund et al., 2004). Poplar contains approximately 840 GT gene models, which is the largest number of genes encoding glycosyltransferases observed among fully sequenced genomes (Geisler-Lee et al., 2006). The poplar genome annotation is not yet complete on the gene loci level, so we conducted this analysis on the level of gene models, which includes different splice forms from single loci.

Except as noted below, we found the same GT families in rice, Arabidopsis, and poplar (FIG. 5). This result is consistent with a previous analysis that found that all known cell wall related GT families at the time are found in both rice and Arabidopsis (Yokoyama and Nishitani, 2004). The one exception to representation in all three species is GT76, which is absent from the poplar genome annotation. However, we detected a GT76 member in the poplar genome (E<e⁻¹⁰⁰) with a BLASTP search with the single members of the GT76 family from Arabidopsis and rice. Thus the absence in poplar is likely due to the incomplete genome annotation at the time of poplar GT identification. The GT1 family, responsible for glycosylation of secondary metabolites but not cell wall synthesis, is the largest family in all three species. Excluding GT1, the top 5 largest GT families in rice are GT2, GT4, GT8, GT31 and GT47, which is also the case for Arabidopsis and poplar.

Seven GT families appear to have significantly greater representation in the rice genome compared to Arabidopsis and poplar. GT families 5, 28, 30, 33, 37, 43, and 61 contain >2-fold the number of genes in rice versus the two dicots (FIG. 5). The first four of these seven families are not expected to be involved in cell wall synthesis (FIG. 4). GT5s are glycogen glucosyltransferases; GT28s are diacylglycerol galactosyltransferases; GT30s are mannooctulosonic acid transferases; and GT33s are involved transfer of mannose residues to endoplasmic reticulum associated-proteins (O′Reilly et al., 2006). The final three families on this list are known or hypothesized to catalyze synthesis of cell wall polysaccharides. GT37s include orthologs of the Arabidopsis FUT proteins, which possess α-fucosyltransferase activity involved in xyloglucan synthesis (Perrin et al., 1999). The increase in number of genes in this family in rice compared to Arabidopsis has been noted previously (Yokoyama and Nishitani, 2004). Whether the family possess the same activity in grasses, which possess far lower quantities of xyloglucan, remains to be seen. In the GT43 family, the Arabidopsis gene, IRX9, has recently been implicated in synthesis of xylan in secondary cell walls (Lee et al., 2007a; Pena et al., 2007). Mitchell et al. also identified genes in the GT43 and GT61 families as having higher expression in cereals than in dicots, though those authors did not note the relative increase in number of genes in those families (Mitchell et al., 2007). This coarse analysis of the numbers of genes in various GT families begins to suggest gene families for further exploration toward understanding the synthesis of grass cell walls. Subsequent analyses provide further information toward choosing specific genes for reverse genetic analysis.

Although the same GT families are present in the reference monocot rice and dicots, Arabidopsis and poplar, we hypothesized that within each GT family that “rice-diverged” GTs with significantly different primary sequences compared to dicots may have evolved since the last common ancestor between rice and dicots. Orthology detection (and conversely detection of genes that lack orthologs) is critically important for accurate functional annotation, and has been widely used to facilitate studies on comparative and evolutionary genomics (Chen et al., 2007). We hypothesize that differences in primary sequence might be a proxy for functional divergence in some cases. The ongoing individual gene duplication events in the rice genome provides duplicated genes that can serve as raw materials for genesis of new genes (Yu et al., 2005). A large part (about one third, data not shown) of rice GTs are involved in tandem and segmental duplication events, and substantial clustering of rice GTs is evident on different chromosomes. Thus, some rice-diverged GTs may have evolved after duplications, through a process known as neo-functionalization in which duplicated genes obtain novel functions compared to ancestral genes (Zhang, 2003). In support of this approach with respect to cell wall-related genes, phylogenetic analysis of the Csl genes within the GT2 family of rice and Arabidopsis led to the identification of rice-diverged Cs1 gene families Cs1F and Cs1H (Hazen et al., 2002). Subsequent heterologous expression studies demonstrated that the Cs1F gene family is involved in synthesis of the Type II wall-specific mixed linkage glucan polysaccharide (Burton et al., 2006).

We computationally identified rice-diverged GTs by detecting which rice GTs lack orthologs in sequenced dicots. Several ortholog identification methods are now available, including methods such as, reciprocal smallest distance (Wall et al., 2003), Inparanoid (Remm et al., 2001) and BLASTP (Altschul et al., 1990), among others. Inparanoid exhibited the best overall performance, with both low false negative and false positive rates, within a orthology detection strategy assessing experiment on divergent eukaryotic genomes (Chen et al., 2007). Inparanoid is an automated method for finding orthologs and “in-paralogs” from two species. It functions by detecting ortholog clusters with two-way best pairwise matches then it adds related, in-paralogs, predicted to have diverged since speciation (Remm et al., 2001).

We used Inparanoid Version 2.0 to identify rice GT orthologs in the completely sequenced dicots, Arabidopsis (family: Brassicaceae), poplar (Salicaceae), medick (Medicago truncatula, Fabaceae) and castor bean (Ricinus communis, Euphorbiaceae). Based on orthology search in these selected dicots, 282 rice GTs (36.7%) lacked orthologs and were therefore considered to be rice-diverged GTs. One hundred and ninety seven (70%) of these are expressed based on FL-cDNA or EST evidence. From the analysis of Chen et al., we expect that the number of rice-diverged GTs may be high, due to Inparanoid's rate of the false negative of ortholog identification in their tests (false negative rate=0.17) (Chen et al., 2007). In addition, a smaller number of rice-diverged GTs may have been missed in our analysis based on the identification of false positives by Inparanoid (false positive rate=0.07) (Chen et al., 2007).

We speculate that the putative rice-diverged GTs that we have identified may also be grass-diverged due to the high level of genomic colinearity among grass species (Devos and Gale, 2000). In the future we plan to test the generality of this analysis by comparing dicot and rice GTs with other grasses, including Brachypodium, sorghum, and maize, as annotation for these recently sequenced genomes becomes available.

As will be discussed further below for specific cases, some of the genes that we have identified as “rice-diverged” were also identified by Mitchell et al. as “rice-diverged rice orthologs of Arabidopsis genes”. Mitchell et al. used BLASTP (bit score 200) to identify rice-Arabidopsis ortholog pairs (Mitchell et al., 2007). According the analysis of Chen et al., BLASTP has a high false positive rate (0.5) compared with other ortholog detection methods (Chen et al., 2007). While this was appropriate for Mitchell et al., who otherwise might have missed a number of preferentially grass-expressed genes, it explains why this study and that previous one have identified the same genes using apparently opposite methods.

Digital Expression Analysis. Phylogenetic trees can provide a context to identify members within gene families that have unique properties, including unique expression patterns (Dardick et al., 2007; Jung et al., 2008). Gene expression patterns can inform hypothesis regarding which gene family members are expected to perform distinct or similar roles. Predominant, or higher expression, of one or more gene family members under a particular set of conditions may indicate a role for the predominantly expressed gene in the process under examination. For example, we recently found evidence that gene family members predominantly expressed in the light were more likely to have a role in light responses compared with genes in the same family that were lowly expressed in the light (Jung et al., 2008b). Thus, we sought to further refine the list of rice-diverged genes for reverse genetic analysis using three classes of publicly available transcriptome information, EST, MPSS and microarray data.

EST Analysis. We analyzed rice EST data using the TIGR Rice Gene Expression Anatomy Viewer and Digital Northern (http://rice.plantbiology.msu.edu/dnav.shtml), which provides the number of ESTs from different rice tissues mapped onto the TIGR gene models to estimate gene expression levels (Jung et al., submitted). This analysis revealed that one or more EST has been recorded for 628 (81.7%) of 769 rice GT gene models, providing strong indication that most rice GTs are expressed. In contrast, just less than 60% of all TIGR version 5 gene models have EST evidence (Jung et al. 2008b). However, the frequency of total ESTs for rice GT gene models varied greatly from 1 to 770, suggesting that the expression levels among rice GTs vary dramatically.

TIGR Digital Northern data covers ESTs isolated from 20 rice tissue sources (anther, callus, endosperm, flower, immature seed, leaf, mixed tissues, panicle, phloem, pistil, root, root tip, seed, seedling, sheath, shoot, stem, suspension cells, unknown samples and whole plant), but rice GTs were found to only expressed in 12 tissues (Table 2). The absence of expression evidence in other tissues may be due to the small number of ESTs sequenced in the libraries from these plant parts. For example the phloem tissue library only contains eight ESTs. Among the plant materials that show evidence of GT expression, callus has the largest number of expressed GTs (408), followed by shoot (395). In leaf tissue, only 188 (24.4%) rice GTs have EST evidence, although the leaf library has the largest number of ESTs (204,353). Low representation of diverse GTs in leaf tissue may be due to relative cell wall homogeneity in leaves compared with other tissues or temporal regulation of GT expression such that many GTs involved cell wall synthesis during early developmental stages are no longer expressed in the mature tissues. Alternatively, the difference may be due to different coverage of the actual total of expressed genes from different libraries, for example if leaf libraries are biased due to very high levels of a few genes, such as those involved in photosynthesis. Among the 282 rice-diverged GTs, 46 (16.3%) and 104 (36.9%) are expressed in leaf and shoot, respectively.

TABLE 2
Distribution of expressed GT gene models
among different EST libraries.

| | | No. of Expressed | No. of Expressed |
|---|---|---|---|
| | | Rice Gene | GT Gene |
| EST Library Source | No. of ESTs | Models | Models |
| Total ESTs | | 33,807 | 628 |
| Callus | 184,189 | 20,401 | 408 |
| Shoot | 139,157 | 20,092 | 395 |
| Mixed Tissues | 99,921 | 17,213 | 371 |
| Panicle | 150,845 | 15,052 | 307 |
| Flower | 51,582 | 13,552 | 277 |
| Root | 79,340 | 11,406 | 241 |
| Seed | 26,407 | 9,996 | 203 |
| Unknown Samples | 53,978 | 10,645 | 199 |
| Pistil | 77,110 | 10,725 | 193 |
| Leaf | 204,353 | 10,750 | 188 |
| Anther | 14,156 | 1,191 | 34 |
| Whole Plant | 64,601 | 2,219 | 21 |

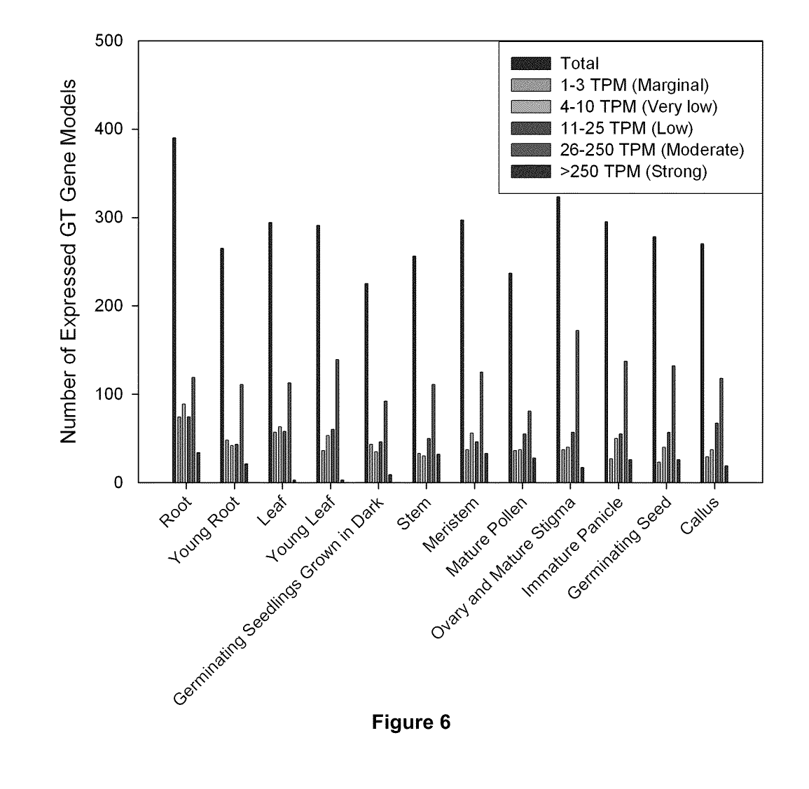

MPSS Analysis. We also extracted information from the Rice MPSS Project (http://mpss.udel.edu/rice/) for each GT gene model. Massively parallel signature sequencing consists of deep, high throughput sequencing of short segments of expressed transcripts and can provide a sensitive, quantitative measure of gene expression for nearly all genes in the genome (Brenner et al., 2000). Data from 18 MPSS libraries representing 12 different tissues/organs of rice was extracted for 17 base pair (bp)-tag signature libraries. MPSS tags were available for 628 (81.7%) GT gene models, providing further evidence that most rice GTs are expressed. As in the EST data, substantial differences were found in abundance of different rice GT gene models in tags per million (TPM), with expression varying from marginal (1-3 TPM) to strong (>250 TPM) expression. The distribution of expressed GT gene models among different MPSS libraries at different expression levels is shown in FIG. 6. A large percentage (30%-50%) of rice GTs exhibited moderate expression (26-250 TPM), while only a few genes were expressed at a high level (>250 TPM).

In general, the EST counts data and MPSS TPM data for GTs are in reasonable agreement, though differences exist. Often MPSS data suggest that a higher percentage of GTs are expressed in a particular tissue in comparison with the EST results. For example, MPSS analysis indicates that the root has the largest number of expressed GT gene models (390, 50.7%), but the EST data only provide evidence for 241 GT gene models (31.2%). Similarly in leaf tissue, MPSS data indicate that there are 294 (38.2%) expressed rice GTs, a larger number GTs than is found in EST leaf-derived libraries (188). The exception is callus tissue, for which MPSS data indicate a lower number of GTs are expressed compared with EST data, 270 (35.1%) versus 408, respectively. There are two technical possibilities to explain this difference. The first is that ESTs from genes with low or specific expression in specific tissues and/or development stages are difficult to detect by lower throughput EST sequencing. Alternatively, marginal and very lowly expressed tags (1-3 and 4-10 TPM) identified by MPSS may represent false positive signals. If GTs with marginal and very low expression are excluded from the MPSS analysis, the number of expressed GTs is similar between EST and MPSS libraries. For example 188 and 174 expressed GTs in leaf tissue based on EST and MPSS, respectively, and 241 and 227 in root, respectively. However this method of raising the threshold for what is expressed by MPSS does not address the root cause of the difference, which can only be addressed by even deeper expression analysis through sequencing.

Microarray Analysis. In addition to sequence based expression analysis methods, we also used publicly available data from rice microarrays, which rely on hybridization of transcript-derived sequences to arrayed DNA oligonucleotides (oligos). Microarrays allow biologists to measure the amounts of individual transcripts for tens of thousands of genes simultaneously, thus providing a high-throughput tool for analyzing gene expression at the whole genome level. Four platforms of rice whole genome oligo arrays have been developed and several hundred datasets from them are available in the public microarray database, NCBI Gene Expression Omnibus (GEO, http://www.ncbi.nlm.nih.gov/geo/). Depending on the array design, these array data are applicable for analyzing expression of subsets of genes of interest. Microarray data from 430 hybridizations, including those from the rice Affymetrix (148), NSF 20K (114) and BGI/Yale platforms (168), are available in a phylogenetic context in the Rice GT Database.

Here, for comparison with the EST and MPSS data sets, we used the rice Affymetrix microarray data of Jain et al. to profile expression patterns for different tissues and developmental stages (Jain et al., 2007). The rice tissues and developmental stages included in this dataset include seedling, root, mature leaf, young leaf, shoot apical meristem (SAM), and various stages of panicle (P1-P6) and seed (S1-55) development (Jain et al., 2007). Following whole-chip data processing, we extracted and averaged the log₂ signal values for the 634 rice GTs represented on the array. These data are represented as a heatmap in the context of the rice GT phylogenetic tree. Among the 634 rice GTs represented by the Affymetrix microarray, 618 (97.5%) have a log₂ signal value >5 (corresponding spot intensity is 32) in at least one of the rice tissues and developmental stages analyzed, indicating again that most of rice GTs are expressed. The expression levels were different among GT families and GTs within same family. For example, most members in GT75 are rice-diverged in all tissues and developmental stages, while low expression was detected in almost all members in GT1.

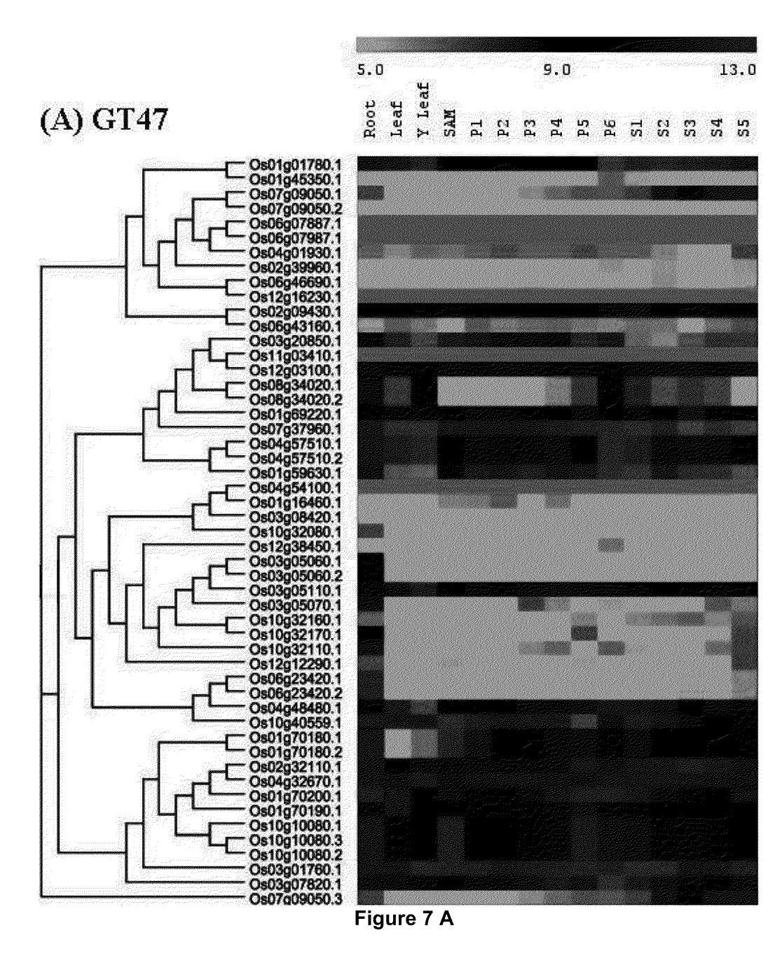

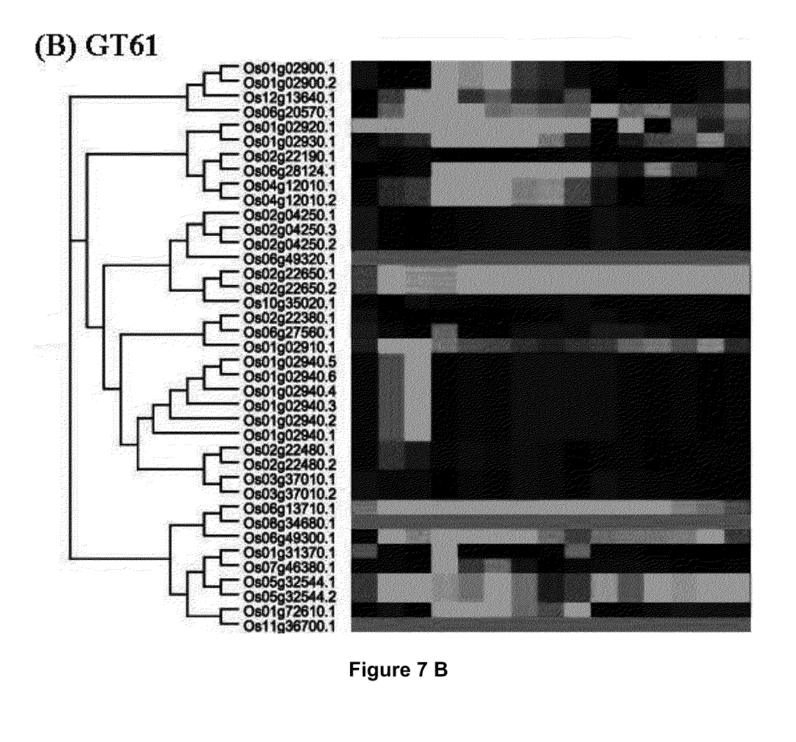

Gene expression of the rice genes in the GT47 and GT61 families are shown in FIG. 7. Mitchell et al. identified genes in the GT47 (13-glucuronyltransferase and heparan synthase) and GT61 (xylosyltransferase) families as likely candidates for involvement in glucuronoarabinxylan synthesis (Mitchell et al., 2007). Due to the high levels of this polysaccharide in rice primary cell walls, we expect these GTs to be rice-diverged in rice. From FIG. 7 it is clear that most of GT47 members are lowly expressed, with only a cluster of nine gene models (6 loci) with high expression. These six loci were also identified by Mitchell et al. as having high expression in monocots relative to dicots; whereas, that study found that other GT47 genes with low expression in rice were found to have similar expression levels between grasses and dicots (Mitchell et al., 2007). Agreement between this study provides mutual support for the potential importance of the group of genes in type II cell wall synthesis, and for the complementary methods used to identify this gene family. Furthermore, among the six rice-diverged GT47 loci, five were identified to be rice-diverged GTs, while 25 out of the other 42 low expressed members have orthologs in dicots. This further supports that only the rice-diverged GT47 genes might be the candidates for the arabinoxylan biosynthesis.

In contrast to the expression of GT47 family members, most GT61 gene models are rice-diverged in at least one tissue or developmental stage. All GT61 members were found to have higher expression in grasses compared to dicots in the Mitchell et al. study. These observations indicate that most GT61 members should be candidates for the glucuronoarabinoxylan biosynthesis. In this family, GTs with similar gene expression patterns appear to cluster together within the phylogenetic tree, suggesting that gene redundancy may be a barrier to functional studies. Simultaneous silencing of multiple genes in this family may be required for loss of function analyses (Miki et al., 2005). Thus, the availability of a large amount of microarray data and other gene expression data in the Rice GT Database, combined with the phylogenetic tree, provides a powerful tool to study the rice GTs expression patterns and functions.

Identification of Rice-Diverged GTs with High Expression in Above Ground Tissues. Most plant biomass under consideration for lignocellulosic biofuel production consists of vegetative, above-ground tissues, such as leaves, stems, shoots, and the progenitor of these tissues, the shoot apical meristem. Thus, identification of rice-diverged GTs with high expression in vegetative, above-ground tissues and elucidation their function is likely to assist effort to alter the composition of lignocellulosic biomass from grasses. To identify potential grass-diverged genes that show consistent expression in above ground tissues, we identified rice-diverged genes that show moderate to high gene expression in at least two of the three gene expression datasets previously described, i.e., rice EST, MPSS, and microarrays. The datasets examined consist of leaf and shoot EST libraries; young leaf, leaf, shoot and meristem MPSS libraries; and young leaf, mature leaf, seedling, shoot and meristem hybridizations from the Affymetrix microarray data. For each type of evidence, we selected rice-diverged GTs in the top 25% most rice-diverged genes in at least one vegetative, above ground tissue. The 25% criteria were chosen to represent moderately to rice-diverged genes as almost half of all annotated rice gene models are not represented by ESTs (Jung et al., 2008b). Although somewhat arbitrary, genes in the top 25% for two data types are all rice-diverged. If the list we have generated proves to be valuable for identifying genes centrally involved in above ground cell wall synthesis, relaxing these criteria may continue to allow us to identify good targets for study.

As listed in Table 3, this analysis identified 33 GT loci, representing 45 gene models, as rice-diverged and rice-diverged. GTs from 14 families are represented. Thirty-six of the gene models (80%) have FL-cDNA support and all of the others have EST support. A number of GTs on this list are not expected to have direct roles in cell wall synthesis, including GT1s, which glycosylate small molecule acceptors; GT4s, which include sucrose synthases; GT20s, which act on trehalose; and the GT29 and GT31 genes, which have not been characterized in plants though similar enzymes in animals are involved in protein glycosylation. All of the remaining GTs are from families that have either been shown to be involved in cell wall synthesis, including the members of the GT2, GT8, GT37, GT43, GT48, and GT77 families, or have been implicated or hypothesized to be involved in cell wall synthesis, including the members of the GT47, GT61, and GT75 families. References that elucidate the connections or putative connections of these proteins with cell wall synthesis are provided. Of particular relevance to type II cell wall synthesis, the list includes two members of the Cs1F gene family (GT2), which is involved in mixed linkage glucan synthesis (Burton et al., 2006). Furthermore, the families of a number of listed genes have been connected with xylan synthesis, including the members of the GT8, GT43, GT47, GT61 families. The GT77 family has been shown to be involved with accumulation of arabinan in type I walls (Egelund et al., 2007), but it may also be relevant to glucuronoarabinan synthesis in type II cell walls. In summary, we expect a number of the rice-divereged, rice-diverged GTs to play important roles in the synthesis of Type II specific cell wall in vegetative, above-ground tissues, distinguishing these genes among the hundreds of rice GTs as prime targets for functional studies in grasses.

TABLE 3
List of rice-diverged GTs with high expression in vegetative, above ground tissues.

| | CAZy | | |
|---|---|---|---|
| TIGR ID | Family | TIGR Annotation | Cell Wall Reference |
| Os01g53350 | GT1 | anthocyanidin 5,3-O-glucosyltransferase, putative, | |
| | | expressed | |
| Os02g11110 | GT1 | flavonol-3-O-glycoside-7-O-glucosyltransferase 1, | |
| | | putative, expressed | |
| Os02g11640 | GT1 | flavonol-3-O-glycoside-7-O-glucosyltransferase 1, | |
| | | putative, expressed | |
| Os02g28900 | GT1 | cytokinin-O-glucosyltransferase 2, putative, expressed | |
| Os04g25440 | GT1 | cytokinin-O-glucosyltransferase 2, putative, expressed | |
| Os11g04860 | GT1 | indole-3-acetate beta-glucosyltransferase, putative, | |
| | | expressed | |
| Os02g49332 | GT2 | CslE2 - cellulose synthase-like family E, expressed | (Hazen et al., 2002) |
| Os07g36630 | GT2 | CslF8 - cellulose synthase-like family F; beta1,3; 1,4 | (Burton et al., 2006) |
| | | glucan synthase, expressed | |
| Os08g06380 | GT2 | CslF6 - cellulose synthase-like family F; beta1,3; 1,4 | (Burton et al., 2006) |
| | | glucan synthase, expressed | |
| Os02g51060 | GT2 | CslA6 - cellulose synthase-like family A; mannan | (Liepman et al., 2007) |
| | | synthase, expressed | |
| Os03g15840 | GT4 | glycosyl transferase, group 1 family protein, putative, | |
| | | expressed | |
| Os03g16140 | GT4 | digalactosyldiacylglycerol synthase 2, putative, | |
| | | expressed | |
| Os11g05990 | GT4 | digalactosyldiacylglycerol synthase 1, putative, | |
| | | expressed | |
| Os03g11330 | GT8 | transferase, transferring glycosyl groups, putative, | (Lee et al., 2007b) |
| | | expressed | |
| Os05g35200 | GT8 | secondary cell wall-related glycosyltransferase family 8, | (Lee et al., 2007b) |
| | | putative, expressed | |
| Os06g12280 | GT8 | glycosyl transferase family 8 protein, expressed | (Lee et al., 2007b) |
| Os02g54820 | GT20 | trehalose-6-phosphate synthase, putative, expressed | |
| Os05g44210 | GT20 | alpha,alpha-trehalose-phosphate synthase, putative, | |
| | | expressed | |
| Os08g34580 | GT20 | trehalose-6-phosphate synthase, putative, expressed | |
| Os12g05550 | GT29 | sialyltransferase-like protein, putative, expressed | |
| Os08g02370 | GT31 | transferase, transferring glycosyl groups, putative, | |
| | | expressed | |
| Os02g52560 | GT37 | galactoside 2-alpha-L-fucosyltransferase, putative, | (Perrin et al., 1999) |
| | | expressed | |
| Os07g49370 | GT43 | beta3-glucuronyltransferase, putative, expressed | (Lee et al., 2007a; |
| | | | Pena et al., 2007) |
| Os01g70190 | GT47 | secondary cell wall-related glycosyltransferase family | (Mitchell et al., 2007) |
| | | 47, putative, expressed | |
| Os04g57510 | GT47 | exostosin-like, putative, expressed | (Mitchell et al., 2007) |
| Os02g58560 | GT48 | CALS1, putative, expressed | (Hong et al., 2001) |
| Os06g02260 | GT48 | callose synthase catalytic subunit, putative, expressed | (Hong et al., 2001) |
| Os02g22380 | GT61 | glycosyltransferase, putative, expressed | (Mitchell et al., 2007) |
| Os06g27560 | GT61 | HGA4, putative, expressed | (Mitchell et al., 2007) |
| Os06g28124 | GT61 | glycosyltransferase, putative, expressed | (Mitchell et al., 2007) |
| Os03g40270 | GT75 | alpha-1,4-glucan-protein synthase, putative, expressed | (Drakakaki et al., 2006) |
| Os03g63270 | GT77 | regulatory protein, putative, expressed | (Egelund et al., 2007; |
| | | | Egelund et al., 2006) |
| Os07g19444 | GT77 | regulatory protein, putative, expressed | (Egelund et al., 2007; |
| | | | Egelund et al., 2006) |

Mutant Line Resources to Study Functions of GTs. Gene indexed mutant rice plants interrupting or activating expression of GTs may in many cases serve as useful resources for determining the gene functions. Several approaches have been undertaken to develop rice mutant lines in which genes are randomly tagged by DNA insertion elements, such as the Tos17 retrotransposition and T-DNA insertion (An et al., 2003; Miyao et al., 2003). For rice GTs, we gathered mutant line information from available mutant line libraries (Table 4). Among these mutant libraries, NIAS Tos17, OTL Tos17 and T-DNA, and RMD T-DNA mutant lines have phenotype information available in their database website, and the hyperlinks to these phenotypes are also available in the Rice GT Database. Information about phenotypes in GTs gene indexed mutant lines, available from above public databases, suggest candidate GT genes associated with rice cell wall biosynthesis. For example, the rice GT Os01g54620.1 (CESA4, a expressed cellulose synthase gene) in GT2 has a Tos17 knockout line NE1042 in the NIAS library, showing a brittle, withering and dwarf phenotype. The homozygous mutant plant progeny of AT4G18780.1 (CESA8), the Arabidopsis ortholog of this rice GT, were severely dwarfed and sterile (Persson et al., 2007). The leaves were dark green, indicating an increase in chloroplasts per leaf area of the mutants, which was probably due to the reduced cell size (Persson et al., 2007). These phenotypes suggest a role for the rice GT, Os01g54620.1, in cell wall biosynthesis. Thus the availability of mutant lines and the corresponding phenotype information for some of these lines in our database will be helpful for the further functional approaches of rice GTs.

TABLE 4
Summary information for rice GT mutant lines.

| | No. of GTs with | No. of |
|---|---|---|
| Mutant Library | Mutant Lines | Mutant Lines |
| NIAS Tos17* | 75 | 276 |
| OTL Tos17* | 71 | 190 |
| UCD Ds | 67 | 124 |
| RMD T-DNA* | 157 | 196 |
| TRIM T-DNA | 108 | 118 |
| OTL T-DNA* | 141 | 122 |
| Postech T-DNA | 533 | 991 |
| Postech AC | 429 | 954 |

*The phenotypic information of these indicated libraries is available for the mutant lines on the library website. Hyperlinks are also provided in the Rice GT Database

In this study we identified 609 rice GT genes, representing 769 gene models, and created the Rice GT Database to provide a logical format to host and analyze diverse sets of functional genomic information in a phylogenetic context. Rather than analyzing rice GTs one by one, this database allows simultaneous visualization of all the rice GTs families and subfamilies. This format allows for comparison of the features of rice GTs between and within different families. Using this database we identified 33 rice-diverged GT genes with high expression in vegetative, above-ground tissues. We hypothesize that many of these GTs will have important roles in the biosynthesis of grass-specific cell wall components and thus are prime candidates for further functional analysis. We plan to update this database semiannually and add additional features to the database including: links to PubMed citations, protein-protein interaction data from experimental determination or computational prediction, new mutant lines and corresponding phenotype information for both GTs and their interacting proteins, MPSS data from new libraries, and new microarray expression data. We anticipate this database will provide a useful service to the plant cell wall researchers and accelerate biofuel research.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A cell comprising a modified or altered enzymatic activity of a rice-diverged glycosyltransferase (GT).

2. The cell of claim 1 wherein the rice-diverged GT comprises an amino acid sequence that comprises an equal to or more than 80% sequence similarity with an amino acid sequence chosen from the group consisting of SEQ ID NOs:1-269.

3. The cell of claim 2 wherein the rice-diverged GT comprises an amino acid sequence that comprises an equal to or more than 90% sequence similarity with an amino acid sequence chosen from the group consisting of SEQ ID NOs:1-269.

4. The cell of claim 3 wherein the rice-diverged GT comprises an amino acid sequence that comprises an equal to or more than 95% sequence similarity with an amino acid sequence chosen from the group consisting of SEQ ID NOs:1-269.

5. The cell of claim 4 wherein the rice-diverged GT comprises an amino acid sequence that comprises an equal to or more than 99% sequence similarity with an amino acid sequence chosen from the group consisting of SEQ ID NOs:1-269.

6. The cell of claim 2 wherein the rice-diverged GT comprises an amino acid sequence that comprises an equal to or more than 80% sequence similarity with an amino acid sequence chosen from the group consisting of SEQ ID NOs:1-30.

7. The cell of claim 6 wherein the rice-diverged GT comprises an amino acid sequence that comprises an equal to or more than 90% sequence similarity with an amino acid sequence chosen from the group consisting of SEQ ID NOs:1-30.

8. The cell of claim 7 wherein the rice-diverged GT comprises an amino acid sequence that comprises an equal to or more than 95% sequence similarity with an amino acid sequence chosen from the group consisting of SEQ ID NOs:1-30.

9. The cell of claim 8 wherein the rice-diverged GT comprises an amino acid sequence that comprises an equal to or more than 99% sequence similarity with an amino acid sequence chosen from the group consisting of SEQ ID NOs:1-30.

10. The cell of claim 1 wherein the rice-diverged GT is highly expressed in the vegetative above ground plant tissue.

11. The cell of claim 1 wherein the expression of the rice-diverge GT is increased or reduced as compared to a wild-type cell.

12. The cell of claim 11 wherein the expression of the rice-diverge GT is reduced and the cell is knocked-out for the gene encoding the rice-diverge GT.

13. The cell of claim 1 wherein the cell is a plant cell.

14. The cell of claim 13 wherein the plant cell is a monocot plant cell.

15. The cell of claim 1, wherein the rice-diverged GT is involved in synthesis of cellulose or cell wall synthesis.

16. The cell of claim 15, wherein the rice-diverged GT is involved in synthesis of a type I wall-specific or enriched component.

17. The cell of claim 15, wherein the rice-diverged GT is involved in synthesis of a type II wall-specific or enriched component.

18. The cell of claim 15, wherein the rice-diverged GT is involved in synthesis of glucuronoarabinoxylan.

19. A method of identifying or determining a rice-diverged GT of a plant or cell, comprising: (a) providing the amino acid sequence of a monocot gene not known to be rice-diverged GT, and (b) identifying the gene as lacking a dicot ortholog.

20. The method of claim 19 further comprising: determining the expression level of the gene in vegetative above ground plant tissue.

21. The method of claim 20 further comprising: constructing a plant cell that is reduced or increased in the expression of the gene.
